(19) United States
(12) Patent Application Publication
(10) Pub. No.: US 2024/0368668 A1
(43) Pub. Date: Nov. 7, 2024

(54) SUBSTRATES AND BIOMARKERS OF ADAMTS7

(71) Applicant: The Broad Institute, Inc., Cambridge, MA (US)

(72) Inventors: BRYAN MACDONALD, Cambridge, MA (US); Alessandro Arduini, Cambridge, MA (US); Nadine Elowe, Cambridge, MA (US)

(21) Appl. No.: 18/690,382

(22) PCT Filed: Sep. 9, 2022
(86) PCT No.: PCT/US22/76195

§ 371 Date: Mar. 8, 2024

Related U.S. Application Data
(60) Provisional application No. 63/242,809, filed on Sep. 10, 2021.

(51) Int. Cl. C12Q 1/37; G01N 33/68

(52) U.S. Cl. CPC C12Q 1/37; G01N 33/68

(57) ABSTRACT

Provided herein are methods and compositions related to the treatment or prevention of vascular disease and/or heart disease using biomarkers of ADAMTS7 activity and antagonists of ADAMTS7.

### RELATED APPLICATION

This application claims the benefit of U.S. Provisional Application No. 63/242,809, filed on Sep. 10, 2021, the contents of which are fully incorporated by reference herein.

### BACKGROUND

ADAMTS7 (A disintegrin and metalloproteinase with thrombospondin motifs 7) is a large secreted protease with poorly defined substrates. Variants at the ADAMTS7 locus have been identified in population based genome wide association studies (GWAS) for coronary artery disease (CAD), with a risk haplotype including a coding variant in the prodomain, rs3825807 p.Ser214Pro. The ADAMTS7 Ser214 risk variant was shown to increase prodomain processing and maturation, correlating with an increase in COMP (Cartilage oligomeric matrix protein) degradation and vascular smooth muscle cell migration. Additionally, the Ser214 risk allele was associated with an unstable atherosclerotic plaque phenotype and an increase in secondary cardiac events. ADAMTS7 catalytic inhibition has been proposed as a therapeutic strategy for treating CAD. However, no verified endogenous substrates and substrate cleavage sites have been identified which hinders the development of ADAMTS7 activity-based biomarkers. There is currently an unmet need to identify potential substrates and cleavage sites of ADAMTS7 to facilitate development ADAMTS7 activity-based biomarkers and treatment of CAD.

### SUMMARY

The present disclosure provides, in some embodiments, compositions and methods for measuring ADAMTS7 activity. The compositions and methods can be useful for the prevention and/or treatment of a variety of diseases (e.g., coronary artery disease) and for identification of potential antagonists of ADAMTS7.

In certain aspects, provided herein are methods of measuring ADAMTS7 (A disintegrin and metalloproteinase with thrombospondin motifs 7) activity in a subject comprising determining whether a level of cleaved protein in serum of the subject is above a threshold level, wherein a level of the cleaved protein above the threshold level is indicative of elevated ADAMTS7 activity.

For example, pre-cleaved protein is expressed in the vasculature of the subject.

In some embodiments, the cleaved protein is encoded by a gene listed in Table 3. Exemplary cleavage sites of the cleaved protein are listed in Table 3.

In some embodiments, the cleaved protein is cleaved fibulin protein. An exemplary cleaved fibulin protein is cleaved EGF-containing fibulin-like extracellular matrix protein 1 (EFEMP1) (e.g., cleaved at cleavage site 123.124 (ASAA|AVAG) (SEQ ID NO: 1) or cleaved at cleavage site 124.125 (SAAA|VAGP) (SEQ ID NO: 2)).

In certain aspects, provided herein are methods of measuring ADAMTS7 (A disintegrin and metalloproteinase with thrombospondin motifs 7) activity in a subject comprising determining whether a level of auto-cleaved ADAMTS7 in serum of the subject is above a threshold level, wherein a level of the auto-cleaved ADAMTS7 above the threshold level is indicative of elevated ADAMTS7 activity.

For example, pre-cleaved ADAMTS7 is expressed in the vasculature of the subject.

In certain embodiments, the auto-cleaved ADAMTS7 is cleaved at a cleavage site that is at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90% identical to a cleavage site listed in Table 6.

In some embodiments, the auto-cleaved ADAMTS7 is human auto-cleaved ADAMTS7 (e.g., cleaved at cleavage site 1080.1081 (SYGP|SEEP) (SEQ ID NO: 3)).

In certain aspects, provided herein are methods of treating or preventing vascular disease and/or heart disease in a subject, comprising: (a) determining whether serum of the subject comprises a level of a cleaved protein above a threshold level; and (b) if the serum is characterized by a level above the threshold level, administering an antagonist of ADAMTS7 (A disintegrin and metalloproteinase with thrombospondin motifs 7) to the subject.

For example, pre-cleaved protein is expressed in the vasculature of the subject.

In some embodiments, determining whether the level of the cleaved protein is above a threshold level comprises measuring the level of the cleaved protein in the serum.

In some embodiments, the cleaved protein is encoded by a gene listed in Table 3. Exemplary cleavage sites of the cleaved protein are listed in Table 3.

In some embodiments, the cleaved protein is cleaved fibulin protein. An exemplary cleaved fibulin protein is cleaved EGF-containing fibulin-like extracellular matrix protein 1 (EFEMP1) (e.g., cleaved at cleavage site 123.124 (ASAA|AVAG (SEQ ID NO: 1)) or cleaved EFEMP1 protein is cleaved at cleavage site 124.125 (SAAA|VAGP) (SEQ ID NO: 2)).

In certain aspects, provided herein are methods of treating or preventing vascular disease and/or heart disease in a subject, comprising: (a) determining whether serum of the subject comprises a level of auto-cleaved of ADAMTS7 (A disintegrin and metalloproteinase with thrombospondin motifs 7) above a threshold level; and (b) if the serum is characterized by a level above the threshold level, administering an antagonist of ADAMTS7 to the subject.

For example, pre-cleaved ADAMTS7 is expressed in the vasculature of the subject.

In certain embodiments, determining whether the level of the auto-cleaved ADAMTS7 is above a threshold level comprises measuring the level of the auto-cleaved ADAMTS7 in the serum.

In certain embodiments, the auto-cleaved ADAMTS7 is cleaved at a cleavage site that is at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90% identical to a cleavage site listed in Table 6.

In some embodiments, the auto-cleaved ADAMTS7 is human auto-cleaved ADAMTS7 (e.g., cleaved at cleavage site 1080.1081 (SYGP|SEEP) (SEQ ID NO: 3)).

In certain embodiments, the method further comprises conjointly administering an additional cardiovascular therapeutic agent to the subject. In some embodiments, the antagonist of ADAMTS7 enhances the effects of the additional cardiovascular therapeutic agent relative to the additional cardiovascular therapeutic agent alone.

In some embodiments, the heart disease is coronary artery disease.

In certain aspects, provided herein are methods of identifying an antagonist of ADAMTS7 (A disintegrin and metalloproteinase with thrombospondin motifs 7), comprising: (a) contacting a cell sample with a test agent; (b) measuring a level of a cleaved protein of the cell sample; and (c) identifying the test agent as an antagonist of ADAMTS7 if the level of the cleaved protein is decreased as compared to a level of the cleaved protein of a cell sample not contacted with the test agent.

In some embodiments, the cleaved protein level of the cell sample not contacted with the test agent is the cleaved protein level in the cell sample prior to contact with the test agent.

In certain embodiments, the cleaved protein level of the cell sample not contacted with the test agent is the cleaved protein level of a corresponding control cell sample.

In some embodiments, the cleaved protein is encoded by a gene listed in Table 3. Exemplary cleavage sites of the cleaved protein are listed in Table 3.

In some embodiments, the cleaved protein is cleaved fibulin protein. An exemplary cleaved fibulin protein is cleaved EGF-containing fibulin-like extracellular matrix protein 1 (EFEMP1) (e.g., cleaved at cleavage site 123.124 (ASAA|AVAG) (SEQ ID NO: 1) or cleaved at cleavage site 124.125 (SAAA|VAGP) (SEQ ID NO: 2)).

In certain aspects, provided herein are methods of identifying an antagonist of ADAMTS7 (A disintegrin and metalloproteinase with thrombospondin motifs 7)), comprising: (a) contacting a cell sample with a test agent; (b) measuring a level of auto-cleaved ADAMTS7 of the cell sample; and (c) identifying the test agent as an antagonist of ADAMTS7 if the level of the auto-cleaved ADAMTS7 is decreased as compared to a level of the auto-cleaved ADAMTS7 is of a cell sample not contacted with the test agent.

In some embodiments, the auto-cleaved ADAMTS7 level of the cell sample not contacted with the test agent is the auto-cleaved ADAMTS7 level in the cell sample prior to contact with the test agent.

In certain embodiments, the auto-cleaved ADAMTS7 level of the cell sample not contacted with the test agent is the auto-cleaved ADAMTS7 level of a corresponding control cell sample.

In certain embodiments, the auto-cleaved ADAMTS7 is cleaved at a cleavage site that is at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90% identical to a cleavage site listed in Table 6.

In some embodiments, the auto-cleaved ADAMTS7 is human auto-cleaved ADAMTS7 (e.g., cleaved at cleavage site 1080.1081 (SYGP|SEEP) (SEQ ID NO: 3)).

### BRIEF DESCRIPTION OF THE DRAWINGS

FIG. 1 shows ADAMTS7 substrate identification through terminal amine isotopic labeling of substrates (TAILS) proteomics study overview. Sample processing for TMT10 TAILS proteomics to identify neo-N-termini from the active ADAMTS7 enzyme condition. Quantitation of the isobaric tag spectra allows for comparative ratios between the WT active enzyme and the EQ catalytic mutant control or the non-specific Luciferase control. Candidate peptide cleavage sites significantly regulated for both WT/EQ and WT/Luc comparisons represent high confidence ADAMTS7 cleavage sites from each TAILS experiment. Independent TAILS experiments from endothelial and smooth muscle cells identified similar cleavage site preferences and many of the same substrate cleavage sites representing potential biomarkers for ADAMTS7 activity.

FIG. 2A shows exemplary volcano plots of regulated TAILS peptides and secretome regulated proteins. Panels A shows comparison of WT/EQ, WT/Luc and EQ/Luc regulated peptides from one of three independent TAILS experiments: TAILS SMC1 (p<0.01). Mouse ADAMTS7 total protein (red diamonds) was significantly upregulated in the WT/Luc and EQ/Luc comparisons.

FIG. 2B shows a comparison of WT/EQ, WT/Luc and EQ/Luc regulated peptides from one of three independent TAILS experiments: TAILS SMC2 (p<0.05). Mouse ADAMTS7 total protein (red diamonds) was significantly upregulated in the WT/Luc and EQ/Luc comparisons.

FIG. 2C shows a comparison of WT/EQ, WT/Luc and EQ/Luc regulated peptides from one of three independent TAILS experiments: TAILS HUVEC (p<0.05). Mouse ADAMTS7 total protein (red diamonds) was significantly upregulated in the WT/Luc and EQ/Luc comparisons.

FIG. 2D shows a comparison of WT/EQ, WT/Luc and EQ/Luc regulated proteins from the total secretome analysis: TAILS SMC1 (p<0.01). Mouse ADAMTS7 total protein (red diamonds) was significantly upregulated in the WT/Luc and EQ/Luc comparisons.

FIG. 2E shows a comparison of WT/EQ, WT/Luc and EQ/Luc regulated proteins from the total secretome analysis: TAILS SMC2 (p<0.05). Mouse ADAMTS7 total protein (red diamonds) was significantly upregulated in the WT/Luc and EQ/Luc comparisons.

FIG. 2F shows a comparison of WT/EQ, WT/Luc and EQ/Luc regulated proteins from the total secretome analysis. TAILS HUVEC (p<0.05). Mouse ADAMTS7 total protein (red diamonds) was significantly upregulated in the WT/Luc and EQ/Luc comparisons.

FIG. 3A shows exemplary ADAMTS7 auto-cleavage sites detected in TAILS experiments. Panel A, ADAMTS7 protein domains and locations of the TAILS significantly regulated WT/EQ peptides indicating auto-cleavage events. Abbreviated ADAMTS7 domains: signal peptide (SP), disintegrin (Dis), thrombospondin repeats (T), cysteine-rich (Cys-rich), protease and lacunin (PL). FIG. 3A discloses SEQ ID NO: 4.

FIG. 3B shows Panel B, ADAMTS7 auto-cleavage peptide total intensities pooled from all TAILS experiments to show relative abundance of each event.

FIG. 3C shows cleavage sites were analyzed using iceLogo to generate logos adjusted for the abundance of each amino acid in humans for ADAMTS7 autocleavage sites: Panel C, SMC1 auto iceLogo (p<0.01, +FC>1, n=41).

FIG. 3D shows cleavage sites were analyzed using iceLogo to generate logos adjusted for the abundance of each amino acid in humans for ADAMTS7 autocleavage sites: Panel D, SMC2 auto iceLogo (p<0.05, +FC>1, n=43).

FIG. 3E shows cleavage sites were analyzed using iceLogo to generate logos adjusted for the abundance of each amino acid in humans for ADAMTS7 autocleavage sites: Panel E. HUVEC auto iceLogo (p<0.05, +FC>1, n=34).

FIG. 3F shows cleavage sites were analyzed using iceLogo to generate logos adjusted for the abundance of each amino acid in humans for ADAMTS7 autocleavage sites: Panel F, all unique sties ADAMTS7 auto-cleavage iceLogo (n=75).

FIG. 4A shows exemplary volcano plots of TAILS regulated peptides visualizing the high confidence cleavage sites. Panels Ashows a comparison of WT/EQ and WT/Luc regulated peptides from three independent TAILS experiments after removal of mouse ADAMTS7 peptides: TAILS SMC1 (p<0.01). Regulated peptides meeting all criterial for the high confidence candidate cleavage sites are shown in green for each TAILS experiment.

FIG. 4B shows a comparison of WT/EQ and WT/Luc regulated peptides from one of three independent TAILS experiments after removal of mouse ADAMTS7 peptides: TAILS SMC2 (p<0.05). Regulated peptides meeting all criterial for the high confidence candidate cleavage sites are shown in green for each TAILS experiment.

FIG. 4C shows a comparison of WT/EQ and WT/Luc regulated peptides from one of three independent TAILS experiments after removal of mouse ADAMTS7 peptides: TAILS HUVEC (p<0.05). Regulated peptides meeting all criterial for the high confidence candidate cleavage sites are shown in green for each TAILS experiment.

FIG. 5A shows exemplary ADAMTS7 TAILS high confidence cleavage sites from one of three independent experiments (SMC1). Histograms showing the overlap between significantly regulated candidate cleavage sites from the SMC1 (Panel A), SMC2 (Panel C) and HUVEC (Panel E) TAILS experiments.

FIG. 5B shows candidate cleavage sites (SMC1) present in both the WT/EQ and WT/Luc comparisons were consistently associated with ADAMTS7 activity and are defined as high confidence cleavage sites. Analysis of the cleavage sites using iceLogo shows the similarities between independent TAILS experiments for SMC1 (Panel B), SMC2 (Panel D) and HUVEC (Panel F).

FIG. 5C shows exemplary ADAMTS7 TAILS high confidence cleavage sites from one of three independent experiments (SMC2). Histograms showing the overlap between significantly regulated candidate cleavage sites from the SMC1 (Panel A), SMC2 (Panel C) and HUVEC (Panel E) TAILS experiments.

FIG. 5D shows candidate cleavage sites (SMC2) present in both the WT/EQ and WT/Luc comparisons were consistently associated with ADAMTS7 activity and are defined as high confidence cleavage sites. Analysis of the cleavage sites using iceLogo shows the similarities between independent TAILS experiments for SMC1 (Panel B), SMC2 (Panel D) and HUVEC (Panel F).

FIG. 5E shows exemplary ADAMTS7 TAILS high confidence cleavage sites from one of three independent experiments (HUVEC). Histograms showing the overlap between significantly regulated candidate cleavage sites from the SMC1 (Panel A), SMC2 (Panel C) and HUVEC (Panel E) TAILS experiments.

FIG. 5F shows candidate cleavage sites (HUVEC) present in both the WT/EQ and WT/Luc comparisons were consistently associated with ADAMTS7 activity and are defined as high confidence cleavage sites. Analysis of the cleavage sites using iceLogo shows the similarities between independent TAILS experiments for SMC1 (Panel B), SMC2 (Panel D) and HUVEC (Panel F).

FIG. 6A shows TAILS high confidence substrate site specificity compared with ADAMTS7 auto-cleavage sites. TAILS candidate substrate cleavage sites and ADAMTS7 auto-cleavage sites analyzed using iceLogo to generate heatmaps adjusted for the abundance of each amino acid in humans. Panel A, SMC1 candidate heatmap (p<0.01, +FC>1, n=179).

FIG. 6B shows Panel B, SMC2 candidate heatmap (p<0.05, +FC>1, n=200),

FIG. 6C shows Panel C, HUVEC candidate heatmap (p<0.05, +FC>1, n=118).

FIG. 6D shows Panel D, the heat map including all unique ADAMTS7 auto-cleavage sites (n=75) resembles the TAILS high confidence substrates.

FIG. 6E shows an Amino acid frequency plot generated by WebLogo showing the similar distribution between experiments, however no amino acid was present more than 30% at any given position at the cleavage site.

FIG. 6F shows an Amino acid frequency plot generated by WebLogo showing the similar distribution between experiments, however no amino acid was present more than 30% at any given position at the cleavage site.

FIG. 6G shows an Amino acid frequency plot generated by WebLogo showing the similar distribution between experiments, however no amino acid was present more than 30% at any given position at the cleavage site.

FIG. 6H shows an Amino acid frequency plot generated by WebLogo showing the similar distribution between experiments, however no amino acid was present more than 30% at any given position at the cleavage site.

FIG. 7A shows ADAMTS7 TAILS Discovery Set Overlap Analysis. Panel A, Venn diagram showing the overlap of unique candidate cleavage sites from SMC1 and SMC2 high confidence sites.

FIG. 7B shows Panel B, Venn diagram showing the overlap from all SMC and HUVEC TAILS datasets.

FIG. 7C shows Panel C, Gene assignment of the 91 unique candidate cleavage sites identified from multiple TAILS experiments, including 24 unique sites from 16 different genes identified in all three TAILS datasets.

FIG. 8A shows validation of TAILS substrate EFEMP1 and cleavage site preference. Panel A, EFEMP1/Fibulin-3 protein domains, amino acid sequence of the atypical EGF repeat linker and location of the ADAMTS7 cleavage sites. Abbreviated EFEMP1 domains: signal peptide (SP), N-terminal region (N), EGF repeats (E). FIG. 8A discloses SEQ ID NOS 719, 1 and 2, respectively, in order of appearance.

FIG. 8B shows Panel B, concentrated media from HUVEC expressing Ad-Luc, Ad-mWT or Ad-mEQ assessed by western blot under non-reducing conditions. Anti-EFEMP1 antibody recognizes an epitope C-terminal to the ADAMTS7 cleavage sites.

FIG. 8C shows Panel C, quantitation of semi-tryptic or semi-chymotryptic peptides from HUVEC media matching novel cleavage sites from the endogenous EFEMP1 protein. The totalvarea was greater for the 123.124 cleavage site compared to the adjacent 124.125 cleavage site. Additional cleavage events observed were alsovfound in the Luc and EQ controls.

FIG. 8D shows Panel D, in vitro cleavage of HA-EFEMP1 by purified full-length mouse ADAMTS7 S3A assessed by western blot. The antibodies to the N-terminal HA epitope and C-terminal EFEMP1 epitope recognized the EFEMP1 more strongly under non-reducing conditions. A band at 100 kDa under non-reducing conditions is consistent with a purified HA-EFEMP1 dimer, which was also sensitive to ADAMTS7 cleavage.

FIG. 8E shows Panel E, overnight digest of HA-EFEMP1 by mouse ADAMTS7 S3A assessed by Coomassie staining.

FIG. 8F shows Panel F, quantitation of semi-tryptic or semi-chymotryptic peptides from the atypical EGF1 repeat region from HA-EFEMP1 showing a consistent preference for the 123.124 cleavage site.

FIG. 9A shows proteomics sample input, media processing and TAILS workflow. Media collected from human coronary artery smooth muscle cells (SMC) or human umbilical vein endothelial cells (HUVEC) expressing control Luciferase (Luc), active mouse ADAMTS7 (WT) or catalytic mutant mouse ADAMTS7 E373Q (EQ) from three separate experiments. 20 ml of media collected from each 15 cm tissue culture dish. Panel A, SMC1 media was pooled for each condition and split into technical replicates after concentration to generate technical replicates.

FIG. 9B shows Panel B, expression of full-length (FL) ADAMTS7-3xFLAG constructs was verified in the conditioned media (CM) and whole cell lysate (WCL) by direct anti-Flag HRP western blot detection. * indicates mucin domain cleaved degradation product detected by c-terminal Flag tags.

FIG. 9C shows Panel C, Replicates from SMC2 were collected from 3 dishes and processed separately.

FIG. 9D shows Panel D, expression in the media from SMC2 replicates was verified by western blot.

FIG. 9E shows Panel E, Replicates from HUVEC were collected from 2 dishes and processed separately.

FIG. 9F shows Panel F, expression in the media from HUVEC replicates was verified by western blot.

FIG. 9G shows Panel G, media preparation workflow for each replicate to generate input for total secretome and TAILS proteomics experiments.

FIG. 9H shows Panel H, sample processing for TMT10 TAILS proteomics to identify neo-N-termini from the active ADAMTS7 enzyme condition. SMC1 TAILS experiment was digested with Trypsin only. SMC2 and HUVEC were digested with AspN or Trypsin before negative selection.

FIG. 10A shows exemplary TAILS replicate correlation plots and heatmaps for regulated peptides. Similarity of TAILS experimental replicates analyzed by Pearson (linear relationships) and Spearman (monotonic relationships) correlation plots generated by Protigy.

FIG. 10B shows exemplary TAILS replicate correlation plots and heatmaps for regulated peptides. Similarity of TAILS experimental replicates analyzed by Pearson (linear relationships) and Spearman (monotonic relationships) correlation plots generated by Protigy.

FIG. 10C shows exemplary TAILS replicate correlation plots and heatmaps for regulated peptides. Similarity of TAILS experimental replicates analyzed by Pearson (linear relationships) and Spearman (monotonic relationships) correlation plots generated by Protigy.

FIG. 10D shows Heatmap cluster analysis of regulated peptides from TAILS experiments demonstrating greater clustering of EQ and Luc compared to WT replicates associated with ADAMTS7 activity.

FIG. 10E shows Heatmap cluster analysis of regulated peptides from TAILS experiments demonstrating greater clustering of EQ and Luc compared to WT replicates associated with ADAMTS7 activity.

FIG. 10F shows Heatmap cluster analysis of regulated peptides from TAILS experiments demonstrating greater clustering of EQ and Luc compared to WT replicates associated with ADAMTS7 activity.

FIG. 11A shows exemplary secretome replicate correlation plots and heatmaps for regulated proteins. Similarity of secretome experimental replicates analyzed by Pearson (linear relationships) and Spearman (monotonic relationships) correlation plots generated by Protigy.

FIG. 11B shows exemplary secretome replicate correlation plots and heatmaps for regulated proteins. Similarity of secretome experimental replicates analyzed by Pearson (linear relationships) and Spearman (monotonic relationships) correlation plots generated by Protigy.

FIG. 11C shows exemplary secretome replicate correlation plots and heatmaps for regulated proteins. Similarity of secretome experimental replicates analyzed by Pearson (linear relationships) and Spearman (monotonic relationships) correlation plots generated by Protigy.

FIG. 11D shows Heatmap cluster analysis of regulated proteins from secretome experiments demonstrating greater clustering of WT and EQ compared to Luc replicates. The secretome heatmap dendrogram differs from the TAILS heatmap dendrogram and may be a product of Ad-ADAMTS7 expression rather than ADAMTS7 activity.

FIG. 11E shows Heatmap cluster analysis of regulated proteins from secretome experiments demonstrating greater clustering of WT and EQ compared to Luc replicates. The secretome heatmap dendrogram differs from the TAILS heatmap dendrogram and may be a product of Ad-ADAMTS7 expression rather than ADAMTS7 activity.

FIG. 11F shows Heatmap cluster analysis of regulated proteins from secretome experiments demonstrating greater clustering of WT and EQ compared to Luc replicates. The secretome heatmap dendrogram differs from the TAILS heatmap dendrogram and may be a product of Ad-ADAMTS7 expression rather than ADAMTS7 activity.

FIG. 12A shows analysis of overlapping regulated proteins from each secretome experiment. Comparison of regulated proteins within each ADAMTS7 secretome experiment. Significantly upregulated proteins (log FC>1) and down-regulated proteins (log FC<−1) shown in red. List of proteins regulated in EQ/Luc alone are not shown. A. SMC1 Venn diagram.

FIG. 12B shows analysis of overlapping regulated proteins from each secretome experiment. Comparison of regulated proteins within each ADAMTS7 secretome experiment. Significantly upregulated proteins (log FC>1) and down-regulated proteins (log FC<−1) shown in red. List of proteins regulated in EQ/Luc alone are not shown. B. SMC2 Venn diagram.

FIG. 12C shows analysis of overlapping regulated proteins from each secretome experiment. Comparison of regulated proteins within each ADAMTS7 secretome experiment. Significantly upregulated proteins (log FC>1) and down-regulated proteins (log FC<−1) shown in red. List of proteins regulated in EQ/Luc alone are not shown. C. HUVEC Venn diagram.

FIG. 13 shows candidate assessment from SMC2 WT/EQ and SMC2 WT/Luc TAILS comparisons after removal of auto-cleavage sites. Detailed breakdown of candidates from volcano plots shown in FIG. 4, Panel B to illustrate the 210 high confidence substrate cleavage sites and remaining unqualified regulated peptides. Panel from FIG. 5, Panel C is included to show the distribution of SMC2 regulated peptides. A majority of WT/EQ regulated peptides met all the criteria for high confidence substrate cleavage sites, while roughly half of the WT/Luc regulated peptides were categorized as high confidence. For simplicity numbers reflect unique substrate cleavage sites, however the volcano plots contain all regulated peptides including multiple identifications for ADAM9_69 and COL1A2_113 within the SMC2 TAILS dataset.

FIG. 14A shows exemplary ADAMTS7 cleavage site specificity from TAILS experiments. Stringent cleavage site logo plots generated by WebLogo and amino acid counts for the TAILS high confidence candidate substrate cleavage sites and ADAMTS7 auto-cleavage sites. Panel A, SMC1 candidates (p<0.01, +FC>1, n=179).

FIG. 14B shows exemplary ADAMTS7 cleavage site specificity from TAILS experiments. Stringent cleavage site logo plots generated by WebLogo and amino acid counts for the TAILS high confidence candidate substrate cleavage sites and ADAMTS7 auto-cleavage sites. Panel B, SMC1 candidates (p<0.01, +FC>1, n=179).

FIG. 14C shows exemplary ADAMTS7 cleavage site specificity from TAILS experiments. Stringent cleavage site logo plots generated by WebLogo and amino acid counts for the TAILS high confidence candidate substrate cleavage sites and ADAMTS7 auto-cleavage sites. Panel C, HUVEC candidates (p<0.05, +FC>1, n=118).

FIG. 14D shows exemplary ADAMTS7 cleavage site specificity from TAILS experiments. Stringent cleavage site logo plots generated by WebLogo and amino acid counts for the TAILS high confidence candidate substrate cleavage sites and ADAMTS7 auto-cleavage sites. Panel D, all unique ADAMTS7 auto-cleavage sites (n=75).

FIG. 15A shows exemplary purified HA-EFEMP1 in vitro cleavage and background cleavage. Quantitation of semi-tryptic or semi-chymotryptic HA-EFEMP1 peptides from the ADAMTS7 in vitro cleavage assay. A subset of this data at the atypical EGF1 repeat was presented in FIG. 7. ADAMTS7 specific cleavage sites at 123.124 and 124.125 are indicated by green arrows.

FIG. 15B shows background cleavage in purified HA-EFEMP1 within the atypical EGF1 repeat in the absence of enzyme shown by western blot under non-reducing conditions. A region below the full-length HA-EFEMP1 was excised from a parallel Coomassie stained non-reducing gel and sent off for LC-MS/MS identification of EFEMP1 peptides.

FIG. 15C shows the quantitation of semi-tryptic or semi-chymotryptic peptides showing background cleavage from the commercial purified HA-EFEMP1 protein. Background cleavage at positions 123.124 and 124.125 were detected and did not increase in abundance after overnight digestion in control conditions.

### DETAILED DESCRIPTION

### General

In certain aspects, the methods and compositions provided herein are based, in part, on the discovery that certain cleaved substrates in serum of a subject can be used as biomarkers of vascular disease and/or heart disease (e.g., coronary artery disease). Provided herein are methods of measuring ADAMTS7 activity in a subject by determining levels of certain biomarkers above a threshold level in serum of the subject. Exemplary biomarkers include cleaved substrates of ADAMTS7 (e.g., cleaved fibulin proteins (e.g., cleaved EFEMP1)) and/or auto-cleaved ADAMTS7. Also provided herein are methods of treating or preventing vascular disease and/or heart disease (e.g., coronary artery disease) in a subject by determining a level of a biomarker in serum of the subject (e.g., cleaved substrates of ADAMTS7 (e.g., cleaved fibulin proteins (e.g., cleaved EFEMP1)) or auto-cleaved ADAMTS7) and administering an antagonist of ADAMTS7 if the level of the biomarker is above a threshold level. Exemplary antagonists of ADAMTS7 include X. In some embodiments, the antagonist of ADAMTS7 is administered conjointly with an additional cardiovascular therapeutic agent as described herein. In certain aspects, provided herein are methods of identifying an antagonist of ADAMTS7 by (a) contacting a cell sample with a test agent, (b) measuring a level of a biomarker (e.g., cleaved substrates of ADAMTS7 (e.g., cleaved fibulin proteins (e.g., EFEMP1)) or auto-cleaved ADAMTS7) in the cell sample, and (c) identifying the test agent as an antagonist of ADAMTS7 if the level of the biomarker is decreased as compared to a level of the biomarker of a cell sample not contacted with the test agent.

### Definitions

For convenience, certain terms employed in the specification, examples, and appended claims are collected here.

The articles “a” and “an” are used herein to refer to one or to more than one (i.e., to at least one) of the grammatical object of the article. By way of example, “an element” means one element or more than one element.

As used herein, the term “administering” means providing a pharmaceutical agent or composition to a subject, and includes, but is not limited to, administering by a medical professional and self-administering.

The term “agent” refers to any substance, compound (e.g., molecule), supramolecular complex, material, or combination or mixture thereof.

The term “cell sample,” “biological sample,” “tissue sample,” or simply “sample” each refers to a collection of cells. In some embodiments, the cells are obtained from a tissue of a subject. The source of the tissue sample may be solid tissue, as from a fresh, frozen and/or preserved organ, tissue sample, biopsy, or aspirate; blood or any blood constituents, serum, blood; bodily fluids such as cerebral spinal fluid, amniotic fluid, peritoneal fluid or interstitial fluid, urine, saliva, stool, tears; or cells from any time in gestation or development of the subject.

The term “binding” or “interacting” refers to an association, which may be a stable association, between two molecules, due to, for example, electrostatic, hydrophobic, ionic and/or hydrogen-bond interactions under physiological conditions.

The term “measuring” refers to determining the presence, absence, quantity amount, or effective amount of a substance in a sample, including the concentration levels of such substances.

As used herein, the term “subject” means a human or non-human animal selected for treatment or therapy.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

In certain embodiments, therapeutic compounds may be used alone or conjointly administered with another type of therapeutic agent (e.g., cardiovascular therapeutic agent disclosed herein). As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic compounds.

In certain embodiments, conjoint administration of therapeutic compounds with one or more additional therapeutic agent(s) (e.g., one or more additional chemotherapeutic agent(s)) provides improved efficacy relative to each individual administration of the compound (e.g., copper ionophore) or the one or more additional therapeutic agent(s). In certain such embodiments, the conjoint administration provides an additive effect, wherein an additive effect refers to the sum of each of the effects of individual administration of the therapeutic compound and the one or more additional therapeutic agent(s).

The term “ADAMTS-7” (also ADAMTS7, ADAM-TS7, ADAM-TS7) refers to the protein A disintegrin and metalloproteinase with thrombospondin motifs 7. The ADAMTS-7 protein is encoded by the gene ADAMTS-7. The ADAMTS-7 protein comprises human, murine, rat and further mammalian and non-mammalian homologues. Sequence(s) for human ADAMTS-7 are accessible via UniProt Identifier Q9UKP4 (ATS7_HUMAN), for instance human isoform Q9UKP4-1. Sequence(s) for murine ADAMTS-7 are accessible via UniProt Identifier Q68SA9 (ATS7_MOUSE). Different isoforms, variants and SNPs may exist for the different species and are all comprised by the term ADAMTS-7. Also comprised are ADAMTS-7 molecules before and after maturation, i.e., independent of cleavage of one or more pro-domains. In addition, synthetic variants of the ADAMTS-7 protein may be generated and are comprised by the term ADAMTS-7. The protein ADAMTS-7 may furthermore be subject to various modifications, e.g., synthetic or naturally occurring modifications.

The term “ADAMTS-12” (also ADAMTS12, ADAM-TS12, ADAM-TS12) refers to the protein A disintegrin and metalloproteinase with thrombospondin motifs 12. Such proteins preferably include a ADAMTS-12 catalytic domain. The ADAMTS-12 protein is encoded by the gene ADAMTS-12. The ADAMTS-12 protein comprises human, murine, rat and further mammalian and non-mammalian homologues. Sequence(s) for human ADAMTS-12 including the catalytic domains are accessible via UniProt Identifier P58397 (ATS12_HUMAN), for instance human isoform P58397-1. Sequence(s) for murine ADAMTS-12 are accessible via UniProt Identifier Q811B3 (ATS12_MOUSE). Different isoforms and variants may exist for the different species and are all comprised by the term ADAMTS-12. Also comprised are ADAMTS-12 molecules before and after maturation, i.e., independent of cleavage of one or more pro-domains. In addition, synthetic variants of the ADAMTS-12 protein may be generated and are comprised by the term ADAMTS-12. The protein ADAMTS-12 may furthermore be subject to various modifications, e.g., synthetic or naturally occurring modifications.

The terms “ADAMTS-4” and “ADAMTS-5” refer to the protein A disintegrin and metalloproteinase with thrombospondin motifs 4 and 5, respectively. The ADAMTS-4 and -5 proteins are encoded by the genes ADAMTS4 and ADAMTS-5, respectively. These proteins comprises human, murine, rat and further mammalian and non-mamalian homologues. Sequence(s) for human ADAMTS-4/-5 are accessible via UniProt Identifier 075173 (ATS4_HUMAN)/Q9UNA0 (ATS5_HUMAN), respectively. Different isoforms and variants may exist. Recombinant active human ADAMTS-4 and ADAMTS-5 can be manufactured as known in the art.

The terms “MMP2”, “MMP12”, and “MMP15” refer to the 72 kDa type IV collagenase, Macrophage metalloelastase 2 and 12 and Matrix metalloproteinase-15, respectively. The MMP2, MMP12, and MMP15 proteins are encoded by the genes MMP2, MMP12, and MMP15, respectively. The proteins comprises human, murine, rat and further mammalian and non-mamalian homologues. Sequence(s) for human ADAMTS-4/-5 are accessible via UniProt Identifier P08253 (MMP2_HUMAN), P39900 (MMP12_HUMAN) and P51511 (MMP15_HUMAN), respectively. Different isoforms and variants may exist. Recombinant active human ADAMTS-4 and ADAMTS-5 can be manufactured as known in the art.

The term “ADAM17” refers to Disintegrin and metalloproteinase domain-containing protein 17, encoded by the gene ADAM17. The protein comprises human, murine, rat and further mammalian and non-mamalian homologues. Sequence(s) for human ADAM17 are accessible via UniProt Identifier P78536 (ADA17_HUMAN). Different isoforms and variants may exist. Recombinant active human ADAM17 can be manufactured as known in the art.

The term “prodomain” includes parts of ADAMTS-7 or ADAMTS-12 that are relatively N-terminal to the respective protein's functional chain (e.g., parts having metalloprotease function and disintergrin motifs). In some embodiments, prodomain of ADAMTS-7 or ADAMTS-12 includes 75%, 80%, 85%, 90%, 95%, or 100% of the N-terminal part of the respective protein with its signal peptide plus its propeptide. The term “prodomain” also encompasses the parts of the encoded polypeptide that are processed (e.g., cleaved off) before generation of the functional enzymatic chain in the natural environment of the enzyme.

A “furin cleavage site” or furin consensus site is R-x-K/R-R⬇D/S, cf. Shiryaev 2013 PLOS One. The ADAMTS7 prodomain contains multiple Furin protease cleavage sites, the last of which is thought to fully process the zymogen into the active form. Mutational analysis was described by Sommerville 2004 JBC for rat ADAMTS7 with R60A and R217A (referred to as mouse R220A in publication). R60A changes rat ADAMTS7 from LRKR⬇D (SEQ ID NO: 720) to LRKA⬇D (SEQ ID NO: 721) and R217A changes rat ADAMTS7 RQQR⬇S (SEQ ID NO: 722) to RQQA⬇S (SEQ ID NO: 723).

The term “catalytic domain” includes parts of ADAMTS-7 or ADAMTS-12 that have ADAMTS-7 or ADAMTS-12 functionality, respectively, and that are C-terminal to the respective protein's prodomain. In some embodiments, the term “catalytic domain” refers to the peptidase plus disintegrin part of the respective protein (e.g., as characterized by UniProt), potentially also including any residues C-terminal to the respective protein's prodomain and N-terminal to the respective protein's peptidase domain. In some embodiments, the catalytic domain includes 75%, 80%, 85%, 90%, 95%, or 100% of the part of the respective enzyme having its disintegrin domain, its peptidase domain, and any residues it might have between its prodomain and its peptidase domain.

The term “metalloproteinase” refers to a protease enzyme whose catalytic mechanism involves a metal.

The expression “a cleavage site for a protease” refers to any peptide or protein sequence which is recognized and cleaved by the functional protease. A cleavage site for ADAMTS-7 thus refers to any peptide or protein sequence which is recognized and cleaved by functional ADAMTS-7. For example, being natural substrates of ADAMTS-7, the sequences of proteins COMP and TSP1 both comprise cleavage sites for ADAMTS-7. In particular the subsequence DELSSMVLELRGLRT (SEQ ID NO: 724) (derived from TSP1, residues 275-289) constitutes or comprises a cleavage site for ADAMTS-7 and ADAMTS-12.

A “substrate” is a molecule upon which an enzyme acts. For example, the substrate of a proteinase can be a peptide or protein or derivative thereof, which is cleaved by the proteinase.

The term “COMP”, TSP-5 or TSP5 refers to the protein Cartilage oligomeric matrix protein. The COMP protein is encoded by the gene COMP. The COMP protein comprises human, murine, rat and further mammalian and homologues. Sequence(s) for human COMP are accessible via UniProt Identifier P49747 (COMP_HUMAN), for instance human isoform P49747-1. Sequence(s) for murine COMP are accessible via UniProt Identifier Q9R0G6 (COMP_MOUSE). Different isoforms and variants may exist for the different species and are all comprised by the term COMP. Also comprised are COMP molecules before and after maturation, i.e., independent of cleavage of one or more pro-domains. In addition, synthetic variants of the COMP protein may be generated and are comprised by the term COMP. The protein COMP may furthermore be subject to various modifications, e.g, synthetic or naturally occurring modifications. Recombinant human COMP or derivatives thereof can be manufactured.

The term “TSP1” (also THBS1 or TSP) refers to the protein Thrombospondin-1. The TSP1 protein is encoded by the gene THBS1. The TSP1 protein comprises human, murine, rat and further mammalian and non-mammalian homologues. Sequence(s) for human TSP1 are accessible via UniProt Identifier P07996 (TSP1_HUMAN), for instance human isoform P07996-1. Sequence(s) for murine TSP1 are accessible via UniProt Identifier P35441 (TSP1_MOUSE). Different isoforms and variants may exist for the different species and are all comprised by the term TSP1. Also comprised are TSP1 molecules before and after maturation, i.e., independent of cleavage of one or more pro-domains. In addition, synthetic variants of the TSP1 protein may be generated and are comprised by the term TSP1. The protein TSP1 may furthermore be subject to various modifications, e.g, synthetic or naturally occurring modifications. Recombinant human TSP1 or derivatives thereof can be manufactured.

### Biomarkers of ADAMTS7 Activity

ADAMTS7 (A disintegrin and metalloproteinase with thrombospondin motifs 7) belongs to a family of 19 secreted zinc metalloproteinases with a shared organization of a signal peptide, prodomain, metalloproteinase, disintegrin, thrombospondin, cysteine-rich and spacer domains. Additionally, ADAMTS7 has a total of eight thrombospondin type I repeats and a highly glycosylated mucin domain with a chondroitin sulfate glycosaminoglycan (CS-GAG) attachment that set ADAMTS7 and ADAMTS12 apart from their family members. Consequently, the CS-GAG modified ADAMTS7 is both an extracellular protease and a proteoglycan.

Cleaved protein substrates of ADAMTS7 serve as biomarkers of ADAMTS7 activity. In certain aspects, provided herein are methods of determining whether a level of cleaved protein substrate of ADAMTS7 in serum of the subject is above a threshold level, wherein a level of the cleaved protein substrate of ADAMTS7 above the threshold level is indicative of ADAMTS7 activity.

In some embodiments, the cleaved protein substrate is is encoded by a gene listed in Table 3. Exemplary cleavage sites of the cleaved protein substrate are listed in Table 3. In certain embodiments, the cleaved protein substrate is cleaved fibulin protein. An exemplary fibulin proteins includes cleaved fibulin protein is EGF-containing fibulin-like extracellular matrix protein 1 (EFEMP1). In some embodiments, the cleaved EFEMP1 protein is cleaved at cleavage site 123.124 (ASAA|AVAG) (SEQ ID NO: 1). In some embodiments, the cleaved EFEMP1 protein is cleaved at cleavage site 124.125 (SAAA|VAGP) (SEQ ID NO: 2).

In certain embodiments, the cleaved protein substrate is auto-cleaved ADAMTS7.

In certain embodiments, the auto-cleaved ADAMTS7 is cleaved at a cleavage site that is at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90% identical to a cleavage site listed in Table 6.

In some embodiments, the auto-cleaved ADAMTS7 is mouse auto-cleaved ADAMTS7. In some embodiments, the mouse auto-cleaved ADAMTS7 is cleaved at cleavage site 1061.1062 (SYGS|FEEP) (SEQ ID NO: 4). In some embodiments, the auto-cleaved ADAMTS7 is human auto-cleaved ADAMTS7. In some embodiments, the human auto-cleaved ADAMTS7 is cleaved at cleavage site 1080.1081 (SYGP|SEEP) (SEQ ID NO: 3).

In certain embodiments, the threshold level of the biomarker (e.g., cleaved protein substrate of ADAMTS7) in serum of a subject is met if at least 0.5%, 0.6%, 0.7%, 0.8%, 0.9%, 1.0%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% of the serum comprise the biomarker.

In some embodiments, any assay capable of detecting the relevant biomarker (e.g., cleaved protein substrate of ADAMTS7) can be used in the methods provided herein. In some embodiments, the biomarker is detected by isotopic labeling (e.g., TAILS (terminal amine isotopic labeling of substrates)). In some embodiments, the biomarker is detected by immunostaining with a labeled antibody that binds to the biomarker epitope. In some embodiments, the biomarker is detected by immunohistochemistry. In some embodiments, the biomarker is detected by Western Blot. In some embodiments, the mRNAs of the biomarker are detected using qPCR. In some embodiments, the biomarker is detected using fluorescence activated cell sorting (FACS). In some embodiments, the biomarker is detected using microscopy (e.g., fluorescence microscopy). In some embodiments, the biomarker is detected using ELISA.

Any of a variety of antibodies can be used in methods of the detection. Such antibodies include, for example, polyclonal, monoclonal (mAbs), recombinant, humanized or partially humanized, single chain, Fab, and fragments thereof. The antibodies can be of any isotype, e.g., IgM, various IgG isotypes such as IgG1, IgG2a, etc., and they can be from any animal species that produces antibodies, including goat, rabbit, mouse, chicken or the like. The term “an antibody specific for” a protein means that the antibody recognizes a defined sequence of amino acids, or epitope, in the protein, and binds selectively to the protein and not generally to proteins unintended for binding to the antibody. The parameters required to achieve specific binding can be determined routinely, using conventional methods in the art.

In some embodiments, antibodies specific for a biomarker (e.g., cleaved protein substrate of ADAMTS7) are immobilized on a surface (e.g., are reactive elements on an array, such as a microarray, or are on another surface, such as used for surface plasmon resonance (SPR)-based technology, such as Biacore), and proteins in a sample are detected by virtue of their ability to bind specifically to the antibodies. Alternatively, proteins in the sample can be immobilized on a surface, and detected by virtue of their ability to bind specifically to the antibodies. Methods of preparing the surfaces and performing the analyses, including conditions effective for specific binding, are conventional and well-known in the art.

Among the many types of suitable immunoassays are immunohistochemical staining, ELISA, Western blot (immunoblot), immunoprecipitation, radioimmunoassay (RIA), fluorescence-activated cell sorting (FACS), etc. In some embodiments, assays used in methods provided herein can be based on colorimetric readouts, fluorescent readouts, mass spectroscopy, visual inspection, etc.

As mentioned above, a biomarker (e.g., cleaved protein substrate of ADAMTS7) can be measured by measuring nucleic acid amounts (e.g., mRNA amounts and/or genomic DNA). The determination of nucleic acid amounts can be performed by a variety of techniques known to the skilled practitioner. For example, expression levels of nucleic acids, alternative splicing variants, chromosome rearrangement and gene copy numbers can be determined by microarray analysis (see, e.g., U.S. Pat. Nos. 6,913,879, 7,364,848, 7,378,245, 6,893,837 and 6,004,755) and quantitative PCR. Copy number changes may be detected, for example, with the Illumina Infinium II whole genome genotyping assay or Agilent Human Genome CGH Microarray (Steemers et al., 2006). Examples of methods to measure mRNA amounts include reverse transcriptase-polymerase chain reaction (RT-PCR), including real time PCR, microarray analysis, nanostring, Northern blot analysis, differential hybridization, and ribonuclease protection assay. Such methods are well-known in the art and are described in, for example, Sambrook et al., Molecular Cloning: A Laboratory Manual, current edition, Cold Spring Harbor Laboratory, Cold Spring Harbor, N.Y., and Ausubel et al., Current Protocols in Molecular Biology, John Wiley & sons, New York, N. Y.

### Antagonists of ADAMTS7 and Additional Therapeutic Agents

In certain aspects, provided herein are methods of treating vascular disease and/or heart disease in a subject by administering to the subject an antagonist of ADAMTS7 according to a method provided herein. Exemplary antagonists of ADAMTS7 are described in WO 2021/094436 and WO 2021/094434, hereby incorporated by reference in their entirety, and in particular for the ADAMTS7 inhibitors described therein. Antagonists of ADAMTS7 include, but are not limited to, formula (I):

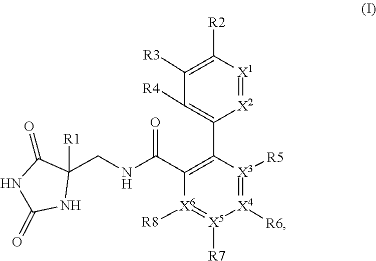

in which
R¹ represents a group selected from hydrogen, (C₁-C₆)-alkyl, (C₃-C₆)-cycloalkyl, 5- to 6-membered heterocycloalkyl, 5- to 10-membered heteroaryl (such as 5- to 6-membered heteroaryl), and phenyl
   wherein said (C₁-C₆)-alkyl, (C₃-C₆)-cycloalkyl, 5- to 6-membered heterocycloalkyl, 5- to 10-membered heteroaryl, and phenyl are optionally substituted with one or two groups independently selected from cyano, halogen, amino, hydroxy, oxo, C₁-C₃-alkyl, (C₁-C₄)-alkoxy, (C₁-C₄)-alkylcarbonyl, mono-(C₁-C₄)-alkylamino, di-(C₁-C₄)-alkylamino, phenyl, (C₁-C₄)-alkylsulfonyl, and (C₃-C₆)-cycloalkyl,
   wherein each said C₁-C₃-alkyl, (C₃-C₆)-cycloalkyl, and (C₁-C₄)-alkoxy is optionally substituted with up to 5 fluorine atoms
R² represents a group independently selected from hydrogen, cyano, halogen, (C₁-C₄)-alkylsulfonyl, (C₁-C₄)-alkyl, (C₃-C₆)-cycloalkyl, and (C₁-C₄)-alkoxy
   wherein said (C₁-C₄)-alkyl, (C₃-C₆)-cycloalkyl, and (C₁-C₄)-alkoxy each is optionally independently substituted with up to five fluorine atoms, cyano, or (C₁-C₂)alkyl, wherein said (C₁-C₂)alkyl is optionally substituted with up to five fluorine atoms
R³, R⁴, R⁵, R⁶, R⁷ and R⁸ represent a group independently selected from hydrogen, halogen, (C₁-C₄)-alkyl and (C₁-C₄)-alkoxy
   wherein said (C₁-C₄)-alkyl and (C₁-C₄)-alkoxy each is optionally independently substituted with up to five fluorine atoms,
with the proviso that at least one of R², R³, R⁴ represents H,
X₁, X₂, X₃, X₄, X₅, X₆, represent N or C with the provisio that in each ring system maximal one X stands for N and R⁵, R⁶, R⁷ and R⁸ are present provided that the designated atom's normal valency under the existing circumstances is not exceeded,
   and pharmaceutically acceptable salts thereof, solvates thereof and the solvates of the salts thereof;
a compound of general formula (I), in which
R¹ represents a group selected from hydrogen, (C₁-C₆)-alkyl, (C₃-C₆)-cycloalkyl, 5- to 6-membered heterocycloalkyl, 5- to 10-membered heteroaryl and phenyl
   wherein said (C₁-C₆)-alkyl, (C₃-C₆)-cycloalkyl, 5- to 6-membered heterocycloalkyl, and 5- to 10-membered heteroaryl, phenyl are optionally substituted with one or two groups independently selected from cyano, halogen, amino, hydroxy, oxo, C₁-C₃-alkyl, (C₁-C₄)-alkoxy, (C₁-C₄)-alkylcarbonyl, mono-(C₁-C₄)-alkylamino, di-(C₁-C₄)-alkylamino, phenyl, (C₁-C₄)-alkylsulfonyl, and (C₃-C₆)-cycloalkyl,
   wherein each said C₁-C₃-alkyl, (C₃-C₆)-cycloalkyl, and (C₁-C₄)-alkoxy is optionally substituted with up to 5 fluorine atoms
R² represents a group independently selected from hydrogen cyano, halogen, (C₁-C₄)-alkylsulfonyl, (C₁-C₄)-alkyl, (C₃-C₆)-cycloalkyl and (C₁-C₄)-alkoxy
   wherein said (C₁-C₄)-alkyl (C₃-C₆)-cycloalkyl and (C₁-C₄)-alkoxy each is optionally independently substituted with up to five fluorine atoms, cyano, or (C₁-C₂)alkyl, wherein said (C₁-C₂)alkyl is optionally substituted with up to five fluorine atoms
R³ and R⁴ represent hydrogen and
R⁵, R⁶, R⁷ and R⁸ represent a group independently selected from hydrogen, halogen, (C₁-C₄)-alkyl and (C₁-C₄)-alkoxy
   wherein said (C₁-C₄)-alkyl and (C₁-C₄)-alkoxy each is optionally independently substituted with up to five fluorine atoms,
X₁, X₂, X₃, X₄, X₅, X₆, represent N or C with the provisio that in each ring system maximal one X stands for N and R⁵, R⁶, R⁷ and R⁸ are present provided that the designated atom's normal valency under the existing circumstances is not exceeded,
   and pharmaceutically acceptable salts thereof, solvates thereof and the solvates of the salts thereof;
a compound of general formula (I), in which R¹ represents a group selected from hydrogen, (C₁-C₆)-alkyl, (C₃-C₆)-cycloalkyl, 5- to 6-membered heterocycloalkyl, 5- to 10-membered heteroaryl and phenyl
   wherein said (C₁-C₆)-alkyl, (C₃-C₆)-cycloalkyl, 5- to 6-membered heterocycloalkyl, and 5- to 10-membered heteroaryl, phenyl are optionally substituted with one or two groups independently selected from cyano, halogen, amino, hydroxy, oxo, C₁-C₃-alkyl, (C₁-C₄)-alkoxy, (C₁-C₄)-alkylcarbonyl, mono-(C₁-C₄)-alkylamino, di-(C₁-C₄)-alkylamino, phenyl, (C₁-C₄)-alkylsulfonyl, and (C₃-C₆)-cycloalkyl,
   wherein each said C₁-C₃-alkyl, (C₃-C₆)-cycloalkyl, and (C₁-C₄)-alkoxy is optionally substituted with up to 5 fluorine atoms
R² represents a group independently selected from hydrogen cyano, halogen, (C₁-C₄)-alkylsulfonyl, (C₁-C₄)-alkyl, (C₃-C₆)-cycloalkyl and (C₁-C₄)-alkoxy
   wherein said (C₁-C₄)-alkyl (C₃-C₆)-cycloalkyl and (C₁-C₄)-alkoxy each is optionally independently substituted with up to five fluorine atoms, cyano, or (C₁-C₂)alkyl, wherein said (C₁-C₂)alkyl is optionally substituted with up to five fluorine atoms.
R³, R⁴, R⁵, R⁶, R⁷ and R⁸ represent a group independently selected from hydrogen, halogen, (C₁-C₄)-alkyl and (C₁-C₄)-alkoxy
   wherein said (C₁-C₄)-alkyl and (C₁-C₄)-alkoxy each is optionally independently substituted with up to five fluorine atoms, with the provision that at least two from R⁵, R⁶, R⁷ and R⁸ represent hydrogen,
X₁, X₂, X₃, X₄, X₅, X₆, represent N or C with the provisio that in each ring system maximal one X stands for N and R⁵, R⁶, R⁷ and R⁸ are present provided that the designated atom's normal valency under the existing circumstances is not exceeded,
   and pharmaceutically acceptable salts thereof, solvates thereof and the solvates of the salts thereof; or a compound of Formula (Ia), (Ib), (Ic), (Id) or (Ie):

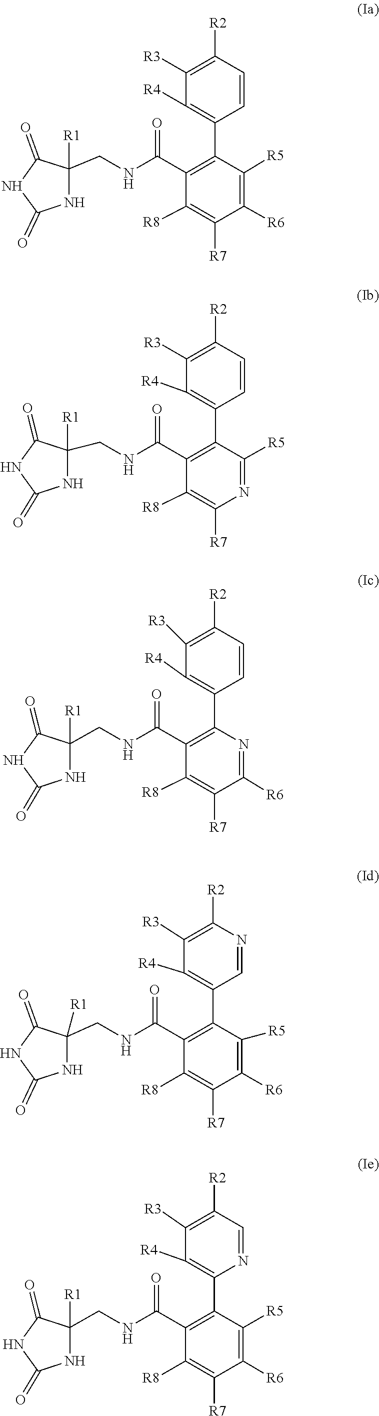

in which
R¹ represents a group selected from hydrogen, (C₁-C₆)-alkyl, (C₃-C₆)-cycloalkyl, 5- to 6-membered heterocycloalkyl, 5- to 10-membered heteroaryl and phenyl
   wherein said (C₁-C₆)-alkyl, (C₃-C₆)-cycloalkyl, 5- to 6-membered heterocycloalkyl, and 5- to 10-membered heteroaryl, phenyl are optionally substituted with one or two groups independently selected from cyano, halogen, amino, hydroxy, oxo, C₁-C₃-alkyl, (C₁-C₄)-alkoxy, (C₁-C₄)-alkylcarbonyl, mono-(C₁-C₄)-alkylamino, di-(C₁-C₄)-alkylamino, phenyl, (C₁-C₄)-alkylsulfonyl, (C₃-C₆)-cycloalkyl,
   wherein each said C₁-C₃-alkyl, (C₃-C₆)-cycloalkyl, and (C₁-C₄)-alkoxy is optionally substituted with up to 5 fluorine atoms
R² represents a group independently selected from hydrogen cyano, halogen, (C₁-C₄)-alkylsulfonyl, (C₁-C₄)-alkyl, (C₃-C₆)-cycloalkyl and (C₁-C₄)-alkoxy
   wherein said (C₁-C₄)-alkyl (C₃-C₆)-cycloalkyl and (C₁-C₄)-alkoxy each is optionally independently substituted with up to five fluorine atoms, cyano, or (C₁-C₂)alkyl, wherein said (C₁-C₂)alkyl is optionally substituted with up to five fluorine atoms.
R³, R⁴, R⁵, R⁶, R⁷ and R⁸ represent a group independently selected from hydrogen, halogen, (C₁-C₄)-alkyl and (C₁-C₄)-alkoxy
   wherein said (C₁-C₄)-alkyl and (C₁-C₄)-alkoxy each is optionally independently substituted with up to five fluorine atoms, with the provision that at least two from R⁵, R⁶, R⁷ and R⁸ represent hydrogen, and pharmaceutically acceptable salts thereof, solvates thereof and the solvates of the salts thereof;
      A compound of formula (I) wherein R² is (C₃-C₆)-cycloalkyl substituted with a trifluormethyl group; or compounds of general Formula (A):

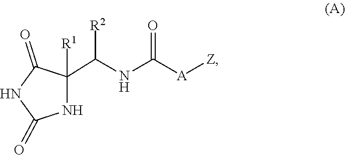

in which
R¹ represents a group selected from hydrogen, (C₁-C₆)-alkyl, (C₃-C₆)-cycloalkyl, 5- to 6-membered heterocycloalkyl, 5- to 6-membered heteroaryl and phenyl,
   wherein said (C₁-C₆)-alkyl, (C₃-C₆)-cycloalkyl, 5- to 6-membered heterocycloalkyl, and 5- to 6-membered heteroaryl, phenyl are optionally substituted with one or two groups independently selected from cyano, halogen, amino, hydroxy, oxo, (C₁-C₃)-alkyl, (C₁-C₄)-alkoxy, (C₁-C₄)-alkylcarbonyl, mono-(C₁-C₄)-alkylamino, di-(C₁-C₄)-alkylamino, (C₁-C₄)-alkylsulfonyl, (C₃-C₆)-cycloalkyl,
   wherein each said (C₁-C₃)-alkyl, (C₃-C₆)-cycloalkyl, and (C₁-C₄)-alkoxy is optionally substituted with up to 5 fluorine atoms,
R² represents a group selected from hydrogen or (C₁-C₄)-alkyl
   wherein said (C₁-C₄)-alkyl is optionally substituted with up to five fluorine atoms,
A represents a group selected from 5-membered heteroaryl
   wherein said 5-membered heteroaryl is optionally substituted with one, two or three groups independently selected from halogen, cyano, hydroxy, methylsulfonyl, (C₃-C₆)-cycloalkyl, (C₁-C₄)-alkyl, and (C₁-C₄)-alkoxy,
   wherein each said (C₃-C₆)-cycloalkyl, (C₁-C₄)-alkyl and (C₁-C₄)-alkoxy is optionally substituted with up to three fluorine atoms,
Z represents a group selected from 6- to 10-membered aryl and 5- to 10-membered heteroaryl,
   wherein said 6- to 10-membered aryl or 5- to 10-membered heteroaryl is optionally substituted with one, two or three groups independently selected from halogen, cyano, hydroxy, (C₁-C₄)-alkylsulfonyl, (C₃-C₆)-cycloalkyl, (C₁-C₄)-alkyl, and (C₁-C₄)-alkoxy,
      wherein each said (C₁-C₄)-alkyl and (C₁-C₄)-alkoxy is optionally substituted with (C₃-C₆)-cycloalkyl and optionally substituted with up to five fluorine atoms,
         and pharmaceutically acceptable salts thereof, solvates thereof and the solvates of the salts thereof; or compounds of general Formula (A) with the proviso that the following compounds (Xa) to (Xi) are excluded

### 4-(4-chlorophenyl)-N-[[4-(1-methylimidazol-2-yl)-2,5-dioxo-imidazolidin-4-yl]methyl]-1H-pyrrole-2-carboxamide

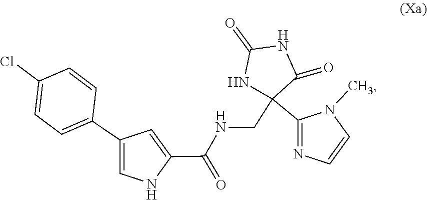 [SMILES: Cn1ccnc1C1(CNC(=O)c2cc(-c3ccc(Cl)cc3)c[nH]2)NC(=O)NC1=O]

### 1-(4-fluorophenyl)-N-[[4-(1-methylimidazol-2-yl)-2,5-dioxo-imidazolidin-4-yl]methyl]pyrazole-4-carboxamide

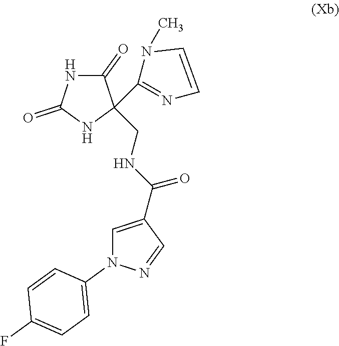 [SMILES: Cn1ccnc1C1(CNC(=O)c2cnn(-c3ccc(F)cc3)c2)NC(=O)NC1=O]

### 1-methyl-N-[4-(1-methylimidazol-2-yl)-2,5-dioxo-imidazolidin-4-yl]methyl]-5-phenyl-pyrrole-2-carboxamide

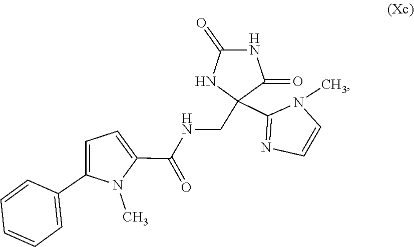 [SMILES: Cn1ccnc1C1(CNC(=O)c2ccc(-c3ccccc3)n2C)NC(=O)NC1=O]

### 4-(5-chloro-2-thienyl)-N-[[4-(1-methylimidazol-2-yl)-2,5-dioxo-imidazolidin-4-yl]methyl]-1H-pyrrole-3-carboxamide

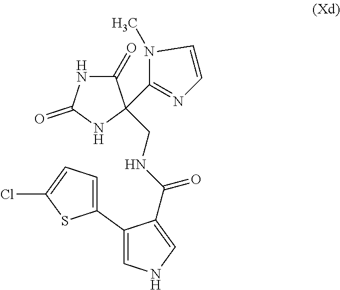 [SMILES: Cn1ccnc1C1(CNC(=O)c2c[nH]cc2-c2ccc(Cl)s2)NC(=O)NC1=O]

### 4-methyl-N-[[4-(1-methylimidazol-2-yl)-2,5-dioxo-imidazolidin-4-yl]methyl]-2-(2-thienyl) thiazole-5-carboxamide

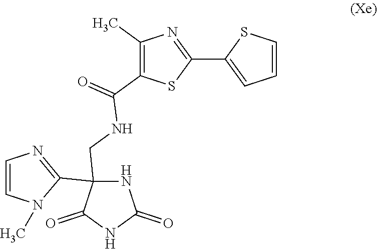 [SMILES: Cc1nc(-c2cccs2)sc1C(=O)NCC1(c2nccn2C)NC(=O)NC1=O]

### N-[[4-(1-methylimidazol-2-yl)-2,5-dioxo-imidazolidin-4-yl]methyl]-2-(4-pyridyl) thiazole-5-carboxamide

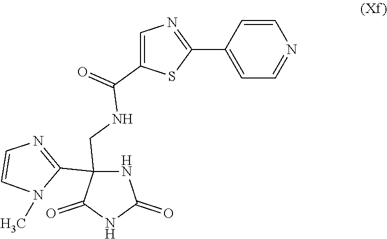 [SMILES: Cn1ccnc1C1(CNC(=O)c2cnc(-c3ccncc3)s2)NC(=O)NC1=O]

### 5-methyl-N-[[4-(1-methylimidazol-2-yl)-2,5-dioxo-imidazolidin-4-yl]methyl]-1-(4-pyridyl) pyrazole-4-carboxamide

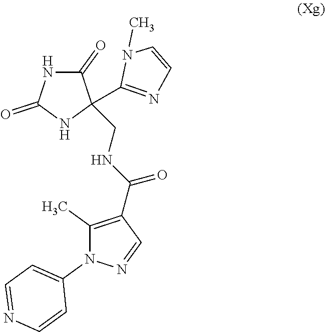 [SMILES: Cc1c(C(=O)NCC2(c3nccn3C)NC(=O)NC2=O)cnn1-c1ccncc1]

### N-[[4-(1-methylimidazol-2-yl)-2,5-dioxo-imidazolidin-4-yl]methyl]-5-(1H-pyrazol-3-yl)thiophene-2-carboxamide

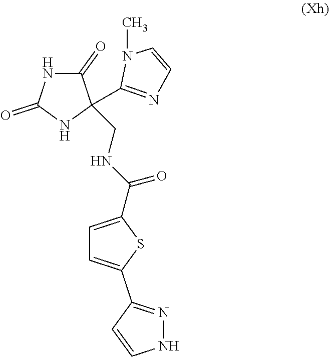 [SMILES: Cn1ccnc1C1(CNC(=O)c2ccc(-c3cc[nH]n3)s2)NC(=O)NC1=O]

### N-[[4-(1-methylimidazol-2-yl)-2,5-dioxo-imidazolidin-4-yl]methyl]-5-phenyl-1H-pyrazole-3-carboxamide

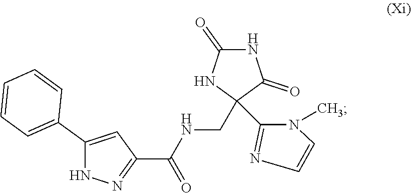 [SMILES: Cn1ccnc1C1(CNC(=O)c2cc(-c3ccccc3)[nH]n2)NC(=O)NC1=O]

or
A compound of general formula (A), in which R′represents a group selected from hydrogen, (C₁-C₆)-alkyl, (C₃-C₆)-cycloalkyl, 5- to 6-membered heterocycloalykl, 5- to 6-membered heteroaryl and phenyl
   wherein said (C₁-C₆)-alkyl (C₃-C₆)-cycloalkyl, 5- to 6-membered heterocycloalkyl, 5- to 6-membered heteroaryl and phenyl are optionally substituted, with one or two groups independently selected from cyano, halogen, amino, hydroxy, oxo, C₁-C₃-alkyl, (C₁-C₄)-alkoxy, (C₁-C₄)-alkylcarbonyl, mono-(C₁-C₄)-alkylamino, di-(C₁-C₄)-alkylamino, (C₁-C₄)-alkylsulfonyl, (C₃-C₆)-cycloalkyl,
   wherein each said (C₁-C₃)-alkyl, (C₃-C₆)-cycloalkyl, and (C₁-C₄)-alkoxy is optionally substituted with up to 5 fluorine atoms,
R² represents a group selected from hydrogen, (C₁-C₄)-alkyl,
   wherein said (C₁-C₄)-alkyl is optionally substituted with up to five halogen atoms,
A represents a group selected from 5-membered aza-heteroaryl,
   wherein said 5-membered aza-heteroaryl is optionally substituted with one, two or three groups independently selected from halogen, cyano, hydroxy, methylsulfonyl, (C₃-C₆)-cycloalkyl, (C₁-C₄)-alkyl, and (C₁-C₄)-alkoxy,
   wherein each said (C₁-C₄)-alkyl, (C₁-C₄)-alkoxy is optionally substituted with up to three fluorine atoms,
Z represents a group selected from Phenyl and 5- to 6-membered heteroaryl,
   optionally substituted, with one, two or three groups independently selected from halogen, cyano, hydroxy, (C₁-C₄)-alkylsulfonyl, (C₃-C₆)-cycloalkyl, (C₁-C₄)-alkyl, and (C₁-C₄)-alkoxy,
   wherein each said (C₁-C₄)-alkyl and (C₁-C₄)-alkoxy is optionally substituted with (C₃-C₆)-cycloalkyl and optionally substituted with up to five fluorine atoms,
      and pharmaceutically acceptable salts thereof, solvates thereof and the solvates of the salts thereof, or a compound of formula (A), in which
R¹ represents a group selected from hydrogen, (C₁-C₆)-alkyl, (C₃-C₆)-cycloalkyl, 5- to 6-membered heterocycloalkyl, 5- to 6-membered heteroaryl and phenyl
   wherein said (C₁-C₆)-alkyl (C₃-C₆)-cycloalkyl, 5- to 6-membered heterocycloalkyl, 5- to 6-membered heteroaryl and phenyl are optionally substituted, with one or two groups independently selected from cyano, halogen, amino, hydroxy, oxo, C₁-C₃-alkyl, (C₁-C₄)-alkoxy, (C₁-C₄)-alkylcarbonyl, mono-(C₁-C₄)-alkylamino, di-(C₁-C₄)-alkylamino, (C₁-C₄)-alkylsulfonyl, (C₃-C₆)-cycloalkyl,
   wherein each said C₁-C₃-alkyl, (C₃-C₆)-cycloalkyl and (C₁-C₄)-alkoxy is optionally substituted with up to 5 fluorine atoms
R² represents a group selected from hydrogen or methyl
A represents a group selected from triazolyl, oxazolyl, isoxazolyl, pyrazolyl, thiazolyl, 1,2,4-oxadiazolyl and 1,3,4-oxadiazolyl optionally substituted with one, two or three groups independently selected from halogen, cyano, hydroxy, methylsulfonyl, (C₃-C₆)-cycloalkyl, (C₁-C₄)-alkyl and (C₁-C₄)-alkoxy wherein each said (C₁-C₄)-alkyl, (C₁-C₄)-alkoxy is optionally substituted with up to three fluorine atoms,
Z represents a group selected from Phenyl and 5- to 6-membered heteroaryl,
   optionally substituted, with one, two or three groups independently selected from halogen, cyano, hydroxy, methylsulfonyl, (C₃-C₆)-cycloalkyl, (C₁-C₄)-alkyl, (C₁-C₄)-alkoxy,
      wherein each said (C₁-C₄)-alkyl and (C₁-C₄)-alkoxy is optionally substituted with, (C₃-C₆)-cycloalkyl and optionally substituted with up to five fluorine atoms, and pharmaceutically acceptable salts thereof, solvates thereof and the solvates of the salts thereof.

In certain embodiments, the antagonists of ADAMTS7 disclosed herein can be conjointly administered with an additional therapeutic agent (e.g., cardiovascular therapeutic agent). Exemplary therapeutic agents include angiotensin-converting enzyme inhibitors, angiotensin-receptor blockers, mineralocorticoid-receptor antagonists, endothelin antagonists, renin inhibitors, calcium blockers, beta-receptor blockers, vasopeptidase inhibitors, Sodium-Glucose-Transport-Antagonists, Metformin, Pioglitazones and Dipeptidyl-peptidase-IV inhibitors. Other therapeutic agents include:
Positive inotropic compounds, such as, e.g., cardiac glycosides (digoxin), beta-adrenergic and dopaminergic agonists, such as isoprenaline, adrenaline, noradrenaline, dopamine or dobutamine and serelaxine;
Vasopressin-receptor antagonists, for example and preferably Conivaptan, Tolvaptan, Lixivaptan, Mozavaptan, Satavaptan, SR-121463, RWJ 676070 or BAY 86-8050, as well as the compounds described in WO 2010/105770, WO 2011/104322 and WO 2016/071212;
Natriuretic peptides, for example and preferably atrial natriuretic peptide (ANP), natriuretic peptide type B (BNP, Nesiritide), natriuretic peptide type C (CNP) or urodilatin;
Activators of cardiac myosin, for example and preferably e.g., Omecamtiv mecarbil (CK-1827452);
Calcium-sensitizers, for example and preferably Levosimendan;
Compounds affecting mitochondrial function and/or production of reactive oxygen species (ROS), for example Bendavia/Elamipritide;
Compounds influencing cardiac energy-metabolism, for example and preferably etomoxir, dichloroacetate, ranolazine, trimetazidine, full or partial adenosine A1 receptor agonists such as GS-9667 (formerly known as CVT-3619), capadenosone and neladenosone;
Compounds with an effect on heart rate, e.g., ivabradine;
Organic nitrates and NO-donors, such as sodium nitroprusside, nitroglycerin, isosorbide mononitrate, isosorbide dinitrate, molsidomine or SIN-1, and inhalational NO;
Compounds that inhibit the degradation of cyclic guanosine monophosphate (cGMP) and/or cyclic adenosine monophosphate (cAMP), such as inhibitors of phosphodiesterases (PDE) 1, 2, 3, 4 and/or 5, in particular PDE 4 inhibitors such as roflumilast or revamilast and PDE 5 inhibitors such as sildenafil, vardenafil, tadalafil, udenafil, dasantafil, avanafil, mirodenafil, lodenafil or PF-00489791;
Compounds increasing cGMP synthesis, such as, e.g., sGC modulators in particular riociguat, nelociguat, vericiguat, cinaciguat and the compounds described in WO 00/06568, WO 00/06569, WO 02/42301, WO 03/095451, WO 2011/147809, WO 2012/004258, WO 2012/028647, WO 2012/059549, WO 2014/068099 and WO 2014/131760 as well as the compounds described in WO 01/19355, WO 01/19780, WO 2012/139888 and WO 2014/012934;
Compounds which inhibit the soluble epoxid-hydrolase (sEH), such as, e.g., N,N′-dicyclohexylurea, 12-(3-Adamantan-1-yl-ureido)-dodecanoic acid or 1-Adamantan-1-yl-3-{5-[2-(2-ethoxyethoxy) ethoxy]pentyl}-urea;
Compounds modulating neurotransmitters, such as, e.g., tricyclic antidepressants such as, e.g., amitryptiline and imaprimine, monoaminooxidase (MAO) inhibitors such as moclobemide, serotonin-noradrenaline-reuptake inhibitors such as venlaflaxine, selective serotonin-reuptake inhibitors such as sertraline or noradrenergic and specific serotonergic antidepressants such as mirtazapine.
Compounds with anxiolytic, sedative and hypnotic properties, so-called tranquilizers such as, e.g., short- as well as mid-long acting benzodiazepines.
Prostacyclin analogs and IP receptor agonists, for example and preferably iloprost, bera-prost, treprostinil, epoprostenol, NS-304, selexipag, or ralinepag;
Compounds which inhibit the signal transduction cascade, in particular from the group of the tyrosine kinase inhibitors and/or from the group of serine/threoninekinase-inhibitors, for example and preferably dasatinib, nilotinib, bosutinib, regorafenib, sorafenib, sunitinib, cediranib, axitinib, telatinib, imatinib, brivanib, pazopanib, vatalanib, gefitinib, erlotinib, lapatinib, canertinib, lestaurtinib, pelitinib, semaxanib, masitinib or tandutinib, Rho kinase inhibitors, such as fasudil, Y-27632, SLx-2119, BF-66851, BF-66852, BF-66853, KI-23095 or BA-1049;
Anti-obstructive agents as used, for example, for the therapy of chronic-obstructive pulmonary disease (COPD) or bronchial asthma, for example and preferably inhalatively or systemically administered beta-receptor mimetics (e.g., bedoradrine) or inhalatively administered anti-muscarinergic substances;
Compounds with a bronchodilatory effect, for example and preferably from the group of β-adrenergic receptor-agonists, such as particularly albuterol, isoproterenol, metaproterenol, terbutaline, formoterol or salmeterol or from the group of anti-cholinergics, such as particularly ipratropiumbromide;
Anti-inflammatory and/or immunosuppressive agents as used, for example for the therapy of chronic-obstructive pulmonary disease (COPD), of bronchial asthma or pulmonary fibrosis, for example and preferably from the group of corticosteroids, such as particularly prednisone, prednisolone, methylprednisolone, triamcinolone, dexamethasone, beclomethasone, betamethasone, flunisolide, budesonide or fluticasone as well as from the group of non-steroidal anti-inflammatory drugs (NSAIDs), such as particularly acetylsalicylic acid (Aspirin), ibuprofen and naproxen, 5-aminosalicylic acid derivatives, leukotriene/leukotriene receptor antagonists, TNF-α inhibitors and chemokine receptor antagonists, such as, e.g., CCR-1, -2 and/or -5 inhibitors. Furthermore, drugs such as pirfenidone, acetylcysteine, azathioprine or BIBF-1120;
Chemotherapeutics as used, for example, for the therapy of neoplasias of the lung or other organs;
Active compounds used for the systemic and/or inhalative treatment of pulmonary disorders, for example for cystic fibrosis (alpha-1-antitrypsin, aztreonam, ivacaftor, lumacaftor, ataluren, amikacin, levofloxacin), chronic obstructive pulmonary diseases (COPD) (LAS40464, PT003, SUN-101), acute respiratory distress syndrome (ARDS) and acute lung injury (ALI) (interferon-beta-1a, traumakines), obstructive sleep apnea (VI-0521), bronchiectasis (mannitol, ciprofloxacin), Bronchiolitis obliterans (cyclosporine, aztreonam) and sepsis (pagibaximab, Voluven, ART-123);
Active compounds used for treating muscular dystrophy, for example idebenone;
Antithrombotic agents, for example and preferably from the group of platelet aggregation inhibiting drugs (platelet aggregation inhibitors, thrombocyte aggregation inhibitors), anticoagulants or compounds with anticoagulant properties or profibrinolytic substances;
Active compounds for lowering blood pressure, for example and preferably from the group of calcium antagonists, angiotensin AII antagonists, ACE inhibitors, NEP inhibitors, vasopeptidase inibitors, and combinations thereof, such as, e.g., sacubitril/valsartan (Entresto®), furthermore nicorandil, endothelin antagonists/endothelin receptor antagonists, such as bosentan, darusentan, ambrisentan, macicentan or sitaxentan, thromboxane A2 (TXA2) antagonists/thromboxane A2 (TBX2) receptor antagonists, renin inhibitors, alpha-receptor blockers, beta-receptor blockers, mineralocorticoid-receptor antagonists, Rho-kinase inhibitors, diuretics as well as further vasoactive compounds/active components such as i.e. adenosine and adenosine receptor agonists.
Compounds which inhibit degradation and remodelling of the extracellular matrix, for example and preferably inhibitors of matrix metalloproteases (MMPs), in particular chymase-inhibitors, stromelysin-inhibitors, collagenase-inhibitors, gelatinase-inhibitors and aggrecanase-inhibitors (in these terms especially MMP-1, MMP-3, MMP-8, MMP-9, MMP-10, MMP-11 and MMP13) as well as inhibitors of the metallo-elastase MMP-12 as well as neutrophil elastase (HNE) inhibitors, for example and preferably sivelestat or DX-890 (Reltran);
Compounds which inhibit the binding of serotonin to its receptor, for example and preferably antagonists of the 5-HT₁-, 5-HT₂ₐ-, 5-HT_{2b}-, 5-HT_{2c}-, 5-HT₃- and 5-HT₄-receptors;
Anti-arrhythmic compounds/active components, for example and preferably sodium channel inhibitors, beta-receptor blockers, potassium channel blockers, calcium antagonists, I_{f}-channel blockers, digitalis, parasympatholytics (vagolytics), sympathomimetics and other anti-arrhythmic drugs such as, e.g., adenosine, adenosine-receptor agonists as well as vernakalant.
Active compounds that alter fat metabolism, for example and preferably from the group of thyroid receptor agonists, cholesterol synthesis inhibitors, for example and preferably HMG-COA-reductase or squalene synthesis inhibitors, ACAT inhibitors, CETP inhibitors, MTP inhibitors, PPAR-alpha-, PPAR-gamma- and/or PPAR-delta-agonists, cholesterol absorption inhibitors, lipase inhibitors, polymeric bile acid adsorbers, bile acid reabsorption inhibitors and lipoprotein (a) antagonists.
Active ingredients which inhibit neoangiogenesis, for example and preferably inhibitors of the VEGF and/or PDGF signalling pathways, inhibitors of the integrin signalling pathways, inhibitors of the angiopoietin-Tie signalling pathways, inhibitors of the PI3K-Akt-mTor signalling pathways, inhibitors of the Ras-Raf-Mek-Erk signalling pathway, inhibitors of the MAPK signalling pathways, inhibitors of the FGF signalling pathways, inhibitors of the sphingosine-1-phosphate signalling pathways, inhibitors of endothelial cell proliferation or apoptosis-inducing active ingredients;
Active ingredients which reduce vascular wall permeability (edema formation), for example and preferably corticosteroids, inhibitors of the ALK1-Smad1/5 signalling pathway, inhibitors of the VEGF and/or PDGF signalling pathways, cyclooxygenase inhibitors, inhibitors of the kallikrein-kinin system or inhibitors of the sphingosine-1-phosphate signalling pathways;
Active ingredients which reduce damage to the retina under oxidative stress, for example and preferably addressing the complement system, especially antagonists of the complement C₅a receptor, or agonists of the 5-HT₁A receptor;
Antioxidants and free-radical scavengers;
Active hypotensive ingredients, for example and preferably from the group of the calcium antagonists, angiotensin AII antagonists, ACE inhibitors, beta-receptor blockers, alpha-receptor blockers, diuretics, phosphodiesterase inhibitors, sGC stimulators, cGMP elevators, aldosterone antagonists, mineralocorticoid-receptor antagonists, ECE inhibitors and vasopeptidase inhibitors;
Antidiabetics and accordingly compounds/active components changing glucose metabolism, for example and preferably from the group of the insulins and/or insulin derivatives, biguanides, sulfonylureas, acarbose, DPP4-inhibitors, meglitinide derivatives, glucosidase inhibitors, GLP 1 receptor agonists/GLP1 analogues, SGLT-2 inhibitors, glucagon antagonists, PPAR-gamma agonists/insulin sensitizers, such as, e.g., thiazolidinediones, CCK1 receptor agonists, leptin receptor agonists, potassium channel antagonists and the inhibitors of hepatic enzymes that are involved in the stimulation of gluconeogenesis and/or glycogenolysis;
Antiinfectives, for example and in particular from the group of the antibacterial, antifungal and/or antiviral substances;
Substances for treatment of glaucoma, for example and in particular from the group of the adrenergics, beta-receptor blockers, carbonic anhydrase inhibitors, parasympathomimetics and prostaglandins;
Pain-reducing compounds such as opiates.

In some embodiments, the additional therapeutic agents are compounds from the group of platelet aggregation inhibiting drugs (platelet aggregation inhibitors, thrombocyte aggregation inhibitors), anticoagulants or compounds with anticoagulant properties or profibrinolytic substances.

In some embodiments, the additional therapeutic agents are compounds from the group of platelet aggregation inhibiting drugs (platelet aggregation inhibitors, thrombocyte aggregation inhibitors), for example and preferably aspirin, clopidogrel, prasugrel, ticlopidine, ticagrelor, cangrelor, elinogrel, tirofiban, PAR1-antagonists such as, e.g., vorapaxar, PAR4-antagonists, EP3-antagonists, such as, e.g., DG041 or inhibitors of adenosine-transport, such as dipyridamole;

In some embodiments, the additional therapeutic agent is a thrombin inhibitor, for example and preferably ximelagatran, melagatran, dabigatran, bivalirudin or Clexane.

In some embodiments, the additional therapeutic agent includes a GPIIb/IIIa antagonist, for example and preferably tirofiban or abciximab.

In some embodiments, the additional therapeutic agent is a factor Xa inhibitor, for example and preferably rivaroxaban, apixaban, edoxaban (DU-176b), darexaban, betrixaban, otamixaban, letaxaban, fidexaban, razaxaban, fondaparinux, idraparinux, as well as thrombin-inhibitors, for example and preferably dabigatran, dual thrombin/factor Xa-inhibitors, such as for example and preferably tanogitran or with factor XI- or factor XIa-inhibitors.

In some embodiments, the additional therapeutic agent are heparin or a low molecular weight (LMW) heparin derivatives, such as i.e. tinzaparin, certoparin, parnaparin, nadroparin, ardeparin, enoxaparin, reviparin, dalteparin, danaparoid, semuloparin (AVE 5026), adomiparin (M118) and EP-42675/ORG42675.

In some embodiments, the additional therapeutic agent is a vitamin K antagonist, for example and preferably coumarines, such as marcumar/phenprocoumon.

In some embodiments, the additional therapeutic agent are pro-fibrinolytic substances, for example and preferably streptokinase, urokinase or plasminogen-activator.

In some embodiments, the additional therapeutic agent are calcium antagonists, angiotensin AII antagonists, ACE inhibitors, endothelin antagonists/endothelin receptor antagonists, thromboxane A2 (TBX2)-antagonists/thromboxane A2 (TBX2) receptor antagonists, renin inhibitors, alpha-receptor blockers, beta-receptor blockers, mineralocorticoid-receptor antagonists, Rho-kinase inhibitors as well as diuretics.

In some embodiments, the additional therapeutic agent is a calcium antagonist, for example and preferably nifedipine, amlodipine, verapamil or diltiazem.

In some embodiments, the additional therapeutic agent is an alpha-1-receptor blocker, for example and preferably prazosin.

In some embodiments, the additional therapeutic agent is a beta-receptor blocker, for example and preferably propranolol, atenolol, timolol, pindolol, alprenolol, oxprenolol, penbutolol, bupranolol, metipranolol, nadolol, mepindolol, carazolol, sotalol, metoprolol, betaxolol, celiprolol, bisoprolol, carteolol, esmolol, labetalol, carvedilol, adaprolol, landiolol, nebivolol, epanolol or bucindolol.

In some embodiments, the additional therapeutic agent is an angiotensin AII antagonist, for example and preferably losartan, candesartan, valsartan, telmisartan or embursatan, irbesartan, olmesartan, eprosartan or azilsartan or a dual angiotensin AII-antagonist/NEP-inhibitor, for example and preferably Entresto (LCZ696, Valsartan/Sacubitril).

In some embodiments, the additional therapeutic agent is an ACE-inhibitor, for example and preferably enalapril, captopril, lisinopril, ramipril, delapril, fosinopril, quinopril, perindopril or trandopril.

In some embodiments, the additional therapeutic agent is an endothelin antagonist/endothelin receptor antagonist, for example and preferably bosentan, darusentan, ambrisentan, avosentan, macicentan, atrasentan or sitaxsentan.

In some embodiments, the additional therapeutic agent is a renin inhibitor, for example and preferably aliskiren, SPP-600 or SPP-800.

In some embodiments, the additional therapeutic agent is a thromboxane A2 (TBX2)-antagonist, for example and preferably seratrodast or KP-496.

In some embodiments, the additional therapeutic agent is a mineralocorticoid-receptor antagonist, for example and preferably spironolactone, eplerenone or finerenone.

In some embodiments, the additional therapeutic agent is a diuretic, for example and preferably furosemide, torasemide bumetanide and piretanide, with potassium-saving diuretics, such as, e.g., amiloride or triamterene as well as with thiazide diuretics, such as, e.g., hydrochlorthiazide, chlorthalidone, xipamide and indapamide. Likewise, the combination with further diuretics is applicable, for example and preferably with bendroflumethiazide, chlorthiazide, hydroflumethiazide, methyclothiazide, polythiazide, trichlormethiazide, metolazone, quinethazone, acetazolamide, dichlorphenamide, methazolamide, glycerol, isosorbide or mannitol.

In some embodiments, the additional therapeutic agent is a Rho-kinase inhibitor, for example and preferably fasudil, Y 27632, SLx-2119, BF-66851, BF-66852, BF-66853, KI-23095, SB-772077, GSK-269962A or BA-1049.

In some embodiments, the additional therapeutic agent are natriuretic peptides, such as, for example “atrial natriuretic peptide” (ANP, Anaritide), “B-type natriuretic peptide”, “brain natriuretic peptide” (BNP, Nesiritide), “C-type natriuretic peptide” (CNP) or Urodilatin;

In some embodiments, the additional therapeutic agent are inhibitors of the endopeptidase (NEP-inhibitors), for example Sacubitril, Omapatrilat or AVE-7688, or as dual combinations (,ARNIs′) with Angiotensin receptor antagonists (for example Valsartan), such as, for example Entresto/LCZ696.

In some embodiments, the additional therapeutic agent are type II antidiabetic drugs, such as inhibitors of the sodium-glucose co-transporter 2 (SGLT2 inhibitors), for example Empagliflozin, Canagliflozin, Dapagliflozin, Ipragliflozin, Tofogliflozin and inhibitors of the dipeptidyl peptidase 4 (DPP-4 inhibitors), for example sitagliptin, saxagliptin, linagliptin, alogliptin.

Substances altering fat metabolism are preferably to be understood as compounds from the group of CETP inhibitors, thyroid receptor agonists, cholesterol synthesis inhibitors such as HMG-COA-reductase or squalene synthesis inhibitors, the ACAT inhibitors, MTP inhibitors, PPAR-alpha, PPAR-gamma and/or PPAR-delta agonists, cholesterol-absorption inhibitors, polymeric bile acid adsorbers, bile acid reabsorption inhibitors, lipase inhibitors as well as the lipoprotein (a) antagonists.

In some embodiments, the additional therapeutic agent is a CETP inhibitor, for example and preferably torcetrapib (CP-529414), anacetrapib, JJT-705 or CETP-vaccine (Avant).

In some embodiments, the additional therapeutic agent is a thyroid receptor agonist, for example and preferably D-thyroxin, 3,5,3′-triiodothyronin (T3), CGS 23425 or axitirome (CGS 26214).

In some embodiments, the additional therapeutic agent is a HMG-COA-reductase inhibitor from the class of statins, for example and preferably lovastatin, simvastatin, pravastatin, fluvastatin, atorvastatin, rosuvastatin or pitavastatin.

In some embodiments, the additional therapeutic agent is a squalene synthesis inhibitor, for example and preferably BMS-188494 or TAK-475.

In some embodiments, the additional therapeutic agent is an ACAT inhibitor, for example and preferably avasimibe, melinamide, pactimibe, eflucimibe or SMP-797.

In some embodiments, the additional therapeutic agent is an MTP inhibitor, for example and preferably implitapide, BMS-201038, R-103757 or JTT-130.

In some embodiments, the additional therapeutic agent is a PPAR-gamma agonist, for example and preferably pioglitazone or rosiglitazone.

In some embodiments, the additional therapeutic agent is a PPAR-delta agonist, for example and preferably GW 501516 or BAY 68-5042.

In some embodiments, the additional therapeutic agent is a cholesterol-absorption inhibitor, for example and preferably ezetimibe, tiqueside or pamaqueside.

In some embodiments, the additional therapeutic agent is a lipase inhibitor, for example and preferably orlistat.

In some embodiments, the additional therapeutic agent is a polymeric bile acid adsorber, for example and preferably cholestyramine, colestipol, colesolvam, CholestaGel or colestimide.

In some embodiments, the additional therapeutic agent is a bile acid reabsorption inhibitor, for example and preferably ASBT (=IBAT) inhibitors, such as AZD-7806, S-8921, AK-105, BARI-1741, SC-435 or SC-635.

In some embodiments, the additional therapeutic agent is a lipoprotein (a) antagonist, for example and preferably gemcabene calcium (CI-1027) or nicotinic acid.

Substances inhibiting signal transduction are preferably to be understood as compounds from the group of the tyrosine-kinase inhibitors and/or serine/threonine-kinase-inhibitors.

In some embodiments, the additional therapeutic agent is a kinase-inhibitor, for example and preferably canertinib, erlotinib, gefitinib, dasatinib, imatinib, lapatinib, lestaurtinib, lonafarnib, nintedanib, nilotinib, bosutinib, axitinib, telatinib, brivanib, pazopanib, pegaptinib, pelitinib, semaxanib, regorafenib, sora-fenib, sunitinib, tandutinib, tipifarnib, vatalanib, cediranib, masitinib, fasudil, lonidamine, leflunomide, BMS-3354825 or Y-27632.

Substances modulating glucose metabolism are preferably to be understood as compounds from the group of insulins, sulfonylureas, acarbose, DPP4-inhibitors, GLP-1 analogues or SGLT-2 inhibitors.

Substances modulating neurotransmitters are preferably to be understood as compounds from the group of tricyclic antidepressants, monoaminooxidase (MAO)-inhibitors, serotonin-noradrenaline-reuptake inhibitors (SNRI) and noradrenergic and specific serotonergic antidepressants (NaSSa).

In some embodiments, the additional therapeutic agent is a tricyclic antidepressant, for example and preferably amitryptilin or imipramin.

In some embodiments, the additional therapeutic agent is a monoaminooxidase (MAO)-inhibitor, for example and preferably moclobemide.

In some embodiments, the additional therapeutic agent is a selective serotonine-noradrenaline reuptake inhibitor (SNRI), for example and preferably venlafaxine.

In some embodiments, the additional therapeutic agent is a selective serotonine reuptake inhibitor (SSRI), such as sertraline.

In some embodiments, the additional therapeutic agent is a noradrenergic and specific serotonergic antidepressants (NaSSa), for example and preferably mirtazapine. Substances with pain-reducing, anxiolytic or sedatative properties are preferably to be understood as compounds from the group of opiates and benzodiazepines.

In some embodiments, the additional therapeutic agent is an opiate, for example and preferably morphine or sulfentanyl or fentanyl.

In some embodiments, the additional therapeutic agent is a benzodiazepine, for example and preferably midazolam or diazepam.

Substances modulating cGMP-synthesis, such as, e.g., sGC-modulators, are preferably to be understood as compounds that stimulate or activate the soluble guanylate cyclase.

In some embodiments, the additional therapeutic agent are sGC modulators, for example and preferably in riociguat, nelociguat, vericiguat, cinciguat and the compounds described in WO 00/06568, WO 00/06569, WO 02/42301, WO 03/095451, WO 2011/147809, WO 2012/004258, WO 2012/028647, WO 2012/059549, WO 2014/068099 and WO 2014/131760 as well as the compounds described in WO 01/19355, WO 01/19780, WO 2012/139888 and WO 2014/012934;

In some embodiments, the additional therapeutic agent are full or partial adenosine A1 receptor agonists, such as, e.g., GS-9667 (formerly known as CVT-3619), capadenosone and neladenosone or compounds affecting mitochondrial function/ROS-production such as i.e. Bendavia/elamipritide.

In some embodiments, the additional therapeutic agent is a TGF-beta antagonist, for example and preferably pirfenidone or fresolimumab.

In some embodiments, the additional therapeutic agent is a TNF-alpha antagonist, for example and preferably adalimumab.

In some embodiments, the additional therapeutic agent are HIF-PH-inhibitors, for example and preferably molidustat or roxadustat.

In some embodiments, the additional therapeutic agent is a serotonin-receptor antagonist, for example and preferably PRX-08066.

### Pharmaceutical Compositions and Administration

In certain embodiments, provided herein are pharmaceutical compositions and methods of using pharmaceutical compositions. In some embodiments, the pharmaceutical compositions provided herein comprise an antagonist of ADAMTS7 and/or another therapeutic agent (e.g., a cardiovascular therapeutic agent).

In certain embodiments, the compositions and methods provided herein may be utilized to treat a subject in need thereof as described herein. In certain embodiments, the subject is a mammal such as a human, or a non-human mammal. In some embodiments, the subject has coronary artery disease. When administered to a subject, such as a human, the composition or the compound is preferably administered as a pharmaceutical composition comprising, for example, a therapeutic compound and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In certain embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

In certain embodiments, the pharmaceutical compositions provided herein comprise a pharmaceutically acceptable carrier. The phrase “pharmaceutically acceptable carrier” as used herein means a pharmaceutically acceptable material, composition or vehicle, such as a liquid or solid filler, diluent, excipient, solvent or encapsulating material. A pharmaceutically acceptable carrier can contain physiologically acceptable agents that act, for example, to stabilize, increase solubility or to increase the absorption of a compound. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a therapeutic compound. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

In certain embodiments, the pharmaceutical compositions provided herein can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound with the carrier and, optionally, one or more accessory ingredients. In general, the formulations are prepared by uniformly and intimately bringing into association a compound with liquid carriers, or finely divided solid carriers, or both, and then, if necessary, shaping the product.

Formulations suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in microencapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the pharmaceutical compositions for administration to the mouth may be presented as a mouthwash, or an oral spray, or an oral ointment.

Alternatively or additionally, compositions can be formulated for delivery via a catheter, stent, wire, or other intraluminal device. Delivery via such devices may be especially useful for delivery to the bladder, urethra, ureter, rectum, or intestine.

Formulations which are suitable for vaginal administration also include pessaries, tampons, creams, gels, pastes, foams or spray formulations containing such carriers as are known in the art to be appropriate.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

In certain embodiments, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinacious biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular subject, composition, and mode of administration, without being toxic to the subject.

If desired, the effective daily dose of the active compound may be administered as one, two, three, four, five, six or more sub-doses administered separately at appropriate intervals throughout the day, optionally, in unit dosage forms. In certain embodiments, the active compound may be administered two or three times daily. In some embodiments, the active compound will be administered once daily.

Actual dosage levels of the therapeutic compound may be varied so as to obtain an amount which is effective to achieve the desired therapeutic response for a particular patient, composition, and mode of administration, without being toxic to the patient.

The selected dosage level will depend upon a variety of factors including the activity of the particular agent employed, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

In certain embodiments, compounds may be used alone or conjointly administered with another type of therapeutic agent (e.g., a cardiovascular therapeutic agent disclosed herein). As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic compounds.

In certain embodiments, conjoint administration of therapeutic compounds with one or more additional therapeutic agent(s) (e.g., one or more additional cardiovascular therapeutic agent) provides improved efficacy relative to each individual administration of the compound (e.g., antagonist of ADAMTS7) or the one or more additional therapeutic agent(s). In certain such embodiments, the conjoint administration provides an additive effect, wherein an additive effect refers to the sum of each of the effects of individual administration of the therapeutic compound and the one or more additional therapeutic agent(s).

Pharmaceutically acceptable salts of compounds in the methods provided herein. In certain embodiments, contemplated salts include, but are not limited to, alkyl, dialkyl, trialkyl or tetra-alkyl ammonium salts. In certain embodiments, contemplated salts include, but are not limited to, L-arginine, benenthamine, benzathine, betaine, calcium hydroxide, choline, deanol, diethanolamine, diethylamine, 2-(diethylamino) ethanol, ethanolamine, ethylenediamine, N-methylglucamine, hydrabamine, 1H-imidazole, lithium, L-lysine, magnesium, 4-(2-hydroxyethyl) morpholine, piperazine, potassium, 1-(2-hydroxyethyl) pyrrolidine, sodium, triethanolamine, tromethamine, and zinc salts. In certain embodiments, contemplated salts include, but are not limited to, Na, Ca, K, Mg, Zn, copper, cobalt, cadmium, manganese, or other metal salts.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Methods of Treating Disease

Provided herein are methods for the treatment and/or prevention of diseases in subjects (e.g., human and animals) such as heart diseases, vascular diseases, and/or cardiovascular diseases, including atherosclerosis, coronary artery disease (CAD), peripheral vascular disease (PAD)/arterial occlusive disease and/or restenosis after angioplasty (including the use of drug-coated or non drug-coated balloons and/or stent-implantation) and/or for the treatment and/or prophylaxis of lung diseases, inflammatory diseases, fibrotic diseases, metabolic diseases, cardiometabolic diseases and/or diseases/disease states affecting the kidneys and/or the central nervous and/or neurological system as well as gastrointestinal and/or urologic and/or ophthalmologic diseases/disease states.

Heart diseases, vascular diseases and/or cardiovascular diseases or disease of the cardiovascular system include acute and chronic heart failure, arterial hypertension, coronary heart disease, stable and instable angina pectoris, myocardial ischemia, myocardial infarction, coronary microvascular dysfunction, microvascular obstruction, no-reflow-phenomenon, shock, atherosclerosis, coronary artery disease, peripheral artery disease, peripheral arterial disease, intermittent claudication, severe intermittent claudication, limb ischemia, critical limb ischemia, hypertrophy of the heart, cardiomyopathies of any etiology (such as, e.g., dilatative cardiomyopathy, restrictive cardiomyopathy, hypertrophic cardiomyopathy, ischemic cardiomyopathy), fibrosis of the heart, atrial and ventricular arrhythmias, transitory and/or ischemic attacks, apoplexy, ischemic and/or hemorrhagic stroke, preeclampsia, inflammatory cardiovascular diseases, metabolic diseases, diabetes, type-I-diabetes, type-II-diabetes, diabetes mellitus, peripheral and autonomic neuropathies, diabetic neuropathies, diabetic microangiopathies, diabetic retinopathy, diabetic ulcera at the extremities, gangrene, CREST-syndrome, hypercholesterolemia, hypertriglyceridemia, lipometabolic disorder, metabolic syndrome, increased levels of fibrinogen and low-density lipoproteins (i.e. LDL), increased concentrations of plasminogen-activator inhibitor 1 (PAI-1), as well as peripheral vascular and cardiac vascular diseases, peripheral circulatory disorders, primary and secondary Raynaud syndrome, disturbances of the microcirculation, arterial pulmonary hypertension, spasms of coronary and peripheral arteries, thromboses, thromboembolic diseases, edema-formation, such as pulmonary edema, brain-edema, renal edema, myocardial edema, myocardial edema associated with heart failure, restenosis after i.e. thrombolytic therapies, percutaneous-transluminal angioplasties (PTA), transluminal coronary angioplasties (PTCA), heart transplantations, bypass-surgeries as well as micro- and macrovascular injuries (e.g., vasculitis), reperfusion-damage, arterial and venous thromboses, microalbuminuria, cardiac insufficiency, endothelial dysfunction.

Heart failure includes more specific or related kinds of diseases such as acute decompensated heart failure, right heart failure, left heart failure, global insufficiency, ischemic cardiomyopathy, dilatative cardiomyopathy, congenital heart defect(s), valve diseases, heart failure related to valve diseases, mitral valve stenosis, mitral valve insufficiency, aortic valve stenosis, aortic valve insufficiency, tricuspid valve stenosis, tricuspid valve insufficiency, pulmonary valve stenosis, pulmonary valve insufficiency, combined valvular defects, inflammation of the heart muscle (myocarditis), chronic myocarditis, acute myocarditis, viral myocarditis, bacterial myocarditis, diabetic heart failure, alcohol-toxic cardiomyopathy, cardiac storage diseases, heart failure with preserved ejection fraction (HFpEF), diastolic heart failure, heart failure with reduced ejection fraction (HFrEF), systolic heart failure.

In the context of the present invention, the terms atrial arrhythmias and ventricular arrhythmias also include more specific and related disease-entitites, such as: Atrial fibrillation, paroxysmal atrial fibrillation, intermittent atrial fibrillation, persistent atrial fibrillation, permanent atrial fibrillation, atrial flutter, sinus arrhythmia, sinus tachycardia, passive heterotopy, active heterotopy, replacement systoles, extrasystoles, disturbances in the conduction of impulses, sick-sinus syndrome, hypersensitive carotis-sinus, tachycardias, AV-node re-entry tachycardias, atrioventricular re-entry tachycardia, WPW-syndrome (Wolff-Parkinson-White syndrome), Mahaim-tachycardia, hidden accessory pathways/tracts, permanent junctional re-entry tachycardia, focal atrial tachycardia, junctional ectopic tachycardia, atrial re-entry tachycardia, ventricular tachycardia, ventricular flutter, ventricular fibrillation, sudden cardiac death.

In the context of the present invention, the term coronary heart disease also includes more specific or related diseases entities, such as: Ischemic heart disease, stable angina pectoris, acute coronary syndrome, instable angina pectoris, NSTEMI (non-ST-segement-elevation myocardial infarction), STEMI (ST-segement-elevation myocardial infarction), ischemic damage of the heart, arrhythmias, and myocardial infarction.

In the context of the present invention, diseases of the central nervous and neurological system or central nervous and neurological diseases/diseases states refer to, e.g., the following diseases/diseases states: Transitory and ischemic attacks, stroke/apoplexy, ischemic and hemorrhagic stroke, depression, anxiety disorder, post-traumatic stress-disorder, poly-neuropathy, diabetic poly-neuropathy, stress-induced hypertension.

In some embodiments, the compositions and methods provided herein are suited for the prophylaxis and/or treatment of poly-cystic kindney-disease (PCKD) and the syndrome of inadequate ADH-secretion (SIADH). Furthermore, the compositions and methods described herein are suited for the treatment and/or prophylaxis of kidney diseases, especially of acute and chronic renal insufficiency as well as of acute and chronic renal failure.

In the context of the present invention, the term acute renal insufficiency/renal failure includes acute presentations of kidney diseases, kidney failure and/or renal insufficiency with or without the dependency on dialysis as well as underlying or related kidney diseases such as renal hypoperfusion, hypotension during dialysis, lack of volume (i.e. dehydration, blood-loss), shock, acute glomerulonephritis, hemolytic-uremic syndrome (HUS), vascular catastrophe (arterial or venous thrombosis or embolism), cholesterol-embolism, acute Bence-Jones-kidney associated with plasmacytoma, acute supravesical or subvesical outlow obstructions, immunologic kidney diseases such as kidney transplant rejection, immuncomplex-induced kidney diseases, tubular dilatation, hyperphosphatemia and/or akute kidney diseases which may be characterized by the need for dialysis. Also included are conditions of partial nephrectomy, dehydration caused by force diuresis, uncontrolled increase in blood pressure accompanied by malignant hypertension, urinary tract obstructions and infections and amyloidosis as well as systemic disorders with glomerular participation such as rheumatologic-immunologic systemic disorders, such as Lupus erythematodes, renal artery thrombosis, renal vein thrombosis, analgesics-induced nephropathy and renal-tubular acidosis as well as radio-opaque substance- as well as drug-induced acute interstitial kidney diseases.

In the context of the present invention the term chronic renal insufficiency/chronic renal failure includes chronic manifestations/presentations of kidney diseases, renal failure and/or renal insufficiency with and without the dependency on dialysis as well as underlying or related kidney diseases such as renal hypoperfusion, hypotension during dialysis, obstructive uropathy, glomerulopathies, glomerular and tubular proteinuria, renal edema, hematuria, primary, secondary as well as chronic glomerulonephritis, membraneous and membraneous-proliferative glomerulonephritis, Alport-syndrome, glomerulosclerosis, tubulointerstitial diseases, nephropathic diseases such as primary and hereditary kidney disease(s), renal inflammation, immunologic kidney diseases such as transplant rejection, immuncomplex-induced kidney diseases, diabetic and non-diabetic nephropathy, pyelonephritis, renal cysts, nephrosclerosis, hypertensive nephrosclerosis and nephrotic syndrome, which are diagnostically characterized by i.e. abnormally reduced creatinine- and/or water-excretion, abnormally increased blood-concentrations of urea, nitrogen, potassium and/or creatinine, altered activity of kidney enzymes, such as, e.g., glutamylsynthase, altered urinary osmolarity or volume, increased microalbuminuria, macroalbuminuria, lesions associated with glomeruli and arterioles, tubular dilatation, hyperphosphatemia and/or the need for dialysis; likewise included are renal cell carcinomas, conditions after partial kidney-resection, dehydration attributed to force diuresis, uncontrolled increase in blood pressure with malignant hypertension, urinary tract obstruction and urinary tract infection and amyloidosis as well as systemic diseases with glomerular participation such as rheumatologic-immunologic systemic diseases, such as lupus erythematodes, as well as renal artery stenosis, renal artery thrombosis, renal vein thrombosis, analgesics-induced nephropathy and renal-tubular acidosis. Furthermore, included are radio-opaque substance- or drug-induced chronic interstitial kidney diseases, metabolic syndrome and dyslipidemia. The current invention also includes the use of the drugs of the current invention for the treatment and/or prophylaxis of after-effects of renal insufficiency such as lung edema, heart failure, uremia, anemia, disturbances in electrolytes (e.g., hyperkalemia, hyponatremia) and disturbances in bone- and carbohydrate-metabolism.

Additionally, compositions and methods provided herein are suited for the treatment and/or prophylaxis of lung diseases (partially also seen as vascular diseases), such as, e.g., pulmonary arterial hypertension (PAH) and other forms of pulmonary hypertension (PH), chronic-obstructive pulmonary disease (COPD), acute respiratory distress syndrome (ARDS), acute lung injury (ALI), lung fibrosis, lung emphysema (e.g., lung emphysema induced by cigarette smoke), cystic fibrosis (CF) as well as for the treatment and/or prophylaxis of alpha-1-antitrypsin deficiency (AATD), acute coronary syndrome (ACS), inflammation of the heart muscle (myocarditis) and other autoimmune diseases of the heart (pericarditis, endocarditis, valvolitis, aortitis, cardiomyopathies), cardiogenic shock, aneurysms, sepsis (SIRS), multiple organ failure (MODS, MOF), inflammatory kidney diseases, chronic bowel diseases (IBD, Crohn's Disease, UC), pancreatitis, peritonitis, rheumatoid diseases, inflammatory skin diseases as well as inflammatory eye diseases.

Furthermore, compositions and methods provided herein can be used for the treatment and/or prophylaxis of asthmatic diseases of different severity with intermittent or persistent courses (refractive asthma, bronchial asthma, allergic asthma, intrinsic asthma, extrinsic asthma, asthma induced by drugs or dust), of different kinds of bronchitis (chronic bronchitis, infectious bronchitis, eosinophilic bronchitis), of bronchiolitis obliterans, bronchiectasia, pneumonia, idiopathic interstitial pneumonia, farmer's lung and related diseases, coughing and common cold diseases (chronic inflammatory cough, iatrogenic cough), inflammations of the nasal mucosa (including drug-induced rhinitis, vasomotor rhinitis and season-dependent allergic rhinitis, e.g., allergic coryza) as well as of polyps.

The compositions described in the current invention also represent active compounds for the treatment of diseases of the central nervous system, characterized by disturbances of the NO/cGMP-system. They are especially suited for improvement of perception, concentration-performance, learning-behaviour or memory-performance after cognitive disturbances as they occur with conditions/illnesses/syndromes such as “mild cognitive impairment”, age-associated learning- and memory-disturbances, age-associated memory-loss, vascular dementia, craniocerebral injury, stroke, dementia occurring after stroke (“post stroke dementia”), post-traumatic craniocerebral injury, general concentration-disturbances, concentration-disturbances affecting children with learning- and memory-problems, Alzheimer's disease, dementia with Lewy-bodies, dementia with degeneration of the frontal lobe including Pick's syndrome, Parkinson's Disease, dementia with corticobasal degeneration, amyotrophic lateral sclerosis (ALS), Huntington's Disease, demyelination, multiple sclerosis, thalamic degeneration, Creutzfeld-Jacob-dementia, HIV-dementia, schizophrenia with dementia or Korsakoff-psychosis. They are also suited for the treatment and or prevention of diseases/disease states of the central nervous system such as conditions of anxiety, tension/pressure and depressions, bipolar disorder, sexual dysfunction due to disturbances in the central nervous system as well as sleep abnormalities and for regulation of pathological disturbances of food-, luxury food- and ‘dependence causing substance’-intake.

Furthermore, the compositions and methods provided herein also suited for the treatment and/or prophylaxis of urologic diseases/disease states such as, e.g., urinary incontinence, stress-induced incontinence, urge incontinence, reflex incontinence and overflow incontinence, detrusor hyperactivity, neurogenic detrusor hyperactivity, idiopathic detrusor hyperacitivity, benign prostate hyperplasia (BPH-syndrome), lower urinary tract symptoms.

The compositions and methods provided herein further suited for the treatment and/or prevention of conditions of pain, such as, e.g., menstrual disorders, dysmenorrhea, endometriosis, preterm delivery, tocolysis.

The compositions and methods provided herein are likewise suited for the treatment and/or prevention of erythematosis, onychomycosis, rheumatic diseases as well as for facilitation of wound healing.

The compositions and methods provided herein are also suited for the treatment and/or prevention of gastrointestinal diseases such as, e.g., diseases/disease states affecting the oesophagus, vomiting, achalasia, gastrooesophageal reflux disease, diseases of the stomach, such as, e.g., gastritis, diseases of the bowel, such as, e.g., diarrhea, constipation, malassimilation syndromes, syndromes of bile acid-loss, Crohn's Disease, Colitis ulcerosa, microscopic colitis, irritable bowel syndrome.

Furthermore, compositions and methods provided herein suited for the treatment and/or prophylactic treatment of fibrotic diseases of inner organs such as lung, heart, kidney, bone marrow, and especially liver as well as dermatological fibrosis and fibrotic eye diseases. In the context of the current invention the term fibrotic diseases includes liver fibrosis, liver cirrhosis, lung fibrosis, endomyocardial fibrosis, cardiomyopathy, nephropathy, glomerulonephritis, interstitial kidney fibrosis, fibrotic damage as a consequence of diabetes, bone marrow fibrosis and similar fibrotic diseases, scleroderma, morphaea, keloids, hypertrophic scarring (also after surgical intervention), naevus, diabetic retinopathy and proliferative vitroretinopathy.

In addition, the compositions and methods provided herein can be used to treat and/or prophylactically treat dyslipidemias (hypercholesterolemia, hypertriglyceridemia, increased concentrations of post-prandial plasma triglycerides, hypo-alphalipoproteinemia, combined hyperlipidemias), metabolic diseases (type I and type II diabetes, metabolic syndrome, overweight, adipositas), nepropathy and neuropathy, cancer (skin cancer, brain tumors, breast cancer, tumors of the bone marrow, leukemias, liposarcoma, carcinoma of the gastrointestinal tract, liver, pancreas, lung, kidney, ureter, prostate and gential tract as well as carcinoma of the lymphoproliferative system such as, e.g., Hodgkin's and Non-Hodgkin's lymphoma), of gastrointestinal and abdominal diseases (glossitis, gingivitis, periodontitis, esophagitis, eosinophilic gastroenteritis, mastocytosis, Crohn's disease, colitis, proctitis, anal pruritis, diarrhea, celiac disease, hepatitis, chronic hepatitis, liver fibrosis, liver zirrhosis, pancreatitis and cholecystitis), skin diseases (allergic skin diseases, psoriasis, acne, eczema, neurodermatitis, multiple kinds of dermatitis, as well as keratitis, bullosis, vasculitis, cellulitis, panniculitis, lupus erythematodes, erythema, lymphoma, skin cancer, Sweet-syndrome, Weber-Christian-syndrome, scarring, wart formation, chilblains), of diseases of the sceletal bones and the joints as well as of sceletal muscle (multiple kinds of arthritis, multiple kinds of arthropathies, scleroderma as well as of further diseases with inflammatory or immunologic components, such as, e.g., paraneoplastic syndrome, rejection reactions after organ transplantations and for wound healing and angiogenesis, especially with chronic wounds.

The compositions and methods provided herein are suited for the treatment and/or prophylactic treatment of ophthalmologic diseases such as, e.g., glaucoma, normotensive glaucoma, increased/high ocular pressure and their combination, of age-related macula degeneration (AMD), dry (non-exudative) AMD, wet (exudative, neovascular) AMD, choroidal neovascularization (CNV), retinal detachment, diabetic retinopathy, atrophic changes of the retinal pigmented epithelium (RPE), hypertrophic changes of the retinal pigmented epithelium, diabetic macula edema, diabetic retinopathy, retinal vein occlusion, choroidal retinal vein occlusion, macula edema, diabetic macula edema, macula edema as a consequence of retinal vein occlusion, angiogenesis at the front-side of the eye such as corneal angiogenesis i.e. after keratitis, cornea transplantation or keratoplasty, corneal angiogenesis due to hypoxia (extensive wearing of contact lenses), Pterygium conjunctivae, sub-retinal edema and intra-retinal edema.

Furthermore, compositions and methods provided herein suited for the treatment and/or prophylactic treatment of increased and high inner ocular pressure as a result of traumatic hyphema, periorbital edema, post-operative viscoelastic retention, intra-ocular inflammation, corticosteroid-use, pupil-block or idiopathic causes such as increased inner ocular pressure after trabeculectomy and due to pre-operative additives.

Furthermore, compositions and methods provided herein suited for the treatment and/or prophylaxis of hepatitis, neoplasms, osteoporosis, glaucoma and gastroparesis. Likewise, compositions and methods provided herein suited for the regulation of cerebral blood circulation and represent useful agents for the treatment and or prophylaxis of migraine. They are also suited for the treatment and prophylaxis of cerebral infarcts such as stroke, cerebral ischemias and traumatic brain injury. Likewise, compositions and methods provided herein can be used for the treatment and/or prophylactic treatment of pain, neuralgias and tinnitus.

The aforementioned, well characterized human diseases may occur in other mammalians with a comparable etiology as well can be treated with the compositions and methods provided herein.

### Methods of Screening Antagonists of ADAMTS7

Some aspects of the disclosure are directed to a method of screening one or more test agents to identify an antagonist of ADAMTS7, comprising contacting a cell sample with a test agent, measuring a level of a cleaved substrate of ADAMTS7 (e.g., cleaved fibulin protein (e.g., EFEMP1) or auto-cleaved ADAMTS7) and identifying the test agent as a antagonist of ADAMTS7 if the level of the cleaved substrate of ADAMTS7 is decreased as compared to a level of cleaved substrate of ADAMTS7 of a corresponding cell sample not contacted with the test agent. The level of cleaved substrate of ADAMTS7 in a corresponding cell sample not contacted with the test agent can be any suitable reference, such as a control sample or a reference sample.

In some embodiments of the invention, the test agent is identified as an antagonist of ADAMTS7 if a level of the cleaved substrate of (e.g., cleaved fibulin protein (e.g., EFEMP1) or auto-cleaved ADAMTS7) is decreased by at least about 5%, 10%, 20%, 30%, 40%, 50%, 60%, 75%, 90%, 99% or more. In some embodiments of the invention, the test agent is identified as an antagonist of ADAMTS7 if a level of the cleaved substrate of (e.g., cleaved fibulin protein (e.g., EFEMP1) or auto-cleaved ADAMTS7) is decreased by at least 1-fold, 2-fold, 3-fold, 5-fold, 6-fold, 7-fold, 8-fold, 9-fold, 10-fold or more.

In some embodiments, any assay capable of detecting expression of the relevant protein (e.g., cleaved substrate of (e.g., cleaved fibulin protein (e.g., EFEMP1) or auto-cleaved ADAMTS7) can be used in the methods provided herein. In some embodiments, the proteins are detected by isotopic labeling (e.g., TAILS (terminal amine isotopic labeling of substrates)). In some embodiments, the proteins are detected by immunostaining with a labeled antibody that binds to the protein epitope. In some embodiments, the proteins are detected by immunohistochemistry. In some embodiments, the proteins are detected by Western Blot. In some embodiments, the mRNAs of the proteins are detected using qPCR. In some embodiments, the proteins are detected using fluorescence activated cell sorting (FACS). In some embodiments, the proteins are detected using microscopy (e.g., fluorescence microscopy). In some embodiments, the proteins are detected using ELISA.

### Exemplification

### Experimental Procedures

ADAMTS7 Expression, Cell Culture and Preparation of the Secreted Proteins—Full-length mouse Adamts7 1-1857 (Uniprot Q68SA9, WT or E373Q catalytic mutant) with a carboxyl terminal 3xFLAG tag was used to generate custom adenoviruses as previously described. Adeno-CMV-Luciferase (Ad-Luc) from Vector Biolabs was used as a negative control. Human Coronary Artery Smooth Muscle Cells (HCA-SMC) were obtained from Lonza and expanded in Lonza SmGM-2 media in 15 cm tissue culture plates. Twenty-four hours before media collection, near confluent plates were transduced with 50 MOI adenovirus and switched to Lonza Basal SM media with volumes of 20 ml per 15 cm plate. Expression of the Flag tagged ADAMTS7 was confirmed from the media using western blot and detected using the anti-Flag M2-HRP antibody (Sigma, A8592). Collected media was processed by adding protease inhibitors (1 mM EDTA and 1 mM PMSF) and clarified by 500 g×5 min spin at 4 C to pellet cell debris. Cell media supernatants were then passed through a 0.22 um filter and kept chilled. Processed media was then concentrated 50-100× using 3 kDa Centricon Plus-70 filter unit, spun at 3,500 g×60 min at 4 C. Approximately 10% of the processed, concentrated media was set aside for total secretome proteolytic analysis. The remaining 90% of the processed, concentrated media was buffer exchanged into 50 mM HEPES pH 8.0, 150 mM NaCl (2×50 ml) using the 3 kDa Centricon Plus-70 filter unit and concentrated to >2 mg/ml to serve as input for the TAILS experiment. Secretome and TAILS input samples were stored at −80° C.

For the first experiment, referred to as SMC1, 120 ml of media from 6×15 cm dishes was pooled from each condition (Luc, WT, EQ) and processed to produce three separate inputs for the TAILS experiment. Luc, WT and EQ inputs from the SMC1 experiment were each divided into triplicate 400 ug samples to generate technical replicate TAILS data. For the second experiment, referred to as SMC2, media was processed separately in triplicate for each condition (Luc1, Luc2, Luc3, WT1, WT2, WT3, EQ1, EQ2, EQ3) using 60 ml of media from 3×15 cm dishes for each process replicate. Inputs from the SMC2 experiment used process replicates of 300 ug each to generate TAILS data. The third experiment used input from cultured Human umbilical vein endothelial cells (HUVEC) from Lifeline Cell Technology grown in VascuLife Media and transduced in Lifeline Basal EC (+bFGF2 10 ng/ml). The HUVEC experiment used the same strategy as the SMC2 experiment, with 40 ml of media from 2×15 cm dishes for each process replicate and used 200 ug each to generate TAILS data. Inputs for process replicate experiments were restricted to the lowest concentration from the 9 parallel samples for SMC2 or HUVEC.

TMT10 isotopic labeling and negative selection of non-labeled peptides-Sample preparation was performed based on the TAILS protocol from the Overall lab with some modifications. From a single experiment, nine parallel protein samples in 50 mM HEPES pH 8.0, 150 mM NaCl were separately denatured with guanidinium chloride (Sigma, G4505) to reach a final concentration of 2.5M guanidinium chloride and 250 mM HEPES and incubated at 65° C. for 15 min. Reduction was achieved through addition of tris(2-carboxylethyl) phosphine (TCEP) at a final concentration of 20 nM TCEP and incubated at 65° C. for 45 min. Alkylation reaction was performed with addition of iodoacetamide (IAA, Sigma, A3221) to a final concentration of 10 mM IAA for 15 min at room temperature in the dark. Following denaturation, reduction and alkylation steps, ten percent of each of the nine parallel protein samples was removed and combined to serve as a pooled reference. Isobaric tags were added to free amine groups using the TMT10plex Isobaric Mass Tagging Kit (ThermoFisher, 90113) with TMT channels randomly assigned to the nine samples and TMT 131 assigned to the pooled reference. Labeling was performed at a ratio of 10:1 label reagent: protein in a final volume of 50% DMSO for 30 min at room temperature, shaking at 850 rpm. A second round of TMT labeling was performed sequentially for 30 min more to increase labeling efficiency. Labeling reactions were quenched with 100 mM ammonium bicarbonate for 15 min at room temperature. Five percent of each reaction was removed to assess preTAILS isobaric labeling efficiencies. Ten TMT labeled samples were then combined and precipitated using 8 volumes of cold acetone and 1 volume of cold methanol in Beckman BK357001 tubes and stored at −80° C. for 3 hours. Samples were then centrifuged in a JA-17 rotor at 14,000 g for 20 min. Supernatants were discarded and the samples were washed twice with 20 ml of ice-cold methanol to remove residual guandinium chloride before trypsin digestion. Pellets were air dried (with SpeedVac briefly when needed), resuspended with 50 mM NaOH and adjusted to 1 mg/ml protein in 50 mM HEPES pH 8.0. SMC1 experiment was digested solely with sequencing grade Trypsin (Promega, V5113) at a ratio of 1:50 protease to protein at 37° C. overnight. For SMC2 and HUVEC experiments, half of the pooled sample was digested with Trypsin and the other half was digested with sequencing grade AspN (Promga, V1621). Five percent of each pooled digestion reaction (preTAILS) was removed to assess negative selection efficiencies. Trypsinized samples (or combined Trypsin/AspN digest products) were adjusted to pH 6-7 and were enriched for TMT blocked N-termini using a hyperbranched polyglycerol aldehyde polymer (HPG-ALD) from the Kizhakkedathu lab, University of British Columbia (Flintbox). HPG-ALD was washed with water and added at 5-fold excess to the digested protein with sodium cyanoborohydride (20 mM final concentration) and incubated at 37° C. overnight. Polymer and polymer bound peptides were retained in 3 kDa Amicon column and the flow-through was collected as input for LC-MS/MS. Five percent of the flow-through (postTAILS) was removed to assess negative selection efficiencies. Enrichment was verified comparing preTAILS and postTAILS isobaric labeling efficiencies for each experiment: SMC1 pre 65.0% vs post 85.5%, SMC2 pre 61.2% vs post 80.9%, HUVEC pre 65.9% vs post 81.7%.

Peptide Fractionation and LC-MS/MS Analysis-Remaining flow-through was desalted on a 30 mg Oasis HLB cartridge. After sample cleanup, the flow-through was separated using basic reverse-phase chromatography on a 2.1×250 mm Zorbax 300 extend-c18 column with a 60 min gradient using 20 mM Ammonium Formate 2% ACN pH 10 buffer A and 20 mM Ammonium Formate 90% ACN pH 10 buffer B. The sample was separated into 96 fractions and concatenated down to 12 by combining every 13th fraction. The 12 fractions were dried in the SpeedVac and then reconstituted in 9 μL of 3%/0.1% ACN/Formic acid. The samples were separated on a Proxeon nanoLC using 3%/0.1% ACN/FA for Buffer A and 90%/0.1% ACN/FA for Buffer B. 4 μL of each fraction were injected and run on a 27 cm c18 column with a 90 min gradient from 6% to 60% Buffer B and run on a Thermo Q-Exactive Plus mass spectrometer. The MS method used was a top 12 method with a Full MS scan at 70,000 resolution and an AGC target of 3e6 from 300-1800 m/z. MS2 scans were collected at 35,000 resolution with an AGC target of 5e4 with a maximum injection time of 120 ms and a dynamic exclusion of 20 seconds. The isolation window used for MS2 acquisition was 0.7 m/z and the scan range was 200-2000 m/z with a normalized collision energy (NCE) of 29 optimized for TMT10 data collection.

Peptide/Protein Quantification, Annotation and Regulated Peptide Analysis—The TAILS data was processed using SpectrumMill. The raw MS files were extracted and searched against the Uniprot human database downloaded on Dec. 28, 2017 with the mouse ADAMTS7 sequence appended. In the SMC2 experiment with the split AspN/Trypsin digest each MS file was searched 4 times. The first search using a tryptic cleavage motif (K.; R.) with TMT10 as a fixed modification of peptide N-termini and Lysine side chains and Acetylated protein N-termini as a variable modification. Then the files were searched again adding acetylated peptide N-termini to the fixed modifications to identify any peptides that are acetylated but aren't the N-terminus of the protein. This process was then repeated using an AspGluN (.D;. E) cleavage motif for SMC2 and HUVEC experiments.

The identified peptides were filtered for redundancy, then by species, and finally peptide score compared to the score of a decoy peptide with all of the interior amino acids of the peptides reversed. This metric is referred to as the delta forward to reverse score and all peptides that scored worse than their reversed counterparts were filtered out. Then the TMT ratios of each sample were then normalized to the average of the natural N-termini present in each sample. This was done by filtering for acetylated peptides with a start amino acid number of 1 or 2 and finding the median ratio of these peptides in each channel. This median was then subtracted from all of the peptides in their respective channels. The resulting TMT ratios were compared to the pooled control using a moderated two sample T-test to identify the peptides with statistically significant differential regulation in the active ADAMTS7 samples compared to the two controls.

Sample processing for the total secretome experiments were performed as previously described. Briefly, the samples were reduced, alkylated and LysC/trypsin digested followed by TMT labeling. Similar to the TAILS experiment, channels were assigned randomly and channel 131 was used as a pooled peptide reference for statistical analysis. Total number of proteins identified from the individual secretome experiments: 2024 (SMC1), 1886 (SMC2) and 2061 (HUVEC). Application of stringency filters (including two or more peptides) resulted in 1847 (SMC1), 1806 (SMC2) and 2031 (HUVEC) fully quantified proteins from the secretome. A moderated two sample T-test was used to compare the three groups similar to the TAILS analysis. Correlation plots and heatmaps of regulated peptides/proteins was performed in Protigy (Proteomics Toolset for Integrated Data Analysis, Broad Institute).

Experimental Design and Statistical Rationale—From the analyzed data, the adjusted p-value (adj.P.val) and log Fold Change (log FC) values were used to identify proteins and substrate cleavage sites enriched for ADAMTS7 proteolytic activity. Results plotting log FC and −log 10 (adj.P.val) data points were visualized in Volcano plots generated by Prism 9. For the SMC1 technical replicate experiment, a p-value <0.01 cut off from for significant hits was applied while a traditional p-value <0.05 cut off for significant hits was used for the SMC2 and HUVEC process replicate experiments. Initial filtering of significant hits from both the adj.P.Val.mWT.over.mEQ and adj.P.Val.mWT.over.Luc identified regulated peptides associated with ADAMTS7 protease activity. No log FC cut-off was applied for initial discovery sets, but positive log FC.mWT.over.mEQ values predicted candidate cleavage sites. Outlier log FC values from SMC1 COL6A1_737, EMB_38, PLEKHH1_260 with incomplete replicate data was excluded from some volcano plots to keep similar data ranges. In some cases, the same substrate cleavage site was identified from multiple peptides. To collapse the discovery set in to a single unique site, the geneSymbol field was concatenated with the StartAA field to generate a cleavage site identifier. Additional filtration of ADAMTS7 auto-catalytic sites and removing significant hits from adj.P.Val.mEQ.over.Luc (associated with overexpression of the catalytically inactive mutant) was performed to generate a high confidence discovery set for each TAILS experiment. Overlap analysis between the SMC1, SMC2 and HUVEC TAILS experiments was performed to identify consistently regulated peptides as candidate ADAMTS7 substrate cleavage sites. Venn diagrams were made manually in Adobe Illustrator. Analysis of cleavage site positions-4 to +4 was performed with Weblogo and iceLogo to generate logo consensus and cleavage site heat maps factoring in the natural abundance of each amino acid in the human proteome.

EFEMP1 Substrate Validation Experiments-Cultured HUVEC with high endogenous EFEMP1 (Fibulin-3) expression were chosen for cell-based validation experiments of TAILS identified cleavage sites. Media from HUVEC transduced with 50 MOI Ad-Luc, Ad-mADAMTS7 WT or Ad-mADAMTS7 EQ was collected in serum free conditions (Lifeline Basal EC+bFGF2 10 ng/ml) and concentrated using 3 kDa Amicon spin columns. Ten percent of the concentrated media was run on a 4-20% Mini-PROTEAN gel (Bio-Rad) and analyzed by western blot to detect the carboxyl region of EFEMP1/Fibulin-3 (antigen 140-209, Novus, NBP2-57871) or ADAMTS7-Flag using M2-HRP. Remaining concentrated media was run on a parallel gel for Coomassie blue staining to collect size specific bands. Gel slices were digested with trypsin or chymotrypsin for mass spectrometry analysis of semi-trypsin and semi-chymotrypsin peptides (Whitehead Institute Proteomics Core Facility). Unique EFEMP1 non-tryptic or non-chymotryptic sites identified more than once from the combined gel slices were compared across Luc, WT and EQ samples. The number of unique mass spectrometry identified peptides representing a potential cleavage site and the combined area for these peptides was used as a semi-quantitative method to identify and compare the proportion of EFEMP1 123.124 and EFEMP1 124.125 cleavage sites. To validate EFEMP1 cleavage in a binary assay, purified recombinant human HA-tagged EFEMP1/Fibulin-3 (R&D, 8416-FB) provided in PBS was dialyzed into TBS pH 8.0 to prevent precipitation with the CaCl₂) in the assay buffer. Purified mouse Adamts7 WT S3A 3xFlag contained a 250 kDa full-length form and 150 kDa truncated Flag tagged form enriched in later SEC fractions as previously described. Peptide sequencing (Tufts University Core Facility) of the lower band or the total purified WT S3A protein identified a potential auto-cleavage site at F1062 (SYGS|FEEP) (SEQ ID NO: 3). Purified mouse ADAMTS7 S3A proteins (0.5 ug) were incubated with HA-EFEMP1/Fibulin-3 (1.0 ug) at 37° C. in an assay buffer containing 50 mM Tris pH 8.0, 150 mM NaCl, 5 mM CaCl₂), 10 uM ZnCl2 and 0.004% Bridj35. The carboxyl region of EFEMP1/Fibulin-3 was detected with Novus antibody NBP-57581 and the amino terminal HA-tag was detected with anti-HA antibody (Cell Signaling, C₂₉F4). Coomassie stained gel slices were submitted for mass spectrometry analysis of semi-trypsin and semi-chymotrypsin peptides (Whitehead Institute Proteomics Core Facility). Analysis was performed similar to the HUVEC validation experiment to identify and compare the proportion of EFEMP1 1223.124 and EFEMP1 124.125 cleavage sites.

### Results

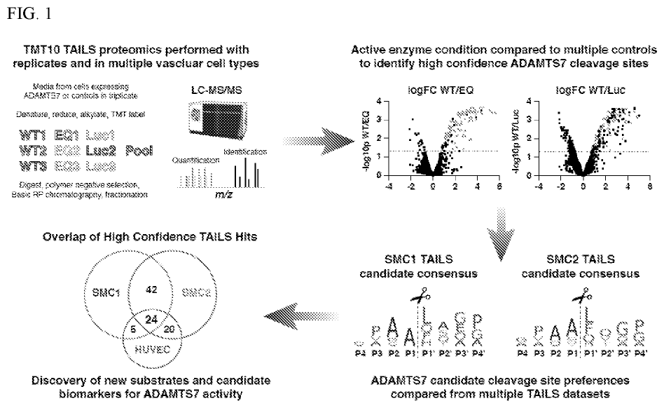

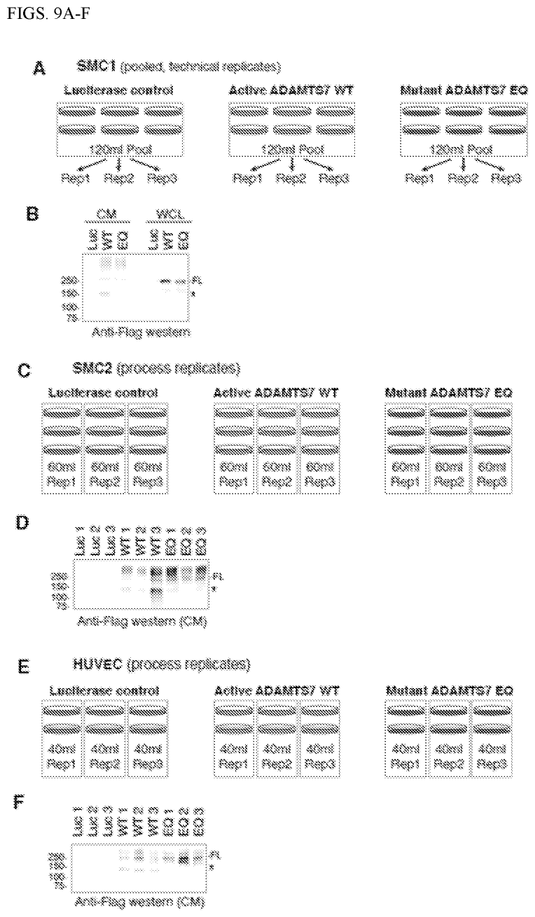

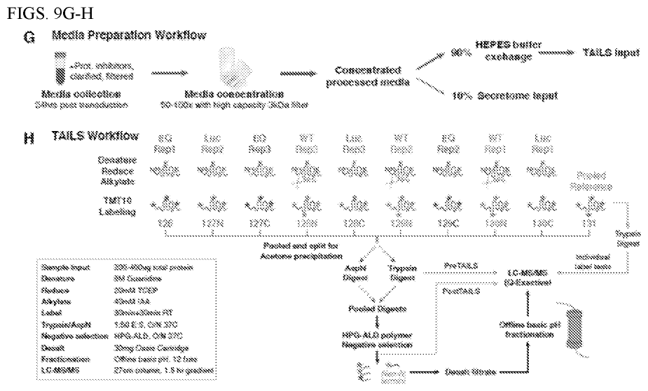

ADAMTS7 TAILS Proteomics from Vascular Smooth Muscle Cell Media—All TAILS experiments were constructed to allow for comparison of the active WT protease condition in triplicate with either of the two negative control groups (FIG. 1). Control groups contained either the expression of a non-specific luciferase or an inactive EQ catalytic mutant protease with a glutamate to glutamine substitution in the HExxH metalloproteinase active site. The study used two different experimental designs to perform TAILS discovery experiments in human coronary artery smooth muscle cells (SMC) (FIG. 9A-D)). In the first experiment, referred to as SMC1, media was pooled from one of three different conditions (Ad-Luc control, Ad-mADAMTS7 WT or Ad-mADAMTS7 EQ) and then split into three technical replicates for each condition to minimize biological variation. In the second experiment, referred to as SMC2, input media from each triplicate condition was processed separately. Greater than 80% of the postTAILS identified spectra contained an isobaric label to enable quantification and allowed for quantitative comparison of 8,818 peptides from 3,152 proteins from SMC1 and 10,964 peptides from 3,579 proteins from SMC2 (Table 1). Total secretome analysis resulted in 1847 SMC1 and 1808 SMC2 fully quantitated proteins.

ADAMTS7 TAILS Proteomics from Vascular Endothelial Cell Media—To identify ADAMTS7 substrate cleavage sites from secreted factors and extracellular matrix proteins originating from a vascular endothelial cell, the study performed a third TAILS experiment using process replicates from adenovirus transduced human umbilical vein endothelial cells (HUVEC) (FIG. 9F-F). Although comparatively less media and protein were used as input for the HUVEC TAILS experiment, the study processed data resulted in 13,276 peptides from 3,826 proteins (Table 1). Parallel secretome analysis was performed again and resulted in 2031 fully quantitated proteins.

TABLE 1
Quantitation statistics of the large scale SMC and HUVEC TAILS experiments.
TMT labeled peptides filtered for redundancy, species, and DeltaForwtoRev > 0.
Peptides with unmodified or acetylated N-termini containing
internal TMT labeled lysine residues were also included in the analysis

| | SMC1 | | SMC2 | | HUVEC | |
|---|---|---|---|---|---|---|
| | Peptides | Proteins | Peptides | Proteins | Peptides | Proteins |
| Quantified TMT | 8818 | 3152 | 10964 | 3579 | 13276 | 3826 |
| labeled peptides | | | | | | |
| Peptides N- | 7325 | 2618 | 6482 | 2349 | 7716 | 2685 |
| terminally labeled | | | | | | |
| with TMT | | | | | | |
| Peptides with | 926 | 483 | 3579 | 1231 | 4873 | 1567 |
| unmodified N- | | | | | | |
| termini | | | | | | |
| N-terminally | 567 | 529 | 903 | 831 | 687 | 636 |
| acetylated peptides | | | | | | |

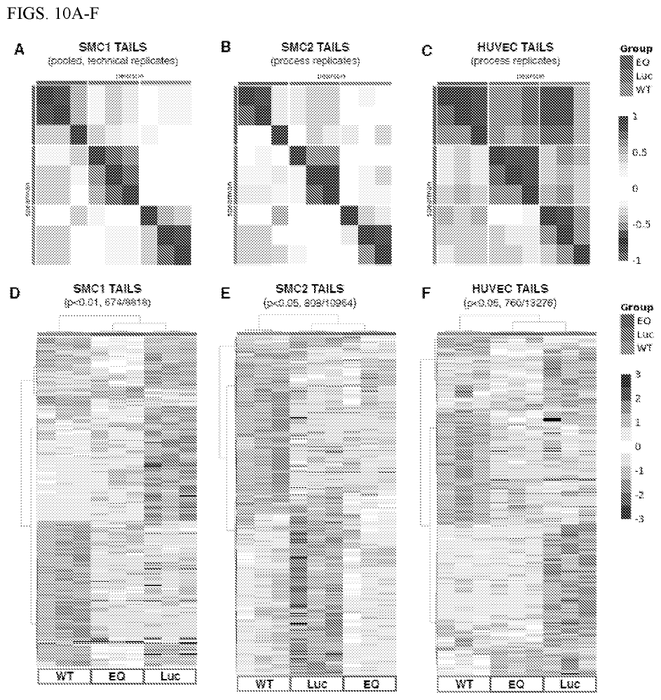

Sample normalization and analysis of regulated peptides/proteins—Before analysis of the triplicate samples for each group (Luc, WT or EQ), the study performed a manual normalization using the median values of TMT labeled natural N-termini peptides. As expected, the technical replicate SMC1 samples showed less inter-sample variation compared to the process replicate SMC2 and HUVEC samples (FIG. 10A-C). Following normalization, the TMT ratio of each channel was compared to the pooled reference channel for statistical analysis and a moderated two sample T-test was used to compare the three groups for each TAILS experiment. Next, the study performed a cluster analysis of the regulated TAILS peptides using a significance cut-off of p<0.01 for the SMC1 technical replicates and p<0.05 for the process replicates (FIG. 10D-F). For each TAILS experiment the study observed more similarity between the Luc control and EQ negative control than to the active WT condition.

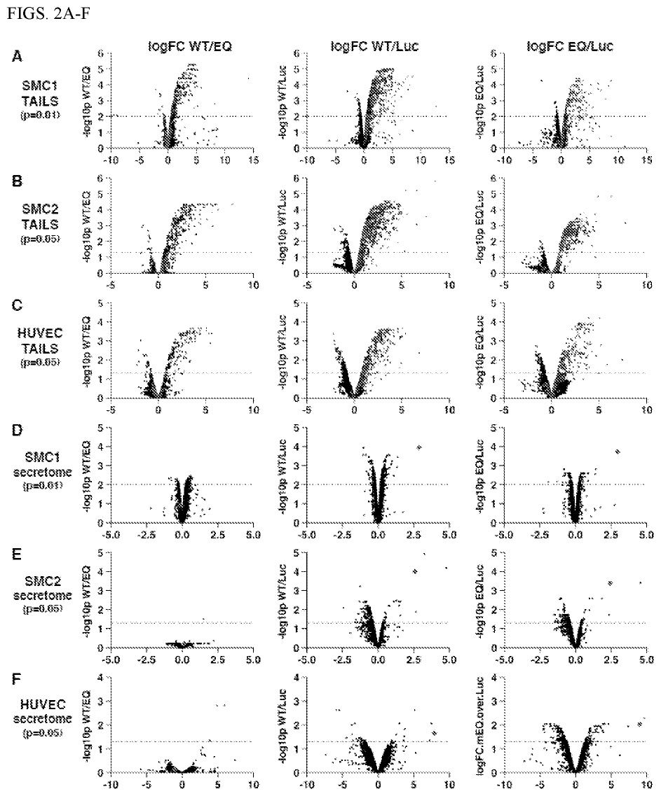

To compare the TAILS regulated peptides between the three experimental conditions, the study plotted the log fold change enrichment (log FC) and adjusted p-values for WT/EQ, WT/Luc and EQ/Luc in volcano plots (FIG. 2A-C). Differentially regulated peptides passing the significance threshold for each experiment were predominantly in the positive log FC side of each TAILS volcano plot. Peptides identified as mouse ADAMTS7 were colored in red for each plot. Significantly regulated peptides in the WT/EQ and WT/Luc comparisons with positive log FC values represented neo N-termini potentially associated with ADAMTS7 catalytic activity. In contrast, the EQ/Luc significant and positive log FC neo N-termini are more likely to be artifact from adenoviral expression of a full-length catalytically inactive protein.

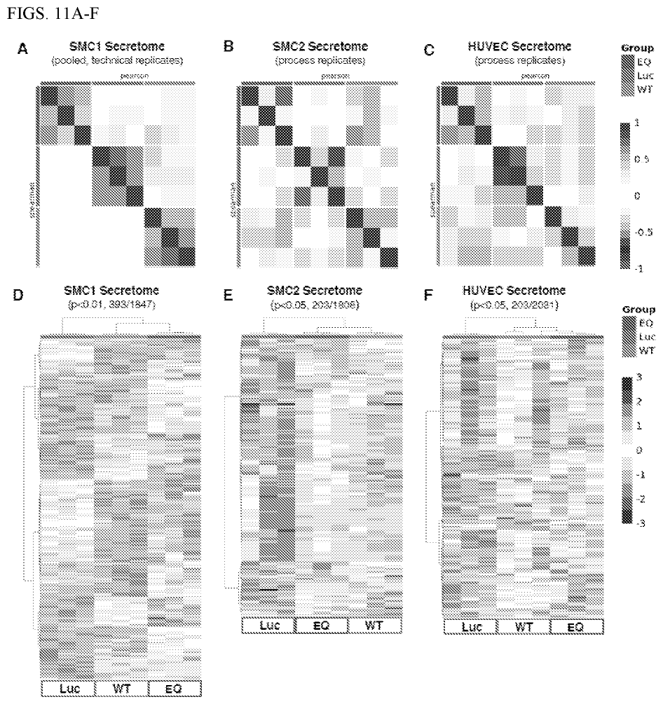

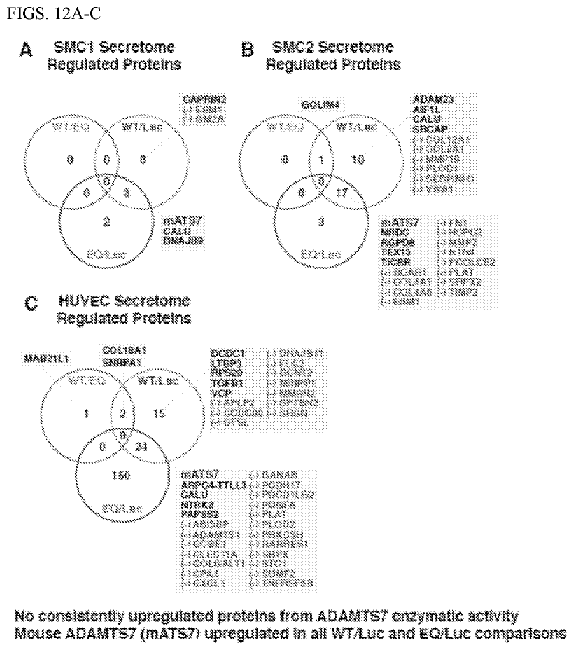

For the total secretome analysis, a standard median normalization was used for the SMC1 technical replicates. SMC2 and HUVEC samples displayed greater diversity from biological replicates, therefore the study applied median and median absolute deviation (M-MAD) to normalize the more variable secretome samples. Even following normalization, the correlation matrixes showed a clear difference between the SMC1 technical replicates and the SMC2 and HUVEC process replicates (FIG. 11A-C). Normalized TMT labeled secretomes were analyzed similar to the TAILS experiment and a moderated two sample T-test was used to compare the three groups for each total secretome experiment. Cluster analysis of the secretome proteins showed more similarity in the secreted proteomes between the WT and EQ expressing samples than the Luc control sample (FIG. 11D-F). Therefore, the secretomes clustered not with ADAMTS7 activity, as with the TAILS experiments, but with ADAMTS7 expression regardless of catalytic activity. Comparative secretome analysis of the WT/EQ, WT/Luc and EQ/Luc conditions was performed using the same significance thresholds from the corresponding TAILS experiments and visualized using volcano plots (FIGS. 2D-F). The position of mouse ADAMTS7 protein was noted in each volcano plot with a red diamond. As expected mouse ADAMTS7 was significantly upregulated in WT/Luc and EQ/Luc comparisons, with a log FC range of 2.5-3 for the SMC experiments and a log FC range of 8-9 for the HUVEC experiment. In contrast there was no significant expression difference in the WT/EQ secretome comparisons for mouse ADAMTS7 indicating a similar level of WT and EQ protein abundance. Next, the study filtered the significantly regulated proteins to log FC>1 (greater than 2-fold upregulated) or <−1 (down regulated more than 2-fold) and looked for commonalities in the WT/EQ, WT/Luc and EQ/Luc comparisons using a Venn diagram for each experiment (FIGS. 12A-C). Using these criteria, very few proteins were associated purely with proteolytic activity from the WT/EQ secretomes. Furthermore, there were no commonalities between the three secretome experiments with the exception of mouse ADAMTS7 in the overlap between WT/Luc and EQ/Luc.

Examination of ADAMTS7 auto-cleavage events—Next, the study focused on the regulated peptides from mouse ADAMTS7 in the TAILS experiments. Of the significantly regulated peptides for the WT/EQ comparisons associated with ADAMTS7 activity from each experiment, around 17% corresponded to mouse ADAMTS7 peptides and nearly all with positive log FC value (FIGS. 2A-C). The WT/EQ ADAMTS7 peptides that were significantly enriched were also found in the WT/Luc comparisons associated with ADAMTS7 activity. Therefore, these ADAMTS7 neo N-termini from the WT/EQ comparison were likely due to cis or trans auto-cleavage events. On the other hand, there were EQ/Luc regulated peptides that were also present in the WT/Luc comparisons, possibly representing proteolysis from other cellular proteases. Consistent with this hypothesis, the total number of WT/EQ and EQ/Luc significant ADAMTS7 log FC+ peptides added up to roughly the number of WT/Luc log FC+ peptides (Table 2). WT/EQ significantly enriched peptides filtered against EQ/Luc significant peptides resulted in the study list of ADAMTS7 auto-cleavage sites from each TAILS experiment (Table 4).

TABLE 2
Summary of regulated peptides in SMC and HUVEC TAILS experiments.
Significantly regulated quantitated peptides from WT/EQ, WT/Luc and EQ/Luc comparisons
presented from each TAILS experiment. Positive Log fold change (FC+) and negative Log
fold change (FC−) regulated peptides indicate significantly upregulated or downregulated
peptides from each comparison. After removal of ADAMTS7 peptides, the candidate FC+
represent potential ADAMTS7 substrate cleavage sites. High confidence candidates were
present in both WT/EQ and WT/Luc comparisons from each TAILS experiment.

| | | SMC1 | | | SMC2 | | | HUVEC | |
|---|---|---|---|---|---|---|---|---|---|
| | 8818 | p < 0.01 | | 10964 | p < 0.05 | | 13276 | p < 0.05 | |
| | WT/ | WT/ | EQ/ | WT/ | WT/ | EQ/ | WT/ | WT/ | EQ/ |
| | EQ | Luc | Luc | EQ | Luc | Luc | EQ | Luc | Luc |
| Significant | 312 | 646 | 217 | 314 | 755 | 333 | 213 | 524 | 349 |
| All LogFC+ | 297 | 590 | 194 | 302 | 681 | 321 | 191 | 376 | 251 |
| All LogFC− | 15 | 56 | 23 | 12 | 74 | 12 | 22 | 148 | 98 |
| ADAMTS7 | 57 | 201 | 133 | 54 | 250 | 157 | 36 | 157 | 136 |
| FC+ | | | | | | | | | |
| ADAMTS7 | 3 | 0 | 0 | 1 | 0 | 0 | 3 | 1 | 0 |
| FC− | | | | | | | | | |
| Candidates | 240 | 389 | 61 | 248 | 431 | 164 | 155 | 219 | 115 |
| FC+ | | | | | | | | | |
| Candidates | 12 | 56 | 23 | 11 | 74 | 12 | 19 | 147 | 98 |
| FC− | | | | | | | | | |
| Dup Cand | 2 | 3 | 0 | 3 | 4 | 0 | 1 | 1 | 1 |
| FC+ | | | | | | | | | |
| Novel Cand | 238 | 386 | 61 | 245 | 427 | 164 | 154 | 218 | 114 |
| FC+ | | | | | | | | | |
| High | 207 | | | 210 | | | 127 | | |
| confidence | | | | | | | | | |

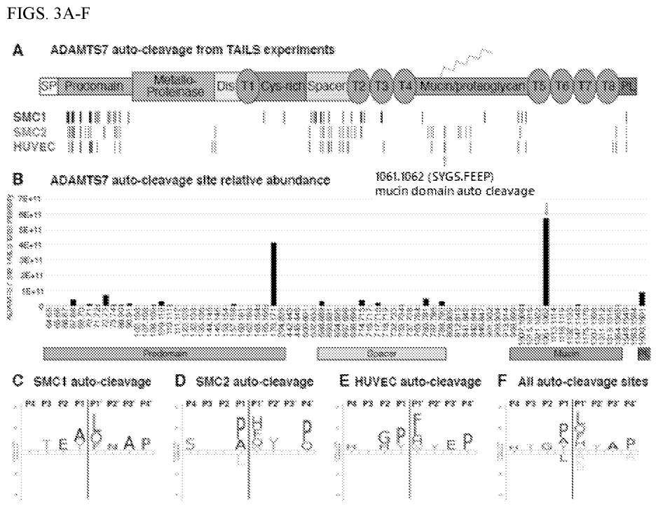

The study compared the locations of the prospective auto-cleavage events from each TAILS experiment and found a third to nearly half of the unique sites were located in the prodomain (residues 21-220). Only 11 of the ADAMTS7 sites were common to all experiments, with 7 in the prodomain, 3 in the spacer domain and 1 in the PLAC domain (FIG. 3A). The most abundant prospective auto-cleavage sites based on spectra total intensity were found at 170.171 (HAQP|HVVY) (SEQ ID NO: 5) in the prodomain, 1061.1062 (SYSG|FEEP) (SEQ ID NO: 6) in the mucin domain and 1600.1601 (EDCE|LVEP (SEQ ID NO: 7)) in the PLAC domain (FIG. 3B). The sequence context for these sites share little similarity with each other prompting the question of site specificity for ADAMTS7. The study analyzed the prospective mouse ADAMTS7 cleavage sites (filtered to log FC>1) using iceLogo to generate a sequence logo from each TAILS experiment (FIG. 3C-F). Based on the plots from individual experiments or the combined 75 unique sites from all three experiments, a slight preference for proline or alanine at P1 and leucine at P1′ flanking the cleavage sites was present, but not strictly required, suggesting broad specificity for ADAMTS7 cleavage.

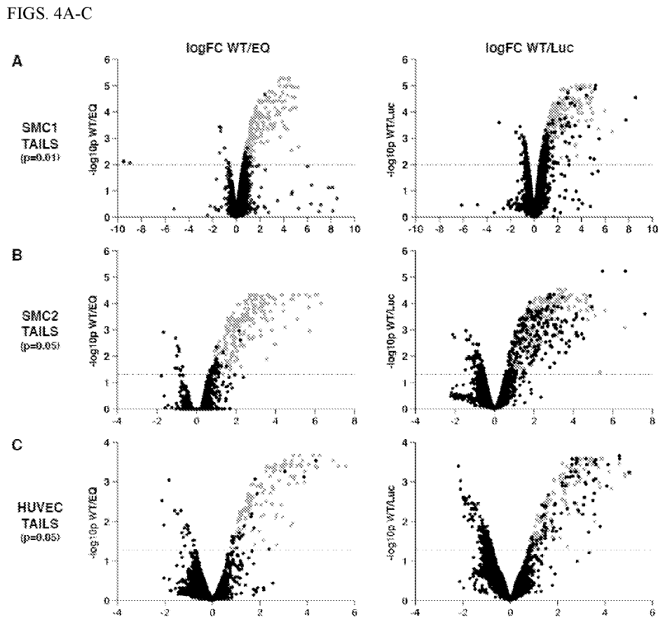

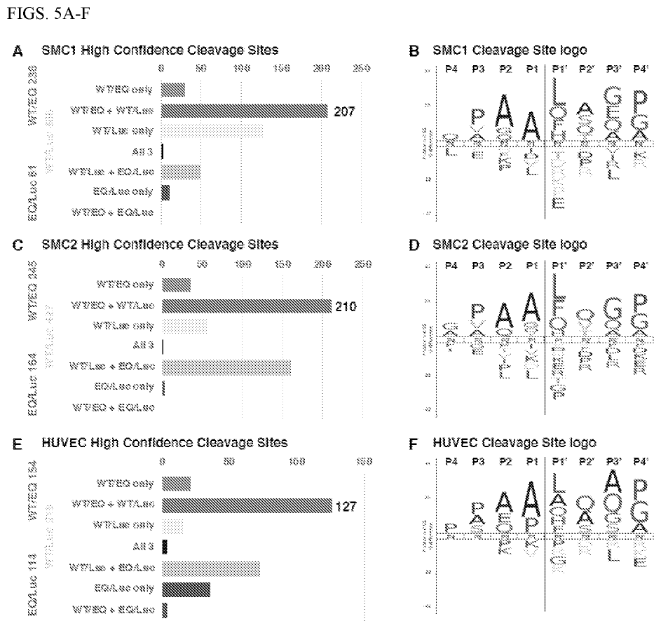

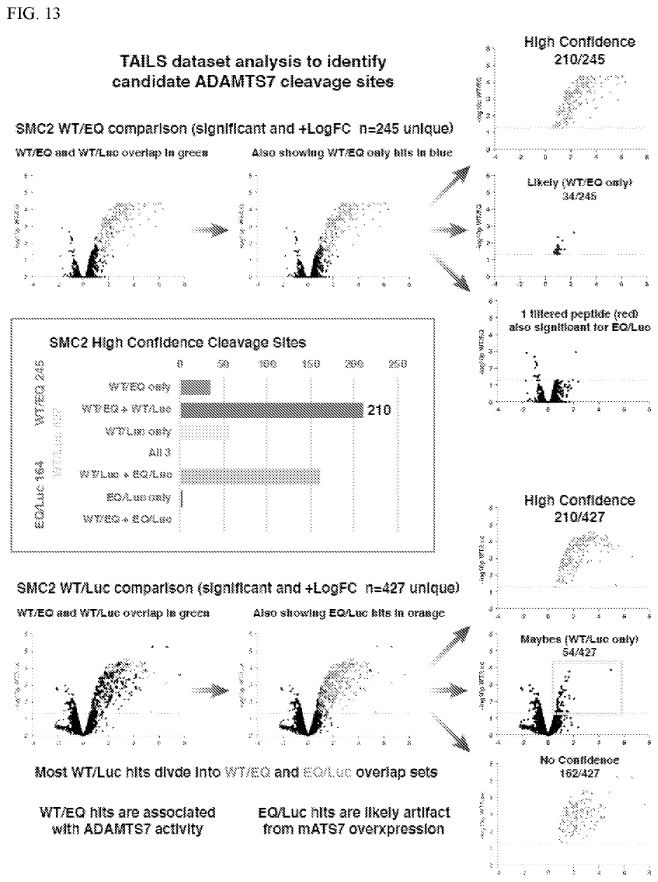

ADAMTS7 substrate cleavage sites identified by TAILS—To generate a high confidence list of substrates from each TAILS experiment, the study applied a series of requirements and filters to the significant hits from the WT/EQ comparisons. First, the study excluded any ADAMTS7 sites or any sites with a log FC less than zero from the WT/EQ significant hits (Table 2). Second, the study performed the same exclusions to the WT/Luc significant hits as a separate comparison for ADAMTS7 function and used the filtered overlap from the WT/EQ and WT/Luc as a stringent constraint for ADAMTS7 catalytic activity. Lastly, the study removed any sites that were significantly upregulated in the EQ/Luc comparisons or any duplicate identifications from multiple peptides to generate a unique list of high confidence substrate cleavage sites. Volcano plots with the mouse ADAMTS7 peptides removed from the dataset show the high confidence substrate cleavage site regulated peptides (labeled in green) within the upper right quadrant (FIG. 4A-C). Histograms illustrate the overlap of significantly regulated unique cleavage sites from each of the comparisons and display a similar trend for the independent TAILS experiments (FIG. 5A, C, E). It is noteworthy that the WT/EQ high confidence regulated peptides on average were more significant and had higher log FC values compared to WT/EQ only regulated peptides which lacked independent verification within each TAILS dataset. In the case of the SMC2 TAILS dataset, the WT/EQ only average log FC was 1.0 versus the WT/EQ high confidence average of log FC of 2.5 (FIG. 13). While a predominance of high confidence hits were present in the WT/EQ regulated peptides, the WT/Luc regulated peptides contained a mixture of overlap with the activity associated WT/EQ comparison and the EQ/Luc regulated peptides likely associated with artifact from ADAMTS7 overexpression.

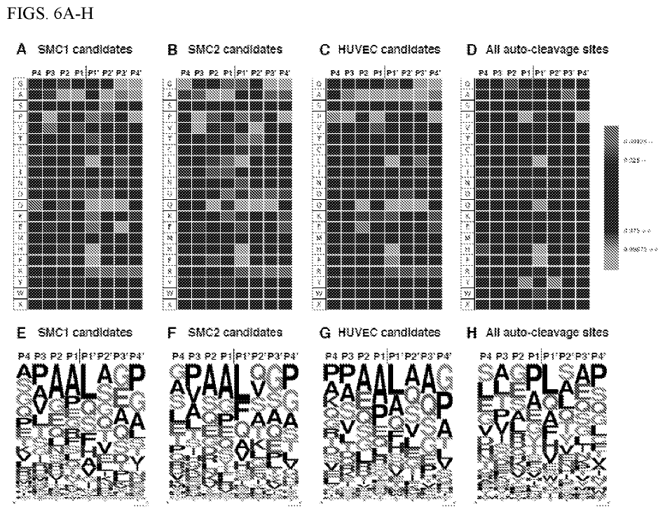

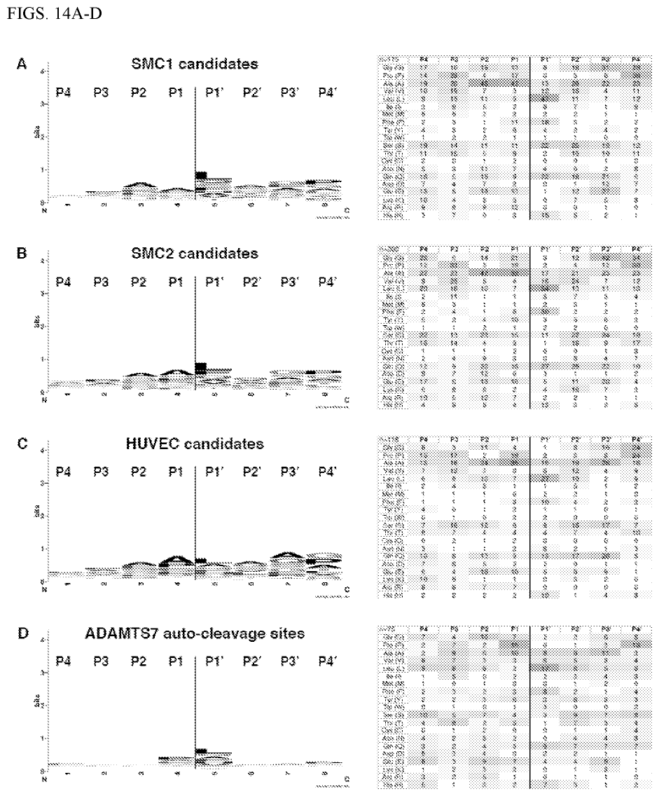

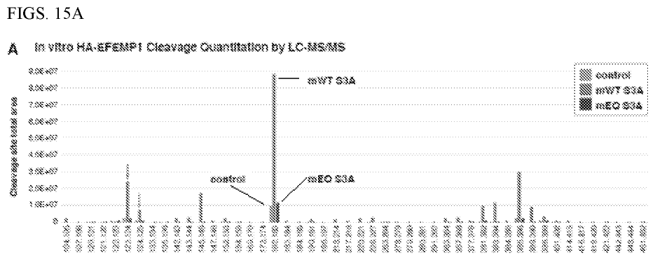

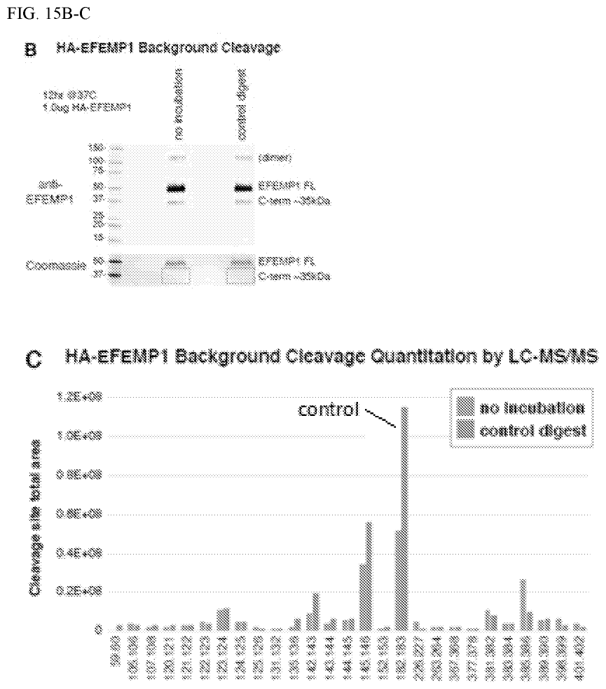

From the original number of WT/EQ significantly regulated peptides, 66% of the SMC1, 67% of the SMC2 and 60% of the HUVEC significant hits passed all these criteria (Table 2 and Table 5). Next, the study compared the substrate logo and heat maps of high confidence substrate cleavage sites from the SMC and HUVEC TAILS experiments by applying the same criteria from the mouse ADAMTS7 auto-cleavage analysis. Based on the iceLogo plots, AA|L at the P2, P1 and P1′ positions was most commonly observed (FIGS. 5B, D, F); however, each of these amino acids was present at no more than 30% in their respective positions (FIG. 14). Although a strict consensus was not apparent from the study individual high confidence datasets, similarities were evident in candidate substrate heat maps and amino acid frequency plots from different TAILS experiments (FIG. 6).

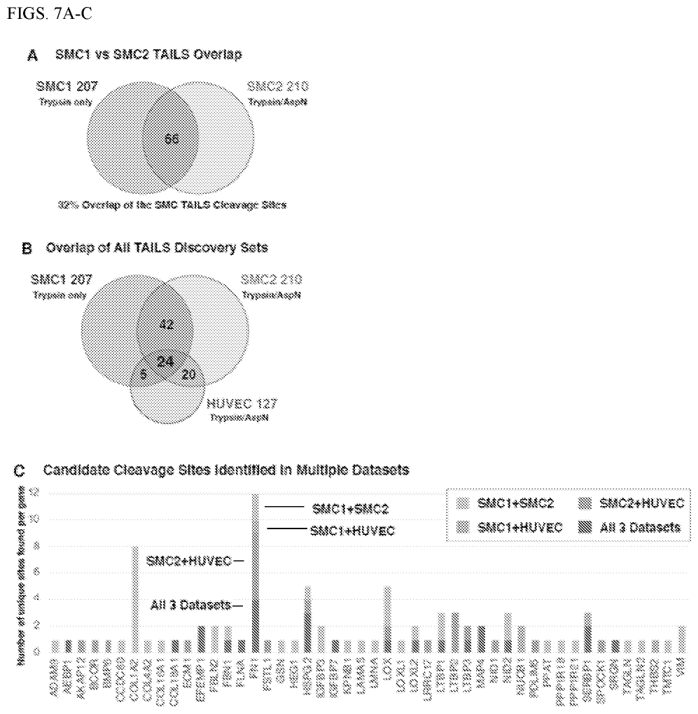

TAILS discovery set overlap analysis—By virtue of having multiple independent TAILS discovery experiments for ADAMTS7, the study compared the list of high confidence substrate cleavage sites to emphasize those that were found more than once. From the comparison of substrate cleavage sites from SMC1 (207 identified solely by trypsin digests) and SMC2 (210 identified from half trypsin or half AspN digests), 66 were identical resulting in an overlap of 32% of the identified sites (FIG. 7A). Although both SMC samples were digested with trypsin, 13 of the 66 unique sites were identified by AspN regulated peptides from the SMC2 TAILS experiment rather than the comparable semi-tryptic peptide from the SMC1 TAILS experiment, demonstrating that multiple peptides can identify the same cleavage site. Sorting the overlapping SMC cleavage sites into common genes identified multiple cleavage sites in several candidate substrates, including 8 for COL1A2 (Collagen type I alpha-2 chain), 6 for FN1 (Fibronectin), 4 for HSPG2 (Basement membrane-specific heparan sulfate proteoglycan core protein/Perlecan) and 4 for LOX (Protein-lysine 6-oxidase). Comparison of the candidate sites from SMC2 and HUVEC (127, also identified from half trypsin or half AspN digests) resulted in 44 identical cleavage sites, including additional sites in FN1, HSPG2 and LOX not present in the SMC1 TAILS high confidence cleavage site list (FIG. 7B). Of these 44 shared sites, the 20 that were unique to the SMC2/HUVEC comparison were predominantly from the AspN digestion (15 semi-AspN peptides and 5 purely semi-tryptic peptides), which may explain why these sites were not identified in the SMC1 trypsin only digestion TAILS. In total there were 91 unique sites identified multiple times in the study TAILS discovery experiments for ADAMTS7 substrates (Table 5). When the individual sites are grouped into their corresponding gene, 48 potential substrates emerged (FIG. 7C). The most unique cleavage sites from multiple datasets were identified from FN1, including 4 unique cleavage sites from significantly regulated peptides in all TAILS experiments. Furthermore, both FN1 and LOX are amongst the CAD risk loci categorized as non-lipid vascular remodeling pathway genes along with ADAMTS7.

Analyzing the overlap between all three TAILS discovery sets identified 24 unique cleavage sites encoded by 16 different genes (Table 3). Most ADAMTS7 candidate substrates from this list were primarily localized in the extracellular region, with the exception of proteins with defined roles in the cytoskeletal (FLNA and MAP4) and nuclear (SERBP1) intracellular regions. The 24 cleavage sites were found in a variety of substrate protein domains and were commonly found in N-terminal regions or unstructured linker regions. Remarkably some of the unique cleavage sites were found at adjacent positions in the same candidate substrate, as was the case with EFEMP1 and MAP4. In both cases the log FC ratios favored the more N-terminal cleavage site, which may indicate either an initial preference for the first site or a sequential cleavage event while the enzyme remained associated with the cleaved substrate. By analyzing the overlap between independent TAILS discovery sets, the study have identified reproducible substrate cleavage sites which the study used to prioritize validation experiments to confirm ADAMTS7 protease specificity.

TABLE 3
ADAMTS7 candidate substrates identified in all TAILS experiments.
High confidence ADAMTS7 cleavage sites consistently detected in all TAILS discovery datasets. Fold
change enrichment with ADAMTS7 activity (logFC WT/EQ) and TAILS peptide ID total intensity
displaying relative abundance in each TAILS experiment is shown for the 24 candidate sites. Sequence
context for each cleavage site is shown (P4 through P4′) and location within the target candidate protein.

| | SMC1 | | SMC2 | | HUVEC | HUVEC | | | |
|---|---|---|---|---|---|---|---|---|---|
| Gene | logFC | SMC1 total | logFC | SMC2 total | logFC | total | | SEQ | Substrate |
| (TAILS_id) | WT/EQ | Intensity | WT/EQ | Intensity | WT/EQ | Intensity | Cleavage site | ID NO: | location |
| AEBP1_37 | 3.41 | 4.54E+07 | 3.48 | 8.64E+07 | 4.34 | 5.26E+06 | EIEE.FLEG | 8 | N-terminal |
| | | | | | | | | | region |
| COL18A1_1505 | 1.42 | 1.68E+09 | 1.47 | 1.11E+10 | 3.88 | 1.34E+11 | EVAA.LQPP | 9 | NC1 hinge |
| | | | | | | | | | region |
| EFEMP1_124 | 4.43 | 3.19E+09 | 3.27 | 2.58E+10 | 3.31 | 2.92E+11 | ASAA.AVAG | 1 | Within |
| | | | | | | | | | atypical EGF1 |
| EFEMP1_125 | 2.25 | 2.03E+09 | 2.42 | 7.46E+09 | 2.45 | 9.33E+10 | SAAA.VAGP | 2 | Within |
| | | | | | | | | | atypical EGF1 |
| FBN1_29 | 4.49 | 1.26E+10 | 3.57 | 2.40E+10 | 3.91 | 1.85E+09 | ADAN.LEAG | 10 | N-term/ |
| | | | | | | | | | propeptide |
| FLNA_1285 | 5.06 | 5.65E+08 | 5.72 | 1.38E+09 | 3.81 | 2.92E+09 | DARA.LTQT | 11 | Filiamin 11 |
| FN1_36 | 3.34 | 4.40E+10 | 3.69 | 1.54E+11 | 3.45 | 1.76E+11 | QAQQ.MVQP | 12 | N-terminal |
| | | | | | | | | | region |
| FN1_40 | 4.27 | 3.67E+09 | 2.66 | 3.29E+09 | 2.96 | 1.37E+10 | MVQP.QSPV | 13 | N-terminal |
| | | | | | | | | | region |
| FN1_1143 | 0.86 | 1.36E+08 | 2.80 | 9.55E+08 | 3.68 | 4.67E+09 | VVSG.LTPG | 14 | Fibronectin |
| | | | | | | | | | type-III 6 |
| FN1_1656 | 1.52 | 4.82E+09 | 1.86 | 2.00E+10 | 1.63 | 2.23E+10 | KWLP.SSSP | 15 | Fibronectin |
| | | | | | | | | | type-III 12 |
| HSPG2_275 | 2.49 | 2.35E+07 | 2.10 | 1.16E+08 | 0.77 | 2.67E+07 | APQP.LLPG | 16 | Linker (LDL- |
| | | | | | | | | | A1/A2) |
| HSPG2_1863 | 2.03 | 1.64E+08 | 1.90 | 1.42E+08 | 2.53 | 4.95E+08 | ASGT.LSAP | 17 | Ig-like C2- |
| | | | | | | | | | type 3 |
| HSPG2_2331 | 2.04 | 7.64E+07 | 1.91 | 2.66E+08 | 1.73 | 2.45E+09 | GTQG.ANLA | 18 | Ig-like C2- |
| | | | | | | | | | type 8 |
| IGFBP7_101 | 3.69 | 5.68E+07 | 2.57 | 4.97E+08 | 1.89 | 1.12E+08 | KAGA.AAGG | 19 | IGFBP N- |
| | | | | | | | | | term region |
| LOX_124 | 3.62 | 1.65E+08 | 3.45 | 1.64E+09 | 3.35 | 8.74E+07 | TARH.WFQA | 20 | N-term/ |
| | | | | | | | | | propeptide |
| LOXL2_37 | 3.28 | 5.20E+09 | 3.30 | 9.09E+09 | 3.48 | 1.61E+09 | YPEY.FQQP | 21 | N-terminal |
| | | | | | | | | | region |
| LTBP1_393 | 3.89 | 9.30E+08 | 6.30 | 6.93E+09 | 3.33 | 9.19E+08 | AADT.LTAT | 22 | Linker |
| | | | | | | | | | (EGF1/2) |
| LTBP3_239 | 2.62 | 1.25E+08 | 1.59 | 8.70E+08 | 1.57 | 5.41E+08 | PPEA.SVQV | 23 | Linker |
| | | | | | | | | | (EGF1/TB1) |
| MAP4_815 | 1.19 | 5.58E+08 | 1.70 | 5.60E+08 | 2.51 | 6.08E+08 | TTAA.AVAS | 24 | Pro-rich |
| | | | | | | | | | linker |
| MAP4_816 | 1.62 | 1.57E+08 | 2.51 | 8.83E+08 | 2.05 | 2.73E+08 | TAAA.VAST | 25 | Pro-rich |
| | | | | | | | | | linker |
| NID2_291 | 4.04 | 2.94E+09 | 3.16 | 6.67E+09 | 2.90 | 5.96E+08 | LSAA.HSSV | 26 | Linker |
| | | | | | | | | | (NIDO/EGF1) |
| SERBP1_60 | 2.10 | 2.09E+08 | 1.62 | 2.49E+08 | 0.94 | 2.20E+08 | QAAA.QTNS | 27 | N-terminal |
| | | | | | | | | | region |
| SERBP1_116 | 2.55 | 4.56E+08 | 3.25 | 7.27E+09 | 2.44 | 4.39E+09 | RPDQ.QLQG | 28 | N-terminal |
| | | | | | | | | | region |
| SRGN_59 | 4.33 | 2.70E+09 | 3.66 | 2.86E+09 | 1.17 | 2.32E+08 | PMFE.LLPG | 29 | N-term (after |
| | | | | | | | | | disulfide) |

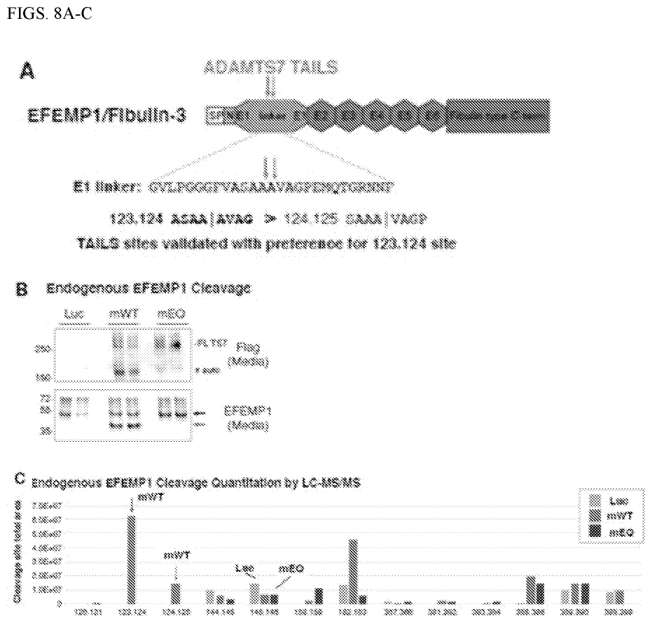

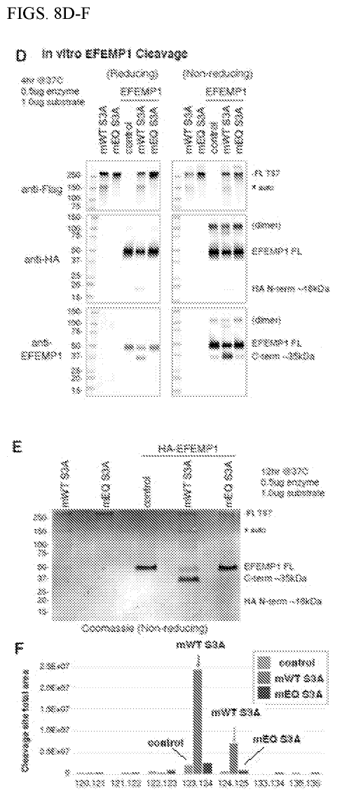

Validation of ADAMTS7 preference for EFEMP1 cleavage at adjacent sites-EFEMP1 is a secreted extracellular matrix protein with multiple EGF domains and carboxyl terminal Fibulin domain. The first EGF domain is atypical and contains an extended linker region with documented sensitivity to proteases; and is also the location of candidate ADAMTS7 123.124 and 124.125 cleavage sites (FIG. 8A). By comparison, a previous TAILS experiment with ADAMTS3 reported EFEMP1 cleavage at 122.123 and 123.124, although no EFEMP1 cleavage sites were identified in TAILS experiments for paralogs ADAMTS2 or ADAMTS14. MMP3 and MMP7 are reported to cleave EFEMP1 at 124.125, and notably there was an observed background cleavage at 123.124 within experiments from the same study. Data from the study TAILS experiments consistently showed a higher log FC and total intensities for the 123.124 site, predicting 2-3 fold more cleavage at 123.124 (ASAA|AVAG) (SEQ ID NO: 1) compared to the adjacent 124.125 (SAAA|VAGP) (SEQ ID NO: 2) site (Table 3). To verify that EFEMP1 is a substrate for ADAMTS7 and to compare the relative amount of cleavage at the 123.124 and 124.125 sites, the study performed two key validation experiments.

The study first examined the specificity of ADAMTS7 cleavage to endogenously expressed EFEMP1. HUVEC express higher levels of EFEMP1 compared to SMC, as shown by 7-8 fold higher total intensities of EFEMP1 spectra from the secretome experiments. Similar to the initial HUVEC discovery experiment, the study expressed full-length mouse ADAMTS7 WT and EQ using adenovirus and examined the concentrated media. As expected ADAMTS7 WT migrated at two bands matching the observed autocleavage event in the mucin domain (FIG. 8B). Using an antibody to an EFEMP1 epitope c-terminal to the candidate ADAMTS7 cleavage sites, the study detected a single lower mobility band consistent with cleavage within the atypical first EGF domain. Mass spectrometry identification of the cut bands digested with trypsin or chymotrypsin identified semi-tryptic and semi-chymotryptic peptides indicating cleavage at 123.124 and 124.125 sites from the Ad-ADAMTS7 WT sample. Importantly, these sites were not detected in the Luc or EQ samples while the study methods were sensitive enough to identify cleavage outside of the first EGF repeat in all samples which were presumably independent of ADAMTS7 activity. Using the area for each of these observed spectra cumulatively, the study assigned a relative abundance to each of these detected cleavage products and observed a preference for 123.124 over 124.125 (FIG. 8 (′).

Next, the study analyzed the site and preference of EFEMP1 cleavage by ADAMTS7 in a binary in vitro system. The study obtained commercially purified full-length epitope tagged HA-EFEMP1 and combined it with the study purified full-length mouse ADAMTS7 S3A WT or ADAMTS7 S3A EQ for 4 hours at 37° C. Mobility of EFEMP1 was detected by western blot using samples in reducing or non-reducing conditions. Full-length HA-EFEMP1 was detected at the expected position and a lower band was detected matching the predicted HA tagged amino terminus after cleavage by ADAMTS7 in the atypical first EGF domain (FIG. 8D). The antibody to the EFEMP1 carboxyl terminus provided a stronger signal under non-reducing compared to reducing conditions. Here the study could detect an abundant lower band at 40 kDa consistent with ADAMTS7 cleavage under reducing and non-reducing conditions. A faint band at this position was also present in the no enzyme and ADAMTS7 EQ control reactions under non-reducing conditions raising the possibility of background cleavage from the commercial EFEMP1. The study performed a second experiment with digestion for 12 hours and examined the products by Coomassie staining (FIG. 8E). In the WT treated condition the study could detect two lower bands matching to the amino and carboxyl terminal fragments of EFEMP1, consistent with ADAMTS7 cleavage restricted to the atypical first EGF domain. Corresponding bands from this gel were analyzed by mass spectrometry to investigate the relative abundance of these events. Here the study identified the suspected background cleavage under conditions with no enzyme or with ADAMTS7 S3A EQ treatment, however these occurrences were relatively minor compared to the activity stimulated by ADAMTS7 S3A WT (FIG. 8F and FIG. 15). From this binary in vitro system, the study were able to demonstrate ADAMTS7 mediated cleavage of EFEMP1 in the atypical first EGF domain and confirm a preference for 123.124 over 124.125 cleavage.

In total, the study detection and quantitation of both endogenous and purified EFEMP1 are consistent with the findings from three independent TAILS discovery experiments to identify new ADAMTS7 substrates.

TABLE 4
Annotated ADAMTS7 auto-cleavage sites from each TAILS experiment

| | | | | SMC1_ | | | | | | SMC2_ |
|---|---|---|---|---|---|---|---|---|---|---|
| | | | SEQ | adj.P.Val. | SMC1_ | SMC1_ | | | SEQ | adj.P.Val. |
| ATS7_ | previous_ | SMC1_ | ID | mWT. | logFC.mWT. | total | previous_ | SMC2_ | ID | mWT.over. |
| StartAA | aa | sequence | NO: | over.mEQ | over.mEQ | Intensity | aa | sequence | NO: | mEQ |
| 65 | (T) | TQASSAFYQLQYQ | 30 | 1.33E−03 | 1.31 | 1.21E+09 | | | | |
| | | GR | | | | | | | | |
| 66 | (T) | QASSAFYQLQYQG | 31 | 6.85E−05 | 2.52 | 1.91E+08 | (T) | QASSAFYQ | 31 | 1.07E-04 |
| | | R | | | | | | LQYQGR | | |
| 67 | (Q) | ASSAFYQLQYQGR | 32 | 4.16E−05 | 2.18 | 3.56E+08 | | | | |
| 68 | (A) | SSAFYQLQYQGR | 33 | 1.29E−03 | 2.58 | 2.09E+10 | (A) | SSAFYQLQ | 33 | 2.80E-03 |
| | | | | | | | | YQGR | | |
| 70 | | | | | | | (S) | AFYQLQY | 74 | 7.27E-05 |
| | | | | | | | | QGR | | |
| 71 | (A) | FYQLQYQGR | 34 | 1.11E−05 | 4.63 | 2.79E+09 | (A) | FYQLQYQ | 34 | 9.03E-03 |
| | | | | | | | | GR | | |
| 72 | (F) | YQLQYQGR | 35 | 4.22E−05 | 2.04 | 3.36E+09 | (F) | YQLQYQG | 35 | 1.07E-04 |
| | | | | | | | | R | | |
| 73 | (Y) | QLQYQGR | 36 | 3.26E−04 | 1.31 | 7.50E+08 | (Y) | QLQYQGR | 36 | 2.88E-02 |
| 74 | | | | | | | (Q) | LQYQGR | 75 | 6.32E-05 |
| 90 | (P) | YLmAPGFVSEIR | 37 | 9.71E−03 | 1.53 | 3.02E+08 | (P) | YLMAPGF | 76 | 8.76E-04 |
| | | | | | | | | VS | | |
| 91 | (Y) | LMAPGFVSEIR | 38 | 7.30E−05 | 2.91 | 6.29E+09 | (Y) | LmAPGFVS | 77 | 4.91E-02 |
| 103 | | | | | | | (R) | HSTLGHA | 78 | 2.24E-02 |
| | | | | | | | | HIQTSVPT | | |
| | | | | | | | | CHLLG | | |
| 108 | | | | | | | (G) | HAHIQTSV | 79 | 8.39E-04 |
| | | | | | | | | PTCHLLG | | |
| 109 | (H) | AHIQTSVPTCHLLG | 39 | 1.11E−03 | 1.18 | 2.28E+09 | | | | |
| | | DVQDPELEGGFAA | | | | | | | | |
| | | ISACDGLR | | | | | | | | |
| 110 | (A) | HIQTSVPTCHLLGD | 40 | 4.22E−05 | 2.03 | 2.72E+09 | (A) | HIQTSVPT | 80 | 1.27E-03 |
| | | VQDPELEGGFAAIS | | | | | | CHLLG | | |
| | | ACDGLR | | | | | | | | |
| 111 | (H) | IQTSVPTCHLLGDV | 41 | 9.46E−03 | 0.65 | 2.75E+09 | | | | |
| | | QDPELEGGFAAISA | | | | | | | | |
| | | CDGLR | | | | | | | | |
| 112 | | | | | | | | | | |
| 123 | (G) | DVQDPELEGGFAA | 42 | 5.85E−03 | 0.89 | 2.40E+08 | | | | |
| | | ISACDGLR | | | | | | | | |
| 133 | (G) | FAAISACDGLR | 43 | 1.30E−04 | 3.55 | 4.75E+08 | | | | |
| 136 | (A) | ISACDGLR | 44 | 5.60E−06 | 3.71 | 8.04E+08 | | | | |
| 145 | | | | | | | (G) | VFQLSNE | 81 | 3.31E-04 |
| 146 | | | | | | | (V) | FQLSNE | 82 | 1.14E-04 |
| 154 | (Y) | FIEPLDGVSAQPGH | 45 | 4.56E−03 | 1.50 | 3.79E+08 | | | | |
| | | AQPHVVYKHQGSR | | | | | | | | |
| 158 | (P) | LDGVSAQPGHAQP | 46 | 5.13E−05 | 4.31 | 1.30E+10 | | | | |
| | | HVVYKHQGSR | | | | | | | | |
| 161 | (G) | VSAQPGHAQPHVV | 47 | 3.14E−04 | 3.10 | 2.12E+08 | | | | |
| | | YKHQGSR | | | | | | | | |
| 163 | | | | | | | (S) | AQPGHAQ | 83 | 5.38E-05 |
| | | | | | | | | PHVVYKH | | |
| | | | | | | | | QGSR | | |
| 164 | (A) | QPGHAQPHVVYK | 48 | 1.13E−04 | 2.68 | 6.78E+08 | (A) | QPGHAQP | 48 | 7.72E-05 |
| | | HQGSR | | | | | | HVVYKHQ | | |
| | | | | | | | | GSR | | |
| 166 | | | | | | | (P) | GHAQPHV | 84 | 4.65E-05 |
| | | | | | | | | VYKHQGS | | |
| | | | | | | | | R | | |
| 171 | (P) | HVVYKHQGSR | 49 | 1.62E−03 | 8.26 | 8.84E+10 | (P) | HVVYKHQ | 49 | 6.64E-04 |
| | | | | | | | | GSR | | |
| 205 | (Q) | qREHWEQQQQKR | 50 | 6.15E−05 | 3.44 | 1.45E+08 | | | | |
| 443 | | | | | | | | | | |
| 446 | | | | | | | (P) | SVLPGVLY | 85 | 1.55E-02 |
| 601 | (K) | LHKWVPVPNDDNP | 51 | 1.12E−03 | 1.12 | 2.61E+08 | | | | |
| | | CELHCR | | | | | | | | |
| 633 | (A) | VVDGTPCYQSR | 52 | 6.42E−05 | 3.40 | 4.60E+08 | | | | |
| 689 | | | | | | | (T) | FKETEGQG | 86 | 4.65E-05 |
| | | | | | | | | YV | | |
| 691 | (K) | ETEGQGYVDIGLIP | 53 | 5.91E−03 | 1.55 | 4.03E+07 | | | | |
| | | AGAR | | | | | | | | |
| 695 | (G) | QGYVDIGLIPAGAR | 54 | 1.13E−05 | 3.34 | 1.09E+09 | | | | |
| 698 | (Y) | VDIGLIPAGAR | 55 | 5.49E−06 | 4.97 | 5.73E+08 | | | | |
| 699 | (V) | DIGLIPAGAR | 56 | 1.93E−05 | 4.14 | 1.81E+08 | | | | |
| 715 | (E) | VAEAANFLALR | 57 | 1.22E−04 | 5.15 | 3.88E+10 | (E) | VAEAANF | 57 | 1.29E-03 |
| | | | | | | | | LALR | | |
| 717 | (A) | EAANFLALR | 58 | 1.13E−05 | 3.41 | 1.87E+08 | | | | |
| 718 | | | | | | | (E) | AANFLALR | 87 | 4.00E-04 |
| 719 | (A) | ANFLALR | 59 | 1.45E−04 | 4.76 | 1.09E+08 | (A) | ANFLALR | 59 | 4.65E-05 |
| 733 | | | | | | | | | | |
| 734 | (F) | LNGGWTIQWNGD | 60 | 4.40E−04 | 3.70 | 1.01E+09 | (F) | LnGGWTIQ | 88 | 8.39E-04 |
| | | YR | | | | | | WNG | | |
| 738 | | | | | | | (G) | WTIQWNG | 89 | 4.65E-05 |
| 764 | | | | | | | (N) | LTSPGPTS | 90 | 7.68E-03 |
| 781 | (F) | QEKNPGVHYQYTI | 61 | 8.61E−04 | 1.93 | 2.83E+09 | (F) | qEKNPGVH | 61 | 1.51E-04 |
| | | QR | | | | | | YQYTIQR | | |
| 788 | | | | | | | (V) | HYQYTIQR | 91 | 6.72E-03 |
| 790 | | | | | | | (Y) | QYTIQR | 92 | 8.80E-05 |
| 809 | (W) | HYGPWSKCTVTCG | 62 | 5.18E−04 | 2.88 | 1.61E+08 | (W) | HYGPWSK | 62 | 7.93E-04 |
| | | TGVQR | | | | | | CTVTCGTG | | |
| | | | | | | | | VQR | | |
| 813 | | | | | | | (P) | WSKCTVT | 93 | 6.99E-05 |
| | | | | | | | | CGTGVQR | | |
| 842 | (E) | EYCNTLNRPDER | 63 | 1.90E−04 | 1.40 | 1.27E+08 | (E) | EYCNTLNR | 94 | 1.66E-02 |
| | | | | | | | | P | | |
| 843 | (E) | YCNTLNRPDER | 64 | 1.56E−04 | 3.03 | 2.61E+08 | (E) | YCNTLNRP | 95 | 7.49E-05 |
| 847 | (T) | LNRPDER | 65 | 3.85E−05 | 3.04 | 1.55E+08 | | | | |
| 902 | (A) | LELSACEHLPRPLA | 66 | 2.62E−03 | 0.87 | 5.55E+08 | | | | |
| | | ETPCNR | | | | | | | | |
| 904 | (E) | LSACEHLPR | 67 | 1.30E−04 | 2.56 | 3.84E+08 | | | | |
| 914 | | | | | | | | | | |
| 999 | (L) | FNEVDFIPNQLAPR | 68 | 1.13E−04 | 3.72 | 8.85E+08 | | | | |
| 1008 | | | | | | | (N) | QLAPR | 96 | 1.41E-04 |
| 1016 | | | | | | | (P) | ASSPKPVSI | 97 | 2.40E-04 |
| | | | | | | | | SNAI | | |
| 1022 | | | | | | | (P) | VSISNAI | 98 | 1.07E-02 |
| 1062 | | N/A Trysin digest | | | | | (S) | FEEPHP | 99 | 6.22E-03 |
| | | only | | | | | | | | |
| 1062 | | N/A Trysin digest | | | | | (S) | FEEPHPDL | 100 | 2.31E-03 |
| | | only | | | | | | V | | |
| 1114 | | | | | | | (P) | LLSEASYS | 101 | 6.72E-03 |
| | | | | | | | | PPGL | | |
| 1119 | | | | | | | (A) | SYSPPGLE | 102 | 5.56E-04 |
| | | | | | | | | QTSINPLA | | |
| | | | | | | | | NFLT | | |
| 1119 | | | | | | | (A) | SYSPPGLE | 103 | 1.93E-03 |
| | | | | | | | | QTSINPLA | | |
| | | | | | | | | NFLTEE | | |
| 1133 | | | | | | | (P) | LANFLTEE | 104 | 7.68E-03 |
| 1148 | | | | | | | (P) | ELGFPSLP | 105 | 1.10E-03 |
| | | | | | | | | WPPASVD | | |
| 1176 | (E) | LLVKEDEQSPPSTP | 69 | 3.05E−04 | 1.87 | 1.29E+08 | | | | |
| | | WSDR | | | | | | | | |
| 1308 | (T) | LTMPGTLLLTVPT | 70 | 3.71E−05 | 14.14 | 5.34E+06 | | | | |
| | | DLR | | | | | | | | |
| 1312 | | | | | | | (P) | GTLLLTVP | 106 | 2.00E-03 |
| | | | | | | | | T | | |
| 1316 | (L) | LTVPTDLR | 71 | 6.78E−05 | 2.34 | 7.35E+08 | | | | |
| 1365 | (P) | LQPSLEEDGDPADP | 72 | 2.28E−05 | 2.71 | 3.62E+09 | | | | |
| | | LPAR | | | | | | | | |
| 1549 | | | | | | | | | | |
| 1584 | | | | | | | (S) | HEAWPESS | 107 | 3.03E-03 |
| | | | | | | | | RPCATE | | |
| 1601 | (E) | LVEPPR | 73 | 1.52E−04 | 5.85 | 6.57E+09 | (E) | LVEPPR | 73 | 4.64E-05 |

| | | | | | | HUVEC_ | HUVEC_ | | |
|---|---|---|---|---|---|---|---|---|---|
| | SMC2_ | SMC2_ | | | SEQ | adj.P.Val. | logFC. | HUVEC_ | |
| ATS7_ | logFC.mWT. | total | previous_ | HUVEC_ | ID | mWT.over. | mWT.over. | total | ADAMTS7_ |
| StartAA | over.mEQ | Intensity | aa | sequence | NO: | mEQ | mEQ | Intensity | subdomain |
| 65 | | | | | | | | | Prodomain |
| 66 | 2.72 | 3.71E+07 | | | | | | | Prodomain |
| 67 | | | | | | | | | Prodomain |
| 68 | 2.53 | 1.57E+10 | (A) | SSAFYQLQ | 33 | 3.25E−04 | 3.71 | 5.15E+09 | Prodomain |
| | | | | YQGR | | | | | |
| 70 | 2.68 | 1.06E+08 | | | | | | | Prodomain |
| 71 | 2.88 | 7.66E+09 | (A) | FYQLQYQG | 34 | 4.09E−04 | 2.66 | 3.40E+09 | Prodomain |
| | | | | R | | | | | |
| 72 | 2.24 | 6.07E+08 | (F) | YQLQYQGR | 35 | 1.35E−03 | 1.73 | 8.20E+08 | Prodomain |
| 73 | 1.00 | 7.09E+10 | | | | | | | Prodomain |
| 74 | 3.35 | 6.42E+09 | | | | | | | Prodomain |
| 90 | 1.41 | 4.72E+09 | (P) | YLmAPGFV | 76 | 6.36E−03 | 2.40 | 1.55E+09 | Prodomain |
| | | | | S | | | | | |
| 91 | 1.06 | 7.54E+09 | (Y) | LmAPGFVS | 77 | 7.18E−04 | 3.03 | 1.43E+09 | Prodomain |
| 103 | 0.85 | 1.61E+09 | | | | | | | Prodomain |
| 108 | 2.00 | 1.03E+09 | | | | | | | Prodomain |
| 109 | | | (H) | AHIQTSVPT | 108 | 2.49E−03 | 1.76 | 5.51E+09 | Prodomain |
| | | | | CHLLG | | | | | |
| 110 | 1.94 | 1.36E+10 | (A) | HIQTSVPTC | 80 | 3.57E−04 | 2.71 | 1.36E+10 | Prodomain |
| | | | | HLLG | | | | | |
| 111 | | | (H) | IQTSVPTCH | 109 | 8.11E−03 | 1.97 | 1.69E+09 | Prodomain |
| | | | | LLG | | | | | |
| 112 | | | (I) | qTSVPTCHL | 110 | 2.74E−03 | 1.52 | 7.99E+07 | Prodomain |
| | | | | LG | | | | | |
| 123 | | | | | | | | | Prodomain |
| 133 | | | (G) | FAAISACD | 43 | 1.26E−03 | 2.42 | 4.62E+07 | Prodomain |
| | | | | GLR | | | | | |
| 136 | | | | | | | | | Prodomain |
| 145 | 2.25 | 1.40E+09 | | | | | | | Prodomain |
| 146 | 3.00 | 1.32E+09 | | | | | | | Prodomain |
| 154 | | | | | | | | | Prodomain |
| 158 | | | | | | | | | Prodomain |
| 161 | | | | | | | | | Prodomain |
| 163 | 3.56 | 6.46E+08 | (S) | AQPGHAQP | 83 | 1.20E−02 | 1.43 | 1.83E+08 | Prodomain |
| | | | | HVVYKHQ | | | | | |
| | | | | GSR | | | | | |
| 164 | 2.58 | 3.00E+09 | | | | | | | Prodomain |
| 166 | 4.67 | 2.20E+09 | | | | | | | Prodomain |
| 171 | 4.26 | 2.22E+11 | (P) | HVVYKHQ | 49 | 1.35E−03 | 5.49 | 1.03E+11 | Prodomain |
| | | | | GSR | | | | | |
| 205 | | | | | | | | | Prodomain |
| 443 | | | (I) | ALPSVLPG | 111 | 9.71E−03 | 1.98 | 4.83E+07 | Metallopro |
| | | | | VLY | | | | | teinase |
| 446 | 2.05 | 8.56E+08 | (P) | SVLPGVLY | 85 | 5.51E−03 | 2.28 | 1.74E+08 | Metallopro |
| | | | | | | | | | teinase |
| 601 | | | | | | | | | Cysteine− |
| | | | | | | | | | rich |
| 633 | | | | | | | | | Cysteine− |
| | | | | | | | | | rich |
| 689 | 4.17 | 1.78E+10 | (T) | FKETEGQG | 86 | 2.14E−04 | 5.11 | 1.37E+10 | Spacer |
| | | | | YV | | | | | |
| 691 | | | | | | | | | Spacer |
| 695 | | | | | | | | | Spacer |
| 698 | | | | | | | | | Spacer |
| 699 | | | | | | | | | Spacer |
| 715 | 2.46 | 1.22E+08 | (E) | VAEAAnFL | 57 | 4.09E−04 | 2.57 | 5.69E+07 | Spacer |
| | | | | ALR | | | | | |
| 717 | | | | | | | | | Spacer |
| 718 | 4.47 | 1.97E+10 | (E) | AANFLALR | 87 | 3.57E−04 | 3.34 | 2.48E+08 | Spacer |
| 719 | 4.41 | 5.37E+08 | | | | | | | Spacer |
| 733 | | | (Y) | FLnGGWTI | 112 | 1.19E−02 | 2.32 | 1.74E+07 | Spacer |
| | | | | QWNG | | | | | |
| 734 | 3.22 | 2.60E+09 | (F) | LnGGWTIQ | 88 | 1.16E−03 | 2.86 | 7.76E+08 | Spacer |
| | | | | WnG | | | | | |
| 738 | 4.20 | 5.12E+08 | (G) | WTIQWNG | 89 | 1.29E−03 | 3.23 | 1.43E+08 | Spacer |
| 764 | 1.24 | 1.19E+09 | (N) | LTSPGPTS | 90 | 9.25E−04 | 2.76 | 9.10E+08 | Spacer |
| 781 | 2.60 | 3.00E+10 | (F) | qEKNPGVH | 61 | 4.09E−04 | 2.90 | 1.40E+10 | Spacer |
| | | | | YQYTIQR | | | | | |
| 788 | 1.49 | 4.67E+08 | (V) | HYQYTIQR | 91 | 5.50E−04 | 2.36 | 2.29E+08 | Spacer |
| 790 | 3.34 | 1.63E+10 | (Y) | QYTIQR | 92 | 2.16E−03 | 1.85 | 1.33E+10 | Spacer |
| 809 | 2.89 | 2.30E+08 | | | | | | | TSR2 |
| 813 | 4.81 | 1.19E+09 | | | | | | | TSR2 |
| 842 | 0.95 | 7.07E+08 | | | | | | | TSR2 |
| 343 | 2.73 | 4.30E+08 | | | | | | | TSR2 |
| 847 | | | | | | | | | TSR2 |
| 902 | | | | | | | | | TSR3 |
| 904 | | | | | | | | | TSR3 |
| 914 | | | (P) | LAETPCNR | 113 | 7.18E−04 | 3.51 | 4.99E+07 | TSR3 |
| 999 | | | | | | | | | Mucin |
| 1008 | 5.69 | 7.81E+09 | | | | | | | Mucin |
| 1016 | 3.14 | 3.32E+08 | (P) | ASSPKPVSI | 97 | 4.97E−03 | 2.97 | 8.59E+07 | Mucin |
| | | | | SNAI | | | | | |
| 1022 | 2.51 | 9.66E+08 | | | | | | | Mucin |
| 1062 | 1.86 | 3.41E+11 | (S) | FEEPHP | 99 | 3.97E−04 | 2.21 | 2.28E+11 | Mucin |
| 1062 | 1.99 | 3.10E+09 | (S) | FEEPHPDL | 100 | 8.96E−04 | 2.86 | 1.10E+09 | Mucin |
| | | | | V | | | | | |
| 1114 | 1.55 | 9.05E+08 | | | | | | | Mucin |
| 1119 | 2.37 | 5.50E+07 | | | | | | | Mucin |
| 1119 | 2.51 | 3.76E+08 | | | | | | | Mucin |
| 1133 | 1.63 | 9.75E+07 | | | | | | | Mucin |
| 1148 | 1.93 | 7.61E+09 | (P) | ELGFPSLP | 114 | 6.23E−04 | 3.40 | 9.85E+08 | Mucin |
| | | | | WPPASV | | | | | |
| 1176 | | | | | | | | | Mucin |
| 1308 | | | (T) | LTmPGTLL | 115 | 1.19E−02 | 1.84 | 1.37E+08 | Mucin |
| | | | | LTVPT | | | | | |
| 1312 | 1.36 | 5.60E+08 | | | | | | | Mucin |
| 1316 | | | | | | | | | Mucin |
| 1365 | | | (P) | LQPSLEED | 116 | 2.79E−03 | 2.12 | 5.06E+08 | Mucin |
| | | | | GDPA | | | | | |
| 1549 | | | (P) | WGQCSAPC | 117 | 1.16E−03 | 2.96 | 2.95E+06 | TSR8 |
| | | | | GGGVQR | | | | | |
| 1584 | 1.56 | 4.80E+08 | (S) | HEAWPESS | 107 | 4.18E−03 | 1.30 | 7.60E+07 | TSR8 |
| | | | | RPCATE | | | | | |
| 1601 | 7.79 | 2.98E+10 | (E) | LVEPPR | 73 | 3.97E−04 | 3.34 | 5.42E+10 | PLAC |

TABLE_5
TAILS high confidence candidate substrate cleavage sites for each experiment
and annotated overlap analysis of cleavage sites
identified in multiple TAILS experiments

| | | SEQ | adj.P.Val. | adj.P. | adj.P. | logFC. | logFC. | logFC. |
|---|---|---|---|---|---|---|---|---|
| | | ID | mEQ.over. | Val.mWT. | Val.mWT. | mEQ. | mWT. | mWT. |
| TAILS_id | id | NO: | Luc | over.Luc | over.mEQ | over.Luc | over.Lux | over.mEQ |
| ACTB_8 | LVVDNGSGMCK | 118 | 7.65E−01 | 3.90E−05 | 3.82E−05 | −0.13 | 3.45 | 3.57 |
| | AGFAGDDAPR | | | | | | | |
| ACTB_21 | FAGDDAPR | 119 | 8.12E−01 | 3.39E−03 | 4.05E−04 | −0.08 | 1.09 | 1.17 |
| ACTB_105 | LTEAPLNPKANR | 120 | 4.83E−01 | 1.05E−04 | 1.15E−04 | −0.12 | 1.88 | 2.00 |
| ACTG1_8 | LVIDNGSGMCK | 121 | 8.43E−01 | 5.18E−04 | 5.79E−03 | −0.08 | 1.39 | 1.48 |
| | AGFAGDDAPR | | | | | | | |
| ADAM9_69 | VIQAEGKEHIIHL | 122 | 6.02E−01 | 8.48E−05 | 2.32E−04 | −0.10 | 1.45 | 1.55 |
| | ER | | | | | | | |
| AEBP1_37 | FLEGFLSELEPEPR | 123 | 7.76E−01 | 1.61E−04 | 1.29E−04 | −0.18 | 3.23 | 3.41 |
| APLP2_579 | ISETPVDVR | 124 | 1.09E−01 | 1.44E−05 | 3.71E−05 | 0.27 | 2.27 | 2.00 |
| ATP8B3_1266 | QGTILR | 125 | 6.93E−02 | 1.10E−04 | 1.71E−05 | 1.03 | 4.08 | 3.05 |
| BCO1_29 | QGTLLR | 126 | 6.93E−02 | 1.10E−04 | 1.71E−05 | 1.03 | 4.08 | 3.05 |
| CCDC80_66 | LEEPNLQPLQR | 127 | 7.75E−01 | 1.14E−05 | 1.05E−04 | 0.14 | 3.84 | 3.70 |
| CDC37_43 | FQKEKEELDR | 128 | 6.53E−01 | 1.92E−04 | 1.47E−04 | −0.12 | 1.75 | 1.88 |
| CDH11_213 | QTGIIR | 129 | 6.93E−02 | 1.10E−04 | 1.71E−05 | 1.03 | 4.08 | 3.05 |
| CDH11_467 | HQEAKVPVAIR | 130 | 9.56E−01 | 9.19E−04 | 8.37E−04 | −0.03 | 2.02 | 2.06 |
| CLEC11A_50 | HLQEALGLPAGR | 131 | 1.73E−01 | 5.83E−04 | 1.07E−03 | −0.34 | 1.08 | 1.42 |
| COL16A1_269 | qSEGKVYTR | 132 | 1.38E−01 | 6.57E−05 | 1.01E−05 | −0.69 | 3.93 | 4.63 |
| COL18A1_1505 | LQPPVVQLHDSN | 133 | 9.64E−01 | 2.91E−04 | 1.96E−04 | −0.01 | 1.41 | 1.42 |
| | PYPR | | | | | | | |
| COL1A2_26 | QEETVR | 134 | 8.38E−01 | 1.42E−03 | 1.37E−03 | 0.05 | 0.98 | 0.92 |
| COL1A2_80 | qYDGKGVGLGP | 135 | 3.24E−01 | 1.44E−05 | 3.85E−05 | 0.19 | 2.55 | 2.36 |
| | GPMGLmGPR | | | | | | | |
| COL1A2_91 | GPMGLMGPR | 136 | 8.43E−01 | 2.85E−03 | 1.54E−03 | −0.07 | 1.09 | 1.16 |
| COL1A2_113 | FQGPAGEPGEPG | 137 | 1.35E−02 | 4.84E−05 | 3.85E−05 | −0.61 | 1.66 | 2.27 |
| | QTGPAGAR | | | | | | | |
| COL1A2_270 | VGNAGPAGPAGPR | 138 | 3.65E−01 | 1.73E−03 | 2.77E−05 | 0.70 | 3.51 | 2.82 |
| COL1A2_305 | LTGAKGAAGLP | 139 | 4.51E−01 | 9.19E−05 | 2.40E−04 | −0.16 | 1.46 | 1.63 |
| | GVAGAPGLPGPR | | | | | | | |
| COL1A2_389 | SAGPPGPPGLR | 140 | 7.08E−02 | 3.89E−04 | 3.69E−03 | 0.39 | 1.12 | 0.72 |
| COL1A2_425 | ASGPAGVR | 141 | 3.18E−01 | 2.52E−04 | 2.36E−04 | −0.17 | 1.17 | 1.34 |
| COL1A2_630 | VGTAGPSGPSGL | 142 | 2.51E−01 | 4.22E−04 | 4.27E−04 | −0.27 | 1.18 | 1.45 |
| | PGER | | | | | | | |
| COL1A2_681 | VGAPGPAGATGDR | 143 | 8.04E−02 | 1.55E−04 | 1.04E−04 | −0.34 | 1.27 | 1.60 |
| COL1A2_731 | AAGQPGAKGER | 144 | 4.80E−01 | 3.30E−03 | 6.19E−03 | −0.18 | 0.84 | 1.03 |
| COL1A2_866 | LLGAPGILGLPGSR | 145 | 8.39E−01 | 1.99E−03 | 9.02E−04 | −0.24 | 3.48 | 3.72 |
| COL1A2_960 | AGAPGPHGPVGP | 146 | 4.17E−01 | 5.02E−04 | 1.71E−05 | −0.35 | 2.56 | 2.91 |
| | AGKHGNR | | | | | | | |
| COL1A2_1028 | LQGLPGIAGHHG | 147 | 2.46E−01 | 2.05E−04 | 1.30E−04 | −0.28 | 1.51 | 1.79 |
| | DQGAPGSVGPA | | | | | | | |
| | GPR | | | | | | | |
| COL1A2_1037 | HHGDQGAPGSV | 148 | 1.73E−01 | 4.82E−04 | 4.40E−04 | −0.79 | 2.30 | 3.09 |
| | GPAGPR | | | | | | | |
| COL1A2_1127 | SLRPKDYEVDAT | 149 | 1.80E−01 | 2.82E−04 | 4.59E−04 | −0.45 | 2.23 | 2.68 |
| | LKSLNNQIETLLT | | | | | | | |
| | PEGSR | | | | | | | |
| COL4A2_71 | YNGPPGLQGFPG | 150 | 1.63E−01 | 9.07E−05 | 1.55E−04 | −0.37 | 1.77 | 2.14 |
| | LQGR | | | | | | | |
| COL4A2_172 | YALPKEER | 151 | 1.88E−01 | 9.07E−05 | 1.17E−05 | 0.76 | 5.97 | 5.21 |
| COL5A1_544 | SQAQAILQQAR | 152 | 3.86E−01 | 6.07E−03 | 1.17E−03 | −0.21 | 0.87 | 1.08 |
| COL8A1_90 | MKEIQPAPR | 153 | 1.72E−01 | 4.38E−04 | 1.69E−04 | −0.25 | 1.10 | 1.35 |
| DNAJA4_12 | ISALTR | 154 | 5.26E−01 | 4.71E−05 | 1.30E−04 | 0.12 | 2.05 | 1.92 |
| ECM1_43 | YAAPPSPPLSR | 155 | 8.64E−01 | 4.44E−03 | 6.46E−03 | −0.04 | 0.63 | 0.68 |
| ECM1_134 | FGDQSHPEPESW | 156 | 5.21E−01 | 2.94E−04 | 3.85E−05 | 0.28 | 3.02 | 2.74 |
| | NAAQHCQQDR | | | | | | | |
| EFEMP1_124 | AVAGPEMQTGR | 157 | 1.60E−01 | 6.06E−05 | 6.12E−06 | −0.59 | 3.84 | 4.43 |
| EFEMP1_125 | VAGPEMQTGR | 158 | 2.93E−01 | 6.42E−05 | 2.54E−04 | −0.33 | 1.92 | 2.25 |
| EFEMP1_135 | NNFVIR | 159 | 8.29E−01 | 3.47E−04 | 9.80E−04 | 0.06 | 1.43 | 1.37 |
| EFHD2_67 | QGIGEPQSPSR | 160 | 7.23E−01 | 5.72E−03 | 2.07E−03 | −0.09 | 0.81 | 0.90 |
| EHBP1L1_298 | TAPTPAPR | 161 | 2.07E−01 | 3.31E−03 | 2.94E−03 | −0.22 | 1.01 | 1.23 |
| FBLN2_259 | ALGPPAPVQAKAR | 162 | 9.90E−01 | 3.74E−03 | 5.18E−03 | −0.01 | 1.29 | 1.29 |
| FBLN2_260 | LGPPAPVQAKAR | 163 | 2.79E−01 | 4.93E−05 | 4.40E−04 | −0.74 | 3.46 | 4.21 |
| FBN1_29 | LEAGNVKETR | 164 | 6.58E−01 | 4.71E−05 | 5.30E−06 | 0.21 | 4.70 | 4.49 |
| FBN1_53 | LKGPNVCGSR | 165 | 9.34E−01 | 1.81E−05 | 1.17E−05 | −0.04 | 4.89 | 4.93 |
| FLNA_612 | SVEGPSQAKIEC | 166 | 3.84E−01 | 3.23E−04 | 9.02E−04 | 0.19 | 1.87 | 1.69 |
| | DDKGDGSCDVR | | | | | | | |
| FLNA_695 | FTVDAKHGGKA | 167 | 5.54E−01 | 7.17E−05 | 6.85E−05 | −0.25 | 4.79 | 5.04 |
| | PLR | | | | | | | |
| FLNA_1285 | LTQTGGPHVKAR | 168 | 9.24E−01 | 1.10E−04 | 7.08E−05 | −0.11 | 4.95 | 5.06 |
| FN1_36 | MVQPQSPVAVS | 169 | 3.34E−01 | 1.11E−04 | 1.30E−04 | 0.39 | 3.73 | 3.34 |
| | QSKPGCYDnGKH | | | | | | | |
| | YQINQQWER | | | | | | | |
| FN1_40 | qSPVAVSQSKPG | 170 | 5.88E−01 | 4.71E−05 | 4.40E−04 | −0.40 | 3.87 | 4.27 |
| | CYDnGKHYQINQ | | | | | | | |
| | QWER | | | | | | | |
| FN1_45 | VSQSKPGCYDN | 171 | 1.10E−01 | 8.00E−05 | 3.63E−04 | 0.49 | 3.70 | 3.20 |
| | GKHYQINQQWER | | | | | | | |
| FN1_47 | qSKPGCYDnGKH | 172 | 4.91E−01 | 5.87E−05 | 2.86E−04 | 0.19 | 3.08 | 2.89 |
| | YQINQQWER | | | | | | | |
| FN1_279 | TSSGSGPFTDVR | 173 | 5.66E−01 | 2.86E−05 | 4.60E−04 | 0.17 | 2.15 | 1.98 |
| FN1_281 | SGSGPFTDVR | 174 | 5.24E−02 | 3.59E−05 | 1.05E−04 | 0.56 | 2.44 | 1.88 |
| FN1_282 | GSGPFTDVR | 175 | 8.65E−01 | 2.22E−04 | 2.32E−04 | −0.15 | 3.74 | 3.89 |
| FN1_886 | NQESTPVVIQQE | 176 | 1.19E−01 | 1.45E−04 | 3.92E−03 | 0.49 | 1.70 | 1.21 |
| | TTGTPR | | | | | | | |
| FN1_1143 | LTPGVEYVYTIQ | 177 | 9.92E−01 | 2.76E−03 | 3.01E−03 | 0.00 | 0.85 | 0.86 |
| | VLR | | | | | | | |
| FN1_1656 | SSSPVTGYR | 178 | 6.76E−01 | 1.90E−04 | 2.41E−04 | −0.08 | 1.44 | 1.52 |
| FN1_1715 | LVQTAVTNIDR | 179 | 5.64E−02 | 4.82E−05 | 1.78E−04 | 0.52 | 2.47 | 1.95 |
| FN1_1715 | LVQTAVTNIDRP | 180 | 1.69E−01 | 4.71E−05 | 3.71E−05 | −0.22 | 1.65 | 1.88 |
| | KGLAFTDVDVD | | | | | | | |
| | SIKIAWESPQGQ | | | | | | | |
| | VSR | | | | | | | |
| FN1_1731 | FTDVDVDSIKIA | 181 | 8.04E−01 | 8.48E−05 | 1.55E−04 | −0.11 | 2.59 | 2.70 |
| | WESPQGQVSR | | | | | | | |
| FN1_2143 | FEEHGFR | 182 | 4.56E−01 | 3.23E−04 | 2.49E−04 | −0.13 | 1.11 | 1.24 |
| FN1_2217 | FQDTSEYIISCHP | 183 | 7.81E−01 | 1.86E−04 | 4.71E−04 | 0.14 | 2.41 | 2.27 |
| | VGTDEEPLQFR | | | | | | | |
| FN1_2250 | LTGLTR | 184 | 5.26E−01 | 4.71E−05 | 1.30E−04 | 0.12 | 2.05 | 1.92 |
| GALNT2_31 | LAGGAGGGAGR | 185 | 3.70E−02 | 2.44E−03 | 2.90E−04 | −0.43 | 0.76 | 1.19 |
| GSN_35 | SQAGAPQGR | 186 | 9.66E−01 | 2.18E−04 | 4.54E−04 | 0.05 | 4.42 | 4.37 |
| GSN_413 | HIANVER | 187 | 2.54E−01 | 8.32E−05 | 1.27E−04 | −0.20 | 1.99 | 2.19 |
| HEG1_47 | LAGAGLELQLER | 188 | 8.23E−01 | 3.02E−05 | 4.16E−05 | 0.09 | 3.25 | 3.15 |
| HEG1_49 | GAGLELQLER | 189 | 5.94E−02 | 2.98E−04 | 2.07E−03 | 0.51 | 1.72 | 1.20 |
| HLA−C_52 | VDDTQFVR | 190 | 1.51E−01 | 1.66E−03 | 4.48E−03 | 0.35 | 1.14 | 0.78 |
| HSPG2_275 | LLPGSVR | 191 | 1.73E−01 | 1.95E−05 | 6.85E−05 | 0.38 | 2.87 | 2.49 |
| HSPG2_1863 | LSAPVVSIHPPQL | 192 | 4.36E−01 | 1.71E−04 | 1.45E−04 | −0.23 | 1.80 | 2.03 |
| | TVQPGQLAEFR | | | | | | | |
| HSPG2_1937 | SSAGQQVAR | 193 | 4.29E−01 | 5.65E−03 | 2.74E−03 | −0.15 | 0.79 | 0.94 |
| HSPG2_2331 | ANLAYPAGSTQPIR | 194 | 9.35E−01 | 1.13E−04 | 1.12E−04 | 0.03 | 2.07 | 2.04 |
| HSPG2_2333 | LAYPAGSTQPIR | 195 | 9.82E−01 | 2.39E−04 | 4.09E−05 | 0.01 | 1.93 | 1.92 |
| HSPG2_2688 | VADSGEYVCR | 196 | 6.19E−01 | 1.14E−05 | 4.16E−05 | 0.14 | 3.16 | 3.02 |
| HSPG2_3250 | SPLPWQHR | 197 | 9.86E−01 | 7.39E−04 | 1.18E−03 | 0.00 | 1.00 | 1.01 |
| HSPG2_3523 | SKVGGHLR | 198 | 2.47E−01 | 1.58E−03 | 1.14E−03 | −0.33 | 1.10 | 1.43 |
| HSPG2_3523 | SKVGGHLRPGIV | 199 | 3.54E−01 | 5.65E−04 | 1.10E−03 | −0.17 | 0.94 | 1.11 |
| | QSGGVVR | | | | | | | |
| HSPG2_3542 | IAHVELADAGQYR | 200 | 7.86E−01 | 8.95E−03 | 9.10E−03 | −0.07 | 0.60 | 0.67 |
| HSPG2_3647 | qGKVKAFAHLQ | 201 | 3.18E−01 | 5.64E−03 | 2.18E−04 | −0.26 | 0.96 | 1.22 |
| | VPER | | | | | | | |
| HSPG2_3672 | SFLPLPTIKDAYR | 202 | 8.42E−01 | 1.47E−03 | 3.30E−03 | 0.06 | 1.09 | 1.03 |
| HSPG2_3813 | SSGFIGCVR | 203 | 1.59E−01 | 1.26E−03 | 1.00E−03 | −0.28 | 0.81 | 1.09 |
| HSPG2_3969 | SGGKSGPVEDFV | 204 | 2.47E−01 | 5.17E−03 | 1.18E−03 | −0.28 | 0.79 | 1.08 |
| | SLAmVGGHLEFR | | | | | | | |
| HSPG2_4004 | SAEPLALGR | 205 | 7.01E−02 | 1.57E−03 | 6.87E−04 | −0.37 | 0.88 | 1.25 |
| HSPG2_4021 | LNKDGSLR | 206 | 9.23E−01 | 6.62E−03 | 9.71E−03 | −0.02 | 0.70 | 0.73 |
| HSPG2_4075 | GCVGEVSVNGKR | 207 | 9.48E−01 | 2.32E−03 | 2.62E−03 | 0.02 | 0.77 | 0.75 |
| HSPG2_4133 | DGFKGDLCEHEE | 208 | 4.06E−01 | 3.55E−03 | 2.91E−03 | −0.14 | 0.63 | 0.76 |
| | NPCQLR | | | | | | | |
| HTRA1_33 | LAAGCPDR | 209 | 8.26E−01 | 1.44E−05 | 1.90E−04 | 0.09 | 2.87 | 2.78 |
| IFI30_43 | YKTGNLYLR | 210 | 9.96E−01 | 3.06E−05 | 3.82E−05 | 0.00 | 3.19 | 3.19 |
| IGFBP3_31 | SAGLGPVVR | 211 | 9.06E−01 | 1.31E−05 | 7.08E−05 | −0.04 | 2.46 | 2.49 |
| IGFBP3_32 | AGLGPVVR | 212 | 2.28E−01 | 1.14E−05 | 5.30E−06 | 0.28 | 4.06 | 3.78 |
| IGFBP3_200 | FSSESKR | 213 | 7.56E−01 | 7.39E−05 | 2.16E−05 | −0.09 | 2.33 | 2.41 |
| IGFBP7_101 | AAGGPGVSGVC | 214 | 2.45E−01 | 9.94E−06 | 2.66E−05 | 0.29 | 3.99 | 3.69 |
| | VCKSR | | | | | | | |
| INS_32 | GSHLVEALYLVC | 215 | 6.90E−01 | 1.07E−03 | 5.79E−03 | −0.13 | 1.09 | 1.22 |
| | GER | | | | | | | |
| KPNB1_37 | LVELSR | 216 | 3.83E−01 | 3.55E−04 | 3.17E−04 | −0.25 | 1.52 | 1.77 |
| LAMA5_484 | QVLPAGQIVNCD | 217 | 9.01E−01 | 5.69E−03 | 6.08E−03 | −0.05 | 1.20 | 1.25 |
| | CSAAGTQGNACR | | | | | | | |
| LASP1_123 | HEEFEKSR | 218 | 1.21E−01 | 7.88E−03 | 2.78E−03 | −0.53 | 0.87 | 1.40 |
| LGALS1_53 | HGDANTIVCNSK | 219 | 1.22E−01 | 1.96E−04 | 4.09E−05 | 0.94 | 4.83 | 3.89 |
| | DGGAWGTEQR | | | | | | | |
| LGALS3BP_447 | FQAPSDYR | 220 | 1.39E−01 | 1.14E−05 | 3.71E−05 | 0.57 | 4.70 | 4.13 |
| LIPG_42 | qTEVKPSVR | 221 | 9.14E−01 | 1.34E−05 | 5.30E−06 | −0.03 | 3.88 | 3.91 |
| LMNA_408 | qTQGGGSVTKKR | 222 | 6.64E−01 | 1.14E−05 | 1.13E−05 | 0.14 | 4.36 | 4.22 |
| LOX_58 | LSLGSQYQPQR | 223 | 5.39E−01 | 1.46E−03 | 1.17E−03 | −0.11 | 0.88 | 0.99 |
| LOX_80 | ANASAQQPR | 224 | 4.97E−01 | 1.43E−04 | 4.16E−05 | −0.16 | 2.02 | 2.18 |
| LOX_81 | NASAQQPR | 225 | 1.08E−01 | 1.45E−04 | 6.24E−05 | −0.30 | 1.38 | 1.69 |
| LOX_123 | HWFQAGYSTSR | 226 | 4.27E−01 | 1.02E−04 | 4.03E−04 | −0.22 | 1.60 | 1.82 |
| LOX_124 | WFQAGYSTSR | 227 | 8.92E−01 | 1.24E−05 | 1.30E−04 | 0.08 | 3.70 | 3.62 |
| LOX_126 | QAGYSTSR | 228 | 9.64E−01 | 1.54E−04 | 1.52E−03 | 0.02 | 1.61 | 1.59 |
| LOXL1_219 | VASAGVIYPYQPR | 229 | 6.92E−01 | 5.38E−03 | 9.71E−03 | −0.10 | 0.99 | 1.09 |
| LOXL2_37 | FQQPAPEYHQPQ | 230 | 7.90E−01 | 3.92E−05 | 3.87E−05 | 0.09 | 3.37 | 3.28 |
| | APANVAKIQLR | | | | | | | |
| LOXL2_38 | qQPAPEYHQPQA | 231 | 6.16E−01 | 1.08E−04 | 6.68E−04 | 0.11 | 1.37 | 1.26 |
| | PANVAKIQLR | | | | | | | |
| LOXL2_318 | AYKPEQPL VR | 232 | 9.93E−01 | 1.42E−03 | 3.69E−03 | 0.00 | 0.78 | 0.78 |
| LOXL4_25 | qSLGTTKLR | 233 | 7.49E−01 | 8.78E−03 | 6.34E−03 | −0.07 | 0.68 | 0.75 |
| LTBP1_393 | LTATNFR | 234 | 6.05E−01 | 1.22E−05 | 1.71E−05 | 0.14 | 4.03 | 3.89 |
| LTBP1_494 | SVQIHQVSR | 235 | 9.85E−01 | 2.84E−04 | 1.33E−03 | −0.01 | 2.61 | 2.63 |
| LTBP1_540 | HQQVIPHVYPVA | 236 | 4.96E−01 | 7.55E−05 | 1.13E−04 | 0.13 | 2.83 | 2.70 |
| | AKTQLGR | | | | | | | |
| LTBP1_546 | HVYPVAAKTQLGR | 237 | 4.52E−01 | 4.66E−03 | 2.89E−03 | −1.05 | 3.62 | 4.67 |
| LTBP1_1597 | SEQYTPEADPYFI | 238 | 8.76E−01 | 1.26E−03 | 2.31E−03 | 0.06 | 1.16 | 1.10 |
| | QDR | | | | | | | |
| LTBP2_485 | SVQIHQVAQVR | 239 | 7.13E−02 | 2.83E−04 | 2.43E−04 | −0.42 | 1.80 | 2.23 |
| LTBP2_533 | SGEPPRPLPPAAPR | 240 | 2.75E−01 | 5.53E−04 | 6.13E−03 | 0.28 | 1.34 | 1.06 |
| LTBP2_677 | SLGPGTCTLPLAQR | 241 | 8.39E−01 | 1.63E−03 | 1.52E−03 | 0.05 | 0.90 | 0.85 |
| LTBP2_760 | SSGALPGPAER | 242 | 1.32E−01 | 5.55E−05 | 5.31E−04 | 0.29 | 1.54 | 1.24 |
| LTBP2_1023 | GKCTNLEGSFR | 243 | 8.96E−01 | 1.52E−03 | 8.56E−03 | 0.03 | 0.74 | 0.71 |
| LTBP2_1371 | SGQKGHAPCSSV | 244 | 1.21E−01 | 2.43E−04 | 3.42E−03 | 0.31 | 1.24 | 0.93 |
| | LGR | | | | | | | |
| LTBP2_1560 | TTYTECCCQDGE | 245 | 2.41E−01 | 7.53E−04 | 9.22E−03 | 0.23 | 0.94 | 0.71 |
| | AWSQQCALCPPR | | | | | | | |
| LTBP2_1584 | SSEVYAQLCNVAR | 246 | 1.92E−01 | 8.68E−04 | 1.28E−03 | 0.32 | 1.40 | 1.08 |
| LTBP2_1602 | EAGVHFR | 247 | 5.33E−02 | 2.06E−03 | 9.73E−03 | 0.64 | 1.63 | 0.99 |
| LTBP3_239 | SVQVHR | 248 | 7.55E−01 | 3.89E−04 | 4.00E−04 | −0.10 | 2.52 | 2.62 |
| LTBP4_197 | VHQVER | 249 | 1.27E−01 | 7.61E−03 | 1.11E−03 | −0.42 | 0.75 | 1.17 |
| MAN2A1_51 | LQEKIDHLER | 250 | 5.00E−01 | 4.23E−05 | 2.86E−04 | 0.18 | 2.04 | 1.86 |
| MAP1B_1065 | LTTPTKQLGAQS | 251 | 3.65E−01 | 9.88E−04 | 3.05E−04 | −0.20 | 1.11 | 1.30 |
| | PGR | | | | | | | |
| MAP4_815 | AVASTGPSSR | 252 | 6.15E−01 | 8.87E−04 | 2.67E−03 | 0.10 | 1.29 | 1.19 |
| MAP4_816 | VASTGPSSR | 253 | 3.86E−01 | 2.22E−04 | 7.15E−05 | −0.18 | 1.44 | 1.62 |
| MGAT4B_28 | LSGQKGDVVDVYQR | 254 | 4.97E−01 | 6.80E−04 | 9.38E−04 | −0.23 | 1.43 | 1.66 |
| MMP1_24 | LETQEQDVDLV | 255 | 8.20E−01 | 3.85E−04 | 1.57E−03 | −0.08 | 2.23 | 2.31 |
| | QKYLEKYYNLK | | | | | | | |
| | nDGR | | | | | | | |
| MMP2_406 | GHAmGLEHSQD | 256 | 5.57E−01 | 4.03E−05 | 4.31E−04 | 0.31 | 3.40 | 3.09 |
| | PGALMAPIYTYT | | | | | | | |
| | KnFR | | | | | | | |
| MRFAP1_39 | IASLTR | 257 | 5.26E−01 | 4.71E−05 | 1.30E−04 | 0.12 | 2.05 | 1.92 |
| MYH15_403 | nSSELVKCLIHPR | 258 | 2.54E−01 | 4.38E−04 | 2.66E−05 | 0.76 | 5.47 | 4.71 |
| NID2_291 | HSSVPLGR | 259 | 9.25E−01 | 6.95E−05 | 3.33E−04 | −0.08 | 3.96 | 4.04 |
| NID2_374 | VGGPDLKGQVE | 260 | 8.16E−01 | 3.47E−05 | 2.77E−05 | −0.14 | 4.80 | 4.94 |
| | PWDER | | | | | | | |
| NID2_467 | LEDNIGSNTEVF | 261 | 8.00E−01 | 5.73E−05 | 3.85E−05 | −0.09 | 2.58 | 2.66 |
| | TYNAANKETCE | | | | | | | |
| | HNHR | | | | | | | |
| NID2_478 | FTYNAANKETCE | 262 | 4.89E−01 | 3.47E−05 | 6.92E−05 | −0.29 | 3.81 | 4.10 |
| | HNHR | | | | | | | |
| NID2_951 | YAYPGAR | 263 | 1.90E−01 | 3.06E−05 | 7.12E−05 | −0.58 | 3.35 | 3.92 |
| NUCB1_228 | LKEVWEELDGL | 264 | 7.76E−01 | 6.95E−05 | 1.22E−04 | 0.07 | 1.70 | 1.63 |
| | DPNR | | | | | | | |
| NUCB1_317 | FLASTQR | 265 | 7.37E−01 | 9.94E−06 | 1.13E−05 | −0.11 | 4.76 | 4.87 |
| NUCB1_389 | LQQAVLHMEQR | 266 | 7.09E−01 | 5.96E−05 | 6.57E−04 | 0.15 | 2.25 | 2.09 |
| OS9_54 | qSQSSDVVIVSSK | 267 | 7.76E−01 | 1.34E−04 | 1.01E−04 | −0.14 | 2.67 | 2.81 |
| | YKQR | | | | | | | |
| OS9_55 | SQSSDVVIVSSK | 268 | 8.07E−01 | 2.31E−04 | 2.60E−04 | −0.06 | 2.23 | 2.29 |
| | YKQR | | | | | | | |
| PAPPA_41 | AAGPATCATR | 269 | 3.17E−01 | 1.14E−05 | 9.22E−05 | 0.35 | 3.83 | 3.48 |
| PDLIM4_104 | IDPEIQDGSPTTSR | 270 | 5.93E−01 | 2.01E−03 | 5.93E−04 | −0.13 | 1.00 | 1.13 |
| PEX6_883 | LSAITR | 271 | 5.26E−01 | 4.71E−05 | 1.30E−04 | 0.12 | 2.05 | 1.92 |
| PISD_42 | HSVGDR | 272 | 8.04E−01 | 5.56E−03 | 1.42E−03 | 0.22 | 2.28 | 2.07 |
| POLR3G_88 | LFCSHWSHIPGFK | 273 | 1.00E+00 | 4.14E−03 | 3.09E−03 | 0.00 | 1.51 | 1.51 |
| POSTN_413 | FSDDTLSMDQR | 274 | 9.42E−01 | 1.20E−04 | 3.49E−04 | −0.02 | 2.29 | 2.31 |
| POSTN_754 | IITGPEIKYTR | 275 | 8.07E−01 | 8.70E−04 | 8.08E−05 | 0.10 | 1.77 | 1.68 |
| POSTN_790 | FIEGGDGHLFED | 276 | 7.61E−02 | 2.05E−03 | 7.86E−04 | −0.31 | 0.87 | 1.18 |
| | EEIKR | | | | | | | |
| POSTN_807 | LLQGDTPVR | 277 | 7.67E−01 | 5.07E−04 | 6.15E−04 | −0.07 | 1.10 | 1.17 |
| PPAT_255 | IVEISR | 278 | 3.83E−01 | 3.55E−04 | 3.17E−04 | −0.25 | 1.52 | 1.77 |
| PPP1R18_295 | LSETLTR | 279 | 7.47E−01 | 1.14E−05 | 1.01E−05 | −0.08 | 3.60 | 3.68 |
| PPP1R21_646 | IGTLTR | 280 | 5.26E−01 | 4.71E−05 | 1.30E−04 | 0.12 | 2.05 | 1.92 |
| PSAP_171 | FmANIPLLLYPQ | 281 | 8.10E−01 | 7.04E−03 | 7.33E−03 | 0.10 | 1.13 | 1.03 |
| | DGPR | | | | | | | |
| PXDN_1341 | SYQEDKPTKKTR | 282 | 1.05E−01 | 5.53E−04 | 2.12E−03 | 1.36 | 6.62 | 5.26 |
| PXDN_1393 | QKTITDLR | 283 | 8.03E−01 | 2.86E−05 | 1.13E−05 | 0.10 | 4.88 | 4.77 |
| QSOX1_558 | VAAAPELAMGA | 284 | 4.93E−01 | 5.29E−03 | 1.72E−03 | −0.19 | 0.94 | 1.13 |
| | LELESR | | | | | | | |
| SAGE1_484 | ITHSVR | 285 | 7.34E−01 | 1.25E−04 | 1.54E−04 | −0.11 | 2.22 | 2.33 |
| SCIN_519 | LASITR | 286 | 5.26E−01 | 4.71E−05 | 1.30E−04 | 0.12 | 2.05 | 1.92 |
| SDC4_106 | LEENEVIPKR | 287 | 1.41E−01 | 3.05E−04 | 5.33E−04 | −0.35 | 1.32 | 1.67 |
| SERBP1_56 | QAAAQTNSNAA | 288 | 7.48E−01 | 9.94E−06 | 1.08E−05 | −0.09 | 3.97 | 4.06 |
| | GKQLR | | | | | | | |
| SERBP1_60 | qTNSNAAGKQLR | 289 | 3.13E−01 | 4.71E−05 | 4.43E−05 | −0.19 | 1.90 | 2.10 |
| SERBP1_116 | qLQGEGKIIDR | 290 | 4.93E−01 | 4.71E−05 | 1.69E−04 | −0.20 | 2.35 | 2.55 |
| SERPINH1_27 | AAAPGTAEKLSP | 291 | 2.01E−02 | 3.04E−05 | 2.19E−05 | −0.69 | 2.62 | 3.30 |
| | KAATLAER | | | | | | | |
| SMOC1_35 | LISDRDPQCNLH | 292 | 6.48E−01 | 3.89E−04 | 1.59E−04 | 0.14 | 1.75 | 1.62 |
| | CSR | | | | | | | |
| SPOCK1_126 | HWVGPSNLVK | 293 | 7.22E−02 | 1.20E−03 | 9.71E−03 | 0.36 | 1.29 | 0.93 |
| SRGN_50 | LEEKGPMFELLP | 294 | 7.80E−01 | 3.20E−05 | 2.16E−05 | −0.07 | 3.21 | 3.28 |
| | GESNKIPR | | | | | | | |
| SRGN_51 | EEKGPMFELLPG | 295 | 6.03E−01 | 9.38E−04 | 9.80E−04 | −0.13 | 1.66 | 1.79 |
| | ESNKIPR | | | | | | | |
| SRGN_59 | LLPGESNKIPR | 296 | 7.49E−01 | 9.94E−06 | 2.28E−05 | −0.10 | 4.23 | 4.33 |
| SRGN_60 | LPGESNKIPR | 297 | 5.21E−01 | 4.21E−05 | 2.72E−04 | 0.32 | 3.57 | 3.25 |
| SRGN_62 | GESNKIPR | 298 | 1.47E−01 | 2.81E−05 | 6.92E−05 | −0.45 | 2.82 | 3.26 |
| SRGN_129 | FHDNLR | 299 | 9.36E−01 | 5.73E−05 | 4.16E−05 | 0.05 | 4.01 | 3.96 |
| STC1_37 | QNSAEVVR | 300 | 7.14E−01 | 2.98E−05 | 6.99E−05 | −0.22 | 4.56 | 4.78 |
| STC2_239 | HGEAGHHLPEPSSR | 301 | 8.59E−01 | 8.27E−03 | 2.54E−04 | −0.11 | 1.59 | 1.70 |
| STC2_244 | HHLPEPSSR | 302 | 2.95E−01 | 2.91E−04 | 1.92E−03 | −0.26 | 1.09 | 1.35 |
| SYNJ1_1350 | ITGLTR | 303 | 5.26E−01 | 4.71E−05 | 1.30E−04 | 0.12 | 2.05 | 1.92 |
| SYNM_968 | LSALTR | 304 | 5.26E−01 | 4.71E−05 | 1.30E−04 | 0.12 | 2.05 | 1.92 |
| TAGLN_168 | LQEGKHVIGLQm | 305 | 8.64E−01 | 1.37E−04 | 1.54E−04 | −0.08 | 2.73 | 2.81 |
| | GSNR | | | | | | | |
| TAGLN_178 | QMGSNR | 306 | 3.47E−01 | 1.44E−05 | 1.95E−05 | −0.30 | 3.65 | 3.95 |
| TAGLN2_167 | LQEGKNVIGLQm | 307 | 9.63E−01 | 1.14E−05 | 9.76E−06 | −0.01 | 4.26 | 4.27 |
| | GTNR | | | | | | | |
| TG_1017 | GASALLR | 308 | 6.93E−02 | 1.10E−04 | 1.71E−05 | 1.03 | 4.08 | 3.05 |
| TGFBI_24 | GPAKSPYQLVLQ | 309 | 7.66E−01 | 2.77E−03 | 7.79E−03 | 0.05 | 0.77 | 0.71 |
| | HSR | | | | | | | |
| TGFBI_43 | qHGPNVCAVQK | 310 | 5.73E−01 | 8.08E−04 | 5.19E−03 | 0.10 | 0.85 | 0.75 |
| | VIGTNR | | | | | | | |
| TGFBI_50 | AVQKVIGTNR | 311 | 1.58E−01 | 3.02E−03 | 9.75E−03 | 0.33 | 0.96 | 0.64 |
| TGFBI_208 | GIVTVNCAR | 312 | 2.34E−01 | 6.24E−03 | 6.22E−03 | 0.30 | 0.89 | 0.59 |
| TGFBI_276 | TLLAPTnEAFEKI | 313 | 6.81E−01 | 3.42E−03 | 6.19E−03 | 0.09 | 0.75 | 0.66 |
| | PSETLNR | | | | | | | |
| TGFBI_497 | VLTPPmGTVMD | 314 | 4.32E−01 | 2.21E−03 | 9.10E−03 | 0.14 | 0.75 | 0.61 |
| | VLKGDNR | | | | | | | |
| TGFBI_524 | SAGLTETLNR | 315 | 4.18E−01 | 4.95E−03 | 7.42E−03 | 0.18 | 0.84 | 0.66 |
| TGFBI_534 | EGVYTVFAPTNE | 316 | 6.48E−01 | 7.98E−04 | 2.07E−03 | 0.09 | 0.91 | 0.82 |
| | AFR | | | | | | | |
| THBS1_972 | QNDPNWVVR | 317 | 8.83E−01 | 9.94E−06 | 6.73E−06 | −0.04 | 3.55 | 3.59 |
| THBS2_290 | LSENLKR | 318 | 2.02E−01 | 4.94E−05 | 5.49E−06 | 0.59 | 4.65 | 4.05 |
| TLN1_1501 | HTSALCNSCR | 319 | 4.45E−01 | 3.46E−04 | 7.39E−04 | −0.17 | 1.31 | 1.48 |
| TMEM132A_52 | LLDAPEHFR | 320 | 6.10E−01 | 1.41E−03 | 3.13E−04 | −0.30 | 2.65 | 2.95 |
| TMTC1_585 | LGTLTR | 321 | 5.26E−01 | 4.71E−05 | 1.30E−04 | 0.12 | 2.05 | 1.92 |
| VASN_138 | HIQPGAFDTLDR | 322 | 6.16E−01 | 2.39E−03 | 6.97E−03 | 0.19 | 1.63 | 1.45 |
| VASP_229 | AIAGAKLR | 323 | 9.17E−01 | 4.59E−05 | 8.51E−05 | −0.08 | 4.48 | 4.56 |
| VIM_314 | QESTEYR | 324 | 9.16E−01 | 2.86E−05 | 2.77E−05 | 0.03 | 2.63 | 2.60 |
| VIM_333 | LKGTNESLER | 325 | 9.54E−01 | 2.98E−05 | 5.30E−06 | −0.03 | 4.37 | 4.39 |
| VIM_433 | LVDTHSKR | 326 | 6.53E−01 | 2.81E−05 | 1.67E−05 | 0.12 | 2.91 | 2.79 |

| | | | | | | | TAILS_ | |
|---|---|---|---|---|---|---|---|---|
| | | | | | | | SMC_ | |
| | | | | | | SEQ | Scaleup_ | |
| | accession_ | gene- | | previous_ | | ID | fxns_ | |
| TAILS_id | number | Symbol | StartAA | aa | sequence | NO: | mATS7i | entry_name |
| ACTB_8 | P60709 | ACTB | 8 | (A) | LVVDNGSG | 118 | 2.71E+09 | Actin, |
| | | | | | MCKAGFA | | | cytoplasmic |
| | | | | | GDDAPR | | | 1 |
| ACTB_21 | P60709 | ACTB | 21 | (G) | FAGDDAPR | 119 | 6.18E+09 | Actin, |
| | | | | | | | | cytoplasmic |
| | | | | | | | | 1 |
| ACTB_105 | P60709 | ACTB | 105 | (L) | LTEAPLNP | 120 | 5.77E+09 | Actin, |
| | | | | | KANR | | | cytoplasmic |
| | | | | | | | | 1 |
| ACTG1_8 | P63261 | ACTG1 | 8 | (A) | LVIDNGSG | 121 | 3.71E+08 | Actin, |
| | | | | | MCKAGFA | | | cytoplasmic |
| | | | | | GDDAPR | | | 2 |
| ADAM9_69 | A0AVL1 | ADAM9 | 69 | (Y) | VIQAEGKE | 122 | 2.56E+10 | ADAM9 |
| | | | | | HIIHLER | | | protein |
| AEBP1_37 | Q8IUX7 | AEBP1 | 37 | (E) | FLEGFLSEL | 123 | 4.54E+07 | Adipocyte |
| | | | | | EPEPR | | | enhancer− |
| | | | | | | | | binding |
| | | | | | | | | protein 1 |
| APLP2_579 | Q06481 | APLP2 | 579 | (S) | ISETPVDVR | 124 | 1.83E+08 | Amyloid−like |
| | | | | | | | | protein 2 |
| ATP8B3_1266 | O60423 | ATP8B3 | 1266 | (T) | QGTILR | 125 | 2.77E+08 | Phospholipid- |
| | | | | | | | | transporting |
| | | | | | | | | ATPase IK |
| BCO1_29 | Q9HAY6 | BCO1 | 29 | (L) | QGTLLR | 126 | | Beta, beta− |
| | | | | | | | | carotene |
| | | | | | | | | 15,15′− |
| | | | | | | | 9.25E+07 | dioxygenase |
| CCDC80_66 | Q76M96 | CCDC80 | 66 | (T) | LEEPNLQP | 127 | 7.99E+09 | Coiled−coil |
| | | | | | LQR | | | domain− |
| | | | | | | | | containing |
| | | | | | | | | protein 80 |
| CDC37_43 | Q16543 | CDC37 | 43 | (Q) | FQKEKEEL | 128 | 4.09E+06 | Hsp90 co− |
| | | | | | DR | | | chaperone |
| | | | | | | | | Cdc37 |
| CDH11_213 | P55287 | CDH11 | 213 | (A) | QTGIIR | 129 | 2.77E+08 | Cadherin−11 |
| CDH11_467 | P55287 | CDH11 | 467 | (R) | HQEAKVPV | 130 | 2.90E+07 | Cadherin−11 |
| | | | | | AIR | | | |
| CLEC11A_50 | Q9Y240 | CLEC11A | 50 | (K) | HLQEALGL | 131 | 7.22E+07 | C−type lectin |
| | | | | | PAGR | | | domain |
| | | | | | | | | family 11 |
| | | | | | | | | member A |
| COL16A1_ | Q07092 | COL16A1 | 269 | (P) | qSEGKVYT | 132 | 3.99E+09 | Collagen |
| 269 | | | | | R | | | alpha−1(XVI) |
| | | | | | | | | chain |
| COL18A1_ | P39060 | COL18A1 | 1505 | (A) | LQPPVVQL | 133 | 1.68E+09 | Collagen |
| 1505 | | | | | HDSNPYPR | | | alpha− |
| | | | | | | | | 1(XVIII) |
| | | | | | | | | chain |
| COL1A2_26 | P08123 | COL1A2 | 26 | (L) | QEETVR | 134 | 8.10E+07 | Collagen |
| | | | | | | | | alpha−2(I) |
| | | | | | | | | chain |
| COL1A2_80 | P08123 | COL1A2 | 80 | (A) | qYDGKGVG | 135 | 1.24E+09 | Collagen |
| | | | | | LGPGPMGL | | | alpha−2(I) |
| | | | | | mGPR | | | chain |
| COL1A2_91 | P08123 | COL1A2 | 91 | (P) | GPMGLMG | 136 | 6.70E+07 | Collagen |
| | | | | | PR | | | alpha−2(I) |
| | | | | | | | | chain |
| COL1A2_113 | P08123 | COL1A2 | 113 | (G) | FQGPAGEP | 137 | 2.09E+09 | Collagen |
| | | | | | GEPGQTGP | | | alpha−2(I) |
| | | | | | AGAR | | | chain |
| COL1A2_270 | P08123 | COL1A2 | 270 | (A) | VGNAGPA | 138 | 5.91E+07 | Collagen |
| | | | | | GPAGPR | | | alpha−2(I) |
| | | | | | | | | chain |
| COL1A2_305 | P08123 | COL1A2 | 305 | (G) | LTGAKGAA | 139 | 5.24E+07 | Collagen |
| | | | | | GLPGVAGA | | | alpha−2(I) |
| | | | | | PGLPGPR | | | chain |
| COL1A2_389 | P08123 | COL1A2 | 389 | (G) | SAGPPGPP | 140 | 4.24E+07 | Collagen |
| | | | | | GLR | | | alpha−2(I) |
| | | | | | | | | chain |
| COL1A2_425 | P08123 | COL1A2 | 425 | (G) | ASGPAGVR | 141 | 3.44E+08 | Collagen |
| | | | | | | | | alpha−2(I) |
| | | | | | | | | chain |
| COL1A2_630 | P08123 | COL1A2 | 630 | (A) | VGTAGPSG | 142 | 8.62E+06 | Collagen |
| | | | | | PSGLPGER | | | alpha−2(I) |
| | | | | | | | | chain |
| COL1A2_681 | P08123 | COL1A2 | 681 | (A) | VGAPGPAG | 143 | 4.11E+08 | Collagen |
| | | | | | ATGDR | | | alpha−2(I) |
| | | | | | | | | chain |
| COL1A2_731 | P08123 | COL1A2 | 731 | (G) | AAGQPGA | 144 | 2.50E+08 | Collagen |
| | | | | | KGER | | | alpha−2(I) |
| | | | | | | | | chain |
| COL1A2_866 | P08123 | COL1A2 | 866 | (G) | LLGAPGIL | 145 | 7.88E+09 | Collagen |
| | | | | | GLPGSR | | | alpha−2(I) |
| | | | | | | | | chain |
| COL1A2_960 | P08123 | COL1A2 | 960 | (A) | AGAPGPHG | 146 | 4.57E+08 | Collagen |
| | | | | | PVGPAGKH | | | alpha−2(I) |
| | | | | | GNR | | | chain |
| COL1A2_ | P08123 | COL1A2 | 1028 | (G) | LQGLPGIA | 147 | 1.05E+09 | Collagen |
| 1028 | | | | | GHHGDQG | | | alpha−2(I) |
| | | | | | APGSVGPA | | | chain |
| | | | | | GPR | | | |
| COL1A2_ | P08123 | COL1A2 | 1037 | (G) | HHGDQGA | 148 | 7.86E+09 | Collagen |
| 1037 | | | | | PGSVGPAG | | | alpha−2(I) |
| | | | | | PR | | | chain |
| COL1A2_ | P08123 | COL1A2 | 1127 | (P) | SLRPKDYE | 149 | 1.05E+08 | Collagen |
| 1127 | | | | | VDATLKSL | | | alpha−2(I) |
| | | | | | NNQIETLLT | | | chain |
| | | | | | PEGSR | | | |
| COL4A2_71 | P08572 | COL4A2 | 71 | (G) | YNGPPGLQ | 150 | 6.55E+07 | Collagen |
| | | | | | GFPGLQGR | | | alpha−2(IV) |
| | | | | | | | | chain |
| COL4A2_172 | P08572 | COL4A2 | 172 | (P) | YALPKEER | 151 | 8.09E+07 | Collagen |
| | | | | | | | | alpha−2(IV) |
| | | | | | | | | chain |
| COL5A1_544 | A0A087WXW9 | COL5A1 | 544 | (E) | SQAQAILQ | 152 | 2.24E+08 | Collagen |
| | | | | | QAR | | | alpha−1(V) |
| | | | | | | | | chain |
| COL8A1_90 | P27658 | COL8A1 | 90 | (Y) | MKEIQPAP | 153 | 4.20E+07 | Collagen |
| | | | | | R | | | alpha−1(VIII) |
| | | | | | | | | chain |
| DNAJA4_12 | Q8WW22−3 | DNAJA4 | 12 | (Q) | ISALTR | 154 | 1.01E+08 | Isoform 3 of |
| | | | | | | | | DnaJ |
| | | | | | | | | homolog |
| | | | | | | | | subfamily A |
| | | | | | | | | member4 |
| ECM1_43 | Q16610 | ECM1 | 43 | (G) | YAAPPSPPL | 155 | 3.25E+08 | Extracellular |
| | | | | | SR | | | matrix |
| | | | | | | | | protein 1 |
| ECM1_134 | Q16610 | ECM1 | 134 | (P) | FGDQSHPE | 156 | 4.13E+08 | Extracellular |
| | | | | | PESWNAAQ | | | matrix |
| | | | | | HCQQDR | | | protein 1 |
| EFEMP1_124 | Q12805 | EFEMP1 | 124 | (A) | AVAGPEM | 157 | 3.19E+09 | EGF− |
| | | | | | QTGR | | | containing |
| | | | | | | | | fibulin−like |
| | | | | | | | | extracellular |
| | | | | | | | | matrix |
| | | | | | | | | protein 1 |
| EFEMP1_125 | Q12805 | EFEMP1 | 125 | (A) | VAGPEMQT | 158 | 2.03E+09 | EGF− |
| | | | | | GR | | | containing |
| | | | | | | | | fibulin−like |
| | | | | | | | | extracellular |
| | | | | | | | | matrix |
| | | | | | | | | protein 1 |
| EFEMP1_135 | Q12805 | EFEMP1 | 135 | (R) | NNFVIR | 159 | 1.83E+09 | EGF− |
| | | | | | | | | containing |
| | | | | | | | | fibulin−like |
| | | | | | | | | extracellular |
| | | | | | | | | matrix |
| | | | | | | | | protein 1 |
| EFHD2_67 | Q96C19 | EFHD2 | 67 | (N) | QGIGEPQSP | 160 | 2.69E+07 | EF−hand |
| | | | | | SR | | | domain− |
| | | | | | | | | containing |
| | | | | | | | | protein D2 |
| EHBP1L1_298 | Q8N3D4 | EHBP1L1 | 298 | (D) | TAPTPAPR | 161 | 2.19E+07 | EH domain− |
| | | | | | | | | binding |
| | | | | | | | | protein 1−like |
| | | | | | | | | protein 1 |
| FBLN2_259 | P98095 | FBLN2 | 259 | (A) | ALGPPAPV | 162 | 1.04E+08 | Fibulin−2 |
| | | | | | QAKAR | | | |
| FBLN2_260 | P98095 | FBLN2 | 260 | (A) | LGPPAPVQ | 163 | 7.69E+08 | Fibulin−2 |
| | | | | | AKAR | | | |
| FBN1_29 | P35555 | FBN1 | 29 | (N) | LEAGNVKE | 164 | 1.26E+10 | Fibrillin−1 |
| | | | | | TR | | | |
| FBN1_53 | P35555 | FBN1 | 53 | (A) | LKGPNVCG | 165 | 8.48E+08 | Fibrillin−1 |
| | | | | | SR | | | |
| FLNA_612 | P21333 | FLNA | 612 | (F) | SVEGPSQA | 166 | 8.79E+08 | Filamin−A |
| | | | | | KIECDDKG | | | |
| | | | | | DGSCDVR | | | |
| FLNA_695 | P21333 | FLNA | 695 | (E) | FTVDAKHG | 167 | 3.71E+08 | Filamin−A |
| | | | | | GKAPLR | | | |
| FLNA_1285 | P21333 | FLNA | 1285 | (A) | LTQTGGPH | 168 | 5.65E+08 | Filamin−A |
| | | | | | VKAR | | | |
| FN1_36 | P02751−15 | FN1 | 36 | (Q) | MVQPQSPV | 169 | 4.40E+10 | Isoform 15 of |
| | | | | | AVSQSKPG | | | Fibronectin |
| | | | | | CYDnGKHY | | | |
| | | | | | QINQQWER | | | |
| FN1_40 | P02751−15 | FN1 | 40 | (P) | qSPVAVSQ | 170 | 3.67E+09 | Isoform 15 of |
| | | | | | SKPGCYDn | | | Fibronectin |
| | | | | | GKHYQINQ | | | |
| | | | | | QWER | | | |
| FN1_45 | P02751−15 | FN1 | 45 | (A) | VSQSKPGC | 171 | 5.68E+10 | Isoform 15 of |
| | | | | | YDNGKHY | | | Fibronectin |
| | | | | | QINQQWER | | | |
| FN1_47 | P02751−15 | FN1 | 47 | (S) | qSKPGCYD | 172 | 1.26E+10 | Isoform 15 of |
| | | | | | nGKHYQIN | | | Fibronectin |
| | | | | | QQWER | | | |
| FN1_279 | P02751−15 | FN1 | 279 | (T) | TSSGSGPFT | 173 | 5.94E+09 | Isoform 15 of |
| | | | | | DVR | | | Fibronectin |
| FN1_281 | P02751−15 | FN1 | 281 | (S) | SGSGPFTD | 174 | 5.31E+09 | Isoform 15 of |
| | | | | | VR | | | Fibronectin |
| FN1_282 | P02751−15 | FN1 | 282 | (S) | GSGPFTDV | 175 | 1.05E+10 | Isoform 15 of |
| | | | | | R | | | Fibronectin |
| FN1_886 | P02751−15 | FN1 | 886 | (E) | NQESTPVVI | 176 | 1.42E+09 | Isoform 15 of |
| | | | | | QQETTGTP | | | Fibronectin |
| | | | | | R | | | |
| FN1_1143 | P02751−15 | FN1 | 1143 | (G) | LTPGVEYV | 177 | 1.36E+08 | Isoform 15 of |
| | | | | | YTIQVLR | | | Fibronectin |
| FN1_1656 | P02751−15 | FN1 | 1656 | (P) | SSSPVTGY | 178 | 4.82E+09 | Isoform 15 of |
| | | | | | R | | | Fibronectin |
| FN1_1715 | P02751−15 | FN1 | 1715 | (P) | LVQTAVTN | 179 | 2.15E+09 | Isoform 15 of |
| | | | | | IDR | | | Fibronectin |
| FN1_1715 | P02751−15 | FN1 | 1715 | (P) | LVQTAVTN | 180 | 7.60E+08 | Isoform 15 of |
| | | | | | IDRPKGLA | | | Fibronectin |
| | | | | | FTDVDVDS | | | |
| | | | | | IKIAWESPQ | | | |
| | | | | | GQVSR | | | |
| FN1_1731 | P02751−15 | FN1 | 1731 | (A) | FTDVDVDS | 181 | 9.47E+08 | Isoform 15 of |
| | | | | | IKIAWESPQ | | | Fibronectin |
| | | | | | GQVSR | | | |
| FN1_2143 | P02751−15 | FN1 | 2143 | (I) | FEEHGFR | 182 | 8.90E+07 | Isoform 15 of |
| | | | | | | | | Fibronectin |
| FN1_2217 | P02751−15 | FN1 | 2217 | (P) | FQDTSEYII | 183 | 3.90E+09 | Isoform 15 of |
| | | | | | SCHPVGTD | | | Fibronectin |
| | | | | | EEPLQFR | | | |
| FN1_2250 | P02751−15 | FN1 | 2250 | (T) | LTGLTR | 184 | 1.31E+09 | Isoform 15 of |
| | | | | | | | | Fibronectin |
| GALNT2_31 | Q10471 | GALNT2 | 31 | (A) | LAGGAGG | 185 | 2.03E+08 | Polypeptide |
| | | | | | GAGR | | | N− |
| | | | | | | | | acetylgalacto |
| | | | | | | | | saminyltrans- |
| | | | | | | | | ferase 2 |
| GSN_35 | P06396 | GSN | 35 | (A) | SQAGAPQG | 186 | 3.49E+07 | Gelsolin |
| | | | | | R | | | |
| GSN_413 | P06396 | GSN | 413 | (S) | HIANVER | 187 | 2.44E+09 | Gelsolin |
| HEG1_47 | Q9ULI3 | HEG1 | 47 | (P) | LAGAGLEL | 188 | 6.74E+08 | Protein HEG |
| | | | | | QLER | | | homolog_1 |
| HEG1_49 | Q9ULI3 | HEG1 | 49 | (A) | GAGLELQL | 189 | 9.60E+08 | Protein HEG |
| | | | | | ER | | | homolog_1 |
| HLA−C_52 | A0A140T912 | HLA−C | 52 | (Y) | VDDTQFVR | 190 | 1.21E+10 | HLA class I |
| | | | | | | | | histocompat- |
| | | | | | | | | ibility |
| | | | | | | | | antigen, Cw− |
| | | | | | | | | 6 alpha chain |
| HSPG2_275 | P98160 | HSPG2 | 275 | (P) | LLPGSVR | 191 | 2.35E+07 | Basement |
| | | | | | | | | membrane− |
| | | | | | | | | specific |
| | | | | | | | | heparan |
| | | | | | | | | sulfate |
| | | | | | | | | proteoglycan |
| | | | | | | | | core protein |
| HSPG2_1863 | P98160 | HSPG2 | 1863 | (T) | LSAPVVSIH | 192 | 1.64E+08 | Basement |
| | | | | | PPQLTVQP | | | membrane− |
| | | | | | GQLAEFR | | | specific |
| | | | | | | | | heparan |
| | | | | | | | | sulfate |
| | | | | | | | | proteoglycan |
| | | | | | | | | core protein |
| HSPG2_1937 | P98160 | HSPG2 | 1937 | (H) | SSAGQQVA | 193 | 1.34E+08 | Basement |
| | | | | | R | | | membrane− |
| | | | | | | | | specific |
| | | | | | | | | heparan |
| | | | | | | | | sulfate |
| | | | | | | | | proteoglycan |
| | | | | | | | | core protein |
| HSPG2_2331 | P98160 | HSPG2 | 2331 | (G) | ANLAYPAG | 194 | | Basement |
| | | | | | | | | membrane− |
| | | | | | | | | specific |
| | | | | | STQPIR | | 7.64E+07 | heparan |
| | | | | | | | | sulfate |
| | | | | | | | | proteoglycan |
| | | | | | | | | core protein |
| HSPG2_2333 | P98160 | HSPG2 | 2333 | (N) | LAYPAGST | 195 | 5.74E+07 | Basement |
| | | | | | QPIR | | | membrane− |
| | | | | | | | | specific |
| | | | | | | | | heparan |
| | | | | | | | | sulfate |
| | | | | | | | | proteoglycan |
| | | | | | | | | core protein |
| HSPG2_2688 | P98160 | HSPG2 | 2688 | (S) | VADSGEYV | 196 | 2.03E+07 | Basement |
| | | | | | CR | | | membrane− |
| | | | | | | | | specific |
| | | | | | | | | heparan |
| | | | | | | | | sulfate |
| | | | | | | | | proteoglycan |
| | | | | | | | | core protein |
| HSPG2_3250 | P98160 | HSPG2 | 3250 | (R) | SPLPWQHR | 197 | 1.36E+10 | Basement |
| | | | | | | | | membrane− |
| | | | | | | | | specific |
| | | | | | | | | heparan |
| | | | | | | | | sulfate |
| | | | | | | | | proteoglycan |
| | | | | | | | | core protein |
| HSPG2_3523 | P98160 | HSPG2 | 3523 | (W) | SKVGGHLR | 198 | 3.75E+08 | Basement |
| | | | | | | | | membrane− |
| | | | | | | | | specific |
| | | | | | | | | heparan |
| | | | | | | | | sulfate |
| | | | | | | | | proteoglycan |
| | | | | | | | | core protein |
| HSPG2_3523 | P98160 | HSPG2 | 3523 | (W) | SKVGGHLR | 199 | 1.67E+09 | Basement |
| | | | | | PGIVQSGG | | | membrane− |
| | | | | | VVR | | | specific |
| | | | | | | | | heparan |
| | | | | | | | | sulfate |
| | | | | | | | | proteoglycan |
| | | | | | | | | core protein |
| HSPG2_3542 | P98160 | HSPG2 | 3542 | (R) | IAHVELAD | 200 | 1.56E+09 | Basement |
| | | | | | AGQYR | | | membrane− |
| | | | | | | | | specific |
| | | | | | | | | heparan |
| | | | | | | | | sulfate |
| | | | | | | | | proteoglycan |
| | | | | | | | | core protein |
| HSPG2_3647 | P98160 | HSPG2 | 3647 | (R) | qGKVKAFA | 20 | 6.48E+08 | Basement |
| | | | | | HLQVPER | | | membrane− |
| | | | | | | | | specific |
| | | | | | | | | heparan |
| | | | | | | | | sulfate |
| | | | | | | | | proteoglycan |
| | | | | | | | | core protein |
| HSPG2_3672 | P98160 | HSPG2 | 3672 | (Y) | SFLPLPTIK | 202 | 1.76E+09 | Basement |
| | | | | | DAYR | | | membrane− |
| | | | | | | | | specific |
| | | | | | | | | heparan |
| | | | | | | | | sulfate |
| | | | | | | | | proteoglycan |
| | | | | | | | | core protein |
| HSPG2_3813 | P98160 | HSPG2 | 3813 | (L) | SSGFIGCVR | 203 | 1.02E+08 | Basement |
| | | | | | | | | membrane− |
| | | | | | | | | specific |
| | | | | | | | | heparan |
| | | | | | | | | sulfate |
| | | | | | | | | proteoglycan |
| | | | | | | | | core protein |
| HSPG2_3969 | P98160 | HSPG2 | 3969 | (F) | SGGKSGPV | 204 | 2.28E+08 | Basement |
| | | | | | EDFVSLAm | | | membrane− |
| | | | | | VGGHLEFR | | | specific |
| | | | | | | | | heparan |
| | | | | | | | | sulfate |
| | | | | | | | | proteoglycan |
| | | | | | | | | core protein |
| HSPG2_4004 | P98160 | HSPG2 | 4004 | (R) | SAEPLALG | 205 | 1.03E+10 | Basement |
| | | | | | R | | | membrane− |
| | | | | | | | | specific |
| | | | | | | | | heparan |
| | | | | | | | | sulfate |
| | | | | | | | | proteoglycan |
| | | | | | | | | core protein |
| HSPG2_4021 | P98160 | HSPG2 | 4021 | (R) | LNKDGSLR | 206 | 3.13E+08 | Basement |
| | | | | | | | | membrane− |
| | | | | | | | | specific |
| | | | | | | | | heparan |
| | | | | | | | | sulfate |
| | | | | | | | | proteoglycan |
| | | | | | | | | core protein |
| HSPG2_4075 | P98160 | HSPG2 | 4075 | (R) | GCVGEVSV | 207 | 1.79E+09 | Basement |
| | | | | | NGKR | | | membrane− |
| | | | | | | | | specific |
| | | | | | | | | heparan |
| | | | | | | | | sulfate |
| | | | | | | | | proteoglycan |
| | | | | | | | | core protein |
| HSPG2_4133 | P98160 | HSPG2 | 4133 | (R) | DGFKGDLC | 208 | 4.04E+08 | Basement |
| | | | | | EHEENPCQ | | | membrane− |
| | | | | | LR | | | specific |
| | | | | | | | | heparan |
| | | | | | | | | sulfate |
| | | | | | | | | proteoglycan |
| | | | | | | | | core protein |
| HTRA1_33 | Q92743 | HTRA1 | 33 | (P) | LAAGCPDR | 209 | 1.00E+08 | Serine |
| | | | | | | | | protease |
| | | | | | | | | HTRA1 |
| IFI30_43 | P13284 | IFI30 | 43 | (N) | YKTGNLYL | 210 | 6.01E+06 | Gamma− |
| | | | | | R | | | interferon− |
| | | | | | | | | inducible |
| | | | | | | | | lysosomal |
| | | | | | | | | thiol |
| | | | | | | | | reductase |
| IGFBP3_31 | P17936 | IGFBP3 | 31 | (S) | SAGLGPVV | 211 | 4.82E+08 | Insulin−like |
| | | | | | R | | | growth |
| | | | | | | | | factor− |
| | | | | | | | | binding |
| | | | | | | | | protein 3 |
| IGFBP3_32 | P17936 | IGFBP3 | 32 | (S) | AGLGPVVR | 212 | 2.11E+09 | Insulin−like |
| | | | | | | | | growth |
| | | | | | | | | factor− |
| | | | | | | | | binding |
| | | | | | | | | protein 3 |
| IGFBP3_200 | P17936 | IGFBP3 | 200 | (N) | FSSESKR | 213 | 3.44E+08 | Insulin−like |
| | | | | | | | | growth |
| | | | | | | | | factor− |
| | | | | | | | | binding |
| | | | | | | | | protein 3 |
| IGFBP7_101 | Q16270 | IGFBP7 | 101 | (A) | AAGGPGVS | 214 | 5.68E+07 | Insulin−like |
| | | | | | GVCVCKSR | | | growth |
| | | | | | | | | factor− |
| | | | | | | | | binding |
| | | | | | | | | protein 7 |
| INS_32 | A6XGL2 | INS | 32 | (C) | GSHL VEAL | 215 | 8.57E+07 | Insulin |
| | | | | | YLVCGER | | | |
| KPNB1_37 | Q14974 | KPNB1 | 37 | (F) | L VELSR | 216 | 3.15E+08 | Importin |
| | | | | | | | | subunit beta− |
| | | | | | | | | 1 |
| LAMA5_484 | O15230 | LAMA5 | 484 | | QVLPAGQI | 217 | 4.81E+08 | Laminin |
| | | | | | VNCDCSAA | | | subunit |
| | | | | (E) | GTQGNACR | | | alpha−5 |
| LASP1_123 | Q14847 | LASP1 | 123 | (Y) | HEEFEKSR | 218 | 2.34E+07 | LIM and |
| | | | | | | | | SH3 domain |
| | | | | | | | | protein 1 |
| LGALS1_53 | P09382 | LGALS1 | 53 | (A) | HGDANTIV | 219 | 4.04E+08 | Galectin−1 |
| | | | | | CNSKDGGA | | | |
| | | | | | WGTEQR | | | |
| LGALS3BP | Q08380 | LGALS3BP | 447 | (Y) | FQAPSDYR | 220 | 1.48E+08 | Galectin−3− |
| 447 | | | | | | | | binding |
| | | | | | | | | protein |
| LIPG_42 | B4DTR8 | LIPG | 42 | (T) | qTEVKPSV | 221 | 3.92E+08 | Endothelial |
| | | | | | R | | | lipase |
| LMNA_408 | P02545 | LMNA | 40 | (S) | qTQGGGSV | 222 | 3.42E+09 | Prelamin− |
| | | | | | TKKR | | | A/C |
| LOX_58 | P28300 | LOX | 58 | (L) | LSLGSQYQ | 223 | 2.82E+08 | Protein− |
| | | | | | PQR | | | lysine 6− |
| | | | | | | | | oxidase |
| LOX_80 | P28300 | LOX | 80 | (A) | ANASAQQP | 224 | 8.09E+07 | Protein− |
| | | | | | R | | | lysine 6− |
| | | | | | | | | oxidase |
| LOX_81 | P28300 | LOX | 81 | (A) | NASAQQPR | 225 | 3.49E+07 | Protein− |
| | | | | | | | | lysine 6− |
| | | | | | | | | oxidase |
| LOX_123 | P28300 | LOX | 123 | (R) | HWFQAGY | 226 | 1.50E+08 | Protein− |
| | | | | | STSR | | | lysine 6− |
| | | | | | | | | oxidase |
| LOX_124 | P28300 | LOX | 124 | (H) | WFQAGYST | 227 | 1.65E+08 | Protein− |
| | | | | | SR | | | lysine 6− |
| | | | | | | | | oxidase |
| LOX_126 | P28300 | LOX | 126 | (F) | QAGYSTSR | 228 | 4.18E+06 | Protein− |
| | | | | | | | | lysine 6− |
| | | | | | | | | oxidase |
| LOXL1_219 | H3BUV8 | LOXL1 | 219 | (A) | VASAGVIY | 229 | 2.12E+07 | Lysyl |
| | | | | | PYQPR | | | oxidase |
| | | | | | | | | homolog 1 |
| LOXL2_37 | Q9Y4K0 | LOXL2 | 37 | (Y) | FQQPAPEY | 230 | 5.20E+09 | Lysyl |
| | | | | | HQPQAPAN | | | oxidase |
| | | | | | VAKIQLR | | | homolog 2 |
| LOXL2_38 | Q9Y4K0 | LOXL2 | 38 | (F) | qQPAPEYH | 231 | 2.79E+08 | Lysyl |
| | | | | | QPQAPANV | | | oxidase |
| | | | | | AKIQLR | | | homolog 2 |
| LOXL2_318 | Q9Y4K0 | LOXL2 | 318 | (K) | AYKPEQPL | 232 | 7.35E+09 | Lysyl |
| | | | | | VR | | | oxidase |
| | | | | | | | | homolog 2 |
| LOXL4_25 | Q96JB6 | LOXL4 | 25 | (P) | qSLGTTKL | 233 | 1.42E+08 | Lysyl |
| | | | | | R | | | oxidase |
| | | | | | | | | homolog 4 |
| LTBP1_393 | Q14766 | LTBP1 | 393 | (T) | LTATNFR | 234 | 9.30E+08 | Latent− |
| | | | | | | | | transforming |
| | | | | | | | | growth factor |
| | | | | | | | | beta−binding |
| | | | | | | | | protein 1 |
| LTBP1_494 | Q14766 | LTBP1 | 494 | (A) | SVQIHQVS | 235 | 1.79E+09 | Latent− |
| | | | | | R | | | transforming |
| | | | | | | | | growth factor |
| | | | | | | | | beta−binding |
| | | | | | | | | protein 1 |
| LTBP1_540 | Q14766 | LTBP1 | 540 | (S) | HQQVIPHV | 236 | 1.90E+09 | Latent− |
| | | | | | YPVAAKTQ | | | transforming |
| | | | | | LGR | | | growth factor |
| | | | | | | | | beta−binding |
| | | | | | | | | protein 1 |
| LTBP1_546 | Q14766 | LTBP1 | 546 | (P) | HVYPVAA | 237 | 3.09E+08 | Latent− |
| | | | | | KTQLGR | | | transforming |
| | | | | | | | | growth factor |
| | | | | | | | | beta−binding |
| | | | | | | | | protein 1 |
| LTBP1_1597 | Q14766 | LTBP1 | 1597 | (F) | SEQYTPEA | 238 | 8.25E+08 | Latent− |
| | | | | | DPYFIQDR | | | transforming |
| | | | | | | | | growth factor |
| | | | | | | | | beta−binding |
| | | | | | | | | protein 1 |
| LTBP2_485 | G3V3X5 | LTBP2 | 485 | (A) | SVQIHQVA | 239 | 7.57E+08 | Latent− |
| | | | | | QVR | | | transforming |
| | | | | | | | | growth factor |
| | | | | | | | | beta−binding |
| | | | | | | | | protein 2 |
| LTBP2_533 | G3V3X5 | LTBP2 | 533 | (R) | SGEPPRPLP | 240 | 1.02E+10 | Latent− |
| | | | | | PAAPR | | | transforming |
| | | | | | | | | growth factor |
| | | | | | | | | beta−binding |
| | | | | | | | | protein 2 |
| LTBP2_677 | G3V3X5 | LTBP2 | 677 | (R) | SLGPGTCT | 241 | 2.60E+09 | Latent− |
| | | | | | LPLAQR | | | transforming |
| | | | | | | | | growth factor |
| | | | | | | | | beta−binding |
| | | | | | | | | protein 2 |
| LTBP2_760 | G3V3X5 | LTBP2 | 760 | (R) | SSGALPGP | 242 | 1.84E+09 | Latent− |
| | | | | | AER | | | transforming |
| | | | | | | | | growth factor |
| | | | | | | | | beta−binding |
| | | | | | | | | protein 2 |
| LTBP2_1023 | G3V3X5 | LTBP2 | 1023 | (H) | GKCTNLEG | 243 | 2.82E+08 | Latent− |
| | | | | | SFR | | | transforming |
| | | | | | | | | growth factor |
| | | | | | | | | beta−binding |
| | | | | | | | | protein 2 |
| LTBP2_1371 | G3V3X5 | LTBP2 | 1371 | (Y) | SGQKGHAP | 244 | 6.28E+08 | Latent− |
| | | | | | CSSVLGR | | | transforming |
| | | | | | | | | growth factor |
| | | | | | | | | beta−binding |
| | | | | | | | | protein 2 |
| LTBP2_1560 | G3V3X5 | LTBP2 | 1560 | (R) | TTYTECCC | 245 | 7.12E+09 | Latent− |
| | | | | | QDGEAWS | | | transforming |
| | | | | | QQCALCPP | | | growth factor |
| | | | | | R | | | beta−binding |
| | | | | | | | | protein 2 |
| LTBP2_1584 | G3V3X5 | LTBP2 | 1584 | (R) | SSEVYAQL | 246 | 3.60E+09 | Latent− |
| | | | | | CNVAR | | | transforming |
| | | | | | | | | growth factor |
| | | | | | | | | beta−binding |
| | | | | | | | | protein 2 |
| LTBP2_1602 | G3V3X5 | LTBP2 | 1602 | (R) | EAGVHFR | 247 | 4.66E+08 | Latent− |
| | | | | | | | | transforming |
| | | | | | | | | growth factor |
| | | | | | | | | beta−binding |
| | | | | | | | | protein 2 |
| LTBP3_239 | Q9NS15 | LTBP3 | 239 | (A) | SVQVHR | 248 | 1.25E+08 | Latent− |
| | | | | | | | | transforming |
| | | | | | | | | growth factor |
| | | | | | | | | beta−binding |
| | | | | | | | | protein 3 |
| LTBP4_197 | A0A0C4DH | LTBP4 | 197 | (V) | VHQVER | 249 | 8.92E+07 | Latent− |
| | 07 | | | | | | | transforming |
| | | | | | | | | growth factor |
| | | | | | | | | beta−binding |
| | | | | | | | | protein 4 |
| MAN2A1_51 | Q16706 | MAN2A1 | 51 | (M) | LQEKIDHL | 250 | 7.24E+07 | Alpha− |
| | | | | | ER | | | mannosidase |
| | | | | | | | | 2 |
| MAP1B_1065 | P46821 | MAP1B | 1065 | (F) | LTTPTKQL | 251 | 1.61E+07 | Microtubule− |
| | | | | | GAQSPGR | | | associated |
| | | | | | | | | protein 1B |
| MAP4_815 | P27816 | MAP4 | 815 | (A) | AVASTGPS | 252 | 5.58E+08 | Microtubule− |
| | | | | | SR | | | associated |
| | | | | | | | | protein 4 |
| | | | | | | | | (detected as |
| | | | | | | | | E7EVA0 |
| | | | | | | | | MAP4 1960) |
| MAP4_816 | P27816 | MAP4 | 816 | (A) | VASTGPSS | 253 | 1.57E+08 | Microtubule− |
| | | | | | R | | | associated |
| | | | | | | | | protein 4 |
| | | | | | | | | (detected as |
| | | | | | | | | E7EVA0 |
| | | | | | | | | MAP4 31961) |
| MGAT4B_28 | Q9UQ53 | MGAT4B | 28 | (A) | LSGQKGDV | 254 | 3.91E+07 | Alpha−1,3− |
| | | | | | VDVYQR | | | mannosyl− |
| | | | | | | | | glycoprotein |
| | | | | | | | | 4−beta−N− |
| | | | | | | | | acetylglucos- |
| | | | | | | | | aminyltrans- |
| | | | | | | | | ferase B |
| MMP1_24 | P03956 | MMP1 | 24 | (T) | LETQEQDV | 255 | 7.27E+07 | Interstitial |
| | | | | | DLVQKYLE | | | collagenase |
| | | | | | KYYNLKnD | | | |
| | | | | | GR | | | |
| MMP2_406 | P08253 | MMP2 | 406 | (F) | GHAmGLE | 256 | 1.08E+08 | 72 kDa type |
| | | | | | HSQDPGAL | | | IV |
| | | | | | MAPIYTYT | | | collagenase |
| | | | | | KnFR | | | |
| MRFAP1_39 | A0A087WTY9 | MRFAP1 | 39 | (D) | IASLTR | 257 | 2.02E+08 | MORF4 |
| | | | | | | | | family− |
| | | | | | | | | associated |
| | | | | | | | | protein 1 |
| MYH15_403 | Q9Y2K3 | MYH15 | 403 | (I) | nSSELVKCL | 258 | 5.50E+07 | Myosin−15 |
| | | | | | IHPR | | | |
| NID2_291 | Q14112 | NID2 | 291 | (A) | HSSVPLGR | 259 | 2.94E+09 | Nidogen−2 |
| NID2_374 | Q14112 | NID2 | 374 | (E) | VGGPDLKG | 260 | 1.18E+08 | Nidogen−2 |
| | | | | | QVEPWDER | | | |
| NID2_467 | Q14112 | NID2 | 467 | (G) | LEDNIGSN | 261 | 1.69E+08 | Nidogen−2 |
| | | | | | TEVFTYNA | | | |
| | | | | | ANKETCEH | | | |
| | | | | | NHR | | | |
| NID2_478 | Q14112 | NID2 | 478 | (V) | FTYNAANK | 262 | 1.74E+08 | Nidogen−2 |
| | | | | | ETCEHNHR | | | |
| NID2_951 | Q14112 | NID2 | 951 | (Q) | YAYPGAR | 263 | 3.51E+08 | Nidogen−2 |
| NUCB1_228 | Q02818 | NUCB1 | 228 | (Q) | LKEVWEEL | 264 | 1.30E+08 | Nucleobindin- |
| | | | | | DGLDPNR | | | 1 |
| NUCB1_317 | Q02818 | NUCB1 | 317 | (E) | FLASTQR | 265 | 1.78E+08 | Nucleobindin- |
| | | | | | | | | 1 |
| NUCB1_389 | Q02818 | NUCB1 | 389 | (E) | LQQAVLH | 266 | 1.01E+08 | Nucleobindin- |
| | | | | | MEQR | | | 1 |
| OS9_54 | B4E321 | OS9 | 54 | (G) | qSQSSDVVI | 267 | 1.17E+09 | Protein OS−9 |
| | | | | | VSSKYKQR | | | |
| OS9_55 | B4E321 | OS9 | 55 | (Q) | SQSSDVVI | 268 | 3.03E+08 | Protein OS−9 |
| | | | | | VSSKYKQR | | | |
| PAPPA_41 | Q13219 | PAPPA | 41 | (P) | AAGPATCA | 269 | 5.38E+07 | Pappalysin−1 |
| | | | | | TR | | | |
| PDLIM4_104 | P50479 | PDLIM4 | 104 | (H) | IDPEIQDGS | 270 | 2.81E+07 | PDZ and |
| | | | | | PTTSR | | | LIM domain |
| | | | | | | | | protein 4 |
| PEX6_883 | Q13608 | PEX6 | 883 | (V) | LSAITR | 271 | 2.02E+08 | Peroxisome |
| | | | | | | | | assembly |
| | | | | | | | | factor 2 |
| PISD_42 | F8WCS6 | PISD | 42 | (A) | HSVGDR | 272 | 7.50E+07 | Phosphatidyl |
| | | | | | | | | serine |
| | | | | | | | | decarboxylase |
| | | | | | | | | proenzyme, |
| | | | | | | | | mitochondrial |
| POLR3G_88 | D6RJH6 | POLR3G | 88 | (L) | LFCSHWSH | 273 | 7.00E+07 | DNA− |
| | | | | | IPGFK | | | directed |
| | | | | | | | | RNA |
| | | | | | | | | polymerase |
| | | | | | | | | III subunit |
| | | | | | | | | RPC7 |
| POSTN_413 | Q15063 | POSTN | 413 | (A) | FSDDTLSM | 274 | 1.35E+08 | Periostin |
| | | | | | DQR | | | |
| POSTN_754 | Q15063 | POSTN | 754 | (R) | IITGPEIKYT | 275 | 2.17E+09 | Periostin |
| | | | | | R | | | |
| POSTN_790 | Q15063 | POSTN | 790 | (K) | FIEGGDGH | 276 | 1.69E+08 | Periostin |
| | | | | | LFEDEEIKR | | | |
| POSTN_807 | Q15063 | POSTN | 807 | (R) | LLQGDTPV | 277 | 3.08E+08 | Periostin |
| | | | | | R | | | |
| PPAT_255 | Q06203 | PPAT | 255 | (E) | IVEISR | 278 | 3.15E+08 | Amidophosp |
| | | | | | | | | horibosyl- |
| | | | | | | | | transferase |
| PPP1R18_ | A0A0G2JHC2 | PPP1R18 | 295 | (E) | LSETLTR | 279 | 7.16E+07 | PPP1R18 |
| 295 | | | | | | | | |
| PPP1R21_ | Q6ZMI0 | PPP1R21 | 646 | (L) | IGTLTR | 280 | 1.01E+08 | Protein |
| 646 | | | | | | | | phosphatase |
| | | | | | | | | 1 regulatory |
| | | | | | | | | subunit 21 |
| PSAP_171 | C9JIZ6 | PSAP | 171 | (P) | FmANIPLLL | 281 | 1.43E+08 | Prosaposin |
| | | | | | YPQDGPR | | | |
| PXDN_1341 | Q92626 | PXDN | 1341 | (F) | SYQEDKPT | 282 | 1.16E+09 | Peroxidasin |
| | | | | | KKTR | | | homolog |
| PXDN_1393 | Q92626 | PXDN | 1393 | (M | QKTITDLR | 283 | 8.16E+07 | Peroxidasin |
| | | | | | | | | homolog |
| QSOX1_558 | O00391 | QSOX1 | 558 | (N) | VAAAPELA | 284 | 8.63E+08 | Sulfhydryl |
| | | | | | MGALELES | | | oxidase 1 |
| | | | | | R | | | |
| SAGE1_484 | Q9NXZ1 | SAGE1 | 484 | (T) | ITHSVR | 285 | 1.19E+08 | Sarcoma |
| | | | | | | | | antigen 1 |
| SCIN_519 | Q9Y6U3−2 | SCIN | 519 | (N) | LASITR | 286 | 1.01E+08 | Isoform 2 of |
| | | | | | | | | Adseverin |
| SDC4_106 | P31431 | SDC4 | 106 | (K) | LEENEVIPK | 287 | 1.76E+08 | Syndecan−4 |
| | | | | | R | | | |
| SERBP1_56 | Q8NC51 | SERBP1 | 56 | (A) | QAAAQTNS | 288 | 9.32E+07 | Plasminogen |
| | | | | | NAAGKQL | | | activator |
| | | | | | R | | | inhibitor 1 |
| | | | | | | | | RNA−binding |
| | | | | | | | | protein |
| SERBP1_60 | Q8NC51 | SERBP1 | 60 | (A) | qTNSNAAG | 289 | 2.09E+08 | Plasminogen |
| | | | | | KQLR | | | activator |
| | | | | | | | | inhibitor 1 |
| | | | | | | | | RNA−binding |
| | | | | | | | | protein |
| SERBP1_116 | Q8NC51 | SERBP1 | 116 | (Q) | qLQGEGKII | 290 | 4.56E+08 | Plasminogen |
| | | | | | DR | | | activator |
| | | | | | | | | inhibitor 1 |
| | | | | | | | | RNA−binding |
| | | | | | | | | protein |
| SERPINH127 | P50454 | SERPINH1 | 27 | (A) | AAAPGTAE | 291 | 1.82E+08 | Serpin H1 |
| | | | | | KLSPKAAT | | | |
| | | | | | LAER | | | |
| SMOC1_35 | Q9H4F8 | SMOC1 | 35 | (F) | LISDRDPQC | 292 | 9.50E+07 | SPARC− |
| | | | | | NLHCSR | | | related |
| | | | | | | | | modular |
| | | | | | | | | calcium− |
| | | | | | | | | binding |
| | | | | | | | | protein 1 |
| SPOCK1_126 | Q08629 | SPOCK1 | 126 | (K) | HWVGPSNL | 293 | 8.43E+07 | Testican−1 |
| | | | | | VK | | | |
| SRGN_50 | P10124 | SRGN | 50 | (C) | LEEKGPMF | 294 | 1.42E+09 | Serglycin |
| | | | | | ELLPGESN | | | |
| | | | | | KIPR | | | |
| SRGN_51 | P10124 | SRGN | 51 | (L) | EEKGPMFE | 295 | 3.45E+07 | Serglycin |
| | | | | | LLPGESNKI | | | |
| | | | | | PR | | | |
| SRGN_59 | P10124 | SRGN | 59 | (E) | LLPGESNKI | 296 | 2.70E+09 | Serglycin |
| | | | | | PR | | | |
| SRGN_60 | P10124 | SRGN | 60 | (L) | LPGESNKIP | 297 | 2.92E+07 | Serglycin |
| | | | | | R | | | |
| SRGN_62 | P10124 | SRGN | 62 | (P) | GESNKIPR | 298 | 3.99E+08 | Serglycin |
| SRGN_129 | P10124 | SRGN | 129 | (A) | FHDNLR | 299 | 2.29E+09 | Serglycin |
| STC1_37 | P52823 | STC1 | 37 | (A) | QNSAEVVR | 300 | 1.51E+08 | Stanniocalcin- |
| | | | | | | | | 1 |
| STC2_239 | O76061 | STC2 | 239 | (H) | HGEAGHHL | 301 | 9.79E+07 | Stanniocalcin- |
| | | | | | PEPSSR | | | 2 |
| STC2_244 | O76061 | STC2 | 244 | (G) | HHLPEPSSR | 302 | 1.06E+09 | Stanniocalcin- |
| | | | | | | | | 2 |
| SYNJ1_1350 | A0A0D9SGJ6 | SYNJ1 | 1350 | (F) | ITGLTR | 303 | 3.03E+08 | Synaptojanin- |
| | | | | | | | | 1 |
| SYNM_968 | A0A075B7B1 | SYNM | 968 | (E) | LSALTR | 304 | 1.21E+09 | Desmuslin, |
| | | | | | | | | isoform |
| | | | | | | | | CRA a |
| TAGLN_168 | Q01995 | TAGLN | 168 | (Q) | LQEGKHVI | 305 | 5.04E+08 | Transgelin |
| | | | | | GLQmGSNR | | | |
| TAGLN_178 | Q01995 | TAGLN | 178 | (L) | QMGSNR | 306 | 1.29E+07 | Transgelin |
| TAGLN2_167 | P37802 | TAGLN2 | 167 | (Q) | LQEGKNVI | 307 | 8.44E+07 | Transgelin−2 |
| | | | | | GLQmGTNR | | | |
| TG_1017 | P01266 | TG | 1017 | (A) | GASALLR | 308 | 1.85E+08 | Thyroglobulin |
| TGFBI_24 | Q15582 | TGFBI | 24 | (A) | GPAKSPYQ | 309 | 6.17E+10 | Transforming |
| | | | | | LVLQHSR | | | growth |
| | | | | | | | | factor−beta− |
| | | | | | | | | induced |
| | | | | | | | | protein ig−h3 |
| TGFBI_43 | Q15582 | TGFBI | 43 | (R) | qHGPNVCA | 310 | 3.19E+10 | Transforming |
| | | | | | VQKVIGTN | | | growth |
| | | | | | R | | | factor−beta− |
| | | | | | | | | induced |
| | | | | | | | | protein ig−h3 |
| TGFBI_50 | Q15582 | TGFBI | 50 | (C) | AVQKVIGT | 311 | 5.27E+08 | Transforming |
| | | | | | NR | | | growth |
| | | | | | | | | factor−beta− |
| | | | | | | | | induced |
| | | | | | | | | protein ig−h3 |
| TGFBI_208 | Q15582 | TGFBI | 208 | (N) | GIVTVNCA | 312 | 2.03E+08 | Transforming |
| | | | | | R | | | growth |
| | | | | | | | | factor−beta− |
| | | | | | | | | induced |
| | | | | | | | | protein ig−h3 |
| TGFBI_276 | Q15582 | TGFBI | 276 | (Y) | TLLAPTnEA | 313 | 2.25E+08 | Transforming |
| | | | | | FEKIPSETL | | | growth |
| | | | | | NR | | | factor−beta− |
| | | | | | | | | induced |
| | | | | | | | | protein ig−h3 |
| TGFBI_497 | Q15582 | TGFBI | 497 | (R) | VLTPPmGT | 314 | 3.45E+08 | Transforming |
| | | | | | VMDVLKG | | | growth |
| | | | | | DNR | | | factor−beta− |
| | | | | | | | | induced |
| | | | | | | | | protein ig−h3 |
| TGFBI_524 | Q15582 | TGFBI | 524 | (Q) | SAGLTETL | 315 | 7.48E+07 | Transforming |
| | | | | | NR | | | growth |
| | | | | | | | | factor−beta− |
| | | | | | | | | induced |
| | | | | | | | | protein ig−h3 |
| TGFBI_534 | Q15582 | TGFBI | 534 | (R) | EGVYTVFA | 316 | 6.29E+09 | Transforming |
| | | | | | PTNEAFR | | | growth |
| | | | | | | | | factor−beta− |
| | | | | | | | | induced |
| | | | | | | | | protein ig−h3 |
| THBS1_972 | P07996 | THB S1 | 972 | (S) | QNDPNWV | 317 | 3.65E+08 | Thrombospondin− |
| | | | | | VR | | | 1 |
| THBS2_290 | P35442 | THBS2 | 290 | (Q) | LSENLKR | 318 | 4.15E+08 | Thrombospondin− |
| | | | | | | | | 2 |
| TLN1_1501 | Q9Y490 | TLN1 | 1501 | (K) | HTSALCNS | 319 | 6.47E+06 | Talin−1 |
| | | | | | CR | | | |
| TMEM132A52 | Q24JP5 | TMEM132A | 52 | (E) | LLDAPEHF | 320 | 1.07E+07 | Transmembrane |
| | | | | | R | | | protein |
| | | | | | | | | 132A |
| TMTC1_585 | F8VTQ9 | TMTC1 | 585 | (N) | LGTLTR | 321 | 6.05E+08 | Transmembrane |
| | | | | | | | | and TPR |
| | | | | | | | | repeat− |
| | | | | | | | | containing |
| | | | | | | | | protein 1 |
| VASN_138 | Q6EMK4 | VASN | 138 | (R) | HIQPGAFD | 322 | 4.88E+07 | Vasorin |
| | | | | | TLDR | | | |
| VASP_229 | P50552 | VASP | 229 | (A) | AIAGAKLR | 323 | 3.14E+07 | Vasodilator− |
| | | | | | | | | stimulated |
| | | | | | | | | phosphoprotein |
| VIM_314 | P08670 | VIM | 314 | (K) | QESTEYR | 324 | 4.88E+07 | Vimentin |
| VIM_333 | P08670 | VIM | 333 | (A) | LKGTNESL | 325 | 2.04E+08 | Vimentin |
| | | | | | ER | | | |
| VIM_433 | P08670 | VIM | 433 | (P) | LVDTHSKR | 326 | 1.81E+08 | Vimentin |

SMC2_TAILS HC:

| | | SEQ | | | | | | |
|---|---|---|---|---|---|---|---|---|
| TAILS_ | | ID | adj.P.Val. | adj.P.Val. | adj.P.Val. | logFC.mEQ. | logFC.mWT. | logFC.mWT. |
| ID | id | NO: | mEQ.over.Luc | mWT.over.Luc | mWT.over.mEQ | over.Luc | over.Luc | over.mEQ |
| ACTG1_ | LTEAPLNPKANR | 120 | 7.70E−01 | 1.83E−03 | 5.03E−04 | −0.31 | 1.80 | 2.11 |
| 105 | | | | | | | | |
| ACTN4_ | HAANQSYQYGPSS | 327 | 1.00E+00 | 1.51E−03 | 1.51E−04 | 0.00 | 2.65 | 2.64 |
| 5 | AGnGAGGGGSmG | | | | | | | |
| ADAM | FKVETSNKVL | 328 | 6.31E−01 | 3.16E−04 | 3.74E−04 | −0.29 | 2.45 | 2.74 |
| 10_86 | | | | | | | | |
| ADAM | VIQAEGKEHIIHLE | 122 | 8.91E−01 | 3.64E−03 | 1.38E−02 | 0.21 | 2.29 | 2.09 |
| 9_69 | R | | | | | | | |
| ADAM | VIQAEGKEHIIHL | 329 | 6.21E−01 | 1.47E−02 | 7.68E−03 | −0.30 | 0.91 | 1.21 |
| 9_69 | | | | | | | | |
| ADAM | VIQAEGK | 330 | 5.02E−01 | 6.73E−04 | 5.98E−04 | −0.39 | 1.52 | 1.91 |
| 9_69 | | | | | | | | |
| ADAM | GKEHIIHLER | 331 | 9.79E−01 | 3.80E−04 | 4.85E−05 | −0.18 | 4.13 | 4.31 |
| 9_74 | | | | | | | | |
| ADAM | HVVGPVR | 332 | 9.97E−01 | 2.84E−03 | 1.90E−02 | −0.04 | 1.24 | 1.29 |
| TS12_ | | | | | | | | |
| 53 | | | | | | | | |
| ADNP2_ | SQPVGPVNK | 333 | 9.67E−02 | 4.05E−02 | 4.87E−02 | 4.60 | 5.35 | 0.75 |
| 375 | | | | | | | | |
| AEBP1_ | FLEGFLSELEPEPR | 123 | 8.81E−01 | 7.72E−05 | 3.63E−04 | −0.27 | 3.21 | 3.48 |
| 37 | | | | | | | | |
| AKAP12_ | FTQGKVVGQTTPE | 334 | 9.96E−01 | 6.14E−05 | 1.82E−04 | −0.04 | 2.89 | 2.94 |
| 1106 | SF | | | | | | | |
| AMER2_ | LAGTTIRATACH | 335 | 9.93E−01 | 5.90E−03 | 3.33E−02 | 0.05 | 0.95 | 0.90 |
| 654 | | | | | | | | |
| APLP2_ | LVETHLAR | 336 | 9.79E−01 | 6.98E−04 | 2.05E−04 | 0.11 | 2.70 | 2.59 |
| 452 | | | | | | | | |
| APOA1_ | EQLGPVTQEFW | 337 | 9.93E−01 | 3.15E−04 | 4.02E−03 | −0.07 | 1.80 | 1.88 |
| 86 | | | | | | | | |
| ATP1A2_ | PITAKAIAKGVGIIS | 338 | 9.95E−01 | 1.90E−03 | 3.48E−03 | 0.07 | 1.85 | 1.79 |
| 618 | EGNETVEDIAARLn | | | | | | | |
| | IPmSQVnPREAK | | | | | | | |
| BCOR_ | DFIALR | 339 | 9.79E−01 | 7.21E−05 | 2.68E−04 | 0.09 | 2.58 | 2.49 |
| 258 | | | | | | | | |
| BMP6_ | LKSAPLFmL | 340 | 9.04E−01 | 6.91E−04 | 2.48E−02 | −0.24 | 1.26 | 1.50 |
| 129 | | | | | | | | |
| BMPER_ | LTGSVAKCENEG | 341 | 9.91E−01 | 1.31E−04 | 1.52E−04 | −0.06 | 2.37 | 2.43 |
| 43 | | | | | | | | |
| CALU_ | FTAFLHPEEY | 342 | 9.99E−01 | 2.59E−03 | 4.26E−03 | −0.01 | 2.37 | 2.38 |
| 176 | | | | | | | | |
| CAP1_ | FSAPKPQTSPSPKR | 343 | 9.91E−01 | 6.14E−05 | 4.00E−04 | 0.09 | 2.91 | 2.81 |
| 300 | | | | | | | | |
| CARHS | ASQGPVYK | 344 | 9.91E−01 | 1.08E−02 | 5.62E−03 | 0.07 | 1.08 | 1.00 |
| P1_57 | | | | | | | | |
| CCDC80_ | LEEPNLQPLQR | 127 | 9.83E−01 | 6.28E−05 | 4.00E−03 | −0.13 | 2.08 | 2.21 |
| 66 | | | | | | | | |
| CCDC80_ | QVGNVPLKKAK | 345 | 9.96E−01 | 6.14E−05 | 1.03E−04 | 0.04 | 3.67 | 3.63 |
| 525 | | | | | | | | |
| CCL2_ | INAPVTCCYNFTNR | 346 | 9.78E−01 | 2.10E−03 | 3.36E−03 | −0.11 | 1.35 | 1.46 |
| 28 | | | | | | | | |
| CEP192_ | DKSTAGR | 347 | 2.57E−01 | 1.74E−04 | 6.73E−05 | −0.62 | 2.33 | 2.95 |
| 728 | | | | | | | | |
| COL12 | KGGNTMTG | 348 | 9.32E−01 | 2.09E−02 | 2.89E−02 | −0.15 | 0.80 | 0.95 |
| A1_213 | | | | | | | | |
| COL16 | QSEGKVYTR | 132 | 9.82E−01 | 1.31E−04 | 6.99E−05 | 0.13 | 4.06 | 3.93 |
| A1_269 | | | | | | | | |
| COL18 | LQPPVVQLH | 349 | 9.99E−01 | 4.99E−04 | 2.75E−02 | −0.01 | 1.46 | 1.47 |
| A1_1505 | | | | | | | | |
| COL1A | FQGPPGEPGEPGAS | 350 | 9.91E−01 | 1.76E−02 | 9.87E−03 | −0.07 | 0.87 | 0.94 |
| 1_201 | GPMGPR | | | | | | | |
| COL1A | LTGPIGPPGPAGAP | 351 | 8.80E−01 | 3.17E−03 | 2.89E−02 | −0.21 | 0.91 | 1.12 |
| 1_765 | G | | | | | | | |
| COL1A | LSQQIENIR | 352 | 9.93E−01 | 5.70E−05 | 1.07E−04 | 0.06 | 2.91 | 2.85 |
| 1_1238 | | | | | | | | |
| COLIA | LLQGSNEIEIR | 353 | 6.53E−01 | 2.00E−04 | 3.51E−02 | 0.56 | 2.25 | 1.69 |
| 1_1389 | | | | | | | | |
| COL1A | qYDGKGVGLGPGP | 135 | 7.39E−01 | 9.93E−03 | 3.44E−02 | −0.33 | 0.82 | 1.15 |
| 2_80 | mGLmGPR | | | | | | | |
| COL1A | FQGPAGEPG | 354 | 9.93E−01 | 3.04E−03 | 4.01E−03 | −0.05 | 1.02 | 1.07 |
| 2_113 | | | | | | | | |
| COL1A | FQGPAGEPGEPGQT | 137 | 5.73E−01 | 1.10E−03 | 4.94E−04 | −0.38 | 1.94 | 2.33 |
| 2_113 | GPAGAR | | | | | | | |
| COL1A | VGNAGPAGPAGPR | 138 | 9.97E−01 | 1.31E−03 | 9.82E−05 | −0.05 | 3.42 | 3.47 |
| 2_270 | | | | | | | | |
| COL1A | LTGAKGAAGLPGV | 139 | 6.75E−01 | 5.55E−03 | 4.30E−03 | −0.32 | 1.60 | 1.92 |
| 2_305 | AGAPGLPGPR | | | | | | | |
| COL1A | VmGPPGSR | 355 | 6.75E−01 | 4.13E−04 | 1.30E−02 | −0.76 | 2.79 | 3.55 |
| 2_416 | | | | | | | | |
| COL1A | FGLPGPAGPR | 356 | 9.09E−01 | 8.46E−05 | 2.22E−04 | 0.20 | 3.20 | 3.00 |
| 2_579 | | | | | | | | |
| COL1A | VGTAGPSGPSGLPG | 142 | 8.15E−01 | 3.32E−03 | 3.48E−04 | −0.30 | 2.03 | 2.33 |
| 2_630 | ER | | | | | | | |
| COL1A | FAGPAGAAGQPGA | 357 | 5.50E−01 | 2.33E−03 | 8.22E−04 | −0.40 | 1.48 | 1.88 |
| 2_725 | KGER | | | | | | | |
| COL1A | LLGAPGILGLPGSR | 145 | 6.97E−01 | 8.25E−05 | 1.08E−03 | −0.46 | 2.76 | 3.22 |
| 2_866 | | | | | | | | |
| COL1A | LGAPGILGLPGSR | 358 | 5.59E−01 | 1.31E−03 | 4.32E−04 | −0.41 | 1.63 | 2.05 |
| 2_867 | | | | | | | | |
| COL1A | AVGEPGPLGIAGPP | 359 | 7.26E−01 | 3.24E−04 | 1.29E−04 | −0.29 | 2.61 | 2.90 |
| 2_890 | GAR | | | | | | | |
| COL1A | VGSPGVNGAPGEA | 360 | 5.18E−01 | 9.72E−03 | 3.50E−04 | −0.51 | 1.33 | 1.83 |
| 2_912 | GR | | | | | | | |
| COL1A | AGAPGPHGPVGPA | 146 | 9.81E−01 | 6.74E−04 | 1.83E−03 | −0.16 | 2.71 | 2.87 |
| 2_960 | GKHGNR | | | | | | | |
| COL1A | LQGLPGIAGHHG | 361 | 8.50E−01 | 1.22E−03 | 1.98E−03 | −0.25 | 1.73 | 1.98 |
| 2_1028 | | | | | | | | |
| COL1A | HQGPAGPPGPPGPP | 362 | 9.99E−01 | 1.84E−04 | 1.13E−03 | 0.00 | 1.89 | 1.89 |
| 2_1085 | GPPGVSGGGY | | | | | | | |
| COLIA | LLTPEGSR | 363 | 7.44E−01 | 2.42E−04 | 4.19E−04 | −0.27 | 1.98 | 2.25 |
| 2_1149 | | | | | | | | |
| COL3A | QQGAIGSPGPAGPR | 364 | 9.78E−01 | 1.45E−03 | 2.00E−04 | −0.21 | 3.21 | 3.42 |
| 1_1126 | | | | | | | | |
| COL4A | YNGPPGLQGFPGL | 150 | 7.96E−01 | 4.55E−04 | 1.45E−02 | −0.32 | 1.38 | 1.70 |
| 2_71 | QGR | | | | | | | |
| COL4A | HQGPIGQEGAPGRP | 365 | 9.93E−01 | 1.31E−04 | 1.76E−03 | −0.06 | 1.71 | 1.76 |
| 2_1460 | GSPGLPGmPGR | | | | | | | |
| COL5A | QESQAQAILQQAR | 366 | 4.29E−01 | 2.20E−02 | 4.65E−03 | −0.49 | 0.68 | 1.18 |
| 1_542 | | | | | | | | |
| COL5A | LTGNPGVQGPEGK | 367 | 5.92E−01 | 1.04E−02 | 2.27E−03 | −0.64 | 2.02 | 2.66 |
| 2_574 | LGPLGAPGE | | | | | | | |
| COL6A | FQGCPGQR | 368 | 9.91E−01 | 1.31E−04 | 5.96E−04 | 0.08 | 2.17 | 2.10 |
| 3_2084 | | | | | | | | |
| COL7A | ISSSQR | 369 | 9.97E−01 | 6.62E−05 | 4.49E−04 | −0.03 | 2.38 | 2.41 |
| 1_1009 | | | | | | | | |
| CTSB_ | WAFGAVEAIS | 370 | 9.96E−01 | 8.50E−03 | 2.48E−02 | −0.08 | 1.65 | 1.73 |
| 109 | | | | | | | | |
| DBN1_ | APVEPATA | 371 | 9.97E−01 | 2.52E−04 | 8.83E−04 | 0.03 | 1.61 | 1.57 |
| 434 | | | | | | | | |
| DDX39 | AGLAPR | 372 | 5.94E−01 | 4.15E−04 | 1.41E−04 | −0.95 | 4.74 | 5.69 |
| B_387 | | | | | | | | |
| DIS3L_ | FPELKGIIFMQTAC | 373 | 9.98E−01 | 7.94E−04 | 8.32E−03 | −0.02 | 1.77 | 1.78 |
| 75 | QAVQHQR | | | | | | | |
| ECM1_ | FQEVGYAAPPSPPL | 374 | 9.98E−01 | 3.04E−04 | 1.78E−02 | −0.02 | 1.54 | 1.57 |
| 38 | SR | | | | | | | |
| ECM1_ | AAPPSPPLSR | 375 | 6.88E−01 | 1.56E−04 | 7.49E−05 | −0.28 | 2.25 | 2.54 |
| 44 | | | | | | | | |
| ECM1_ | QEAVPLQK | 376 | 7.95E−01 | 1.48E−04 | 1.07E−04 | −0.22 | 3.12 | 3.34 |
| 108 | | | | | | | | |
| EEF1G_ | QYSGAQVR | 377 | 9.79E−01 | 8.68E−05 | 6.68E−05 | −0.12 | 3.18 | 3.30 |
| 23 | | | | | | | | |
| EFEMP | AVAGPEMQTGR | 157 | 5.88E−01 | 9.45E−05 | 1.29E−04 | −0.46 | 2.81 | 3.27 |
| 1_124 | | | | | | | | |
| EFEMP | VAGPEMQTGR | 158 | 3.01E−01 | 3.12E−03 | 3.99E−03 | −0.99 | 1.43 | 2.42 |
| 1_125 | | | | | | | | |
| EFEMP | qTGRNNFVIR | 378 | 9.28E−01 | 1.45E−03 | 2.80E−03 | −0.14 | 1.03 | 1.17 |
| 1_131 | | | | | | | | |
| EIF4B_ | QTGTSTTSSR | 379 | 7.86E−01 | 1.84E−04 | 4.00E−04 | 0.49 | 5.61 | 5.12 |
| 426 | | | | | | | | |
| EIF4G2_ | qISLRPAQSFLMnKn | 380 | 7.58E−01 | 3.94E−04 | 2.18E−03 | 0.34 | 3.13 | 2.79 |
| 433 | QVPK | | | | | | | |
| EMILIN | QIAPR | 381 | 5.94E−01 | 4.15E−04 | 1.41E−04 | −0.95 | 4.74 | 5.69 |
| 1_47 | | | | | | | | |
| EMILIN | QVSSQGSR | 382 | 9.98E−01 | 2.89E−05 | 4.64E−05 | 0.01 | 3.60 | 3.59 |
| 3_636 | | | | | | | | |
| EMSY_ | QTVVQVLAVK | 383 | 8.36E−01 | 3.84E−03 | 6.27E−03 | 0.20 | 1.13 | 0.92 |
| 943 | | | | | | | | |
| FAM35 | LSENKIR | 384 | 9.93E−01 | 1.73E−04 | 4.85E−05 | 0.08 | 4.98 | 4.89 |
| A_142 | | | | | | | | |
| FAT3_1 | IKLPER | 385 | 9.79E−01 | 2.03E−03 | 4.48E−04 | −0.13 | 1.79 | 1.91 |
| 111 | | | | | | | | |
| FBLN2_ | ALGPPAPVQAKAR | 162 | 7.15E−01 | 7.75E−04 | 2.33E−04 | −0.27 | 1.61 | 1.88 |
| 259 | | | | | | | | |
| FBLN2_ | LGPPAPVQAKAR | 163 | 6.33E−01 | 2.52E−04 | 1.15E−03 | −0.44 | 1.98 | 2.42 |
| 260 | | | | | | | | |
| FBLN2_ | FSQVASNTIPLPLPQ | 386 | 9.42E−01 | 3.21E−04 | 2.39E−03 | −0.15 | 1.74 | 1.89 |
| 665 | PNTCK | | | | | | | |
| FBN1_ | LEAGNVK | 387 | 9.30E−01 | 9.42E−05 | 4.49E−04 | −0.24 | 3.33 | 3.57 |
| 29 | | | | | | | | |
| FBN1_ | LKGPNVCGSR | 165 | 9.03E−01 | 9.27E−05 | 6.99E−05 | −0.25 | 4.64 | 4.89 |
| 53 | | | | | | | | |
| FBXO4 | RKnFLQNVAnAFAC | 388 | 9.81E−01 | 1.73E−04 | 1.71E−03 | −0.14 | 2.49 | 2.63 |
| 7_390 | VImEMLQSIMSGDR | | | | | | | |
| FLJ439 | LQSSLRKWNPL | 389 | 9.53E−01 | 8.25E−05 | 7.49E−05 | 0.17 | 3.59 | 3.42 |
| 44_211 | | | | | | | | |
| FLNA_ | VNKPAEFTV | 390 | 1.11E−01 | 1.78E−04 | 4.65E−05 | −1.45 | 4.67 | 6.12 |
| 689 | | | | | | | | |
| FLNA_ | LTQTGGPHVKAR | 168 | 3.75E−01 | 1.84E−04 | 1.09E−03 | −1.35 | 4.37 | 5.72 |
| 1285 | | | | | | | | |
| FLNC_ | FVGQKNSFTV | 39 | 9.96E−01 | 4.55E−05 | 1.03E−04 | −0.06 | 3.91 | 3.97 |
| 2649 | | | | | | | | |
| FN1_35 | qm VQPQSPVAVSQS | 392 | 9.81E−01 | 1.74E−04 | 5.84E−04 | −0.09 | 1.99 | 2.08 |
| | KPGCY | | | | | | | |
| FN1_36 | MVQPQSPVAVSQS | 393 | 9.84E−01 | 8.25E−05 | 5.51E−05 | −0.11 | 3.59 | 3.69 |
| | KPGCY | | | | | | | |
| FN1_40 | QSPVAVSQSKPGC | 394 | 9.93E−01 | 8.46E−05 | 4.17E−04 | −0.06 | 2.60 | 2.66 |
| | Y | | | | | | | |
| FN1_99 | DKYTGNTYR | 395 | 9.93E−01 | 2.80E−02 | 1.79E−03 | −0.08 | 1.35 | 1.43 |
| FN1_ | TSSGSGPFT | 396 | 9.19E−01 | 1.31E−03 | 4.38E−03 | −0.16 | 1.30 | 1.46 |
| 279 | | | | | | | | |
| FN1_ | SSSGPVEVFIT | 397 | 4.62E−01 | 3.08E−03 | 4.61E−02 | 0.45 | 1.08 | 0.63 |
| 607 | | | | | | | | |
| FN1_ | LTPGVEYVYTIQVL | 177 | 5.18E−01 | 4.39E−04 | 7.72E−05 | −0.49 | 2.31 | 2.80 |
| 1143 | R | | | | | | | |
| FN1_ | SSSPVTGYR | 178 | 5.12E−01 | 2.41E−02 | 1.69E−03 | −0.65 | 1.20 | 1.86 |
| 1656 | | | | | | | | |
| FN1_ | LQPTVEYVVSVYA | 398 | 9.95E−01 | 9.85E−05 | 6.54E−03 | −0.07 | 1.99 | 2.05 |
| 1693 | QNPSG | | | | | | | |
| FN1_ | LVQTAVTNI | 399 | 9.97E−01 | 3.67E−03 | 7.43E−04 | 0.06 | 2.22 | 2.17 |
| 1715 | | | | | | | | |
| FN1_ | LQGLRPGSEYTVSV | 400 | 6.87E−01 | 2.90E−04 | 7.93E−04 | −0.34 | 1.67 | 2.01 |
| 1780 | VALH | | | | | | | |
| FN1_ | LIGTQSTAIPAPT | 401 | 1.88E−01 | 9.35E−05 | 6.84E−04 | 0.71 | 2.43 | 1.73 |
| 1805 | | | | | | | | |
| FN1_ | FVTHPGY | 402 | 8.61E−01 | 4.16E−04 | 1.41E−03 | −0.19 | 1.38 | 1.57 |
| 2113 | | | | | | | | |
| FN1_ | SVGQQmIF | 403 | 9.04E−01 | 1.70E−03 | 6.33E−03 | −0.18 | 1.15 | 1.32 |
| 2136 | | | | | | | | |
| FOXG1_ | LNKCFVKVPRHY | 404 | 9.93E−01 | 5.70E−05 | 1.88E−04 | 0.07 | 3.42 | 3.34 |
| 235 | | | | | | | | |
| FSTL_ | LQKHQETA | 405 | 9.16E−01 | 1.75E−04 | 2.00E−03 | −0.28 | 2.58 | 2.86 |
| 1290 | | | | | | | | |
| FYB2_ | FKVDAC | 406 | 9.06E−01 | 6.14E−05 | 2.00E−04 | −0.24 | 3.50 | 3.74 |
| 408 | | | | | | | | |
| GSN_ | HIANVER | 187 | 9.73E−01 | 5.10E−03 | 2.19E−02 | 0.18 | 1.91 | 1.74 |
| 413 | | | | | | | | |
| HEG1_ | LAGAGLELQL | 407 | 9.68E−01 | 1.71E−03 | 1.11E−02 | 0.17 | 2.19 | 2.01 |
| 47 | | | | | | | | |
| HEG1_ | GLELQLER | 408 | 7.09E−01 | 5.68E−03 | 3.28E−02 | 0.25 | 0.90 | 0.65 |
| 51 | | | | | | | | |
| HEG1_ | FQTKSGTAS | 409 | 7.34E−01 | 5.70E−05 | 9.49E−05 | 0.29 | 3.54 | 3.25 |
| 224 | | | | | | | | |
| HSPG2_ | LLPGSVRPLPCGPQ | 410 | 6.75E−01 | 9.58E−04 | 4.00E−04 | −0.36 | 1.74 | 2.10 |
| 275 | | | | | | | | |
| HSPG2_ | LSAPVVSIHPPQLT | 411 | 5.98E−01 | 2.50E−03 | 3.09E−03 | −0.50 | 1.40 | 1.90 |
| 1863 | VQPGQLA | | | | | | | |
| HSPG2_ | HSSAGQQVAR | 412 | 7.06E−01 | 5.77E−03 | 4.39E−03 | −0.45 | 1.54 | 1.98 |
| 1936 | | | | | | | | |
| HSPG2_ | ANLAYPAGSTQPIR | 194 | 8.60E−01 | 1.74E−04 | 4.51E−04 | −0.19 | 1.72 | 1.91 |
| 2331 | | | | | | | | |
| HSPG2_ | LAYPAGSTQPIR | 195 | 8.12E−01 | 1.11E−03 | 2.31E−03 | −0.22 | 1.19 | 1.41 |
| 2333 | | | | | | | | |
| HSPG2_ | GVAYPVR | 413 | 8.00E−01 | 1.65E−02 | 7.36E−04 | −0.28 | 1.20 | 1.49 |
| 2531 | | | | | | | | |
| HSPG2_ | LAQNALGTAQKQV | 414 | 8.32E−01 | 4.99E−02 | 4.44E−02 | −0.21 | 0.56 | 0.77 |
| 3186 | EVIV | | | | | | | |
| HSPG2_ | FAHLQVPER | 415 | 6.97E−01 | 4.59E−03 | 9.44E−04 | −0.33 | 1.29 | 1.61 |
| 3653 | | | | | | | | |
| IGFBP3_ | SAGLGPVVR | 211 | 6.29E−01 | 1.53E−04 | 8.08E−05 | −0.31 | 2.12 | 2.44 |
| 31 | | | | | | | | |
| IGFBP3_ | AGLGPVVR | 212 | 6.24E−01 | 5.16E−05 | 4.67E−05 | −0.51 | 4.68 | 5.18 |
| 32 | | | | | | | | |
| IGFBP7_ | AAGGPGVSGVCVC | 214 | 9.93E−01 | 7.21E−05 | 1.03E−04 | −0.04 | 2.53 | 2.57 |
| 101 | KSR | | | | | | | |
| IPP_504 | EKYSFE | 416 | 9.96E−01 | 4.55E−05 | 1.08E−03 | 0.09 | 3.90 | 3.81 |
| JAK3_ | GALAPR | 417 | 5.94E−01 | 4.15E−04 | 1.41E−04 | −0.95 | 4.74 | 5.69 |
| 794 | | | | | | | | |
| KCND1_ | RLAKSGTTNAFLQ | 418 | 9.81E−01 | 2.29E−03 | 2.93E−02 | −0.14 | 1.60 | 1.74 |
| 436 | YKQNGGLE | | | | | | | |
| KPNB1_ | LVELSR | 216 | 9.97E−01 | 1.94E−03 | 8.32E−03 | 0.03 | 1.14 | 1.11 |
| 37 | | | | | | | | |
| KRT28_ | SLQFSNGSR | 419 | 8.61E−01 | 2.23E−02 | 2.76E−03 | −0.34 | 1.88 | 2.22 |
| 2 | | | | | | | | |
| LAMA5_ | QVLPAGQIVNC | 420 | 9.97E−01 | 5.95E−03 | 1.52E−03 | −0.05 | 2.14 | 2.19 |
| 484 | | | | | | | | |
| LASP1_ | qSYGGYK | 421 | 9.79E−01 | 2.88E−04 | 3.55E−04 | −0.08 | 1.51 | 1.59 |
| 181 | | | | | | | | |
| LASP1_ | SYGGYKEPAAPVSI | 422 | 9.36E−01 | 2.20E−02 | 4.16E−02 | −0.14 | 0.87 | 1.01 |
| 182 | QR | | | | | | | |
| LMNA_ | qTQGGGSVTKKR | 222 | 7.34E−01 | 7.72E−05 | 6.99E−05 | −0.41 | 4.37 | 4.77 |
| 408 | | | | | | | | |
| LOX_5 | LSLGSQYQPQR | 223 | 9.81E−01 | 1.58E−04 | 3.36E−04 | −0.08 | 1.68 | 1.77 |
| 8 | | | | | | | | |
| LOX_6 | LGSQYQPQR | 423 | 6.27E−01 | 2.58E−03 | 4.51E−04 | −0.34 | 1.25 | 1.59 |
| 0 | | | | | | | | |
| LOX_1 | HWFQAGYSTSR | 226 | 1.00E+00 | 5.83E−04 | 4.91E−02 | 0.00 | 1.37 | 1.37 |
| 23 | | | | | | | | |
| LOX_1 | WFQAGYSTSR | 227 | 9.88E−01 | 2.94E−05 | 2.64E−04 | −0.11 | 3.35 | 3.45 |
| 24 | | | | | | | | |
| LOX_1 | FQAGYSTSR | 424 | 9.81E−01 | 3.76E−05 | 1.29E−04 | 0.11 | 3.45 | 3.33 |
| 25 | | | | | | | | |
| LOX_1 | QAGYSTSR | 228 | 8.42E−01 | 2.88E−04 | 6.77E−05 | −0.26 | 2.77 | 3.03 |
| 26 | | | | | | | | |
| LOXL1_ | SVSASAFASTYR | 425 | 9.30E−01 | 1.28E−04 | 2.13E−04 | −0.19 | 2.71 | 2.90 |
| 157 | | | | | | | | |
| LOXL1_ | AVASAGVIYPYQP | 426 | 9.85E−01 | 1.56E−04 | 6.73E−03 | −0.11 | 1.72 | 1.83 |
| 218 | R | | | | | | | |
| LOXL1_ | VASAGVIYPYQPR | 229 | 9.85E−01 | 3.33E−04 | 8.81E−03 | 0.09 | 1.56 | 1.47 |
| 219 | | | | | | | | |
| LOXL2_ | FQQPAPEYHQPQA | 230 | 9.97E−01 | 4.55E−05 | 3.55E−04 | −0.03 | 3.27 | 3.30 |
| 37 | PANVAKIQLR | | | | | | | |
| LOXL2_ | qQPAPEYHQPQAPA | 231 | 9.91E−01 | 2.93E−04 | 5.48E−04 | 0.06 | 1.74 | 1.68 |
| 38 | NVAKIQLR | | | | | | | |
| LOXL2_ | HQPQAPANVAKIQ | 427 | 9.91E−01 | 1.21E−03 | 4.46E−04 | 0.11 | 2.62 | 2.51 |
| 45 | LR | | | | | | | |
| LRRC1 | qVSGRPPVIKPEV | 428 | 9.81E−01 | 7.08E−05 | 4.65E−05 | 0.09 | 2.79 | 2.69 |
| 7_225 | | | | | | | | |
| LTBP1_ | LTATNFR | 234 | 9.88E−01 | 8.46E−04 | 9.66E−05 | 0.29 | 6.60 | 6.30 |
| 393 | | | | | | | | |
| LTBP1_ | HQQVIPHVYPVAA | 236 | 9.97E−01 | 1.52E−03 | 1.07E−02 | 0.05 | 1.82 | 1.77 |
| 540 | KTQLGR | | | | | | | |
| LTBP1_ | SEQYTPEA | 429 | 9.97E−01 | 1.78E−03 | 1.41E−02 | −0.03 | 1.44 | 1.47 |
| 1597 | | | | | | | | |
| LTBP2_ | VAGLQPVER | 430 | 9.84E−01 | 1.08E−03 | 4.94E−04 | −0.09 | 1.54 | 1.62 |
| 79 | | | | | | | | |
| LTBP2_ | SAAGEGTLAR | 431 | 8.00E−01 | 7.21E−05 | 5.15E−05 | −0.21 | 2.49 | 2.70 |
| 250 | | | | | | | | |
| LTBP2_ | QHVGLSR | 432 | 9.81E−01 | 9.85E−05 | 4.67E−05 | −0.10 | 2.94 | 3.05 |
| 290 | | | | | | | | |
| LTBP2_ | LVENSVETRPPPWL | 433 | 9.19E−01 | 8.73E−05 | 4.00E−04 | −0.18 | 2.66 | 2.85 |
| 502 | PASPGHSLW | | | | | | | |
| LTBP3_ | LTGSGFR | 434 | 5.04E−01 | 1.33E−04 | 5.38E−05 | −0.92 | 4.80 | 5.72 |
| 103 | | | | | | | | |
| LTBP3_ | FLVPLGPGQISA | 435 | 9.98E−01 | 3.00E−02 | 3.36E−03 | −0.03 | 1.51 | 1.53 |
| 208 | | | | | | | | |
| LTBP3_ | SVQVHR | 248 | 9.93E−01 | 2.67E−03 | 1.10E−03 | −0.07 | 1.53 | 1.59 |
| 239 | | | | | | | | |
| MAP3K | LLTGEIPYK | 436 | 9.37E−01 | 9.25E−05 | 6.55E−04 | −0.15 | 1.84 | 1.99 |
| 13_346 | | | | | | | | |
| MAP4_ | AVASTGPSSR | 252 | 9.98E−01 | 1.76E−03 | 1.09E−03 | −0.02 | 1.68 | 1.70 |
| 815 | | | | | | | | |
| MAP4_ | VASTGPSSR | 253 | 9.97E−01 | 6.62E−05 | 9.94E−05 | 0.02 | 2.53 | 2.51 |
| 816 | | | | | | | | |
| MARC | AESGAK | 437 | 9.97E−01 | 2.52E−04 | 3.09E−04 | −0.02 | 2.42 | 2.44 |
| KS_50 | | | | | | | | |
| MECO | SLEKHMLSHTEER | 438 | 9.93E−01 | 8.33E−03 | 1.59E−02 | −0.05 | 1.86 | 1.91 |
| M_277 | | | | | | | | |
| MEIKI | EYKSFE | 439 | 9.96E−01 | 4.55E−05 | 1.08E−03 | 0.09 | 3.90 | 3.81 |
| N_139 | | | | | | | | |
| MFHAS | FQYLLNHR | 440 | 9.99E−01 | 1.83E−04 | 5.70E−04 | 0.00 | 2.91 | 2.90 |
| 1_610 | | | | | | | | |
| MKI67_ | VVImKRSLRTSAKR | 441 | 9.93E−01 | 5.84E−05 | 1.69E−03 | 0.12 | 3.37 | 3.26 |
| 3045 | | | | | | | | |
| MMP1_ | FVLTEGNPR | 442 | 9.97E−01 | 9.25E−05 | 1.47E−03 | −0.05 | 2.35 | 2.39 |
| 100 | | | | | | | | |
| MMP1_ | FQPGPGIGG | 443 | 6.83E−01 | 2.75E−02 | 3.09E−03 | −0.37 | 1.08 | 1.46 |
| 185 | | | | | | | | |
| MMP2_ | LmAPIYTYTKNFR | 444 | 9.44E−01 | 4.60E−04 | 3.58E−03 | −0.18 | 1.72 | 1.89 |
| 420 | | | | | | | | |
| NBEA_ | LEKVAPLLR | 445 | 9.91E−01 | 3.97E−04 | 4.00E−04 | −0.08 | 2.04 | 2.12 |
| 1892 | | | | | | | | |
| NBEAL | KYESFE | 446 | 9.96E−01 | 4.55E−05 | 1.08E−03 | 0.09 | 3.90 | 3.81 |
| 2_2140 | | | | | | | | |
| NCAM1_ | FQAGLHNALmK | 447 | 8.85E−01 | 5.16E−05 | 6.99E−05 | −0.17 | 3.80 | 3.97 |
| 655 | | | | | | | | |
| NCAPD | FNELSHK | 448 | 6.25E−01 | 2.10E−03 | 5.62E−03 | −0.38 | 1.15 | 1.53 |
| 2_1153 | | | | | | | | |
| NES_14 | LSSSQR | 449 | 9.97E−01 | 6.62E−05 | 4.49E−04 | −0.03 | 2.38 | 2.41 |
| 49 | | | | | | | | |
| NID1_3 | FHQQHPQVI | 450 | 9.44E−01 | 7.93E−04 | 7.25E−04 | −0.22 | 2.47 | 2.69 |
| 60 | | | | | | | | |
| NID2_2 | HSSVPLGR | 259 | 1.00E+00 | 7.94E−05 | 1.51E−04 | 0.00 | 3.16 | 3.16 |
| 91 | | | | | | | | |
| NID2_4 | FTYNAANK | 451 | 9.47E−01 | 6.62E−05 | 7.92E−04 | −0.29 | 4.18 | 4.47 |
| 78 | | | | | | | | |
| NID2_9 | YAYPGAR | 263 | 9.91E−01 | 4.69E−05 | 4.85E−05 | −0.06 | 2.73 | 2.80 |
| 51 | | | | | | | | |
| PAK4_1 | HSEAGGGSG | 452 | 5.44E−01 | 1.47E−03 | 1.71E−03 | 0.69 | 2.47 | 1.78 |
| 41 | | | | | | | | |
| PCDHG | VPGTLIALIKIH | 453 | 8.12E−01 | 6.62E−05 | 1.98E−04 | 0.24 | 4.66 | 4.41 |
| B5_357 | | | | | | | | |
| PDLIM | FTASPASSTTAR | 454 | 9.42E−01 | 5.70E−05 | 1.07E−04 | 0.12 | 2.30 | 2.18 |
| 1_127 | | | | | | | | |
| PDLIM | ASSTTAR | 455 | 9.93E−01 | 3.77E−03 | 5.90E−03 | −0.05 | 0.98 | 1.03 |
| 1_132 | | | | | | | | |
| PDLIM | LSAGKTAVNVPR | 456 | 1.67E−01 | 1.89E−04 | 4.65E−05 | −1.30 | 4.71 | 6.01 |
| 5_194 | | | | | | | | |
| PLIN3_ | VSGAQPILSKL | 457 | 9.97E−01 | 7.21E−05 | 2.51E−02 | 0.07 | 2.32 | 2.25 |
| 75 | | | | | | | | |
| PNMA8 | LLGAARNPRRGR | 458 | 9.93E−01 | 1.73E−04 | 3.76E−04 | −0.09 | 3.77 | 3.86 |
| B_150 | | | | | | | | |
| POSTN_ | IITGPEIKYTR | 275 | 9.90E−01 | 7.21E−05 | 2.51E−04 | −0.09 | 2.51 | 2.60 |
| 727 | | | | | | | | |
| PPAT_2 | IVEISR | 278 | 9.97E−01 | 1.94E−03 | 8.32E−03 | 0.03 | 1.14 | 1.11 |
| 55 | | | | | | | | |
| PPP1R1 | LSETLTR | 279 | 9.97E−01 | 1.56E−04 | 2.58E−04 | 0.03 | 2.35 | 2.32 |
| 8_295 | | | | | | | | |
| PPP1R2 | IGTLTR | 280 | 7.96E−01 | 3.24E−03 | 4.25E−04 | −0.43 | 2.33 | 2.76 |
| 1_646 | | | | | | | | |
| PTPRF_ | MRYEGVVDMFQT | 459 | 9.91E−01 | 1.58E−04 | 3.32E−04 | −0.08 | 2.51 | 2.59 |
| 1859 | VKTLR | | | | | | | |
| QSOX1_ | HKGVAVR | 460 | 5.49E−01 | 1.78E−02 | 4.51E−04 | −0.49 | 1.20 | 1.69 |
| 210 | | | | | | | | |
| RPS11_ | qKQPTIFQNKKR | 461 | 9.91E−01 | 4.55E−05 | 1.07E−04 | 0.10 | 4.51 | 4.41 |
| 11 | | | | | | | | |
| SERBP | qTNSNAAGKQLR | 289 | 9.97E−01 | 3.35E−04 | 1.10E−03 | 0.03 | 1.65 | 1.62 |
| 1_60 | | | | | | | | |
| SERBP | QLQGEGKII | 462 | 9.39E−01 | 9.42E−05 | 5.51E−05 | −0.17 | 3.09 | 3.25 |
| 1_116 | | | | | | | | |
| SHC2_2 | VAKDPINQRACHIL | 463 | 9.97E−01 | 2.64E−03 | 8.42E−03 | 0.04 | 1.89 | 1.85 |
| 67 | | | | | | | | |
| SLC43 | KAPSLE | 464 | 6.75E−01 | 2.29E−03 | 6.56E−04 | 0.45 | 2.31 | 1.85 |
| A1_264 | | | | | | | | |
| SLIT3_ | FHGCIH | 465 | 2.41E−01 | 1.31E−03 | 2.05E−04 | −0.79 | 1.77 | 2.56 |
| 1172 | | | | | | | | |
| SPOCK | LAGGAGPNHGNFL | 466 | 1.46E−01 | 7.73E−05 | 6.32E−05 | −0.65 | 2.03 | 2.69 |
| 1_27 | | | | | | | | |
| SPOCK | SILPICK | 467 | 9.99E−01 | 2.01E−04 | 4.86E−03 | −0.01 | 1.95 | 1.96 |
| 1_240 | | | | | | | | |
| SRGN_ | LLPGESNKIPR | 296 | 9.95E−01 | 6.39E−05 | 4.70E−05 | 0.05 | 3.71 | 3.66 |
| 59 | | | | | | | | |
| STAM2_ | KnGTSSNKNKED | 468 | 9.42E−01 | 4.72E−05 | 4.85E−05 | −0.14 | 2.81 | 2.95 |
| 156 | | | | | | | | |
| STEAP | LTRIRQGWERNSK | 469 | 9.93E−01 | 4.55E−05 | 1.25E−04 | −0.05 | 2.94 | 2.99 |
| 4_446 | H | | | | | | | |
| TAGLN_ | LQEGKHVIGLQmG | 305 | 9.94E−01 | 7.70E−04 | 3.20E−03 | −0.06 | 1.86 | 1.93 |
| 168 | SNR | | | | | | | |
| TAGLN | LQEGKNVIGLQmG | 307 | 9.88E−01 | 8.22E−05 | 4.65E−05 | −0.11 | 4.25 | 4.36 |
| 2_167 | TNR | | | | | | | |
| TGFBI_ | LNSVFK | 470 | 9.98E−01 | 6.80E−05 | 2.00E−04 | −0.02 | 3.27 | 3.29 |
| 416 | | | | | | | | |
| TLN1_1 | AKASVPTIQ | 471 | 9.42E−01 | 6.74E−05 | 3.89E−03 | 0.22 | 2.59 | 2.37 |
| 1004 | | | | | | | | |
| TMTC1_ | LGTLTR | 321 | 7.96E−01 | 3.24E−03 | 4.25E−04 | −0.43 | 2.33 | 2.76 |
| 585 | | | | | | | | |
| TNFAIP | PEHHSVPGEGmRH | 472 | 7.31E−01 | 2.37E−02 | 5.26E−03 | −0.29 | 1.10 | 1.39 |
| 3_651 | PWK | | | | | | | |
| TNPO2_ | VQm VLNNL VEIInR | 473 | 9.93E−01 | 1.48E−03 | 3.58E−03 | 0.06 | 4.08 | 4.02 |
| 744 | PnTPKTLLE | | | | | | | |
| TNS1_ | QEGLAGYQR | 474 | 5.90E−01 | 2.97E−02 | 1.07E−04 | −0.59 | 1.49 | 2.08 |
| 236 | | | | | | | | |
| TPD52L | LSTVGSAISR | 475 | 8.86E−01 | 4.55E−05 | 4.65E−05 | 0.16 | 2.87 | 2.70 |
| 2_144 | | | | | | | | |
| TRGC2_ | HRCIVRHENNK | 476 | 8.62E−01 | 6.62E−05 | 1.14E−04 | 0.23 | 3.16 | 2.93 |
| 86 | | | | | | | | |
| TTN_59 | VKEPPVEFTKPL | 477 | 9.75E−01 | 1.70E−03 | 5.15E−05 | −0.19 | 3.15 | 3.34 |
| 75 | | | | | | | | |
| VAT1_ | AASPPLLR | 478 | 7.15E−01 | 2.89E−05 | 6.32E−05 | 0.27 | 3.22 | 2.95 |
| 42 | | | | | | | | |
| VCAN_ | YTQATH | 479 | 9.97E−01 | 1.32E−03 | 2.80E−02 | −0.03 | 1.15 | 1.18 |
| 2646 | | | | | | | | |
| VCAN_ | SEQQVAAR | 480 | 9.53E−01 | 7.94E−05 | 3.58E−03 | −0.33 | 3.62 | 3.95 |
| 3029 | | | | | | | | |
| VCAN_ | FKNSSSAK | 481 | 1.23E−01 | 1.99E−02 | 8.98E−04 | −0.72 | 0.79 | 1.51 |
| 3367 | | | | | | | | |
| VIM_ | YESVAAKNLQ | 482 | 9.93E−01 | 6.32E−03 | 2.48E−02 | 0.06 | 0.88 | 0.83 |
| 276 | | | | | | | | |
| VIM_ | QESTEYR | 324 | 9.26E−01 | 1.93E−04 | 1.07E−04 | −0.22 | 3.06 | 3.28 |
| 314 | | | | | | | | |
| VIM_ | QVQSLTCEV | 483 | 9.84E−01 | 2.10E−03 | 2.94E−02 | −0.10 | 1.01 | 1.11 |
| 322 | | | | | | | | |
| VIM_ | LKGTNESLER | 325 | 9.91E−01 | 1.33E−04 | 5.98E−05 | −0.09 | 3.75 | 3.84 |
| 333 | | | | | | | | |
| ZNF469_ | KDGHQR | 484 | 8.04E−01 | 2.93E−04 | 4.65E−05 | −0.44 | 5.10 | 5.54 |
| 761 | | | | | | | | |
| ZSCAN | FQQSQGPAVQR | 485 | 9.81E−01 | 1.53E−04 | 1.51E−04 | −0.09 | 2.79 | 2.89 |
| 4_25 | | | | | | | | |

| | | | | | | | ATS7_SMC_ | |
|---|---|---|---|---|---|---|---|---|
| | | | | | | | rd2_TAILS_ | |
| | | | | | | | frxns_ | |
| | | | | | | SEQ | matS7inDB_ | |
| | accession_ | | | previous_ | | ID | AspGluN_retry. | |
| TAILS_id | number | geneSymbol | StartAA | aa | sequence | NO: | totalIntensity | entry_name |
| ACTG1_105 | P63261 | ACTG1 | 105 | (L) | LTEAPLNP | 120 | 1.52E+08 | Actin, |
| | | | | | KANR | | | cytoplasmic |
| | | | | | | | | 2 |
| ACTN4_5 | O43707 | ACTN4 | 5 | (Y) | HAANQSY | 327 | 2.33E+08 | Alpha− |
| | | | | | QYGPSSAG | | | actinin−4 |
| | | | | | nGAGGGGS | | | |
| | | | | | mG | | | |
| ADAM10_86 | O14672 | ADAM10 | 86 | (E) | FKVETSNK | 328 | 1.44E+07 | Disintegrin |
| | | | | | VL | | | and metallo- |
| | | | | | | | | proteinase |
| | | | | | | | | domain− |
| | | | | | | | | containing |
| | | | | | | | | protein 10 |
| ADAM9_69 | A0AVL1 | ADAM9 | 69 | (Y) | VIQAEGKE | 122 | 1.73E+09 | ADAM9 |
| | | | | | HIIHLER | | | protein |
| ADAM9_69 | A0AVL1 | ADAM9 | 69 | (Y) | VIQAEGKE | 329 | 1.94E+09 | ADAM9 |
| | | | | | HITHL | | | protein |
| ADAM9_69 | A0AVL1 | ADAM9 | 69 | (Y) | VIQAEGK | 330 | 9.68E+09 | ADAM9 |
| | | | | | | | | protein |
| ADAM9_74 | A0AVL 1 | ADAM9 | 74 | (E) | GKEHIIHLE | 331 | 3.07E+08 | ADAM9 |
| | | | | | R | | | protein |
| ADAMTS12_ | P58397 | ADAMTS12 | 53 | (Y) | HVVGPVR | 332 | 1.87E+08 | A disintegrin |
| 53 | | | | | | | | and metallo |
| | | | | | | | | proteinase |
| | | | | | | | | with |
| | | | | | | | | thrombospon |
| | | | | | | | | din motifs 12 |
| ADNP2_375 | Q6IQ32 | ADNP2 | 375 | (L) | SQPVGPVN | 333 | 4.60E+09 | Activity− |
| | | | | | K | | | dependent |
| | | | | | | | | neuroprotect |
| | | | | | | | | or homeobox |
| | | | | | | | | protein 2 |
| AEBP1_37 | Q8IUX7 | AEBP1 | 37 | (E) | FLEGFLSEL | 123 | 8.64E+07 | Adipocyte |
| | | | | | EPEPR | | | enhancer− |
| | | | | | | | | binding |
| | | | | | | | | protein 1 |
| AKAP12_ | Q02952 | AKAP12 | 1106 | (P) | FTQGKVVG | 334 | 5.94E+08 | A−kinase |
| 1106 | | | | | QTTPESF | | | anchor |
| | | | | | | | | protein 12 |
| AMER2_654 | Q8N7J2 | AMER2 | 654 | (G) | LAGTTIRA | 335 | 2.57E+08 | APC |
| | | | | | TACH | | | membrane |
| | | | | | | | | recruitment |
| | | | | | | | | protein 2 |
| APLP2_452 | Q06481 | APLP2 | 452 | (Q) | LVETHLAR | 336 | 6.72E+08 | Amyloid−like |
| | | | | | | | | protein 2 |
| APOA1_86 | P02647 | APOA1 | 86 | (R) | EQLGPVTQ | 337 | 1.76E+09 | Apolipoprote |
| | | | | | EFW | | | in A−I |
| ATP1A2_ | B1AKY9 | ATP1A2 | 618 | (H) | PITAKAIAK | 338 | 7.66E+08 | Sodium/ |
| 618 | | | | | GVGIISEGN | | | potassium− |
| | | | | | ETVEDIAA | | | transporting |
| | | | | | RLnIPmSQV | | | ATPase |
| | | | | | nPREAK | | | subunit alpha |
| BCOR_258 | A1A564 | BCOR | 258 | (R) | DFIALR | 339 | 1.00E+08 | BCL−6 |
| | | | | | | | | corepressor |
| BMP6_129 | P22004 | BMP6 | 129 | (R) | LKSAPLFm | 340 | 4.00E+07 | Bone |
| | | | | | L | | | morphogenetic |
| | | | | | | | | protein 6 |
| BMPER_43 | Q8N8U9 | BMPER | 43 | (F) | LTGSVAKC | 341 | 1.92E+09 | BMP−binding |
| | | | | | ENEG | | | endothelial |
| | | | | | | | | regulator |
| | | | | | | | | protein |
| CALU_176 | O43852 | CALU | 176 | (E) | FTAFLHPEE | 342 | 1.25E+09 | Calumenin |
| | | | | | Y | | | |
| CAP1_300 | Q01518 | CAP1 | 300 | (P) | FSAPKPQTS | 343 | 4.47E+08 | Adenylyl |
| | | | | | PSPKR | | | cyclase− |
| | | | | | | | | associated |
| | | | | | | | | protein 1 |
| CARHSP1_ | I3L3X8 | CARHSP1 | 57 | (R) | ASQGPVYK | 344 | 2.14E+08 | Calcium− |
| 57 | | | | | | | | regulated |
| | | | | | | | | heat−stable |
| | | | | | | | | protein 1 |
| CCDC80_66 | Q76M96 | CCDC80 | 66 | (T) | LEEPNLQP | 127 | 3.94E+08 | Coiled−coil |
| | | | | | LQR | | | domain− |
| | | | | | | | | containing |
| | | | | | | | | protein 80 |
| CCDC80_ | Q76M96 | CCDC80 | 525 | (L) | QVGNVPLK | 345 | 3.14E+08 | Coiled−coil |
| 525 | | | | | KAK | | | domain− |
| | | | | | | | | containing |
| | | | | | | | | protein 80 |
| CCL2_28 | P13500 | CCL2 | 28 | (A) | INAPVTCC | 346 | 3.12E+08 | C—C motif |
| | | | | | YNFTNR | | | chemokine 2 |
| CEP192_728 | A0A0A0MR | CEP192 | 728 | (K) | DKSTAGR | 347 | 1.43E+09 | Centrosomal |
| | 42 | | | | | | | protein of |
| | | | | | | | | 192_kDa |
| COL12A1_ | D6RGG3 | COL12A1 | 213 | (Y) | KGGNTMT | 348 | 3.74E+07 | Collagen |
| 213 | | | | | G | | | alpha−1(XII) |
| | | | | | | | | chain |
| COL16A1_ | Q07092 | COL16A1 | 269 | (P) | QSEGKVYT | 132 | 1.58E+09 | Collagen |
| 269 | | | | | R | | | alpha−1(XVI) |
| | | | | | | | | chain |
| COL18A1_ | P39060 | COL18A1 | 1505 | (A) | LQPPVVQL | 349 | 1.11E+10 | Collagen |
| 1505 | | | | | H | | | alpha− |
| | | | | | | | | 1(XVIII) |
| | | | | | | | | chain |
| COL1A1_20 | P02452 | COL1A1 | 201 | (G) | FQGPPGEP | 350 | 1.37E+09 | Collagen |
| 1 | | | | | GEPGASGP | | | alpha−1(I) |
| | | | | | MGPR | | | chain |
| COL1A1_76 | P02452 | COL1A1 | 765 | (G) | LTGPIGPPG | 351 | 1.66E+08 | Collagen |
| 5 | | | | | PAGAPG | | | alpha−1(I) |
| | | | | | | | | chain |
| COL1A1_12 | P02452 | COL1A1 | 1238 | (S) | LSQQIENIR | 352 | 2.47E+07 | Collagen |
| 38 | | | | | | | | alpha−1(I) |
| | | | | | | | | chain |
| COL1A1_13 | P02452 | COL1A1 | 1389 | (L) | LLQGSNEIE | 353 | 3.74E+07 | Collagen |
| 89 | | | | | IR | | | alpha−1(I) |
| | | | | | | | | chain |
| COL1A2_80 | P08123 | COL1A2 | 80 | (A) | qYDGKGVG | 135 | 2.08E+07 | Collagen |
| | | | | | LGPGPmGL | | | alpha−2(I) |
| | | | | | mGPR | | | chain |
| COL1A2_11 | P08123 | COL1A2 | 113 | (G) | FQGPAGEP | 354 | 6.31E+08 | Collagen |
| 3 | | | | | G | | | alpha−2(I) |
| | | | | | | | | chain |
| COL1A2_11 | P08123 | COL1A2 | 113 | (G) | FQGPAGEP | 137 | 1.65E+09 | Collagen |
| 3 | | | | | GEPGQTGP | | | alpha−2(I) |
| | | | | | AGAR | | | chain |
| COL1A2_27 | P08123 | COL1A2 | 270 | | VGNAGPA | 138 | 2.57E+09 | Collagen |
| 0 | | | | | | | | alpha−2(I) |
| | | | | (A) | GPAGPR | | | chain |
| COL1A2_30 | P08123 | COL1A2 | 305 | | LTGAKGAA | 139 | 1.16E+09 | Collagen |
| 5 | | | | | GLPGVAGA | | | alpha−2(I) |
| | | | | (G) | PGLPGPR | | | chain |
| COL1A2_41 | P08123 | COL1A2 | 416 | (G) | VmGPPGSR | 355 | 1.40E+09 | Collagen |
| 6 | | | | | | | | alpha−2(I) |
| | | | | | | | | chain |
| COL1A2_57 | P08123 | COL1A2 | 579 | (E) | FGLPGPAG | 356 | 2.03E+09 | Collagen |
| 9 | | | | | PR | | | alpha−2(I) |
| | | | | | | | | chain |
| COL1A2_63 | P08123 | COL1A2 | 630 | (A) | VGTAGPSG | 142 | 1.32E+08 | Collagen |
| 0 | | | | | PSGLPGER | | | alpha−2(I) |
| | | | | | | | | chain |
| COL1A2_72 | P08123 | COL1A2 | 725 | (G) | FAGPAGAA | 357 | 8.67E+08 | Collagen |
| 5 | | | | | GQPGAKGE | | | alpha−2(I) |
| | | | | | R | | | chain |
| COL1A2_86 | P08123 | COL1A2 | 866 | (G) | LLGAPGIL | 145 | 4.62E+10 | Collagen |
| 6 | | | | | GLPGSR | | | alpha−2(I) |
| | | | | | | | | chain |
| COL1A2_86 | P08123 | COL1A2 | 867 | (L) | LGAPGILG | 358 | 4.34E+08 | Collagen |
| 7 | | | | | LPGSR | | | alpha−2(I) |
| | | | | | | | | chain |
| COL1A2_89 | P08123 | COL1A2 | 890 | (G) | AVGEPGPL | 359 | 1.82E+09 | Collagen |
| 0 | | | | | GIAGPPGA | | | alpha−2(I) |
| | | | | | R | | | chain |
| COL1A2_91 | P08123 | COL1A2 | 912 | (A) | VGSPGVNG | 360 | 5.69E+08 | Collagen |
| 2 | | | | | APGEAGR | | | alpha−2(I) |
| | | | | | | | | chain |
| COL1A2_96 | P08123 | COL1A2 | 960 | | AGAPGPHG | 146 | 3.12E+09 | Collagen |
| 0 | | | | | PVGPAGKH | | | alpha−2(I) |
| | | | | (A) | GNR | | | chain |
| COL1A2_10 | P08123 | COL1A2 | 1028 | (G) | LQGLPGIA | 361 | 9.44E+08 | Collagen |
| 28 | | | | | GHHG | | | alpha−2(I) |
| | | | | | | | | chain |
| COL1A2_10 | P08123 | COL1A2 | 1085 | | HQGPAGPP | 362 | 3.62E+09 | Collagen |
| 85 | | | | | GPPGPPGPP | | | alpha−2(I) |
| | | | | (G) | GVSGGGY | | | chain |
| COL1A2_11 | P08123 | COL1A2 | 1149 | (T) | LLTPEGSR | 363 | 3.21E+08 | Collagen |
| 49 | | | | | | | | alpha−2(I) |
| | | | | | | | | chain |
| COL3A1_11 | P02461 | COL3A1 | 1126 | (G) | QQGAIGSP | 364 | 1.17E+08 | Collagen |
| 26 | | | | | GPAGPR | | | alpha−1(III) |
| | | | | | | | | chain |
| COL4A2_71 | P08572 | COL4A2 | 71 | (G) | YNGPPGLQ | 150 | 2.73E+08 | Collagen |
| | | | | | GFPGLQGR | | | alpha−2(IV) |
| | | | | | | | | chain |
| COL4A2_14 | P08572 | COL4A2 | 1460 | (G) | HQGPIGQE | 365 | 4.73E+08 | Collagen |
| 60 | | | | | GAPGRPGS | | | alpha−2(IV) |
| | | | | | PGLPGmPG | | | chain |
| | | | | | R | | | |
| COL5A1_54 | A0A087WX | COL5A1 | 542 | (A) | QESQAQAI | 366 | 1.37E+08 | Collagen |
| 2 | W9 | | | | LQQAR | | | alpha−1(V) |
| | | | | | | | | chain |
| COL5A2_57 | P05997 | COL5A2 | 574 | (G) | LTGNPGVQ | 367 | 2.02E+08 | Collagen |
| 4 | | | | | GPEGKLGP | | | alpha−2(V) |
| | | | | | LGAPGE | | | chain |
| COL6A3_20 | P12111 | COL6A3 | 2084 | (G) | FQGCPGQR | 368 | 1.34E+08 | Collagen |
| 84 | | | | | | | | alpha−3(VI) |
| | | | | | | | | chain |
| COL7A1_10 | Q02388 | COL7A1 | 1009 | | ISSSQR | 369 | 5.54E+08 | Collagen |
| 09 | | | | | | | | alpha−1(VII) |
| | | | | (G) | | | | chain |
| CTSB 109 | P07858 | CTSB | 109 | (C) | WAFGAVE | 370 | 1.80E+07 | Cathepsin B |
| | | | | | AIS | | | |
| DBN1_434 | Q16643 | DBN1 | 434 | (A) | APVEPATA | 371 | 3.49E+08 | Drebrin |
| DDX39B 38 | Q5STU3 | DDX39B | 387 | (Q) | AGLAPR | 372 | 1.56E+10 | Spliceosome |
| 7 | | | | | | | | RNA |
| | | | | | | | | helicase |
| | | | | | | | | DDX39B |
| DIS3L_75 | Q8TF46 | DIS3L | 75 | (E) | FPELKGIIF | 373 | 2.26E+08 | DIS3−like |
| | | | | | MQTACQA | | | exonuclease |
| | | | | | VQHQR | | | 1 |
| ECM1_38 | Q16610 | ECM1 | 38 | (H) | FQEVGYAA | 374 | 1.82E+09 | Extracellular |
| | | | | | PPSPPLSR | | | matrix |
| | | | | | | | | protein 1 |
| ECM1_44 | Q16610 | ECM1 | 44 | (Y) | AAPPSPPLS | 375 | 1.31E+09 | Extracellular |
| | | | | | R | | | matrix |
| | | | | | | | | protein 1 |
| ECM1_108 | Q16610 | ECM1 | 108 | (P) | QEAVPLQK | 376 | 2.41E+09 | Extracellular |
| | | | | | | | | matrix |
| | | | | | | | | protein 1 |
| EEF1G 23 | P26641 | EEF1G | 23 | (A) | QYSGAQVR | 377 | 1.67E+08 | Elongation |
| | | | | | | | | factor 1− |
| | | | | | | | | gamma |
| EFEMP1_12 | Q12805 | EFEMP1 | 124 | (A) | AVAGPEM | 157 | 2.58E+10 | EGF− |
| 4 | | | | | QTGR | | | containing |
| | | | | | | | | fibulin−like |
| | | | | | | | | extracellular |
| | | | | | | | | matrix |
| | | | | | | | | protein 1 |
| EFEMP1_12 | Q12805 | EFEMP1 | 125 | (A) | VAGPEMQT | 158 | 7.46E+09 | EGF− |
| 5 | | | | | GR | | | containing |
| | | | | | | | | fibulin−like |
| | | | | | | | | extracellular |
| | | | | | | | | matrix |
| | | | | | | | | protein 1 |
| EFEMP1_13 | Q12805 | EFEMP1 | 131 | (M) | qTGRNNFVI | 378 | 1.41E+09 | EGF− |
| 1 | | | | | R | | | containing |
| | | | | | | | | fibulin−like |
| | | | | | | | | extracellular |
| | | | | | | | | matrix |
| | | | | | | | | protein 1 |
| EIF4B_426 | E7EX17 | EIF4B | 426 | (S) | QTGTSTTSS | 379 | 9.39E+08 | Eukaryotic |
| | | | | | R | | | translation |
| | | | | | | | | initiation |
| | | | | | | | | factor 4B |
| EIF4G2_433 | H0Y3P2 | EIF4G2 | 433 | (S) | qISLRPAQS | 380 | 1.16E+08 | Eukaryotic |
| | | | | | FLMnKnQV | | | translation |
| | | | | | PK | | | initiation |
| | | | | | | | | factor 4 |
| | | | | | | | | gamma 2 |
| EMILIN1_47 | Q9Y6C2 | EMILIN1 | 47 | (A) | QIAPR | 381 | 3.91E+09 | EMILIN−1 |
| EMILIN3_63 | Q9NT22 | EMILIN3 | 636 | (E) | QVSSQGSR | 382 | 4.02E+07 | EMILIN−3 |
| 6 | | | | | | | | |
| EMSY_943 | E9PMC9 | EMSY | 943 | (P) | QTVVQVLA | 383 | 7.48E+08 | BRCA2− |
| | | | | | VK | | | interacting |
| | | | | | | | | trans- |
| | | | | | | | | criptional |
| | | | | | | | | repressor |
| | | | | | | | | EMSY |
| FAM35A_14 | Q86V20 | FAM35A | 142 | (L) | LSENKIR | 384 | 8.39E+08 | Protein |
| 2 | | | | | | | | FAM35A |
| FAT3_1111 | E9PQ73 | FAT3 | 1111 | (Q) | IKLPER | 385 | 1.51E+09 | Protocadherin |
| | | | | | | | | Fat 3 |
| FBLN2_259 | P98095 | FBLN2 | 259 | (A) | ALGPPAPV | 162 | 8.11E+08 | Fibulin−2 |
| | | | | | QAKAR | | | |
| FBLN2_260 | P98095 | FBLN2 | 260 | (A) | LGPPAPVQ | 163 | 6.60E+09 | Fibulin−2 |
| | | | | | AKAR | | | |
| FBLN2_665 | P98095 | FBLN2 | 665 | (E) | FSQVASNTI | 386 | 7.99E+07 | Fibulin−2 |
| | | | | | PLPLPQPNT | | | |
| | | | | | CK | | | |
| FBN1_29 | P35555 | FBN1 | 29 | (N) | LEAGNVK | 387 | 2.40E+10 | Fibrillin−1 |
| FBN1_53 | P35555 | FBN1 | 53 | (A) | LKGPNVCG | 165 | 2.58E+09 | Fibrillin−1 |
| | | | | | SR | | | |
| FBXO47_39 | Q5MNV8 | FBXO47 | 390 | (E) | RKnFLQNV | 388 | 2.62E+08 | F−box only |
| 0 | | | | | AnAFACVI | | | protein 47 |
| | | | | | mEMLQSIM | | | |
| | | | | | SGDR | | | |
| FLJ43944_2 | Q6ZRG5 | FLJ43944 | 211 | (C) | LQSSLRKW | 389 | 1.01E+08 | Putative |
| 11 | | | | | NPL | | | uncharact- |
| | | | | | | | | erized |
| | | | | | | | | protein |
| | | | | | | | | FLJ43944 |
| FLNA_689 | P21333 | FLNA | 689 | (A) | VNKPAEFT | 390 | 3.19E+09 | Filamin−A |
| | | | | | V | | | |
| FLNA_1285 | P21333 | FLNA | 1285 | (A) | LTQTGGPH | 168 | 1.38E+09 | Filamin−A |
| | | | | | VKAR | | | |
| FLNC_2649 | Q14315 | FLNC | 2649 | (A) | FVGQKNSF | 391 | 7.42E+07 | Filamin−C |
| | | | | | TV | | | |
| FN1_35 | P02751−15 | FN1 | 35 | (Q) | qm VQPQSP | 392 | 2.65E+10 | Isoform 15 of |
| | | | | | VAVSQSKP | | | Fibronectin |
| | | | | | GCY | | | |
| FN1_36 | P02751−15 | FN1 | 36 | (Q) | MVQPQSPV | 393 | 1.54E+11 | Isoform 15 of |
| | | | | | AVSQSKPG | | | Fibronectin |
| | | | | | CY | | | |
| FN1_40 | P02751−15 | FN1 | 40 | (P) | QSPVAVSQ | 394 | 3.29E+09 | Isoform 15 of |
| | | | | | SKPGCY | | | Fibronectin |
| FN1_99 | P02751−15 | FN1 | 99 | (F) | DKYTGNTY | 395 | 2.24E+10 | Isoform 15 of |
| | | | | | R | | | Fibronectin |
| FN1_279 | P02751−15 | FN1 | 279 | (T) | TSSGSGPFT | 396 | 2.71E+10 | Isoform 15 of |
| | | | | | | | | Fibronectin |
| FN1_607 | P02751−15 | FN1 | 607 | (P) | SSSGPVEVF | 397 | 1.24E+11 | Isoform 15 of |
| | | | | | IT | | | Fibronectin |
| FN1_1143 | P02751−15 | FN1 | 1143 | (G) | LTPGVEYV | 177 | 9.55E+08 | Isoform 15 of |
| | | | | | YTIQVLR | | | Fibronectin |
| FN1_1656 | P02751−15 | FN1 | 1656 | (P) | SSSPVTGY | 178 | 2.00E+10 | Isoform 15 of |
| | | | | | R | | | Fibronectin |
| FN1_1693 | P02751−15 | FN1 | 1693 | (G) | LQPTVEYV | 398 | 1.01E+10 | Isoform 15 of |
| | | | | | VSVYAQNP | | | Fibronectin |
| | | | | | SG | | | |
| FN1_1715 | P02751−15 | FN1 | 1715 | (P) | LVQTAVTN | 399 | 9.37E+10 | Isoform 15 of |
| | | | | | I | | | Fibronectin |
| FN1_1780 | P02751−15 | FN1 | 1780 | (E) | LQGLRPGS | 400 | 4.09E+09 | Isoform 15 of |
| | | | | | EYTVSVVA | | | Fibronectin |
| | | | | | LH | | | |
| FN1_1805 | P02751−15 | FN1 | 1805 | (P) | LIGTQSTAI | 401 | 5.10E+10 | Isoform 15 of |
| | | | | | PAPT | | | Fibronectin |
| FN1_2113 | P02751−15 | FN1 | 2113 | (P) | FVTHPGY | 402 | 5.02E+09 | Isoform 15 of |
| | | | | | | | | Fibronectin |
| FN1_2136 | P02751−15 | FN1 | 2136 | (P) | SVGQQmIF | 403 | 4.18E+09 | Isoform 15 of |
| | | | | | | | | Fibronectin |
| FOXG1_235 | P55316 | FOXG1 | 235 | (S) | LNKCFVKV | 404 | 3.64E+07 | Forkhead box |
| | | | | | PRHY | | | protein G1 |
| FSTL1_290 | Q12841 | FSTL1 | 290 | (E) | LQKHQETA | 405 | 5.17E+08 | Follistatin− |
| | | | | | | | | related |
| | | | | | | | | protein 1 |
| FYB2_408 | Q5VWT5 | FYB2 | | (V) | FKVDAC | 406 | 1.06E+09 | FYN−binding |
| | | | | | | | | protein 2 |
| GSN_413 | P06396 | GSN | 413 | (S) | HIANVER | 187 | 1.39E+09 | Gelsolin |
| HEG1_47 | Q9ULI3 | HEG1 | 47 | (P) | LAGAGLEL | 407 | 1.84E+08 | Protein HEG |
| | | | | | QL | | | homolog 1 |
| HEG1_51 | Q9ULI3 | HEG1 | 51 | (A) | GLELQLER | 408 | 2.73E+08 | Protein HEG |
| | | | | | | | | homolog 1 |
| HEG1_224 | Q9ULI3 | HEG1 | 224 | (A) | FQTKSGTA | 409 | 2.12E+08 | Protein HEG |
| | | | | | S | | | homolog 1 |
| HSPG2_275 | P98160 | HSPG2 | 275 | (P) | LLPGSVRP | 410 | 1.16E+08 | Basement |
| | | | | | LPCGPQ | | | membrane− |
| | | | | | | | | specific |
| | | | | | | | | heparan |
| | | | | | | | | sulfate |
| | | | | | | | | proteoglycan |
| | | | | | | | | core protein |
| HSPG2_186 | P98160 | HSPG2 | 1863 | (T) | LSAPVVSIH | 411 | 1.42E+08 | Basement |
| 3 | | | | | PPQLTVQP | | | membrane− |
| | | | | | GQLA | | | specific |
| | | | | | | | | heparan |
| | | | | | | | | sulfate |
| | | | | | | | | proteoglycan |
| | | | | | | | | core protein |
| HSPG2_193 | P98160 | HSPG2 | 1936 | (A) | HSSAGQQV | 412 | 1.14E+08 | Basement |
| 6 | | | | | AR | | | membrane− |
| | | | | | | | | specific |
| | | | | | | | | heparan |
| | | | | | | | | sulfate |
| | | | | | | | | proteoglycan |
| | | | | | | | | core protein |
| HSPG2_233 | P98160 | HSPG2 | 2331 | (G) | ANLAYPAG | 194 | 2.66E+08 | Basement |
| 1 | | | | | STQPIR | | | membrane− |
| | | | | | | | | specific |
| | | | | | | | | heparan |
| | | | | | | | | sulfate |
| | | | | | | | | proteoglycan |
| | | | | | | | | core protein |
| HSPG2_233 | P98160 | HSPG2 | 2333 | (N) | LAYPAGST | 195 | 3.02E+08 | Basement |
| 3 | | | | | QPIR | | | membrane− |
| | | | | | | | | specific |
| | | | | | | | | heparan |
| | | | | | | | | sulfate |
| | | | | | | | | proteoglycan |
| | | | | | | | | core protein |
| HSPG2_253 | P98160 | HSPG2 | 2531 | (Q) | GVAYPVR | 413 | 7.81E+07 | Basement |
| 1 | | | | | | | | membrane− |
| | | | | | | | | specific |
| | | | | | | | | heparan |
| | | | | | | | | sulfate |
| | | | | | | | | proteoglycan |
| | | | | | | | | core protein |
| HSPG2_318 | P98160 | HSPG2 | 3186 | (C) | LAQNALGT | 414 | 2.61E+08 | Basement |
| 6 | | | | | AQKQVEVI | | | membrane− |
| | | | | | V | | | specific |
| | | | | | | | | heparan |
| | | | | | | | | sulfate |
| | | | | | | | | proteoglycan |
| | | | | | | | | core protein |
| HSPG2_365 | P98160 | HSPG2 | 3653 | (A) | FAHLQVPE | 415 | 2.55E+07 | Basement |
| 3 | | | | | R | | | membrane− |
| | | | | | | | | specific |
| | | | | | | | | heparan |
| | | | | | | | | sulfate |
| | | | | | | | | proteoglycan |
| | | | | | | | | core protein |
| IGFBP3_31 | P17936 | IGFBP3 | 31 | (S) | SAGLGPVV | 211 | 1.02E+09 | Insulin−like |
| | | | | | R | | | growth |
| | | | | | | | | factor− |
| | | | | | | | | binding |
| | | | | | | | | protein 3 |
| IGFBP3_32 | P17936 | IGFBP3 | 32 | (S) | AGLGPVVR | 212 | 5.97E+09 | Insulin−like |
| | | | | | | | | growth |
| | | | | | | | | factor− |
| | | | | | | | | binding |
| | | | | | | | | protein 3 |
| IGFBP7_101 | Q16270 | IGFBP7 | 101 | (A) | AAGGPGVS | 214 | 4.97E+08 | Insulin−like |
| | | | | | GVCVCKSR | | | growth |
| | | | | | | | | factor− |
| | | | | | | | | binding |
| | | | | | | | | protein 7 |
| IPP_504 | Q9Y573 | IPP | 504 | (V) | EKYSFE | 416 | 2.28E+08 | Actin− |
| | | | | | | | | binding |
| | | | | | | | | protein IPP |
| JAK3_794 | P52333 | JAK3 | 794 | (P) | GALAPR | 417 | 7.81E+09 | Tyrosine− |
| | | | | | | | | protein |
| | | | | | | | | kinase JAK3 |
| KCND1_436 | Q9NSA2 | KCND1 | 436 | (I) | RLAKSGTT | 418 | 3.21E+08 | Potassium |
| | | | | | NAFLQYKQ | | | voltage−gated |
| | | | | | NGGLE | | | channel |
| | | | | | | | | subfamily D |
| | | | | | | | | member 1 |
| KPNB1_37 | Q14974 | KPNB1 | 37 | (F) | LVELSR | 216 | 4.57E+08 | Importin |
| | | | | | | | | subunit beta− |
| | | | | | | | | 1 |
| KRT28_2 | Q7Z3Y7 | KRT28 | 2 | (M) | SLQFSNGS | 419 | 2.34E+08 | Keratin, type |
| | | | | | R | | | I cytoskeletal |
| | | | | | | | | 28 |
| LAMA5_484 | O15230 | LAMA5 | 484 | (E) | QVLPAGQI | 420 | 2.51E+09 | Laminin |
| | | | | | VNC | | | subunit |
| | | | | | | | | alpha−5 |
| LASP1_181 | Q14847 | LASP1 | 181 | (A) | qSYGGYK | 421 | 2.02E+08 | LIM and |
| | | | | | | | | SH3_domain |
| | | | | | | | | protein 1 |
| LASP1_182 | Q14847 | LASP1 | 182 | (Q) | SYGGYKEP | 422 | 1.27E+08 | LIM and |
| | | | | | AAPVSIQR | | | SH3_domain |
| | | | | | | | | protein 1 |
| LMNA_408 | P02545 | LMNA | 408 | (S) | qTQGGGSV | 222 | 1.22E+10 | Prelamin− |
| | | | | | TKKR | | | A/C |
| LOX_58 | P28300 | LOX | 58 | (L) | LSLGSQYQ | 223 | 6.15E+07 | Protein− |
| | | | | | PQR | | | lysine 6− |
| | | | | | | | | oxidase |
| LOX_60 | P28300 | LOX | 60 | (S) | LGSQYQPQ | 423 | 3.55E+08 | Protein− |
| | | | | | R | | | lysine 6− |
| | | | | | | | | oxidase |
| LOX_123 | P28300 | LOX | 123 | (R) | HWFQAGY | 226 | 6.25E+08 | Protein− |
| | | | | | STSR | | | lysine 6− |
| | | | | | | | | oxidase |
| LOX_124 | P28300 | LOX | 124 | (H) | WFQAGYST | 227 | 1.64E+09 | Protein− |
| | | | | | SR | | | lysine 6− |
| | | | | | | | | oxidase |
| LOX_125 | P28300 | LOX | 125 | (W) | FQAGYSTS | 424 | 1.92E+10 | Protein− |
| | | | | | R | | | lysine 6− |
| | | | | | | | | oxidase |
| LOX_126 | P28300 | LOX | 126 | (F) | QAGYSTSR | 228 | 1.03E+08 | Protein− |
| | | | | | | | | lysine 6− |
| | | | | | | | | oxidase |
| LOXL1_157 | Q08397 | LOXL1 | 157 | (S) | SVSASAFA | 425 | 9.17E+06 | Lysyl |
| | | | | | STYR | | | oxidase |
| | | | | | | | | homolog 1 |
| LOXL1_218 | Q08397 | LOXL1 | 218 | (A) | AVASAGVI | 426 | 2.92E+08 | Lysyl |
| | | | | | YPYQPR | | | oxidase |
| | | | | | | | | homolog 1 |
| LOXL1_219 | Q08397 | LOXL1 | 219 | (A) | VASAGVIY | 229 | 4.27E+08 | Lysyl |
| | | | | | PYQPR | | | oxidase |
| | | | | | | | | homolog 1 |
| LOXL2_37 | Q9Y4K0 | LOXL2 | 37 | (Y) | FQQPAPEY | 230 | 9.09E+09 | Lysyl |
| | | | | | HQPQAPAN | | | oxidase |
| | | | | | VAKIQLR | | | homolog 2 |
| LOXL2_38 | Q9Y4K0 | LOXL2 | 38 | (F) | qQPAPEYH | 231 | 1.65E+08 | Lysyl |
| | | | | | QPQAPANV | | | oxidase |
| | | | | | AKIQLR | | | homolog 2 |
| LOXL2_45 | Q9Y4K0 | LOXL2 | 45 | (Y) | HQPQAPAN | 427 | 6.08E+08 | Lysyl |
| | | | | | VAKIQLR | | | oxidase |
| | | | | | | | | homolog 2 |
| LRRC17_22 | Q8N6Y2 | LRRC17 | 225 | (P) | qVSGRPPVI | 428 | 9.93E+07 | Leucine−rich |
| 5 | | | | | KPEV | | | repeat− |
| | | | | | | | | containing |
| | | | | | | | | protein 17 |
| LTBP1_393 | Q14766 | LTBP1 | 393 | (T) | LTATNFR | 234 | 6.93E+09 | Latent− |
| | | | | | | | | transforming |
| | | | | | | | | growth factor |
| | | | | | | | | beta−binding |
| | | | | | | | | protein 1 |
| LTBP1_540 | Q14766 | LTBP1 | 540 | (S) | HQQVIPHV | 236 | 8.09E+08 | Latent− |
| | | | | | YPVAAKTQ | | | transforming |
| | | | | | LGR | | | growth factor |
| | | | | | | | | beta−binding |
| | | | | | | | | protein 1 |
| LTBP1_1597 | Q14766 | LTBP1 | 1597 | (F) | SEQYTPEA | 429 | 3.36E+09 | Latent− |
| | | | | | | | | transforming |
| | | | | | | | | growth factor |
| | | | | | | | | beta−binding |
| | | | | | | | | protein 1 |
| LTBP2_79 | G3V3X5 | LTBP2 | 79 | (P) | VAGLQPVE | 430 | 6.41E+07 | Latent− |
| | | | | | R | | | transforming |
| | | | | | | | | growth factor |
| | | | | | | | | beta−binding |
| | | | | | | | | protein 2 |
| LTBP2_250 | G3V3X5 | LTBP2 | 250 | (S) | SAAGEGTL | 431 | 1.86E+08 | Latent− |
| | | | | | AR | | | transforming |
| | | | | | | | | growth factor |
| | | | | | | | | beta−binding |
| | | | | | | | | protein 2 |
| LTBP2_290 | G3V3X5 | LTBP2 | 290 | (Q) | QHVGLSR | 432 | 9.07E+08 | Latent− |
| | | | | | | | | transforming |
| | | | | | | | | growth factor |
| | | | | | | | | beta−binding |
| | | | | | | | | protein 2 |
| LTBP2_502 | G3V3X5 | LTBP2 | 502 | (A) | LVENSVET | 433 | 2.73E+08 | Latent− |
| | | | | | RPPPWLPA | | | transforming |
| | | | | | SPGHSLW | | | growth factor |
| | | | | | | | | beta−binding |
| | | | | | | | | protein 2 |
| LTBP3_103 | Q9NS15 | LTBP3 | 103 | (T) | LTGSGFR | 434 | 3.47E+09 | Latent− |
| | | | | | | | | transforming |
| | | | | | | | | growth factor |
| | | | | | | | | beta−binding |
| | | | | | | | | protein 3 |
| LTBP3_208 | Q9NS15 | LTBP3 | 208 | (A) | FLVPLGPG | 435 | 1.77E+07 | Latent− |
| | | | | | QISA | | | transforming |
| | | | | | | | | growth factor |
| | | | | | | | | beta−binding |
| | | | | | | | | protein 3 |
| LTBP3_239 | Q9NS15 | LTBP3 | 239 | (A) | SVQVHR | 248 | 8.70E+08 | Latent− |
| | | | | | | | | transforming |
| | | | | | | | | growth factor |
| | | | | | | | | beta−binding |
| | | | | | | | | protein 3 |
| MAP3K13_3 | 043283 | MAP3K13 | 346 | (E) | LLTGEIPYK | 436 | 6.05E+08 | Mitogen− |
| 46 | | | | | | | | activated |
| | | | | | | | | protein |
| | | | | | | | | kinase kinase |
| | | | | | | | | kinase 13 |
| MAP4_815 | P27816 | MAP4 | 815 | (A) | AVASTGPS | 252 | 5.60E+08 | Microtubule− |
| | | | | | SR | | | associated |
| | | | | | | | | protein 4 |
| MAP4_816 | P27816 | MAP4 | 816 | (A) | VASTGPSS | 253 | 8.83E+08 | Microtubule− |
| | | | | | R | | | associated |
| | | | | | | | | protein 4 |
| MARCKS_5 | P29966 | MARCKS | 50 | (A) | AESGAK | 437 | 1.28E+08 | Myristoylated |
| 0 | | | | | | | | alanine−rich |
| | | | | | | | | C−kinase |
| | | | | | | | | substrate |
| MECOM_27 | E7EQ57 | MECOM | 277 | (Q) | SLEKHMLS | 438 | 1.07E+10 | MDS1_and |
| 7 | | | | | HTEER | | | EVI1 |
| | | | | | | | | complex |
| | | | | | | | | locus protein |
| | | | | | | | | EVI1 |
| MEIKIN_13 | A0A087WX | MEIKIN | 139 | (A) | EYKSFE | 439 | 2.28E+08 | Meiosis− |
| 9 | M9 | | | | | | | specific |
| | | | | | | | | kinetochore |
| | | | | | | | | protein |
| MFHAS1_61 | Q9Y4C4 | MFHAS1 | 610 | (H) | FQYLLNHR | 440 | 3.08E+07 | Malignant |
| 0 | | | | | | | | fibrous |
| | | | | | | | | histiocytoma− |
| | | | | | | | | amplified |
| | | | | | | | | sequence 1 |
| MKI67_3045 | P46013 | MKI67 | 3045 | (P) | VVImKRSL | 441 | 6.20E+08 | Proliferation |
| | | | | | RTSAKR | | | marker |
| | | | | | | | | protein Ki−67 |
| MMP1_100 | P03956 | MMP1 | 100 | (Q) | FVLTEGNP | 442 | 7.48E+07 | Interstitial |
| | | | | | R | | | collagenase |
| MMP1_185 | P03956 | MMP1 | 185 | (A) | FQPGPGIG | 443 | 6.29E+07 | Interstitial |
| | | | | | G | | | collagenase |
| MMP2_420 | P08253 | MMP2 | 420 | (A) | LmAPIYTY | 444 | 8.07E+07 | 72_kDa type |
| | | | | | TKNFR | | | IV |
| | | | | | | | | collagenase |
| NBEA_1892 | A0A0D9SF2 | NBEA | 1892 | (A) | LEKVAPLL | 445 | 1.60E+08 | Neurobeachin |
| | 8 | | | | R | | | |
| NBEAL2_21 | Q6ZNJ1 | NBEAL2 | 2140 | (E) | KYESFE | 446 | 3.42E+08 | Neurobeachin- |
| 40 | | | | | | | | like protein |
| | | | | | | | | 2 |
| NCAM1_655 | P13591−5 | NCAM1 | 655 | (M) | FQAGLHNA | 447 | 5.05E+08 | Isoform 5 of |
| | | | | | LmK | | | Neural cell |
| | | | | | | | | adhesion |
| | | | | | | | | molecule 1 |
| NCAPD2_11 | Q15021 | NCAPD2 | 1153 | (F) | FNELSHK | 448 | 4.61E+07 | Condensin |
| 53 | | | | | | | | complex |
| | | | | | | | | subunit 1 |
| NES_1449 | P48681 | NES | 1449 | (A) | LSSSQR | 449 | 2.77E+08 | Nestin |
| NID1_360 | P14543 | NID1 | 360 | (T) | FHQQHPQV | 450 | 7.78E+09 | Nidogen−1 |
| | | | | | I | | | |
| NID2_291 | Q14112 | NID2 | 291 | (A) | HSSVPLGR | 259 | 6.67E+09 | Nidogen−2 |
| NID2_478 | Q14112 | NID2 | 478 | (V) | FTYNAANK | 451 | 8.95E+08 | Nidogen−2 |
| NID2_951 | Q14112 | NID2 | 951 | (Q) | YAYPGAR | 263 | 2.03E+09 | Nidogen−2 |
| PAK4_141 | O96013 | PAK4 | 141 | (G) | HSEAGGGS | 452 | 3.77E+06 | Serine/ |
| | | | | | G | | | threonine− |
| | | | | | | | | protein |
| | | | | | | | | kinase PAK 4 |
| PCDHGB5_ | Q9Y5G0 | PCDHGB5 | 357 | (A) | VPGTLIALI | 453 | 1.76E+08 | Protocadherin |
| 357 | | | | | KIH | | | gamma−B5 |
| PDLIMI_12 | O00151 | PDLIM1 | 127 | (P) | FTASPASST | 454 | 5.99E+07 | PDZ and |
| 7 | | | | | TAR | | | LIM domain |
| | | | | | | | | protein 1 |
| PDLIM1_13 | O00151 | PDLIM1 | 132 | (P) | ASSTTAR | 455 | 1.19E+08 | PDZ and |
| 2 | | | | | | | | LIM domain |
| | | | | | | | | protein 1 |
| PDLIM5_19 | Q96HC4 | PDLIM5 | 194 | (A) | LSAGKTAV | 456 | 7.44E+08 | PDZ and |
| 4 | | | | | NVPR | | | LIM domain |
| | | | | | | | | protein 5 |
| PLIN3_75 | O60664 | PLIN3 | 75 | (A) | VSGAQPILS | 457 | 5.73E+07 | Perilipin−3 |
| | | | | | KL | | | |
| PNMA8B_1 | Q9ULN7 | PNMA8B | 150 | (S) | LLGAARNP | 458 | 3.35E+09 | Paraneo- |
| 50 | | | | | RRGR | | | plastic |
| | | | | | | | | antigen−like |
| | | | | | | | | protein 8B |
| POSTN_727 | Q15063−5 | POSTN | 727 | (R) | IITGPEIKYT | 275 | 6.22E+09 | Isoform 5 of |
| | | | | | R | | | Periostin |
| PPAT_255 | Q06203 | PPAT | 255 | (E) | IVEISR | 278 | 4.57E+08 | Amidophospho- |
| | | | | | | | | ribosyltran- |
| | | | | | | | | sferase |
| PPP1R18_29 | A0A0G2JHC | PPP1R18 | 295 | (E) | LSETLTR | 279 | 1.25E+08 | PPP1R18 |
| 5 | 2 | | | | | | | |
| PPP1R21_64 | Q6ZMIO | PPP1R21 | 646 | | IGTLTR | 280 | 2.46E+08 | Protein |
| 6 | | | | | | | | phosphatase |
| | | | | (L) | | | | 1_regulatory |
| | | | | | | | | subunit 21 |
| PTPRF_1859 | P10586 | PTPRF | 1859 | (R) | MRYEGVV | 459 | 1.20E+09 | Receptor− |
| | | | | | DMFQTVKT | | | type tyrosine− |
| | | | | | LR | | | protein |
| | | | | | | | | phosphatase |
| | | | | | | | | F |
| QSOX1_210 | O00391 | QSOX1 | 210 | (Q) | HKGVAVR | 460 | 7.78E+07 | Sulfhydryl |
| | | | | | | | | oxidase 1 |
| RPS11_11 | MOQZC5 | RPS11 | 11 | (Y) | qKQPTIFQN | 461 | 1.03E+09 | 40S |
| | | | | | KKR | | | ribosomal |
| | | | | | | | | protein S11 |
| SERBP1_60 | Q8NC51 | SERBP1 | 60 | (A) | qTNSNAAG | 289 | 2.49E+08 | Plasminogen |
| | | | | | KQLR | | | activator |
| | | | | | | | | inhibitor 1 |
| | | | | | | | | RNA−binding |
| | | | | | | | | protein |
| SERBP1_11 | Q8NC51 | SERBP1 | 116 | (Q) | QLQGEGKII | 462 | 7.27E+09 | Plasminogen |
| 6 | | | | | | | | activator |
| | | | | | | | | inhibitor 1 |
| | | | | | | | | RNA−binding |
| | | | | | | | | protein |
| SHC2_267 | P98077 | SHC2 | 267 | (Y) | VAKDPINQ | 463 | 3.39E+07 | SHC− |
| | | | | | RACHIL | | | transforming |
| | | | | | | | | protein 2 |
| SLC43A1_2 | O75387 | SLC43A1 | 264 | (Q) | KAPSLE | 464 | 1.13E+08 | Large neutral |
| 64 | | | | | | | | amino acids |
| | | | | | | | | transporter |
| | | | | | | | | small subunit |
| | | | | | | | | 3 |
| SLIT3_1172 | A0A0A0MS | SLIT3 | 1172 | (G) | FHGCIH | 465 | 4.11E+08 | Slit homolog |
| | C8 | | | | | | | 3_protein |
| SPOCK1_27 | Q08629 | SPOCK1 | 27 | (A) | LAGGAGPN | 466 | 5.00E+08 | Testican−1 |
| | | | | | HGNFL | | | |
| SPOCK1_24 | Q08629 | SPOCK1 | 240 | (T) | SILPICK | 467 | 2.61E+08 | Testican−1 |
| 0 | | | | | | | | |
| SRGN 59 | P10124 | SRGN | 59 | (E) | LLPGESNKI | 296 | 2.86E+09 | Serglycin |
| | | | | | PR | | | |
| STAM2_156 | O75886 | STAM2 | 156 | (A) | KnGTSSNK | 468 | 2.65E+09 | Signal |
| | | | | | NKED | | | transducing |
| | | | | | | | | adapter |
| | | | | | | | | molecule 2 |
| STEAP4_44 | Q687X5 | STEAP4 | 446 | (T) | LTRIRQGW | 469 | 1.53E+09 | Metallo- |
| 6 | | | | | ERNSKH | | | reductase |
| | | | | | | | | STEAP4 |
| TAGLN_168 | Q01995 | TAGLN | 168 | (Q) | LQEGKHVI | 305 | 1.28E+09 | Transgelin |
| | | | | | GLQmGSNR | | | |
| TAGLN2_16 | P37802 | TAGLN2 | 167 | (Q) | LQEGKNVI | 307 | 6.52E+07 | Transgelin−2 |
| 7 | | | | | GLQmGTNR | | | |
| TGFBI_416 | Q15582 | TGFBI | 416 | (P) | LNSVFK | 470 | 4.05E+07 | Transforming |
| | | | | | | | | growth |
| | | | | | | | | factor−beta− |
| | | | | | | | | induced |
| | | | | | | | | protein ig−h3 |
| TLN1_1004 | Q9Y490 | TLN1 | 1004 | (A) | AKASVPTI | 471 | 6.83E+07 | Talin−1 |
| | | | | | Q | | | |
| TMTC1_585 | F8VTQ9 | TMTC1 | 585 | (N) | LGTLTR | 321 | 1.47E+09 | Transmembrane |
| | | | | | | | | and TPR |
| | | | | | | | | repeat− |
| | | | | | | | | containing |
| | | | | | | | | protein 1 |
| TNFAIP3_65 | D3TTY5 | TNFAIP3 | 651 | (V) | PEHHSVPG | 472 | 9.83E+08 | Truncated |
| 1 | | | | | EGmRHPW | | | tumor |
| | | | | | K | | | necrosis |
| | | | | | | | | factor alpha− |
| | | | | | | | | induced |
| | | | | | | | | protein 3 |
| TNPO2_744 | A0A075B78 | TNPO2 | 744 | (Y) | VQm VLNN | 473 | 2.81E+08 | Transportin−2 |
| | 0 | | | | LVEIInRPnT | | | |
| | | | | | PKTLLE | | | |
| TNS1_236 | A0A087WW | TNS1 | 236 | (A) | QEGLAGYQ | 474 | 1.65E+08 | Tensin−1 |
| | W7 | | | | R | | | |
| TPD52L2_14 | A0A087WY | TPD52L2 | 144 | (A) | LSTVGSAIS | 475 | 2.85E+07 | Tumor |
| 4 | R3 | | | | R | | | protein D54 |
| TRGC2_86 | P03986 | TRGC2 | 86 | (E) | HRCIVRHE | 476 | 6.46E+08 | T−cell |
| | | | | | NNK | | | receptor |
| | | | | | | | | gamma−2 |
| | | | | | | | | chain C |
| | | | | | | | | region |
| TTN_5975 | A0A0A0MR | TTN | 5975 | (F) | VKEPPVEF | 477 | 4.70E+08 | Titin |
| | A3 | | | | TKPL | | | |
| VAT1_42 | Q99536 | VAT1 | 42 | (A) | AASPPLLR | 478 | 1.26E+08 | Synaptic |
| | | | | | | | | vesicle |
| | | | | | | | | membrane |
| | | | | | | | | protein VAT− |
| | | | | | | | | 1_homolog |
| VCAN_2646 | P13611 | VCAN | 2646 | (S) | YTQATH | 479 | 4.64E+08 | Versican core |
| | | | | | | | | protein |
| VCAN_3029 | P13611 | VCAN | 3029 | (A) | SEQQVAAR | 480 | 5.99E+08 | Versican core |
| | | | | | | | | protein |
| VCAN_3367 | P13611 | VCAN | 3367 | (Y) | FKNSSSAK | 481 | 3.21E+08 | Versican core |
| | | | | | | | | protein |
| VIM_276 | P08670 | VIM | 276 | (Q) | YESVAAKN | 482 | 5.15E+08 | Vimentin |
| | | | | | LQ | | | |
| VIM_314 | P08670 | VIM | 314 | (K) | QESTEYR | 324 | 7.47E+08 | Vimentin |
| VIM_322 | P08670 | VIM | 322 | (R) | QVQSLTCE | 483 | 1.12E+09 | Vimentin |
| | | | | | V | | | |
| VIM_333 | P08670 | VIM | 333 | (A) | LKGTNESL | 325 | 1.10E+08 | Vimentin |
| | | | | | ER | | | |
| ZNF469_761 | H3BS19 | ZNF469 | 761 | (A) | KDGHQR | 484 | 8.73E+08 | Zinc finger |
| | | | | | | | | protein 469 |
| ZSCAN4_25 | Q8NAM6 | ZSCAN4 | 25 | (A) | FQQSQGPA | 485 | 2.40E+08 | Zinc finger |
| | | | | | VQR | | | and SCAN |
| | | | | | | | | domain− |
| | | | | | | | | containing |
| | | | | | | | | protein 4 |

HUVEC TAILS HC:

| | | | adj.P. | adj.P. | adj.P. | logFC. | logFC. | logFC. |
|---|---|---|---|---|---|---|---|---|
| | | SEQ | Val. | Val. | Val. | mEQ. | mWT. | mWT. |
| TAILS_ | | ID | mEQ. | mWT. | mWT. | over. | over. | over. |
| id | id | NO: | over.Luc | over.Luc | over.mEQ | Luc | Luc | mEQ |
| ADAMTS_ | qQAGSKTVR | 486 | 9.75E−01 | 4.33E−04 | 3.69E−04 | 0.02 | 3.74 | 3.72 |
| 3_1135 | | | | | | | | |
| ADAMTS_ | AQGAPR | 487 | 5.06E−01 | 3.55E−03 | 8.60E−04 | −0.20 | 1.61 | 1.80 |
| L2_592 | | | | | | | | |
| AEBP1_3 | FLEGFLSE | 123 | 4.16E−01 | 1.05E−03 | 4.09E−04 | −0.58 | 3.77 | 4.34 |
| 7 | LEPEPR | | | | | | | |
| AKAP12_ | FTQGKVVG | 334 | 3.92E−01 | 5.63E−04 | 1.16E−03 | −0.35 | 2.88 | 3.23 |
| 1106 | QTTPESF | | | | | | | |
| ASMT_3 | LSSAGFR | 488 | 2.96E−01 | 2.76E−03 | 5.50E−04 | −0.39 | 2.18 | 2.56 |
| 21 | | | | | | | | |
| BCOR_25 | DFIALR | 339 | 8.77E−01 | 8.29E−04 | 5.25E−04 | 0.08 | 2.95 | 2.87 |
| 8 | | | | | | | | |
| BICD1_2 | NNDDKMnG | 489 | 7.82E−01 | 1.02E−02 | 1.46E−02 | −0.08 | 1.23 | 1.31 |
| 80 | HIHGPLVK | | | | | | | |
| | LnGDYR | | | | | | | |
| BMP6_61 | qSSSGFLYR | 490 | 7.18E−01 | 2.56E−04 | 4.09E−04 | −0.10 | 3.21 | 3.32 |
| BMP6_12 | LKSAPLFmL | 340 | 3.78E−01 | 5.96E−03 | 3.97E−04 | −0.65 | 2.92 | 3.56 |
| 9 | | | | | | | | |
| CLU_267 | FQHPPTEFIR | 491 | 7.80E−01 | 2.89E−03 | 3.02E−03 | −0.08 | 1.56 | 1.64 |
| COL18A1_ | DKFQGVIA | 492 | 5.84E−01 | 7.68E−03 | 4.50E−03 | −0.14 | 1.28 | 1.42 |
| 629 | | | | | | | | |
| COL18A1_ | EETGAAL | 493 | 7.18E−01 | 9.11E−03 | 8.91E−03 | −0.09 | 1.11 | 1.20 |
| 679 | KPR | | | | | | | |
| COL18A1_ | TGAALKPR | 494 | 8.26E−01 | 8.06E−03 | 4.97E−03 | −0.06 | 1.26 | 1.32 |
| 681 | | | | | | | | |
| COL18A1_ | KVQLEAR | 495 | 3.74E−01 | 2.41E−02 | 5.53E−03 | −0.20 | 0.94 | 1.14 |
| 1485 | | | | | | | | |
| COL18A1_ | VAALQPPV | 496 | 1.82E−01 | 2.97E−02 | 6.48E−03 | −0.32 | 0.85 | 1.17 |
| 1502 | VQLH | | | | | | | |
| COL18A1_ | ALQPPVVQLH | 497 | 1.63E−01 | 8.32E−04 | 1.28E−02 | −1.22 | 2.08 | 3.31 |
| 1504 | | | | | | | | |
| COL18A1_ | LQPPVVQLH | 349 | 3.44E−01 | 6.46E−03 | 1.00E−03 | −0.84 | 3.05 | 3.88 |
| 1505 | | | | | | | | |
| COL18A1_ | AAVPIVNLK | 498 | 8.48E−01 | 3.49E−02 | 1.02E−02 | −0.06 | 1.03 | 1.09 |
| 1638 | | | | | | | | |
| COL18A1_ | EALFSGSEGP | 499 | 9.62E−01 | 1.90E−02 | 6.23E−03 | −0.02 | 1.13 | 1.14 |
| 1655 | LKPGAR | | | | | | | |
| COL18A1_ | SGSEGPLKPG | 500 | 7.33E−01 | 1.59E−02 | 3.39E−03 | −0.12 | 1.41 | 1.53 |
| 1659 | AR | | | | | | | |
| COL18A1_ | TEAPSATGQ | 501 | 3.09E−01 | 4.93E−02 | 8.91E−03 | −0.27 | 0.84 | 1.11 |
| 1711 | ASSLLGGR | | | | | | | |
| COL4A1_ | AGSGCGKC | 502 | 1.77E−01 | 4.10E−03 | 1.45E−02 | 0.37 | 1.49 | 1.12 |
| 32 | | | | | | | | |
| CTGF_17 | TVVGPALAA | 503 | 5.23E−01 | 3.76E−04 | 1.08E−02 | −0.45 | 2.94 | 3.39 |
| 3 | YR | | | | | | | |
| CTGF_17 | VVGPALAAYR | 504 | 3.56E−01 | 1.05E−03 | 7.18E−04 | −0.31 | 2.13 | 2.44 |
| 4 | | | | | | | | |
| CTGF_17 | ALAAYR | 505 | 8.62E−01 | 3.55E−03 | 4.48E−03 | −0.04 | 1.54 | 1.58 |
| 8 | | | | | | | | |
| CTGF_18 | TFGPDPTmIR | 506 | 9.59E−01 | 2.62E−04 | 2.14E−04 | −0.02 | 3.40 | 3.42 |
| 7 | | | | | | | | |
| CYP2F1_ | NPEHFLDA | 507 | 3.82E−01 | 2.07E−02 | 8.58E−03 | −0.26 | 1.15 | 1.41 |
| 409 | nQSFKKSP | | | | | | | |
| | AFMPFSAGR | | | | | | | |
| DMRTB1_ | AAPAPVPV | 508 | 3.67E−01 | 2.62E−04 | 4.09E−04 | −0.47 | 3.88 | 4.35 |
| 83 | PAASLRPL | | | | | | | |
| | SPGTPSG | | | | | | | |
| DOCK8_ | AQAGPR | 509 | 5.06E−01 | 3.55E−03 | 8.60E−04 | −0.20 | 1.61 | 1.80 |
| 107 | | | | | | | | |
| ECM1_10 | qEAVPLQK | 376 | 7.73E−01 | 3.44E−04 | 1.92E−03 | 0.09 | 2.21 | 2.12 |
| 8 | | | | | | | | |
| EFEMP1_ | AVAGPEMQTG | 157 | 2.81E−01 | 9.77E−04 | 7.18E−04 | −0.38 | 2.92 | 3.31 |
| 124 | R | | | | | | | |
| EFEMP1_ | VAGPEMQTGR | 158 | 7.94E−01 | 3.27E−03 | 7.18E−04 | −0.11 | 2.34 | 2.45 |
| 125_ | | | | | | | | |
| EFEMP1_ | EMQTGR | 510 | 3.52E−01 | 2.87E−03 | 1.29E−03 | −0.29 | 1.71 | 2.00 |
| 129 | | | | | | | | |
| ERAP2_7 | ISENLKR | 511 | 2.58E−01 | 5.99E−04 | 4.33E−04 | 0.64 | 5.05 | 4.41 |
| 18 | | | | | | | | |
| EWSR1_ | YSQPVQGYGTG | 512 | 9.02E−01 | 4.23E−02 | 1.71E−02 | −0.05 | 1.10 | 1.15 |
| 87 | AY | | | | | | | |
| FAM198B_ | EPSFPEIPL | 513 | 9.58E−01 | 4.04E−03 | 2.49E−03 | 0.02 | 1.53 | 1.51 |
| 78 | | | | | | | | |
| FBN1_29 | LEAGNVK | 387 | 3.40E−01 | 9.96E−03 | 5.44E−04 | −0.34 | 3.57 | 3.91 |
| FLNA_ | LTQTGGPHVKA | 168 | 2.65E−01 | 6.58E−04 | 2.90E−04 | −0.52 | 3.29 | 3.81 |
| 1285 | R | | | | | | | |
| FN1_23 | STGASKSKR | 514 | 2.24E−01 | 1.97E−03 | 2.89E−03 | 0.37 | 1.84 | 1.47 |
| FN1_23 | STGASK | 515 | 6.03E−01 | 3.37E−03 | 2.89E−03 | 0.13 | 1.57 | 1.44 |
| FN1_34 | qQmVQPQSPVA | 516 | 8.53E−01 | 2.02E−02 | 5.42E−03 | −0.06 | 1.37 | 1.43 |
| | VSQSKPGCY | | | | | | | |
| FN1_35 | qmVQPQSPV | 392 | 5.65E−01 | 1.95E−03 | 1.57E−03 | −0.27 | 2.31 | 2.58 |
| | AVSQSKPGC | | | | | | | |
| | Y | | | | | | | |
| FN1_36 | mVQPQSPVA | 393 | 3.15E−01 | 2.74E−04 | 8.41E−04 | −0.47 | 2.98 | 3.45 |
| | VSQSKPGCY | | | | | | | |
| FN1_40 | qSPVAVSQS | 394 | 3.76E−01 | 1.12E−03 | 7.65E−03 | −0.50 | 2.45 | 2.96 |
| | KPGCY | | | | | | | |
| FN1_293 | VYQPQPHPQPP | 517 | 2.92E−01 | 3.76E−02 | 5.70E−03 | −0.29 | 0.96 | 1.24 |
| | PYGHCVT | | | | | | | |
| FN1_607 | SSSGPVEVFIT | 397 | 6.22E−01 | 3.05E−03 | 3.86E−02 | −0.40 | 2.31 | 2.71 |
| FN1_886 | NQESTPVVIQ | 176 | 4.25E−01 | 1.66E−02 | 7.40E−03 | −0.43 | 1.61 | 2.04 |
| | QETTGTPR | | | | | | | |
| FN1_114 | LTPGVEYVYTI | 177 | 4.79E−01 | 2.56E−04 | 3.57E−04 | −0.24 | 3.43 | 3.68 |
| 3 | QVLR | | | | | | | |
| FN1_114 | TPGVEYVYTIQ | 518 | 5.85E−01 | 1.64E−02 | 3.95E−03 | −0.19 | 1.43 | 1.62 |
| 4 | VLR | | | | | | | |
| FN1_162 | LVQTAVTTIP | 519 | 7.18E−01 | 2.07E−02 | 6.48E−03 | −0.16 | 1.40 | 1.56 |
| 4 | APT | | | | | | | |
| FN1_165 | SSSPVTGYR | 178 | 7.67E−01 | 2.47E−03 | 1.17E−03 | −0.08 | 1.56 | 1.63 |
| 6 | | | | | | | | |
| FN1_174 | WESPQGQVSR | 520 | 4.79E−01 | 2.74E−04 | 2.90E−04 | −0.19 | 2.86 | 3.05 |
| 3 | | | | | | | | |
| FN1_178 | LQGLRPGSEYT | 400 | 1.37E−01 | 1.80E−03 | 5.44E−04 | −0.47 | 1.80 | 2.27 |
| 0 | VSVVALH | | | | | | | |
| FN1_180 | LIGTQSTAIP | 401 | 8.56E−01 | 3.85E−03 | 5.42E−03 | 0.05 | 1.47 | 1.42 |
| 5 | APT | | | | | | | |
| FN1_211 | FVTHPGY | 402 | 1.53E−01 | 1.90E−02 | 1.69E−02 | −0.62 | 1.05 | 1.67 |
| 3 | | | | | | | | |
| FSTL1_2 | LQKHQETA | 405 | 4.78E−01 | 1.08E−03 | 4.33E−04 | −0.24 | 2.34 | 2.57 |
| 90 | | | | | | | | |
| GCNT7_7 | HAHLHTPGnCS | 521 | 1.61E−01 | 3.86E−03 | 9.39E−04 | −0.47 | 2.35 | 2.82 |
| 8 | R | | | | | | | |
| GOLM1_ | LSEPQPR | 522 | 4.16E−01 | 5.52E−04 | 2.14E−04 | 0.48 | 4.16 | 3.68 |
| 203 | | | | | | | | |
| HMGA2_ | qGQPAAPAPQ | 523 | 6.75E−01 | 3.01E−03 | 6.77E−03 | 0.13 | 2.30 | 2.17 |
| 16 | KR | | | | | | | |
| HSPG2_2 | LLPGSVRPLP | 410 | 8.74E−01 | 5.98E−02 | 4.57E−02 | −0.03 | 0.74 | 0.77 |
| 75 | CGPQ | | | | | | | |
| HSPG2_2 | LLPGSVR | 191 | 3.56E−01 | 4.62E−02 | 1.58E−01 | 0.25 | 0.99 | 0.74 |
| 75 | | | | | | | | |
| HSPG2_1 | LSAPVVSIHP | 411 | 5.91E−01 | 7.96E−04 | 3.73E−03 | −0.22 | 2.31 | 2.53 |
| 863 | PQLTVQPGQLA | | | | | | | |
| HSPG2_1 | HSSAGQQVAR | 412 | 1.69E−01 | 3.55E−03 | 3.18E−03 | −0.36 | 1.91 | 2.27 |
| 936 | | | | | | | | |
| HSPG2_2 | ANLAYPAGS | 194 | 9.75E−01 | 8.65E−03 | 1.16E−03 | −0.01 | 1.72 | 1.73 |
| 331 | TQPIR | | | | | | | |
| HSPG2_2 | NLAYPAGSTQ | 524 | 2.93E−01 | 3.02E−04 | 4.09E−04 | −0.42 | 3.35 | 3.77 |
| 332 | PIR | | | | | | | |
| HSPG2_2 | ALGVTPTVR | 525 | 7.67E−02 | 3.33E−02 | 1.16E−03 | −0.90 | 1.16 | 2.06 |
| 432 | | | | | | | | |
| HSPG2_2 | VAYPVR | 526 | 2.41E−01 | 2.84E−02 | 8.02E−03 | −0.25 | 0.88 | 1.13 |
| 532 | | | | | | | | |
| IGFBP2_ | VAGGAR | 527 | 6.74E−01 | 5.07E−04 | 3.57E−04 | 0.14 | 2.79 | 2.65 |
| 67 | | | | | | | | |
| IGFBP7_ | AAGGPGVSGVC | 214 | 8.92E−01 | 9.36E−04 | 1.29E−03 | −0.04 | 1.86 | 1.89 |
| 101 | VCKSR | | | | | | | |
| KIF23_79 | NAPPIR | 528 | 6.47E−01 | 4.76E−04 | 3.57E−04 | −0.19 | 3.10 | 3.29 |
| 4 | | | | | | | | |
| LCORL_ | LEALPAGK | 529 | 1.84E−01 | 3.63E−04 | 3.57E−04 | −0.60 | 3.46 | 4.06 |
| 228 | | | | | | | | |
| LOX_84 | AQQPR | 530 | 5.06E−01 | 3.55E−03 | 8.60E−04 | −0.20 | 1.61 | 1.80 |
| LOX_124 | WFQAGYSTSR | 227 | 9.38E−01 | 2.56E−04 | 2.90E−04 | 0.03 | 3.38 | 3.35 |
| LOX_125 | FQAGYSTSR | 424 | 5.22E−01 | 2.56E−04 | 1.07E−03 | 0.24 | 3.82 | 3.58 |
| LOXL2_3 | FQQPAPEYHQP | 230 | 1.64E−01 | 2.71E−04 | 7.19E−04 | −0.65 | 2.82 | 3.48 |
| 7 | QAPAnVAKIQL | | | | | | | |
| | R | | | | | | | |
| LRRC17_ | qVSGRPPVIKP | 428 | 4.15E−01 | 6.88E−04 | 4.33E−04 | −0.22 | 2.12 | 2.34 |
| 225 | EV | | | | | | | |
| LTBP1_3 | LTATNFR | 234 | 5.21E−01 | 2.56E−04 | 3.57E−04 | −0.18 | 3.14 | 3.33 |
| 93 | | | | | | | | |
| LTBP2_7 | VAGLQPVER | 430 | 1.53E−01 | 6.84E−03 | 2.02E−03 | −0.38 | 1.13 | 1.51 |
| 9 | | | | | | | | |
| LTBP2_1 | QQQPAPR | 531 | 4.87E−01 | 7.40E−04 | 4.09E−04 | −0.49 | 4.83 | 5.32 |
| 29 | | | | | | | | |
| LTBP2_2 | SAAGEGTLAR | 431 | 6.02E−01 | 8.60E−04 | 2.41E−03 | 0.12 | 1.84 | 1.71 |
| 50 | | | | | | | | |
| LTBP2_2 | GEGTLAR | 532 | 6.85E−01 | 2.56E−04 | 3.57E−04 | −0.09 | 2.90 | 2.99 |
| 53 | | | | | | | | |
| LTBP2_2 | HVGLSR | 533 | 4.40E−01 | 1.38E−03 | 2.14E−04 | −0.44 | 3.79 | 4.24 |
| 91 | | | | | | | | |
| LTBP2_4 | LEAPLK | 534 | 6.53E−02 | 3.64E−04 | 2.14E−04 | −0.91 | 3.62 | 4.53 |
| 49 | | | | | | | | |
| LTBP2_5 | LVENSVETRPP | 433 | 6.90E−01 | 6.46E−03 | 8.02E−03 | −0.22 | 2.47 | 2.68 |
| 02 | PWLPASPGHS | | | | | | | |
| | LW | | | | | | | |
| LTBP3_1 | LSTGALPPLA | 535 | 4.60E−01 | 2.41E−02 | 1.61E−03 | −0.39 | 1.82 | 2.21 |
| 64 | PEG | | | | | | | |
| LTBP3_2 | HAAFLVPLGPG | 536 | 8.89E−01 | 4.19E−02 | 1.30E−03 | −0.13 | 2.41 | 2.54 |
| 05 | QISA | | | | | | | |
| LTBP3_2 | FLVPLGPGQI | 435 | 2.79E−01 | 5.96E−03 | 2.96E−02 | −0.60 | 1.54 | 2.14 |
| 08 | SA | | | | | | | |
| LTBP3_2 | VQAPPPVVNVR | 537 | 3.84E−01 | 1.20E−03 | 3.57E−04 | −0.39 | 2.72 | 3.11 |
| 21 | | | | | | | | |
| LTBP3_2 | SVQVHR | 248 | 7.38E−01 | 3.33E−03 | 6.36E−03 | −0.10 | 1.47 | 1.57 |
| 39 | | | | | | | | |
| MAP2_13 | LEQPEVER | 538 | 9.02E−01 | 4.58E−03 | 5.27E−03 | −0.03 | 1.48 | 1.51 |
| 14 | | | | | | | | |
| MAP4_81 | AVASTGPSSR | 252 | 2.16E−01 | 5.90E−04 | 3.72E−03 | 0.36 | 2.87 | 2.51 |
| 5 | | | | | | | | |
| MAP4_81 | VASTGPSSR | 253 | 7.92E−01 | 1.04E−03 | 3.87E−03 | 0.08 | 2.13 | 2.05 |
| 6 | | | | | | | | |
| MCFD2_ | HLEGVINKPEA | 539 | 5.80E−01 | 2.15E−02 | 4.55E−02 | 0.14 | 1.13 | 0.99 |
| 55 | | | | | | | | |
| MMP2_ | HAFAPGTGVGG | 540 | 3.99E−01 | 2.85E−02 | 2.51E−03 | −0.32 | 1.31 | 1.63 |
| 93 | | | | | | | | |
| MMRN1_ | LIHTNQA | 541 | 2.07E−01 | 1.02E−02 | 2.19E−03 | −0.35 | 1.25 | 1.60 |
| 290 | | | | | | | | |
| MMRN1_ | LQKGLTEFV | 542 | 3.62E−01 | 3.44E−04 | 2.90E−04 | −0.49 | 4.61 | 5.09 |
| 959 | | | | | | | | |
| NID1_36 | FHQQHPQVI | 450 | 3.32E−01 | 7.51E−03 | 3.79E−02 | −0.30 | 1.48 | 1.78 |
| 0 | | | | | | | | |
| NID2_29 | HSSVPLGR | 259 | 9.13E−01 | 3.69E−03 | 3.84E−03 | −0.10 | 2.80 | 2.90 |
| 1 | | | | | | | | |
| NUCB1_ | LIQTATR | 543 | 3.78E−01 | 6.19E−04 | 1.43E−03 | −0.29 | 2.25 | 2.54 |
| 157 | | | | | | | | |
| NUCB1_ | FLASTQR | 265 | 8.59E−01 | 4.81E−04 | 4.94E−04 | 0.06 | 2.71 | 2.65 |
| 317 | | | | | | | | |
| NUCB1_ | LQQAVLHm | 544 | 5.54E−01 | 2.22E−02 | 4.41E−03 | −0.22 | 1.32 | 1.54 |
| 389 | | | | | | | | |
| PABPC1_ | AAAATPAVR | 545 | 3.39E−01 | 8.37E−03 | 3.67E−03 | −0.35 | 1.50 | 1.85 |
| 466 | | | | | | | | |
| PDLIM5_ | LSAGKTAVNVP | 456 | 9.99E−01 | 1.48E−03 | 2.14E−04 | 0.00 | 4.27 | 4.27 |
| 194 | R | | | | | | | |
| PDLIM5_ | qITGTEHLK | 546 | 1.95E−01 | 4.73E−04 | 3.97E−04 | −0.35 | 2.53 | 2.88 |
| 288 | | | | | | | | |
| PROB1_6 | AGAQPR | 547 | 5.06E−01 | 3.55E−03 | 8.60E−04 | −0.20 | 1.61 | 1.80 |
| 5 | | | | | | | | |
| PSAP_30 | LVEPIKKH | 548 | 8.19E−01 | 4.08E−04 | 4.33E−04 | −0.06 | 2.38 | 2.44 |
| 1 | | | | | | | | |
| PTBP1_3 | AAAAAGR | 549 | 8.41E−01 | 2.65E−02 | 2.37E−02 | 0.06 | 1.11 | 1.06 |
| 76 | | | | | | | | |
| PZP_578 | HAHLQVAAAP | 550 | 6.13E−01 | 2.58E−02 | 1.94E−03 | −0.34 | 2.11 | 2.45 |
| | QSLCALR | | | | | | | |
| QSOX2_5 | EGGTLAR | 551 | 6.85E−01 | 2.56E−04 | 3.57E−04 | −0.09 | 2.90 | 2.99 |
| 80 | | | | | | | | |
| RET_104 | NAPLPR | 552 | 6.47E−01 | 4.76E−04 | 3.57E−04 | −0.19 | 3.10 | 3.29 |
| 5 | | | | | | | | |
| SERBP1_ | qAAAQTNSNAA | 288 | 5.75E−01 | 2.81E−02 | 4.48E−03 | −0.20 | 1.23 | 1.43 |
| 56 | GKQLR | | | | | | | |
| SERBP1_ | qTNSNAAGKQL | 259 | 1.94E−01 | 8.96E−03 | 4.69E−02 | 0.40 | 1.33 | 0.94 |
| 60 | R | | | | | | | |
| SERBP1_ | qLQGEGKII | 462 | 5.96E−01 | 4.05E−04 | 7.19E−04 | 0.13 | 2.57 | 2.44 |
| 116 | | | | | | | | |
| SIK3_44 | AAGQPR | 553 | 5.06E−01 | 3.55E−03 | 8.60E−04 | −0.20 | 1.61 | 1.80 |
| SPOCK1_ | LAGGAGPNHG | 466 | 5.07E−01 | 3.42E−02 | 1.02E−02 | −0.17 | 0.90 | 1.07 |
| 27 | NFL | | | | | | | |
| SRGN_59 | LLPGESNKIPR | 296 | 5.46E−01 | 1.31E−02 | 7.79E−03 | −0.13 | 1.04 | 1.17 |
| ST6GAL1_ | SFQVWNK | 554 | 6.46E−01 | 1.02E−03 | 4.28E−04 | −0.19 | 2.82 | 3.01 |
| 92 | | | | | | | | |
| SYNRG_ | mSSGLPR | 555 | 2.33E−01 | 1.20E−02 | 4.09E−04 | −0.56 | 1.75 | 2.31 |
| 326 | | | | | | | | |
| TF_470 | HTAVGR | 556 | 2.58E−01 | 6.19E−04 | 4.09E−04 | −0.80 | 4.83 | 5.63 |
| THBS1_6 | FGQGVEHATAN | 557 | 9.73E−01 | 8.29E−04 | 4.09E−04 | −0.01 | 2.27 | 2.28 |
| 30 | K | | | | | | | |
| THBS2_2 | LSENLKR | 318 | 2.58E−01 | 5.99E−04 | 4.33E−04 | 0.64 | 5.05 | 4.41 |
| 90 | | | | | | | | |
| THSD4_2 | STAISCIGAYR | 558 | 6.33E−01 | 2.22E−02 | 6.77E−03 | −0.13 | 1.10 | 1.22 |
| 86 | | | | | | | | |
| TLR2_37 | LKNSAC | 559 | 8.69E−01 | 2.71E−04 | 3.87E−04 | 0.05 | 2.73 | 2.68 |
| 7 | | | | | | | | |
| UBE3C_3 | ASASCH | 560 | 7.50E−01 | 2.02E−02 | 8.69E−03 | −0.07 | 1.01 | 1.08 |
| 57 | | | | | | | | |
| UQCRH_ | HGSPNNSERAM | 561 | 9.82E−01 | 2.54E−02 | 7.79E−03 | 0.01 | 1.39 | 1.38 |
| 76 | RAVGEmCKGPG | | | | | | | |
| | AAR | | | | | | | |
| VIM_275 | qYESVAAKNLQ | 562 | 4.24E−01 | 8.60E−03 | 1.35E−03 | −0.34 | 1.72 | 2.06 |
| ZNF611_ | LQPLPPRYLFQ | 563 | 7.66E−01 | 2.41E−02 | 1.76E−02 | −0.20 | 1.77 | 1.97 |
| 40 | mH | | | | | | | |
| ZNF672_ | HLQTHSGEKPF | 564 | 4.79E−01 | 1.61E−03 | 6.62E−03 | −0.43 | 2.28 | 2.71 |
| 217 | K | | | | | | | |
| ZYX_244 | PVSLANTQPR | 565 | 4.34E−01 | 7.49E−03 | 1.81E−02 | 0.18 | 1.20 | 1.02 |

| | | | | | | | ATS7_HU | |
|---|---|---|---|---|---|---|---|---|
| | | | | | | | VEC_ | |
| | | | | | | | TAILS_ | |
| | | | | | | | frxns_ | |
| | | | | | | | mATS7inDB_ | |
| | | | | | | SEQ | AspGluN. | |
| TAILS_ | accession_ | gene | Start | previous_ | | ID | total | entry_ |
| id | number | Symbol | AA | aa | sequence | NO: | Intensity | name |
| ADAMTS3_ | O15072 | ADAMTS3 | 1135 | (A) | qQAGSKTVR | 486 | 1.08E+07 | A |
| 1135 | | | | | | | | disintegrin |
| | | | | | | | | and metallo- |
| | | | | | | | | proteinase |
| | | | | | | | | with |
| | | | | | | | | thrombospondin |
| | | | | | | | | motifs |
| | | | | | | | | 3 |
| ADAMTSL | B1BOD4 | ADAMTSL | 592 | (F) | AQGAPR | 467 | 5.51E+07 | ADAMTS- |
| 2_592 | | 2 | | | | | | like protein |
| | | | | | | | | 2 |
| AEBP1_37 | Q8IUX7 | AEBP1 | 37 | (E) | FLEGFLSELEPE | 123 | 5.26E+06 | Adipocyte |
| | | | | | PR | | | enhancer- |
| | | | | | | | | binding |
| | | | | | | | | protein 1 |
| AKAP12_1 | Q02952 | AKAP12 | 1106 | (P) | FTQGKVVGQTT | 334 | 3.55E+07 | A-kinase |
| 106 | | | | | PESF | | | anchor |
| | | | | | | | | protein 12 |
| ASMT_321 | P46597 | ASMT | 321 | (L) | LSSAGFR | 488 | 2.00E+09 | Acetylserotonin |
| | | | | | | | | O- |
| | | | | | | | | methyl- |
| | | | | | | | | transferase |
| BCOR_258 | A1A564 | BCOR | 258 | (R) | DFIALR | 339 | 1.21E+08 | BCL-6 |
| | | | | | | | | corepressor |
| BICD1_280 | A8MVZ6 | BICD1 | 280 | (P) | NNDDKMnGHIH | 489 | 7.87E+09 | BICD1 |
| | | | | | GPLVKLnGDYR | | | protein |
| BMP6_61 | P22004 | BMP6 | 61 | (P) | qSSSGFLYR | 490 | 2.61E+08 | Bone |
| | | | | | | | | morphogenetic |
| | | | | | | | | protein 6 |
| BMP6_129 | P22004 | BMP6 | 129 | (R) | LKSAPLFmL | 340 | 1.74E+08 | Bone |
| | | | | | | | | morphogenetic |
| | | | | | | | | protein 6 |
| CLU_267 | P10909 | CLU | 267 | (A) | FQHPPTEFIR | 491 | 9.81E+08 | Clusterin |
| COL18A1_ | P39060 | COL18A1 | 629 | (P) | DKFQGVIA | 492 | 2.80E+09 | Collagen |
| 629 | | | | | | | | alpha- |
| | | | | | | | | 1(XVIII) |
| | | | | | | | | chain |
| COL18A1_ | P39060 | COL18A1 | 679 | (R) | EETGAALKPR | 493 | 2.59E+10 | Collagen |
| 679 | | | | | | | | alpha- |
| | | | | | | | | 1(XVIII) |
| | | | | | | | | chain |
| COL18A1_ | P39060 | COL18A1 | 681 | (E) | TGAALKPR | 494 | 1.36E+08 | Collagen |
| 681 | | | | | | | | alpha- |
| | | | | | | | | 1(XVIII) |
| | | | | | | | | chain |
| COL18A1_ | P39060 | COL18A1 | 1485 | (R) | KVQLEAR | 495 | 3.68E+08 | Collagen |
| 1485 | | | | | | | | alpha- |
| | | | | | | | | 1(XVIII) |
| | | | | | | | | chain |
| COL18A1_ | P39060 | COL18A1 | 1502 | (E) | VAALQPPVVQL | 496 | 4.46E+08 | Collagen |
| 1502 | | | | | H | | | alpha- |
| | | | | | | | | 1(XVIII) |
| | | | | | | | | chain |
| COL18A1_ | P39060 | COL18A1 | 1504 | (A) | ALQPPVVQLH | 497 | 7.84E+09 | Collagen |
| 1504 | | | | | | | | alpha- |
| | | | | | | | | 1(XVIII) |
| | | | | | | | | chain |
| COL18A1_ | P39060 | COL18A1 | 1505 | (A) | LQPPVVQLH | 349 | 1.34E+11 | Collagen |
| 1505 | | | | | | | | alpha- |
| | | | | | | | | 1(XVIII) |
| | | | | | | | | chain |
| COL18A1_ | P39060 | COL18A1 | 1638 | (R) | AAVPIVNLK | 498 | 4.87E+09 | Collagen |
| 1638 | | | | | | | | alpha- |
| | | | | | | | | 1(XVIII) |
| | | | | | | | | chain |
| COL18A1_ | P39060 | COL18A1 | 1655 | (W) | EALFSGSEGPLK | 499 | 1.55E+10 | Collagen |
| 1655 | | | | | PGAR | | | alpha- |
| | | | | | | | | 1(XVIII) |
| | | | | | | | | chain |
| COL18A1_ | P39060 | COL18A1 | 1659 | (F) | SGSEGPLKPGAR | 500 | 1.90E+10 | Collagen |
| 1659 | | | | | | | | alpha- |
| | | | | | | | | 1(XVIII) |
| | | | | | | | | chain |
| COL18A1_ | P39060 | COL18A1 | 1711 | (R) | TEAPSATGQASS | 501 | 1.43E+09 | Collagen |
| 1711 | | | | | LLGGR | | | alpha- |
| | | | | | | | | 1(XVIII) |
| | | | | | | | | chain |
| COL4A1_3 | P02462 | COL4A1 | 32 | (C) | AGSGCGKC | 502 | 4.96E+08 | Collagen |
| 2 | | | | | | | | alpha-1(IV) |
| | | | | | | | | chain |
| CTGF_173 | P29279 | CTGF | 173 | (Q) | TVVGPALAAYR | 503 | 3.77E+10 | Connective |
| | | | | | | | | tissue |
| | | | | | | | | growth |
| | | | | | | | | factor |
| CTGF_174 | P29279 | CTGF | 174 | (T) | VVGPALAAYR | 504 | 5.59E+08 | Connective |
| | | | | | | | | tissue |
| | | | | | | | | growth |
| | | | | | | | | factor |
| CTGF_178 | P29279 | CTGF | 178 | (P) | ALAAYR | 505 | 3.90E+08 | Connective |
| | | | | | | | | tissue |
| | | | | | | | | growth |
| | | | | | | | | factor |
| CTGF_187 | P29279 | CTGF | 187 | (D) | TFGPDPTmIR | 506 | 8.03E+08 | Connective |
| | | | | | | | | tissue |
| | | | | | | | | growth |
| | | | | | | | | factor |
| CYP2F1_40 | P24903 | CYP2F1 | 409 | (F) | NPEHFLDAnQSF | 507 | 2.25E+08 | Cytochrome |
| 9 | | | | | KKSPAFMPFSA | | | P450 2F1 |
| | | | | | GR | | | |
| DMRTB1_8 | Q96MA1 | DMRTB1 | 83 | (A) | AAPAPVPVPAA | 508 | 2.00E+08 | Doublesex- |
| 3 | | | | | SLRPLSPGTPSG | | | and mab-3- |
| | | | | | | | | related |
| | | | | | | | | transcription |
| | | | | | | | | factor B1 |
| DOCK8_10 | E9PDJ4 | DOCK8 | 107 | (A) | AQAGPR | 509 | 1.38E+08 | Dedicator of |
| 7 | | | | | | | | cytokinesis |
| | | | | | | | | protein 8 |
| ECM1_108 | Q16610 | ECM1 | 108 | (P) | qEAVPLQK | 376 | 1.47E+08 | Extracellular |
| | | | | | | | | matrix |
| | | | | | | | | protein 1 |
| EFEMP1_1 | Q12805 | EFEMP1 | 124 | (A) | AVAGPEMQTGR | 157 | 2.92E+11 | EGF- |
| 24 | | | | | | | | containing |
| | | | | | | | | fibulin-like |
| | | | | | | | | extracellular |
| | | | | | | | | matrix |
| | | | | | | | | protein 1 |
| EFEMP1_1 | Q12805 | EFEMP1 | 125 | (A) | VAGPEMQTGR | 158 | 9.33E+10 | EGF- |
| 25 | | | | | | | | containing |
| | | | | | | | | fibulin-like |
| | | | | | | | | extracellular |
| | | | | | | | | matrix |
| | | | | | | | | protein 1 |
| EFEMP1_1 | Q12805 | EFEMP1 | 129 | (P) | EMQTGR | 510 | 8.84E+08 | EGF- |
| 29 | | | | | | | | containing |
| | | | | | | | | fibulin-like |
| | | | | | | | | extracellular |
| | | | | | | | | matrix |
| | | | | | | | | protein 1 |
| ERAP2_718 | Q6P179 | ERAP2 | 718 | (D) | ISENLKR | 511 | 3.67E+08 | Endoplasmic |
| | | | | | | | | reticulum |
| | | | | | | | | aminopeptidase |
| | | | | | | | | 2 |
| EWSR1_87 | AOAOD9SF | EWSR1 | 87 | (A) | YSQPVQGYGTG | 512 | 1.50E+08 | RNA- |
| | L3 | | | | AY | | | binding |
| | | | | | | | | protein |
| | | | | | | | | EWS |
| FAM198B_ | Q6UWH4 | FAM198B | 78 | (A) | EPSFPEIPL | 513 | 1.42E+08 | Protein |
| 78 | | | | | | | | FAM198B |
| FBN1_29 | P35555 | FBN1 | 29 | (N) | LEAGNVK | 387 | 1.85E+09 | Fibrillin-1 |
| FLNA_128 | P21333 | FLNA | 1285 | (A) | LTQTGGPHVKA | 169 | 2.92E+09 | Filamin-A |
| 5 | | | | | R | | | |
| FN1_23 | P02751-15 | FN1 | 23 | (P) | STGASKSKR | 514 | 4.64E+10 | Isoform 15 |
| | | | | | | | | of |
| | | | | | | | | Fibronectin |
| FN1_23 | P02751-15 | FN1 | 23 | (P) | STGASK | 515 | 3.30E+08 | Isoform 15 |
| | | | | | | | | of |
| | | | | | | | | Fibronectin |
| FN1_34 | P02751-15 | FN1 | 34 | (A) | qQmVQPQSPVA | 516 | 5.42E+09 | Isoform 15 |
| | | | | | VSQSKPGCY | | | of |
| | | | | | | | | Fibronectin |
| FN1_35 | P02751-15 | FN1 | 35 | (Q) | qmVQPQSPVAV | 392 | 2.07E+10 | Isoform 15 |
| | | | | | SQSKPGCY | | | of |
| | | | | | | | | Fibronectin |
| FN1_36 | P02751-15 | FN1 | 36 | (Q) | mVQPQSPVAVS | 393 | 1.76E+11 | Isoform 15 |
| | | | | | QSKPGCY | | | of |
| | | | | | | | | Fibronectin |
| FN1_40 | P02751-15 | FN1 | 40 | (P) | qSPVAVSQSKPG | 394 | 1.37E+10 | Isoform 15 |
| | | | | | CY | | | of |
| | | | | | | | | Fibronectin |
| FN1_293 | P02751-15 | FN1 | 293 | (A) | VYQPQPHPQPPP | 517 | 7.01E+10 | Isoform 15 |
| | | | | | YGHCVT | | | of |
| | | | | | | | | Fibronectin |
| FN1_607 | P02751-15 | FN1 | 607 | (P) | SSSGPVEVFIT | 397 | 4.88E+09 | Isoform 15 |
| | | | | | | | | of |
| | | | | | | | | Fibronectin |
| FN1_886 | P02751-15 | FN1 | 886 | (E) | NQESTPVVIQQE | 176 | 8.05E+09 | Isoform 15 |
| | | | | | TTGTPR | | | of |
| | | | | | | | | Fibronectin |
| FN1_1143 | P02751-15 | FN1 | 1143 | (G) | LTPGVEYVYTIQ | 177 | 4.67E+09 | Isoform 15 |
| | | | | | VLR | | | of |
| | | | | | | | | Fibronectin |
| FN1_1144 | P02751-15 | FN1 | 1144 | (L) | TPGVEYVYTIQ | 518 | 2.58E+08 | Isoform 15 |
| | | | | | VLR | | | of |
| | | | | | | | | Fibronectin |
| FN1_1624 | P02751-8 | FN1 | 1624 | (P) | LVQTAVTTIPAP | 519 | 6.33E+09 | Isoform 15 |
| | | | | | T | | | of |
| | | | | | | | | Fibronectin |
| FN1_1656 | P02751-15 | FN1 | 1656 | (P) | SSSPVTGYR | 178 | 2.23E+10 | Isoform 15 |
| | | | | | | | | of |
| | | | | | | | | Fibronectin |
| FN1_1743 | P02751-15 | FN1 | 1743 | (A) | WESPQGQVSR | 520 | 9.68E+08 | Isoform 15 |
| | | | | | | | | of |
| | | | | | | | | Fibronectin |
| FN1_1780 | P02751-15 | FN1 | 1780 | (E) | LQGLRPGSEYT | 400 | 2.82E+09 | Isoform 15 |
| | | | | | VSVVALH | | | of |
| | | | | | | | | Fibronectin |
| FN1_1805 | P02751-15 | FN1 | 1805 | (P) | LIGTQSTAIPAPT | 401 | 4.74E+10 | Isoform 15 |
| | | | | | | | | of |
| | | | | | | | | Fibronectin |
| FN1_2113 | P02751-15 | FN1 | 2113 | (P) | FVTHPGY | 402 | 2.71E+09 | Isoform 15 |
| | | | | | | | | of |
| | | | | | | | | Fibronectin |
| FSTL1_290 | Q12841 | FSTL1 | 290 | (E) | LQKHQETA | 405 | 1.45E+08 | Follistatin- |
| | | | | | | | | related |
| | | | | | | | | protein 1 |
| GCNT7_78 | A0A087WZ | GCNT7 | 78 | (I) | HAHLHTPGnCS | 521 | 2.10E+08 | Beta-1,3- |
| | E6 | | | | R | | | galactosyl- |
| | | | | | | | | O-glycosyl- |
| | | | | | | | | glycoprotein |
| | | | | | | | | beta-1,6-N- |
| | | | | | | | | acetylglucos |
| | | | | | | | | aminyl- |
| | | | | | | | | transferase 7 |
| GOLM1_20 | Q8NBJ4 | GOLM1 | 203 | (A) | LSEPQPR | 522 | 2.04E+09 | Golgi |
| 3 | | | | | | | | membrane |
| | | | | | | | | protein 1 |
| HMGA2_16 | F5H2A4 | HMGA2 | 16 | (A) | qGQPAAPAPQK | 523 | 3.64E+08 | High |
| | | | | | R | | | mobility |
| | | | | | | | | group |
| | | | | | | | | protein |
| | | | | | | | | HMGI-C |
| HSPG2_275 | P98160 | HSPG2 | 275 | (P) | LLPGSVRPLPCG | 410 | 2.67E+07 | Basement |
| | | | | | PQ | | | membrane- |
| | | | | | | | | specific |
| | | | | | | | | heparan |
| | | | | | | | | sulfate |
| | | | | | | | | proteoglycan |
| | | | | | | | | core |
| | | | | | | | | protein |
| HSPG2_275 | P98160 | HSPG2 | 275 | (P) | LLPGSVR | 191 | 1.19E+08 | Basement |
| | | | | | | | | membrane- |
| | | | | | | | | specific |
| | | | | | | | | heparan |
| | | | | | | | | sulfate |
| | | | | | | | | proteoglycan |
| | | | | | | | | core |
| | | | | | | | | protein |
| HSPG2_186 | P98160 | HSPG2 | 1863 | (T) | LSAPVVSIHPPQ | 411 | 4.95E+08 | Basement |
| 3 | | | | | LTVQPGQLA | | | membrane- |
| | | | | | | | | specific |
| | | | | | | | | heparan |
| | | | | | | | | sulfate |
| | | | | | | | | proteoglycan |
| | | | | | | | | core |
| | | | | | | | | protein |
| HSPG2_193 | P98160 | HSPG2 | 1936 | (A) | HSSAGQQVAR | 412 | 2.96E+08 | Basement |
| 6 | | | | | | | | membrane- |
| | | | | | | | | specific |
| | | | | | | | | heparan |
| | | | | | | | | sulfate |
| | | | | | | | | proteoglycan |
| | | | | | | | | core |
| | | | | | | | | protein |
| HSPG2_233 | P98160 | HSPG2 | 2331 | (G) | ANLAYPAGSTQ | 194 | 2.45E+09 | Basement |
| 1 | | | | | PIR | | | membrane- |
| | | | | | | | | specific |
| | | | | | | | | heparan |
| | | | | | | | | sulfate |
| | | | | | | | | proteoglycan |
| | | | | | | | | core |
| | | | | | | | | protein |
| HSPG2_233 | P98160 | HSPG2 | 2332 | (A) | NLAYPAGSTQPI | 524 | 6.45E+08 | Basement |
| 2 | | | | | R | | | membrane- |
| | | | | | | | | specific |
| | | | | | | | | heparan |
| | | | | | | | | sulfate |
| | | | | | | | | proteoglycan |
| | | | | | | | | core |
| | | | | | | | | protein |
| HSPG2_243 | P98160 | HSPG2 | 2432 | (P) | ALGVTPTVR | 525 | 1.09E+08 | Basement |
| 2 | | | | | | | | membrane- |
| | | | | | | | | specific |
| | | | | | | | | heparan |
| | | | | | | | | sulfate |
| | | | | | | | | proteoglycan |
| | | | | | | | | core |
| | | | | | | | | protein |
| HSPG2_253 | P98160 | HSPG2 | 2532 | (G) | VAYPVR | 526 | 2.65E+08 | Basement |
| 2 | | | | | | | | membrane- |
| | | | | | | | | specific |
| | | | | | | | | heparan |
| | | | | | | | | sulfate |
| | | | | | | | | proteoglycan |
| | | | | | | | | core |
| | | | | | | | | protein |
| IGFBP2_67 | P18065 | IGFBP2 | 67 | (A) | VAGGAR | 527 | 2.12E+08 | Insulin-like |
| | | | | | | | | growth |
| | | | | | | | | factor- |
| | | | | | | | | binding |
| | | | | | | | | protein 2 |
| IGFBP7_10 | Q16270 | IGFBP7 | 101 | (A) | AAGGPGVSGVC | 214 | 1.12E+08 | Insulin-like |
| 1 | | | | | VCKSR | | | growth |
| | | | | | | | | factor- |
| | | | | | | | | binding |
| | | | | | | | | protein 7 |
| KIF23_794 | H7BYN4 | KIF23 | 794 | (R) | NAPPIR | 528 | 2.62E+09 | Kinesin-like |
| | | | | | | | | protein |
| LCORL_22 | C9JI46 | LCORL | 228 | (H) | LEALPAGK | 529 | 7.54E+07 | Ligand- |
| 8 | | | | | | | | dependent |
| | | | | | | | | nuclear |
| | | | | | | | | receptor |
| | | | | | | | | corepressor- |
| | | | | | | | | like protein |
| LOX_84 | P28300 | LOX | 84 | (S) | AQQPR | 530 | 2.75E+07 | Protein- |
| | | | | | | | | lysine 6- |
| | | | | | | | | oxidase |
| LOX_124 | P28300 | LOX | 124 | (H) | WFQAGYSTSR | 227 | 8.74E+07 | Protein- |
| | | | | | | | | lysine 6- |
| | | | | | | | | oxidase |
| LOX_125 | P28300 | LOX | 125 | (W) | FQAGYSTSR | 424 | 1.50E+09 | Protein- |
| | | | | | | | | lysine 6- |
| | | | | | | | | oxidase |
| LOXL2_37 | Q9Y4K0 | LOXL2 | 37 | (Y) | FQQPAPEYHQP | 230 | 1.61E+09 | Lysyl |
| | | | | | QAPAnVAKIQLR | | | oxidase |
| | | | | | | | | homolog 2 |
| LRRC17_2 | Q8N6Y2 | LRRC17 | 225 | (P) | qVSGRPPVIKPE | 428 | 2.18E+08 | Leucine- |
| 25 | | | | | V | | | rich repeat- |
| | | | | | | | | containing |
| | | | | | | | | protein 17 |
| LTBP1_393 | Q14766-4 | LTBP1 | 393 | (T) | LTATNFR | 234 | 9.19E+08 | Isoform 4 of |
| | | | | | | | | Latent- |
| | | | | | | | | transforming |
| | | | | | | | | growth |
| | | | | | | | | factor beta- |
| | | | | | | | | binding |
| | | | | | | | | protein 1 |
| LTBP2_79 | Q14767 | LTBP2 | 79 | (P) | VAGLQPVER | 430 | 9.87E+08 | Latent- |
| | | | | | | | | transforming |
| | | | | | | | | growth |
| | | | | | | | | factor beta- |
| | | | | | | | | binding |
| | | | | | | | | protein 2 |
| LTBP2_129 | Q14767 | LTBP2 | 129 | (G) | QQQPAPR | 531 | 4.46E+09 | Latent- |
| | | | | | | | | transforming |
| | | | | | | | | growth |
| | | | | | | | | factor beta- |
| | | | | | | | | binding |
| | | | | | | | | protein 2 |
| LTBP2_250 | Q14767 | LTBP2 | 250 | (S) | SAAGEGTLAR | 431 | 9.28E+08 | Latent- |
| | | | | | | | | transforming |
| | | | | | | | | growth |
| | | | | | | | | factor beta- |
| | | | | | | | | binding |
| | | | | | | | | protein 2 |
| LTBP2_253 | Q14767 | LTBP2 | 253 | (A) | GEGTLAR | 532 | 3.80E+10 | Latent- |
| | | | | | | | | transforming |
| | | | | | | | | growth |
| | | | | | | | | factor beta- |
| | | | | | | | | binding |
| | | | | | | | | protein 2 |
| LTBP2_291 | Q14767 | LTBP2 | 291 | (Q) | HVGLSR | 533 | 1.91E+09 | Latent- |
| | | | | | | | | transforming |
| | | | | | | | | growth |
| | | | | | | | | factor beta- |
| | | | | | | | | binding |
| | | | | | | | | protein 2 |
| LTBP2_449 | Q14767 | LTBP2 | 449 | (L) | LEAPLK | 534 | 2.87E+08 | Latent- |
| | | | | | | | | transforming |
| | | | | | | | | growth |
| | | | | | | | | factor beta- |
| | | | | | | | | binding |
| | | | | | | | | protein 2 |
| LTBP2_502 | Q14767 | LTBP2 | 502 | (A) | LVENSVETRPPP | 433 | 3.30E+07 | Latent- |
| | | | | | WLPASPGHSLW | | | transforming |
| | | | | | | | | growth |
| | | | | | | | | factor beta- |
| | | | | | | | | binding |
| | | | | | | | | protein 2 |
| LTBP3_164 | Q9NS15 | LTBP3 | 164 | (A) | LSTGALPPLAPE | 535 | 1.18E+08 | Latent- |
| | | | | | G | | | transforming |
| | | | | | | | | growth |
| | | | | | | | | factor beta- |
| | | | | | | | | binding |
| | | | | | | | | protein 3 |
| LTBP3_205 | Q9NS15 | LTBP3 | 205 | (Q) | HAAFLVPLGPG | 536 | 8.70E+07 | Latent- |
| | | | | | QISA | | | transforming |
| | | | | | | | | growth |
| | | | | | | | | factor beta- |
| | | | | | | | | binding |
| | | | | | | | | protein 3 |
| LTBP3_208 | Q9NS15 | LTBP3 | 208 | (A) | FLVPLGPGQISA | 435 | 4.31E+07 | Latent- |
| | | | | | | | | transforming |
| | | | | | | | | growth |
| | | | | | | | | factor beta- |
| | | | | | | | | binding |
| | | | | | | | | protein 3 |
| LTBP3_221 | Q9NS15 | LTBP3 | 221 | (E) | VQAPPPVVNVR | 537 | 6.41E+08 | Latent- |
| | | | | | | | | transforming |
| | | | | | | | | growth |
| | | | | | | | | factor beta- |
| | | | | | | | | binding |
| | | | | | | | | protein 3 |
| LTBP3_239 | Q9NS15 | LTBP3 | 239 | (A) | SVQVHR | 248 | 5.41E+08 | Latent- |
| | | | | | | | | transforming |
| | | | | | | | | growth |
| | | | | | | | | factor beta- |
| | | | | | | | | binding |
| | | | | | | | | protein 3 |
| MAP2_131 | P11137 | MAP2 | 1314 | (A) | LEQPEVER | 538 | 5.56E+09 | Microtubule |
| 4 | | | | | | | | -associated |
| | | | | | | | | protein 2 |
| MAP4_815 | P27816 | MAP4 | 815 | (A) | AVASTGPSSR | 252 | 6.08E+08 | Microtubule |
| | | | | | | | | -associated |
| | | | | | | | | protein 4 |
| MAP4_816 | P27816 | MAP4 | 816 | (A) | VASTGPSSR | 253 | 2.73E+08 | Microtubule |
| | | | | | | | | -associated |
| | | | | | | | | protein 4 |
| MCFD2_55 | Q8NI22 | MCFD2 | 55 | (E) | HLEGVINKPEA | 539 | 2.54E+08 | Multiple |
| | | | | | | | | coagulation |
| | | | | | | | | factor |
| | | | | | | | | deficiency |
| | | | | | | | | protein 2 |
| MMP2_193 | P08253 | MMP2 | 193 | (A) | HAFAPGTGVGG | 540 | 5.68E+08 | 72 kDa type |
| | | | | | | | | IV |
| | | | | | | | | collagenase |
| MMRN1_2 | Q13201 | MMRN1 | 290 | (S) | LIHTNQA | 541 | 1.35E+09 | Multimerin- |
| 90 | | | | | | | | 1 |
| MMRN1_9 | Q13201 | MMRN1 | 959 | (L) | LQKGLTEFV | 542 | 1.07E+08 | Multimerin- |
| 59 | | | | | | | | 1 |
| NID1_360 | P14543 | NID1 | 360 | (T) | FHQQHPQVI | 450 | 5.26E+08 | Nidogen-1 |
| NID2_291 | Q14112 | NID2 | 291 | (A) | HSSVPLGR | 259 | 5.96E+08 | Nidogen-2 |
| NUCB1_15 | Q02818 | NUCB1 | 157 | (L) | LIQTATR | 543 | 1.68E+08 | Nucleobindin- |
| 7 | | | | | | | | 1 |
| NUCB1_31 | Q02818 | NUCB1 | 317 | (E) | FLASTQR | 265 | 1.67E+08 | Nucleobindin- |
| 7 | | | | | | | | 1 |
| NUCB1_38 | Q02818 | NUCB1 | 389 | (E) | LQQAVLHm | 544 | 4.02E+08 | Nucleobindin- |
| 9 | | | | | | | | 1 |
| PABPC1_4 | E7ERJ7 | PABPC1 | 466 | (A) | AAAATPAVR | 545 | 3.57E+08 | Polyadenylate- |
| 66 | | | | | | | | binding |
| | | | | | | | | protein |
| PDLIM5_1 | Q96HC4 | PDLIM5 | 194 | (A) | LSAGKTAVNVP | 456 | 1.84E+08 | PDZ and |
| 94 | | | | | R | | | LIM |
| | | | | | | | | domain |
| | | | | | | | | protein 5 |
| PDLIM5_2 | Q96HC4 | PDLIM5 | 288 | (A) | qITGTEHLK | 546 | 2.73E+08 | PDZ and |
| 88 | | | | | | | | LIM |
| | | | | | | | | domain |
| | | | | | | | | protein 5 |
| PROB1_65 | E7EW31 | PROB1 | 65 | (G) | AGAQPR | 547 | 1.38E+08 | Proline-rich |
| | | | | | | | | basic |
| | | | | | | | | protein 1 |
| PSAP_301 | C9JIZ6 | PSAP | 301 | (E) | LVEPIKKH | 548 | 8.06E+08 | Prosaposin |
| PTBP1_376 | AOAOUIRR | PTBP1 | 376 | (A) | AAAAAGR | 549 | 2.41E+09 | Polypyrimidine |
| | M4 | | | | | | | tract- |
| | | | | | | | | binding |
| | | | | | | | | protein 1 |
| PZP_578 | P20742 | PZP | 578 | (S) | HAHLQVAAAPQ | 550 | 5.67E+07 | Pregnancy |
| | | | | | SLCALR | | | zone protein |
| QSOX2_58 | Q6ZRP7 | QSOX2 | 580 | (S) | EGGTLAR | 551 | 1.27E+10 | Sulfhydryl |
| 0 | | | | | | | | oxidase 2 |
| RET_1045 | P07949 | RET | 1045 | (N) | NAPLPR | 552 | 1.31E+09 | Proto- |
| | | | | | | | | oncogene |
| | | | | | | | | tyrosine- |
| | | | | | | | | protein |
| | | | | | | | | kinase |
| | | | | | | | | receptor Ret |
| SERBP1_56 | Q8NC51 | SERBP1 | 56 | (A) | qAAAQTNSNAA | 288 | 4.86E+07 | Plasminogen |
| | | | | | GKQLR | | | activator |
| | | | | | | | | inhibitor 1 |
| | | | | | | | | RNA- |
| | | | | | | | | binding |
| | | | | | | | | protein |
| SERBP1_60 | Q8NC51 | SERBP1 | 60 | (A) | qTNSNAAGKQL | 289 | 2.20E+08 | Plasminogen |
| | | | | | R | | | activator |
| | | | | | | | | inhibitor 1 |
| | | | | | | | | RNA- |
| | | | | | | | | binding |
| | | | | | | | | protein |
| SERBP1_11 | Q8NC51 | SERBP1 | 116 | (Q) | qLQGEGKII | 462 | 4.39E+09 | Plasminogen |
| 6 | | | | | | | | activator |
| | | | | | | | | inhibitor 1 |
| | | | | | | | | RNA- |
| | | | | | | | | binding |
| | | | | | | | | protein |
| SIK3_44 | HOY494 | SIK3 | 44 | (P) | AAGQPR | 553 | 3.30E+08 | Serine/ |
| | | | | | | | | threonine- |
| | | | | | | | | protein |
| | | | | | | | | kinase SIK3 |
| SPOCK1_2 | Q08629 | SPOCK1 | 27 | (A) | LAGGAGPNHGN | 466 | 1.92E+09 | Testican-1 |
| 7 | | | | | FL | | | |
| SRGN_59 | P10124 | SRGN | 59 | (E) | LLPGESNKIPR | 296 | 2.32E+08 | Serglycin |
| ST6GAL1_ | P15907 | ST6GAL1 | 92 | (A) | SFQVWNK | 554 | 1.52E+07 | Beta- |
| 92 | | | | | | | | galactoside |
| | | | | | | | | alpha-2,6- |
| | | | | | | | | sialyltransferase |
| | | | | | | | | 1 |
| SYNRG_32 | Q9UMZ2 | SYNRG | 326 | (L) | mSSGLPR | 555 | 4.94E+08 | Synergin |
| 6 | | | | | | | | gamma |
| TF_470 | P02787 | TF | 470 | (C) | HTAVGR | 556 | 6.65E+08 | Serotransferrin |
| THBS1_630 | P07996 | THB S1 | 630 | (P) | FGQGVEHATAN | 557 | 4.11E+08 | Thrombospondin- |
| | | | | | K | | | 1 |
| THBS2_290 | P35442 | THB S2 | 290 | (Q) | LSENLKR | 318 | 1.83E+08 | Thrombospondin- |
| | | | | | | | | 2 |
| THSD4_28 | Q6ZMPO | THSD4 | 286 | (R) | STAISCIGAYR | 558 | 4.36E+07 | Thrombospondin |
| 6 | | | | | | | | type- |
| | | | | | | | | 1 domain- |
| | | | | | | | | containing |
| | | | | | | | | protein 4 |
| TLR2_377 | O60603 | TLR2 | 377 | (Y) | LKNSAC | 559 | 2.83E+07 | Toll-like |
| | | | | | | | | receptor 2 |
| UBE3C_35 | Q15386 | UBE3C | 357 | (P) | ASASCH | 560 | 6.28E+07 | Ubiquitin- |
| 7 | | | | | | | | protein |
| | | | | | | | | ligase E3C |
| UQCRH_76 | A0A087WT | UQCRH | 76 | (L) | HGSPNNSERAM | 561 | 3.01E+08 | Cytochrome |
| | F2 | | | | RAVGEmCKGPG | | | b-c1 |
| | | | | | AAR | | | complex |
| | | | | | | | | subunit 6, |
| | | | | | | | | mitochondrial |
| VIM_275 | P08670 | VIM | 275 | (Q) | qYESVAAKNLQ | 562 | 6.77E+07 | Vimentin |
| ZNF611_40 | MOQXQ6 | ZNF611 | 40 | (S) | LQPLPPRYLFQm | 563 | 6.47E+07 | Zinc finger |
| | | | | | H | | | protein 611 |
| ZNF672_21 | Q499Z4 | ZNF672 | 217 | (R) | HLQTHSGEKPF | 564 | 8.08E+07 | Zinc finger |
| 7 | | | | | K | | | protein 672 |
| ZYX_244 | Q15942 | ZYX | 244 | (Q) | PVSLANTQPR | 565 | 9.45E+08 | Zyxin |

TAILS HC Overlap

| | SMC1 | SMC1 | | | | | SMC2 | SMC2 | |
|---|---|---|---|---|---|---|---|---|---|
| | adj. | logFC. | | | | SMC1 | adj.P. | logFC. | |
| | P.Val. | mWT. | pre− | | SEQ | TAILS. | Val. | mWT. | pre- |
| TAILS_ | mWT.over. | over. | vious_ | SMC1 | ID | total | mWT.over. | over. | vious_ |
| id | mEQ | mEQ | aa | sequence | NO: | Intensity | mEQ | mEQ | aa |
| ADAM9_6 | 2.32E−04 | 1.55 | (Y) | VIQAEGK | 122 | 2.56E+10 | 1.38E−02 | 2.09 | (Y) |
| 9 | | | | EHIIHLER | | | | | |
| ADAM9_6 | | | | | | | 7.68E−03 | 1.21 | (Y) |
| 9 | | | | | | | | | |
| ADAM9_6 | | | | | | | 5.98E−04 | 1.91 | (Y) |
| 9 | | | | | | | | | |
| AEBP1_37 | 1.29E−04 | 3.41 | (E) | FLEGFLSE | 123 | 4.54E+07 | 3.63E−04 | 3.48 | (E) |
| | | | | LEPEPR | | | | | |
| AKAP12_1 | | | | | | | 1.82E−04 | 2.94 | (P) |
| 106 | | | | | | | | | |
| BCOR_258 | | | | | | | 2.68E−04 | 2.49 | (R) |
| BMP6_129 | | | | | | | 2.48E−02 | 1.50 | (R) |
| CCDC80_6 | 1.05E−04 | 3.70 | (T) | LEEPNLQ | 127 | 7.99E+09 | 4.00E−03 | 2.21 | (T) |
| 6 | | | | PLQR | | | | | |
| COL16A1_ | 1.01E−05 | 4.63 | (P) | qSEGKVY | 132 | 3.99E+09 | 6.99E−05 | 3.93 | (P) |
| 269 | | | | TR | | | | | |
| COL18A1_ | 1.96E−04 | 1.42 | (A) | LQPPVVQ | 133 | 1.68E+09 | 2.75E−02 | 1.47 | (A) |
| 1505 | | | | LHDSNPY | | | | | |
| | | | | PR | | | | | |
| COL1A2_8 | 3.85E−05 | 2.36 | (A) | qYDGKGV | 135 | 1.24E+09 | 3.44E−02 | 1.15 | (A) |
| 0 | | | | GLGPGPM | | | | | |
| | | | | GLmGPR | | | | | |
| COL1A2_1 | 3.85E−05 | 2.27 | (G) | FQGPAGE | 137 | 2.09E+09 | 4.94E−04 | 2.33 | (G) |
| 13 | | | | PGEPGQT | | | | | |
| | | | | GPAGAR | | | | | |
| COL1A2_1 | | | | | | | 4.01E−03 | 1.07 | (G) |
| 13 | | | | | | | | | |
| COL1A2_2 | 2.77E−05 | 2.82 | (A) | VGNAGPA | 138 | 5.91E+07 | 9.82E−05 | 3.47 | (A) |
| 70 | | | | GPAGPR | | | | | |
| COL1A2_3 | 2.40E−04 | 1.63 | (G) | LTGAKGA | 139 | 5.24E+07 | 4.30E−03 | 1.92 | (G) |
| 05 | | | | AGLPGVA | | | | | |
| | | | | GAPGLPG | | | | | |
| | | | | PR | | | | | |
| COL1A2_6 | 4.27E−04 | 1.45 | (A) | VGTAGPS | 142 | 8.62E+06 | 3.48E−04 | 2.33 | (A) |
| 30 | | | | GPSGLPG | | | | | |
| | | | | ER | | | | | |
| COL1A2_8 | 9.02E−04 | 3.72 | (G) | LLGAPGIL | 145 | 7.88E+09 | 1.08E−03 | 3.22 | (G) |
| 66 | | | | GLPGSR | | | | | |
| COL1A2_9 | 1.71E−05 | 2.91 | (A) | AGAPGPH | 146 | 4.57E+08 | 1.83E−03 | 2.87 | (A) |
| 60 | | | | GPVGPAG | | | | | |
| | | | | KHGNR | | | | | |
| COL1A2_1 | 1.30E−04 | 1.79 | (G) | LQGLPGI | 147 | 1.05E+09 | 1.98E−03 | 1.98 | (G) |
| 028 | | | | AGHHGD | | | | | |
| | | | | QGAPGSV | | | | | |
| | | | | GPAGPR | | | | | |
| COL4A2_7 | 1.55E−04 | 2.14 | (G) | YNGPPGL | 150 | 6.55E+07 | 1.45E−02 | 1.70 | (G) |
| 1 | | | | QGFPGLQ | | | | | |
| | | | | GR | | | | | |
| ECM1_108 | | | | | | | 1.07E−04 | 3.34 | (P) |
| EFEMP1_1 | 6.12E−06 | 4.43 | (A) | AVAGPE | 157 | 3.19E+09 | 1.29E−04 | 3.27 | (A) |
| 24 | | | | MQTGR | | | | | |
| EFEMP1_1 | 2.54E−04 | 2.25 | (A) | VAGPEM | 158 | 2.03E+09 | 3.99E−03 | 2.42 | (A) |
| 25 | | | | QTGR | | | | | |
| FBLN2_25 | 5.18E−03 | 1.29 | (A) | ALGPPAP | 162 | 1.04E+08 | 2.33E−04 | 1.88 | (A) |
| 9 | | | | VQAKAR | | | | | |
| FBLN2_26 | 4.40E−04 | 4.21 | (A) | LGPPAPV | 163 | 7.69E+08 | 1.15E−03 | 2.42 | (A) |
| 0 | | | | QAKAR | | | | | |
| FBN1_29 | 5.30E−06 | 4.49 | (N) | LEAGNVK | 164 | 1.26E+10 | 4.49E−04 | 3.57 | (N) |
| | | | | ETR | | | | | |
| FBN1_53 | 1.17E−05 | 4.93 | (A) | LKGPNVC | 165 | 8.48E+08 | 6.99E−05 | 4.89 | (A) |
| | | | | GSR | | | | | |
| FLNA_128 | 7.08E−05 | 5.06 | (A) | LTQTGGP | 168 | 5.65E+08 | 1.09E−03 | 5.72 | (A) |
| 5 | | | | HVKAR | | | | | |
| FN1_35 | | | | | | | 5.84E−04 | 2.08 | (Q) |
| FN1_36 | 1.30E−04 | 3.34 | (Q) | MVQPQSP | 169 | 4.40E+10 | 5.51E−05 | 3.69 | (Q |
| | | | | VAVSQSK | | | | | |
| | | | | PGCYDnG | | | | | |
| | | | | KHYQINQ | | | | | |
| | | | | QWER | | | | | |
| FN1_40 | 4.40E−04 | 4.27 | (P) | qSPVAVS | 170 | 3.67E+09 | 4.17E−04 | 2.66 | (P) |
| | | | | QSKPGCY | | | | | |
| | | | | DnGKHYQ | | | | | |
| | | | | INQQWER | | | | | |
| FN1_279 | 4.60E−04 | 1.98 | (T) | TSSGSGPF | 173 | 5.94E+09 | 4.38E−03 | 1.46 | (T) |
| | | | | TDVR | | | | | |
| FN1_607 | | | | | | | 4.61E−02 | 0.63 | (P) |
| FN1_886 | 3.92E−03 | 1.21 | (E) | NQESTPV | 176 | 1.42E+09 | | | |
| | | | | VIQQETT | | | | | |
| | | | | GTPR | | | | | |
| FN1_1143 | 3.01E−03 | 0.86 | (G) | LTPGVEY | 177 | 1.36E+08 | 7.72E−05 | 2.80 | (G) |
| | | | | VYTIQVL | | | | | |
| | | | | R | | | | | |
| FN1_1656 | 2.41E−04 | 1.52 | (P) | SSSPVTG | 178 | 4.82E+09 | 1.69E−03 | 1.86 | (P) |
| | | | | YR | | | | | |
| FN1_1715 | 1.78E−04 | 1.95 | (P) | LVQTAVT | 179 | 2.15E+09 | 7.43E−04 | 2.17 | (P) |
| | | | | NIDR | | | | | |
| FN1_1715 | 3.71E−05 | 1.88 | (P) | LVQTAVT | 180 | 7.60E+08 | | | |
| | | | | NIDRPKG | | | | | |
| | | | | LAFTDVD | | | | | |
| | | | | VDSIKIA | | | | | |
| | | | | WESPQGQ | | | | | |
| | | | | VSR | | | | | |
| FN1_1780 | | | | | | | 7.93E−04 | 2.01 | (E) |
| FN1_1805 | | | | | | | 6.84E−04 | 1.73 | (P) |
| FN1_2113 | | | | | | | 1.41E−03 | 1.57 | (P) |
| FSTL1_290 | | | | | | | 2.00E−03 | 2.86 | (E) |
| GSN_413 | 1.27E−04 | 2.19 | (S) | HIANVER | 187 | 2.44E+09 | 2.19E−02 | 1.74 | (S) |
| HEG1_47 | 4.16E−05 | 3.15 | (P) | LAGAGLE | 188 | 6.74E+08 | 1.11E−02 | 2.01 | (P) |
| | | | | LQLER | | | | | |
| HSPG2_27 | 6.85E−05 | 2.49 | (P) | LLPGSVR | 191 | 2.35E+07 | 4.00E−04 | 2.10 | (P) |
| 5 | | | | | | | | | |
| HSPG2_18 | 1.45E−04 | 2.03 | (T) | LSAPVVSI | 192 | 1.64E+08 | 3.09E−03 | 1.90 | (T) |
| 63 | | | | HPPQLTV | | | | | |
| | | | | QPGQLAE | | | | | |
| | | | | FR | | | | | |
| HSPG2_19 | | | | | | | 4.39E−03 | 1.98 | (A) |
| 36 | | | | | | | | | |
| HSPG2_23 | 1.12E−04 | 2.04 | (G) | ANLAYPA | 194 | 7.64E+07 | 4.51E−04 | 1.91 | (G) |
| 31 | | | | GSTQPIR | | | | | |
| HSPG2_23 | 4.09E−05 | 1.92 | (N) | LAYPAGS | 195 | 5.74E+07 | 2.31E−03 | 1.41 | (N) |
| 33 | | | | TQPIR | | | | | |
| IGFBP3_31 | 7.08E−05 | 2.49 | (S) | SAGLGPV | 211 | 4.82E+08 | 8.08E−05 | 2.44 | (S) |
| | | | | VR | | | | | |
| IGFBP3_32 | 5.30E−06 | 3.78 | (S) | AGLGPVV | 212 | 2.11E+09 | 4.67E−05 | 5.18 | (S) |
| | | | | R | | | | | |
| IGFBP7_10 | 2.66E−05 | 3.69 | (A) | AAGGPGV | 214 | 5.68E+07 | 1.03E−04 | 2.57 | (A) |
| 1 | | | | SGVCVCK | | | | | |
| | | | | SR | | | | | |
| KPNB1_37 | 3.17E−04 | 1.77 | (F) | LVELSR | 216 | 3.15E+08 | 8.32E−03 | 1.11 | (F) |
| LAMA5_4 | 6.08E−03 | 1.25 | (E) | QVLPAGQ | 217 | 4.81E+08 | 1.52E−03 | 2.19 | (E) |
| 84 | | | | IVNCDCS | | | | | |
| | | | | AAGTQG | | | | | |
| | | | | NACR | | | | | |
| LMNA_40 | 1.13E−05 | 4.22 | (S) | qTQGGGS | 222 | 3.42E+09 | 6.99E−05 | 4.77 | (S) |
| 8 | | | | VTKKR | | | | | |
| LOX_58 | 1.17E−03 | 0.99 | (L) | LSLGSQY | 223 | 2.82E+08 | 3.36E−04 | 1.77 | (L) |
| | | | | QPQR | | | | | |
| LOX_123 | 4.03E−04 | 1.82 | (R) | HWFQAG | 226 | 1.50E+08 | 4.91E−02 | 1.37 | (R) |
| | | | | YSTSR | | | | | |
| LOX_124 | 1.30E−04 | 3.62 | (H) | WFQAGY | 227 | 1.65E+08 | 2.64E−04 | 3.45 | (H) |
| | | | | STSR | | | | | |
| LOX_125 | | | | | | | 1.29E−04 | 3.33 | (W) |
| LOX_126 | 1.52E−03 | 1.59 | (F) | QAGYSTS | 228 | 4.18E+06 | | | |
| | | | | R | | | | | |
| LOXL1_21 | 9.71E−03 | 1.09 | (A) | VASAGVI | 229 | 2.12E+07 | 8.81E−03 | 1.47 | (A) |
| 9 | | | | YPYQPR | | | | | |
| LOXL2_37 | 3.87E−05 | 3.28 | (Y) | FQQPAPE | 230 | 5.20E+09 | 3.55E−04 | 3.30 | (Y) |
| | | | | YHQPQAP | | | | | |
| | | | | ANVAKIQ | | | | | |
| | | | | LR | | | | | |
| LOXL2_38 | 6.68E−04 | 1.26 | (F) | qQPAPEY | 231 | 2.79E+08 | 5.48E−04 | 1.68 | (F) |
| | | | | HQPQAPA | | | | | |
| | | | | NVAKIQL | | | | | |
| | | | | R | | | | | |
| LRRC17_2 | | | | | | | 4.65E−05 | 2.69 | (P) |
| 25 | | | | | | | | | |
| LTBP1_15 | 2.31E−03 | 1.10 | (F) | SEQYTPE | 238 | 8.25E+08 | 1.41E−02 | 1.47 | (F) |
| 97 | | | | ADPYFIQ | | | | | |
| | | | | DR | | | | | |
| LTBP1_39 | 1.71E−05 | 3.89 | (T) | LTATNFR | 234 | 9.30E+08 | 9.66E−05 | 6.30 | (T) |
| 3 | | | | | | | | | |
| LTBP1_54 | 1.13E−04 | 2.70 | (S) | HQQVIPH | 236 | 1.90E+09 | 1.07E−02 | 1.77 | (S) |
| 0 | | | | VYPVAAK | | | | | |
| | | | | TQLGR | | | | | |
| LTBP2_79 | | | | | | | 4.94E−04 | 1.62 | (P) |
| LTBP2_25 | | | | | | | 5.15E−05 | 2.70 | (S) |
| 0 | | | | | | | | | |
| LTBP2_50 | | | | | | | 4.00E−04 | 2.85 | (A) |
| 2 | | | | | | | | | |
| LTBP3_20 | | | | | | | 3.36E−03 | 1.53 | (A) |
| 8 | | | | | | | | | |
| LTBP3_23 | 4.00E−04 | 2.62 | (A) | SVQVHR | 248 | 1.25E+08 | 1.10E−03 | 1.59 | (A) |
| 9 | | | | | | | | | |
| MAP4_815 | 2.67E−03 | 1.19 | (A) | AVASTGP | 252 | 5.58E+08 | 1.09E−03 | 1.70 | (A) |
| | | | | SSR | | | | | |
| MAP4_816 | 7.15E−05 | 1.62 | (A) | VASTGPS | 253 | 1.57E+08 | 9.94E−05 | 2.51 | (A) |
| | | | | SR | | | | | |
| NID1_360 | | | | | | | 7.25E−04 | 2.69 | (T) |
| NID2_291 | 3.33E−04 | 4.04 | (A | HSSVPLG | 259 | 2.94E+09 | 1.51E−04 | 3.16 | (A) |
| | | | | R | | | | | |
| NID2_478 | 6.92E−05 | 4.10 | (V) | FTYNAAN | 262 | 1.74E+08 | 7.92E−04 | 4.47 | (V) |
| | | | | KETCEHN | | | | | |
| | | | | HR | | | | | |
| NID2_951 | 7.12E−05 | 3.92 | (Q) | YAYPGAR | 263 | 3.51E+08 | 4.85E−05 | 2.80 | (Q) |
| NUCB1_31 | 1.13E−05 | 4.87 | (E) | FLASTQR | 265 | 1.78E+08 | | | |
| 7 | | | | | | | | | |
| NUCB1_38 | 6.57E−04 | 2.09 | (E) | LQQAVLH | 266 | 1.01E+08 | | | |
| 9 | | | | MEQR | | | | | |
| PDLIM5_1 | | | | | | | 4.65E−05 | 6.01 | (A) |
| 94 | | | | | | | | | |
| PPAT_255 | 3.17E−04 | 1.77 | (E) | IVEISR | 278 | 3.15E+08 | 8.32E−03 | 1.11 | (E) |
| PPP1R18_2 | 1.01E−05 | 3.68 | (E) | LSETLTR | 279 | 7.16E+07 | 2.58E−04 | 2.32 | (E) |
| 95 | | | | | | | | | |
| PPP1R21_6 | 1.30E−04 | 1.92 | (L) | IGTLTR | 280 | 1.01E+08 | 4.25E−04 | 2.76 | (L) |
| 46 | | | | | | | | | |
| SERBP1_5 | 1.08E−05 | 4.06 | (A) | QAAAQT | 288 | 9.32E+07 | | | |
| 6 | | | | NSNAAGK | | | | | |
| | | | | QLR | | | | | |
| SERBP1_6 | 4.43E−05 | 2.10 | (A) | qTNSNAA | 289 | 2.09E+08 | 1.10E−03 | 1.62 | (A) |
| 0 | | | | GKQLR | | | | | |
| SERBP1_1 | 1.69E−04 | 2.55 | (Q) | qLQGEGK | 290 | 4.56E+08 | 5.51E−05 | 3.25 | (Q) |
| 16 | | | | IIDR | | | | | |
| SPOCK1_2 | | | | | | | 6.32E−05 | 2.69 | (A) |
| 7 | | | | | | | | | |
| SRGN_59 | 2.28E−05 | 4.33 | (E) | LLPGESN | 296 | 2.70E+09 | 4.70E−05 | 3.66 | (E) |
| | | | | KIPR | | | | | |
| TAGLN_16 | 1.54E−04 | 2.81 | (Q) | LQEGKHV | 305 | 5.04E+08 | 3.20E−03 | 1.93 | (Q) |
| 8 | | | | IGLQmGS | | | | | |
| | | | | NR | | | | | |
| TAGLN2_1 | 9.76E−06 | 4.27 | (Q) | LQEGKNV | 307 | 8.44E+07 | 4.65E−05 | 4.36 | (Q) |
| 67 | | | | IGLQmGT | | | | | |
| | | | | NR | | | | | |
| THBS2_29 | 5.49E−06 | 4.05 | (Q) | LSENLKR | 318 | 4.15E+08 | | | |
| 0 | | | | | | | | | |
| TMTC1_58 | 1.30E−04 | 1.92 | (N) | LGTLTR | 321 | 6.05E+08 | 4.25E−04 | 2.76 | (N) |
| 5 | | | | | | | | | |
| VIM_314 | 2.77E−05 | 2.60 | (K) | QESTEYR | 324 | 4.88E+07 | 1.07E−04 | 3.28 | (K) |
| VIM_333 | 5.30E−06 | 4.39 | (A) | LKGTNES | 325 | 2.04E+08 | 5.98E−05 | 3.84 | (A) |
| | | | | LER | | | | | |

| | | | | HUVEC | | | | | | |
|---|---|---|---|---|---|---|---|---|---|---|
| | | | SMC2 | adj.P. | HUVEC | | | | HUVEC | |
| | | SEQ | TAILS. | Val. | logFC. | pre- | | SEQ | TAILS. | |
| TAILS_ | SMC2 | ID | total | mWT. | mWT. | vious_ | HUVEC | ID | total | entry_ |
| id | sequence | NO: | Intensity | over.mEQ | over.mEQ | aa | sequence | NO: | Intensity | name |
| ADAM9_ | VIQAEG | 122 | 1.73E+09 | | | | | | | ADAM9 |
| 69 | KEHIIHL | | | | | | | | | protein |
| | ER | | | | | | | | | |
| ADAM9_ | VIQAEG | 329 | 1.94E+09 | | | | | | | ADAM9 |
| 69 | KEHIIHL | | | | | | | | | protein |
| ADAM9_ | VIQAEG | 330 | 9.68E+09 | | | | | | | ADAM9 |
| 69 | K | | | | | | | | | protein |
| AEBP1_3 | FLEGFLS | 123 | 8.64E+07 | 4.09E−04 | 4.34 | (E) | FLEGFLS | 123 | 5.26E+06 | Adipocyte |
| 7 | ELEPEPR | | | | | | ELEPEPR | | | enhancer- |
| | | | | | | | | | | binding |
| | | | | | | | | | | protein 1 |
| AKAP12_ | FTQGKV | 334 | 5.94E+08 | 1.16E−03 | 3.23 | (P) | FTQGKV | 334 | 3.55E+07 | A-kinase |
| 1106 | VGQTTP | | | | | | VGQTTP | | | anchor |
| | ESF | | | | | | ESF | | | protein 12 |
| BCOR_25 | DFIALR | 339 | 1.00E+08 | 5.25E−04 | 2.87 | (R) | DFIALR | 339 | 1.21E+08 | BCL-6 |
| 8 | | | | | | | | | | corepressor |
| BMP6_12 | LKSAPLF | 340 | 4.00E+07 | 3.97E−04 | 3.56 | (R) | LKSAPLF | 340 | 1.74E+08 | Bone |
| 9 | mL | | | | | | mL | | | morpho- |
| | | | | | | | | | | genetic |
| | | | | | | | | | | protein 6 |
| CCDC80_ | LEEPNL | 127 | 3.94E+08 | | | | | | | Coiled- |
| 66 | QPLQR | | | | | | | | | coil |
| | | | | | | | | | | domain- |
| | | | | | | | | | | containing |
| | | | | | | | | | | protein 80 |
| COL16A1_ | QSEGKV | 132 | 1.58E+09 | | | | | | | Collagen |
| 269 | YTR | | | | | | | | | alpha- |
| | | | | | | | | | | 1(XVI) |
| | | | | | | | | | | chain |
| COL18A1_ | LQPPVV | 349 | 1.11E+10 | 1.00E−03 | 3.88 | (A) | LQPPVV | 349 | 1.34E+11 | Collagen |
| 1505 | QLH | | | | | | QLH | | | alpha- |
| | | | | | | | | | | 1(XVIII) |
| | | | | | | | | | | chain |
| COL1A2_ | qYDGKG | 135 | 2.08E+07 | | | | | | | Collagen |
| 80 | VGLGPG | | | | | | | | | alpha-2(I) |
| | PmGLmG | | | | | | | | | chain |
| | PR | | | | | | | | | |
| COL1A2_ | FQGPAG | 137 | 1.65E+09 | | | | | | | Collagen |
| 113 | EPGEPG | | | | | | | | | alpha-2(I) |
| | QTGPAG | | | | | | | | | chain |
| | AR | | | | | | | | | |
| COL1A2_ | FQGPAG | 354 | 6.31E+08 | | | | | | | Collagen |
| 113 | EPG | | | | | | | | | alpha-2(I) |
| | | | | | | | | | | chain |
| COL1A2_ | VGNAGP | 138 | 2.57E+09 | | | | | | | Collagen |
| 270 | AGPAGP | | | | | | | | | alpha-2(I) |
| | R | | | | | | | | | chain |
| COL1A2_ | LTGAKG | 139 | 1.16E+09 | | | | | | | Collagen |
| 305 | AAGLPG | | | | | | | | | alpha-2(I) |
| | VAGAPG | | | | | | | | | chain |
| | LPGPR | | | | | | | | | |
| COL1A2_ | VGTAGP | 142 | 1.32E+08 | | | | | | | Collagen |
| 630 | SGPSGLP | | | | | | | | | alpha-2(I) |
| | GER | | | | | | | | | chain |
| COL1A2_ | LLGAPGI | 145 | 4.62E+10 | | | | | | | Collagen |
| 866 | LGLPGS | | | | | | | | | alpha-2(I) |
| | R | | | | | | | | | chain |
| COL1A2_ | AGAPGP | 146 | 3.12E+09 | | | | | | | Collagen |
| 960 | HGPVGP | | | | | | | | | alpha-2(I) |
| | AGKHGN | | | | | | | | | chain |
| | R | | | | | | | | | |
| COL1A2_ | LQGLPGI | 361 | 9.44E+08 | | | | | | | Collagen |
| 1028 | AGHHG | | | | | | | | | alpha-2(I) |
| | | | | | | | | | | chain |
| COL4A2_ | YNGPPG | 150 | 2.73E+08 | | | | | | | Collagen |
| 71 | LQGFPG | | | | | | | | | alpha- |
| | LQGR | | | | | | | | | 2(IV) |
| | | | | | | | | | | chain |
| ECM1_10 | QEAVPL | 376 | 2.41E+09 | 1.92E−03 | 2.12 | (P) | qEAVPL | 376 | 1.47E+08 | Extracellular |
| 8 | QK | | | | | | QK | | | matrix |
| | | | | | | | | | | protein 1 |
| EFEMP1_ | AVAGPE | 157 | 2.58E+10 | 7.18E−04 | 3.31 | (A) | AVAGPE | 157 | 2.92E+11 | EGF- |
| 124 | MQTGR | | | | | | MQTGR | | | containing |
| | | | | | | | | | | fibulin- |
| | | | | | | | | | | like |
| | | | | | | | | | | extracellular |
| | | | | | | | | | | matrix |
| | | | | | | | | | | protein 1 |
| EFEMP1_ | VAGPEM | 158 | 7.46E+09 | 7.18E−04 | 2.45 | (A) | VAGPEM | 158 | 9.33E+10 | EGF- |
| 125 | QTGR | | | | | | QTGR | | | containing |
| | | | | | | | | | | fibulin- |
| | | | | | | | | | | like |
| | | | | | | | | | | extracellular |
| | | | | | | | | | | matrix |
| | | | | | | | | | | protein 1 |
| FBLN2_2 | ALGPPA | 162 | 8.11E+08 | | | | | | | Fibulin-2 |
| 59 | PVQAKA | | | | | | | | | |
| | R | | | | | | | | | |
| FBLN2_2 | LGPPAP | 163 | 6.60E+09 | | | | | | | Fibulin-2 |
| 60 | VQAKAR | | | | | | | | | |
| FBN1_29 | LEAGNV | 387 | 2.40E+10 | 5.44E−04 | 3.91 | (N) | LEAGNV | 387 | 1.85E+09 | Fibrillin-1 |
| | K | | | | | | K | | | |
| FBN1_53 | LKGPNV | 165 | 2.58E+09 | | | | | | | Fibrillin-1 |
| | CGSR | | | | | | | | | |
| FLNA_12 | LTQTGG | 168 | 1.38E+09 | 2.90E−04 | 3.81 | (A) | LTQTGG | 168 | 2.92E+09 | Filamin-A |
| 85 | PHVKAR | | | | | | PHVKAR | | | |
| FN1_35 | qmVQPQ | 392 | 2.65E+10 | 1.57E−03 | 2.58 | (Q) | qmVQPQ | 392 | 2.07E+10 | Isoform |
| | SPVAVS | | | | | | SPVAVS | | | 15 of |
| | QSKPGC | | | | | | QSKPGC | | | Fibronectin |
| | Y | | | | | | Y | | | |
| FN1_36 | MVQPQS | 393 | 1.54E+11 | 8.41E−04 | 3.45 | (Q) | mVQPQS | 393 | 1.76E+11 | Isoform |
| | PVAVSQ | | | | | | PVAVSQ | | | 15 of |
| | SKPGCY | | | | | | SKPGCY | | | Fibronectin |
| FN1_40 | QSPVAV | 394 | 3.29E+09 | 7.65E−03 | 2.96 | (P) | qSPVAVS | 394 | 1.37E+10 | Isoform |
| | SQSKPG | | | | | | QSKPGC | | | 15 of |
| | CY | | | | | | Y | | | Fibronectin |
| FN1_279 | TSSGSGP | 396 | 2.71E+10 | 3.86E−02 | 2.71 | (P) | SSSGPVE | 397 | 4.88E+09 | Isoform |
| | FT | | | | | | VFIT | | | 15 of |
| | | | | | | | | | | Fibronectin |
| FN1_607 | SSSGPVE | 397 | 1.24E+11 | 7.40E−03 | 2.04 | (E) | NQESTP | 176 | 8.05E+09 | Isoform |
| | VFIT | | | | | | VVIQQE | | | 15 of |
| | | | | | | | TTGTPR | | | Fibronectin |
| FN1_886 | LTPGVE | 177 | 9.55E+08 | 3.57E−04 | 3.68 | (G) | LTPGVE | 177 | 4.67E+09 | Isoform |
| | YVYTIQ | | | | | | YVYTIQ | | | 15 of |
| | | | | | | | VLR | | | Fibronectin |
| FN1_1143 | VLR | | | | | | | | | Isoform |
| | | | | | | | | | | 15 of |
| | | | | | | | | | | Fibronectin |
| FN1_1656 | SSSPVTG | 178 | 2.00E+10 | 1.17E−03 | 1.63 | (P) | SSSPVTG | 178 | 2.23E+10 | Isoform |
| | YR | | | | | | YR | | | 15 of |
| | | | | | | | | | | Fibronectin |
| FN1_1715 | LVQTAV | 399 | 9.37E+10 | | | | | | | Isoform |
| | TNI | | | | | | | | | 15 of |
| | | | | | | | | | | Fibronectin |
| FN1_1715 | | | | | | | | | | Isoform |
| | | | | | | | | | | 15 of |
| | | | | | | | | | | Fibronectin |
| FN1_1780 | LQGLRP | 400 | 4.09E+09 | 5.44E−04 | 2.27 | (E) | LQGLRP | 400 | 2.82E+09 | Isoform |
| | GSEYTV | | | | | | GSEYTV | | | 15 of |
| | SVVALH | | | | | | SVVALH | | | Fibronectin |
| FN1_1805 | LIGTQST | 401 | 5.10E+10 | 5.42E−03 | 1.42 | (P) | LIGTQST | 40 | 4.74E+10 | Isoform |
| | AIPAPT | | | | | | AIPAPT | | | 15 of |
| | | | | | | | | | | Fibronectin |
| FN1_2113 | FVTHPG | 402 | 5.02E+09 | 1.69E−02 | 1.67 | (P) | FVTHPG | 402 | 2.71E+09 | Isoform |
| | Y | | | | | | | | | 15 of |
| | | | | | | | Y | | | Fibronectin |
| FSTL1_29 | LQKHQE | 405 | 5.17E+08 | 4.33E−04 | 2.57 | (E) | LQKHQE | 403 | 1.45E+08 | Follistatin |
| 0 | TA | | | | | | TA | | | -related |
| | | | | | | | | | | protein 1 |
| GSN_413 | HIANVE | 187 | 1.39E+09 | | | | | | | Gelsolin |
| | R | | | | | | | | | |
| HEG1_47 | LAGAGL | 407 | 1.84E+08 | | | | | | | Protein |
| | ELQL | | | | | | | | | HEG |
| | | | | | | | | | | homolog 1 |
| HSPG2_2 | LLPGSV | 410 | 1.16E+08 | 4.57E−02 | 0.77 | (P) | LLPGSV | 410 | 2.67E+07 | Basement |
| 75 | RPLPCGP | | | | | | RPLPCGP | | | membrane |
| | Q | | | | | | Q | | | -specific |
| | | | | | | | | | | heparan |
| | | | | | | | | | | sulfate |
| | | | | | | | | | | proteoglycan |
| | | | | | | | | | | core |
| | | | | | | | | | | protein |
| HSPG2_1 | LSAPVV | 41. | 1.42E+08 | 3.73E−03 | 2.53 | (T) | LSAPVV | 411 | 4.95E+08 | Basement |
| 863 | SIHPPQL | | | | | | SIHPPQL | | | membrane |
| | TVQPGQ | | | | | | TVQPGQ | | | -specific |
| | LA | | | | | | LA | | | heparan |
| | | | | | | | | | | sulfate |
| | | | | | | | | | | proteoglycan |
| | | | | | | | | | | core |
| | | | | | | | | | | protein |
| HSPG2_1 | HSSAGQ | 412 | 1.14E+08 | 3.18E−03 | 2.27 | (A) | HSSAGQ | 412 | 2.96E+08 | Basement |
| 936 | QVAR | | | | | | QVAR | | | membrane |
| | | | | | | | | | | -specific |
| | | | | | | | | | | heparan |
| | | | | | | | | | | sulfate |
| | | | | | | | | | | proteoglycan |
| | | | | | | | | | | core |
| | | | | | | | | | | protein |
| HSPG2_2 | ANLAYP | 194 | 2.66E+08 | 1.16E−03 | 1.73 | (G) | ANLAYP | 194 | 2.45E+09 | Basement |
| 331 | AGSTQPI | | | | | | AGSTQPI | | | membrane |
| | R | | | | | | R | | | -specific |
| | | | | | | | | | | heparan |
| | | | | | | | | | | sulfate |
| | | | | | | | | | | proteoglycan |
| | | | | | | | | | | core |
| | | | | | | | | | | protein |
| HSPG2_2 | LAYPAG | 195 | 3.02E+08 | | | | | | | Basement |
| 333 | STQPIR | | | | | | | | | membrane |
| | | | | | | | | | | -specific |
| | | | | | | | | | | heparan |
| | | | | | | | | | | sulfate |
| | | | | | | | | | | proteoglycan |
| | | | | | | | | | | core |
| | | | | | | | | | | protein |
| IGFBP3_3 | SAGLGP | 211 | 1.02E+09 | | | | | | | Insulin- |
| 1 | VVR | | | | | | | | | like |
| | | | | | | | | | | growth |
| | | | | | | | | | | factor- |
| | | | | | | | | | | binding |
| | | | | | | | | | | protein 3 |
| IGFBP3_3 | AGLGPV | 212 | 5.97E+09 | | | | | | | Insulin- |
| 2 | VR | | | | | | | | | like |
| | | | | | | | | | | growth |
| | | | | | | | | | | factor- |
| | | | | | | | | | | binding |
| | | | | | | | | | | protein 3 |
| IGFBP7_1 | AAGGPG | 214 | 4.97E+08 | 1.29E−03 | 1.89 | (A) | AAGGPG | 214 | 1.12E+08 | Insulin- |
| 01 | VSGVCV | | | | | | VSGVCV | | | like |
| | CKSR | | | | | | CKSR | | | growth |
| | | | | | | | | | | factor- |
| | | | | | | | | | | binding |
| | | | | | | | | | | protein 7 |
| KPNB1_3 | LVELSR | 216 | 4.57E+08 | | | | | | | Importin |
| 7 | | | | | | | | | | subunit |
| | | | | | | | | | | beta-1 |
| LAMA5_ | QVLPAG | 420 | 2.51E+09 | | | | | | | Laminin |
| 484 | QIVNC | | | | | | | | | subunit |
| | | | | | | | | | | alpha-5 |
| LMNA_4 | qTQGGG | 222 | 1.22E+10 | | | | | | | Prelamin- |
| 08 | SVTKKR | | | | | | | | | A/C |
| LOX_58 | LSLGSQ | 223 | 6.15E+07 | | | | | | | Protein- |
| | YQPQR | | | | | | | | | lysine 6- |
| | | | | | | | | | | oxidase |
| LOX_123 | HWFQAG | 226 | 6.25E+08 | | | | | | | Protein- |
| | YSTSR | | | | | | | | | lysine 6- |
| | | | | | | | | | | oxidase |
| LOX_124 | WFQAGY | 227 | 1.64E+09 | 2.90E−04 | 3.35 | (H) | WFQAG | 227 | 8.74E+07 | Protein- |
| | STSR | | | | | | YSTSR | | | lysine 6- |
| | | | | | | | | | | oxidase |
| LOX_125 | FQAGYS | 424 | 1.92E+10 | 1.07E−03 | 3.58 | (W) | FQAGYS | 424 | 1.50E+09 | Protein- |
| | TSR | | | | | | TSR | | | lysine 6- |
| | | | | | | | | | | oxidase |
| LOX_126 | | | | | | | | | | Protein- |
| | | | | | | | | | | lysine 6- |
| | | | | | | | | | | oxidase |
| LOXL1_2 | VASAGV | 229 | 4.27E+08 | | | | | | | Lysyl |
| 19 | IYPYQPR | | | | | | | | | oxidase |
| | | | | | | | | | | homolog 1 |
| LOXL2_3 | FQQPAP | 230 | 9.09E+09 | 7.19E−04 | 3.48 | (Y) | FQQPAP | 230 | 1.61E+09 | Lysyl |
| 7 | EYHQPQ | | | | | | EYHQPQ | | | oxidase |
| | APANVA | | | | | | APAnVA | | | homolog 2 |
| | KIQLR | | | | | | KIQLR | | | |
| LOXL2_3 | qQPAPEY | 231 | 1.65E+08 | | | | | | | Lysyl |
| 8 | HQPQAP | | | | | | | | | oxidase |
| | ANVAKI | | | | | | | | | homolog 2 |
| | QLR | | | | | | | | | |
| LRRC17_ | qVSGRPP | 428 | 9.93E+07 | 4.33E−04 | 2.34 | (P) | qVSGRPP | 428 | 2.18E+08 | Leucine- |
| 225 | VIKPEV | | | | | | VIKPEV | | | rich |
| | | | | | | | | | | repeat- |
| | | | | | | | | | | containing |
| | | | | | | | | | | protein 17 |
| LTBP1_1 | SEQYTPE | 429 | 3.36E+09 | | | | | | | Latent- |
| 597 | A | | | | | | | | | transforming |
| | | | | | | | | | | growth |
| | | | | | | | | | | factor |
| | | | | | | | | | | beta- |
| | | | | | | | | | | binding |
| | | | | | | | | | | protein 1 |
| LTBP1_3 | LTATNF | 234 | 6.93E+09 | 3.57E−04 | 3.33 | (T) | LTATNF | 234 | 9.19E+08 | Latent- |
| 93 | R | | | | | | R | | | transforming |
| | | | | | | | | | | growth |
| | | | | | | | | | | factor |
| | | | | | | | | | | beta- |
| | | | | | | | | | | binding |
| | | | | | | | | | | protein 1 |
| LTBP1_5 | HQQVIP | 236 | 8.09E+08 | | | | | | | Latent- |
| 40 | HVYPVA | | | | | | | | | transforming |
| | AKTQLG | | | | | | | | | growth |
| | R | | | | | | | | | factor |
| | | | | | | | | | | beta- |
| | | | | | | | | | | binding |
| | | | | | | | | | | protein 1 |
| LTBP2_7 | VAGLQP | 430 | 6.41E+07 | 2.02E−03 | 1.51 | (P) | VAGLQP | 430 | 9.87E+08 | Latent- |
| 9 | VER | | | | | | VER | | | transforming |
| | | | | | | | | | | growth |
| | | | | | | | | | | factor |
| | | | | | | | | | | beta- |
| | | | | | | | | | | binding |
| | | | | | | | | | | protein 2 |
| LTBP2_2 | SAAGEG | 431 | 1.86E+08 | 2.41E−03 | 1.71 | (S) | SAAGEG | 431 | 9.28E+08 | Latent- |
| 50 | TLAR | | | | | | TLAR | | | transforming |
| | | | | | | | | | | growth |
| | | | | | | | | | | factor |
| | | | | | | | | | | beta- |
| | | | | | | | | | | binding |
| | | | | | | | | | | protein 2 |
| LTBP2_5 | LVENSV | 433 | 2.73E+08 | 8.02E−03 | 2.68 | (A) | LVENSV | 433 | 3.30E+07 | Latent- |
| 02 | ETRPPP | | | | | | ETRPPP | | | transforming |
| | WLPASP | | | | | | WLPASP | | | growth |
| | GHSLW | | | | | | GHSLW | | | factor |
| | | | | | | | | | | beta- |
| | | | | | | | | | | binding |
| | | | | | | | | | | protein 2 |
| LTBP3_2 | FLVPLGP | 435 | 1.77E+07 | 2.96E−02 | 2.14 | (A) | FLVPLGP | 435 | 4.31E+07 | Latent- |
| 08 | GQISA | | | | | | GQISA | | | transforming |
| | | | | | | | | | | growth |
| | | | | | | | | | | factor |
| | | | | | | | | | | beta- |
| | | | | | | | | | | binding |
| | | | | | | | | | | protein 3 |
| LTBP3_2 | SVQVHR | 248 | 8.70E+08 | 6.36E−03 | 1.57 | (A) | SVQVHR | 248 | 5.41E+08 | Latent- |
| 39 | | | | | | | | | | transforming |
| | | | | | | | | | | growth |
| | | | | | | | | | | factor |
| | | | | | | | | | | beta- |
| | | | | | | | | | | binding |
| | | | | | | | | | | protein 3 |
| MAP4_81 | AVASTG | 252 | 5.60E+08 | 3.72E−03 | 2.51 | (A) | AVASTG | 252 | 6.08E+08 | Microtubule- |
| 5 | PSSR | | | | | | PSSR | | | associated |
| | | | | | | | | | | protein 4 |
| MAP4_81 | VASTGP | 253 | 8.83E+08 | 3.87E−03 | 2.05 | (A) | VASTGP | 253 | 2.73E+08 | Microtubule- |
| 6 | SSR | | | | | | SSR | | | associated |
| | | | | | | | | | | protein 4 |
| NID1_360 | FHQQHP | 450 | 7.78E+09 | 3.79E−02 | 1.78 | (T) | FHQQHP | 450 | 5.26E+08 | Nidogen-1 |
| | QVI | | | | | | QVI | | | |
| NID2_291 | HSSVPL | 259 | 6.67E+09 | 3.84E−03 | 2.90 | (A) | HSSVPL | 259 | 5.96E+08 | Nidogen-2 |
| | GR | | | | | | GR | | | |
| NID2_478 | FTYNAA | 451 | 8.95E+08 | | | | | | | Nidogen-2 |
| | NK | | | | | | | | | |
| NID2_951 | YAYPGA | 263 | 2.03E+09 | | | | | | | Nidogen-2 |
| | R | | | | | | | | | |
| NUCB1_3 | | | | 4.94E−04 | 2.65 | (E) | FLASTQ | 265 | 1.67E+08 | Nucleobin |
| 17 | | | | | | | R | | | din-1 |
| NUCB1_3 | | | | 4.41E−03 | 1.54 | (E) | LQQAVL | 544 | 4.02E+08 | Nucleobin |
| 89 | | | | | | | Hm | | | din-1 |
| PDLIM5_ | LSAGKT | 456 | 7.44E+08 | 2.14E−04 | 4.27 | (A) | LSAGKT | 456 | 1.84E+08 | PDZ and |
| 194 | AVNVPR | | | | | | AVNVPR | | | LIM |
| | | | | | | | | | | domain |
| | | | | | | | | | | protein 5 |
| PPAT_25 | IVEISR | 278 | 4.57E+08 | | | | | | | Amidopho |
| 5 | | | | | | | | | | sphoribos |
| | | | | | | | | | | yltransfera |
| | | | | | | | | | | se |
| PPP1R18_ | LSETLTR | 279 | 1.25E+08 | | | | | | | PPP1R18 |
| 295 | | | | | | | | | | |
| PPP1R21_ | IGTLTR | 280 | 2.46E+08 | | | | | | | Protein |
| 646 | | | | | | | | | | phosphata |
| | | | | | | | | | | se 1 |
| | | | | | | | | | | regulatory |
| | | | | | | | | | | subunit 21 |
| SERBP1_ | | | | 4.48E−03 | 1.43 | (A) | qAAAQT | 288 | 4.86E+07 | Plasminogen |
| 56 | | | | | | | NSNAAG | | | activator |
| | | | | | | | KQLR | | | inhibitor 1 |
| | | | | | | | | | | RNA- |
| | | | | | | | | | | binding |
| | | | | | | | | | | protein |
| SERBP1_ | qTNSNA | 289 | 2.49E+08 | 4.69E−02 | 0.94 | (A) | qTNSNA | 289 | 2.20E+08 | Plasminogen |
| 60 | AGKQLR | | | | | | AGKQLR | | | activator |
| | | | | | | | | | | inhibitor 1 |
| | | | | | | | | | | RNA- |
| | | | | | | | | | | binding |
| | | | | | | | | | | protein |
| SERBP1_ | QLQGEG | 462 | 7.27E+09 | 7.19E−04 | 2.44 | (Q) | qLQGEG | 462 | 4.39E+09 | Plasminogen |
| 116 | KII | | | | | | KII | | | activator |
| | | | | | | | | | | inhibitor 1 |
| | | | | | | | | | | RNA- |
| | | | | | | | | | | binding |
| | | | | | | | | | | protein |
| SPOCK1_ | LAGGAG | 466 | 5.00E+08 | 1.02E−02 | 1.07 | (A) | LAGGAG | 466 | 1.92E+09 | Testican-1 |
| 27 | PNHGNF | | | | | | PNHGNF | | | |
| | L | | | | | | L | | | |
| SRGN_59 | LLPGESN | 296 | 2.86E+09 | 7.79E−03 | 1.17 | (E) | LLPGES | 296 | 2.32E+08 | Serglycin |
| | KIPR | | | | | | NKIPR | | | |
| TAGLN_1 | LQEGKH | 305 | 1.28E+09 | | | | | | | Transgelin |
| 68 | VIGLQm | | | | | | | | | |
| | GSNR | | | | | | | | | |
| TAGLN2 | LQEGKN | 307 | 6.52E+07 | | | | | | | Transgelin |
| 167 | VIGLQm | | | | | | | | | -2 |
| | GTNR | | | | | | | | | |
| THBS2_2 | | | | 4.33E−04 | 4.41 | (Q) | LSENLK | 318 | 1.83E+08 | Thrombo- |
| 90 | | | | | | | R | | | spondin-2 |
| TMTC1_5 | LGTLTR | 321 | 1.47E+09 | | | | | | | Trans- |
| 85 | | | | | | | | | | membrane |
| | | | | | | | | | | and TPR |
| | | | | | | | | | | repeat- |
| | | | | | | | | | | containing |
| | | | | | | | | | | protein 1 |
| VIM_314 | QESTEY | 324 | 7.47E+08 | | | | | | | Vimentin |
| | R | | | | | | | | | |
| VIM_333 | LKGTNE | 325 | 1.10E+08 | | | | | | | Vimentin |
| | SLER | | | | | | | | | |

TABLE 6
Human ADAMTS7 Conversion

| | | SEQ | | | | | | | SEQ | |
|---|---|---|---|---|---|---|---|---|---|---|
| Mouse ADAMTS7 | | ID | SMC1 | SMC2 | HUVEC | | Human ADAMTS7 | | ID | Site |
| site | | NO: | TAILS | TAILS | TAILS | Subdomain | site | | NO: | conservation |
| 64.65 | DVST.TQAS | 566 | X | | | Prodomain | 74.75 | DVSV.RRDA | 637 | |
| 65.66 | VSTT.QASS | 567 | X | X | | Prodomain | 75.76 | VSVR.RDAP | 638 | |
| 66.67 | STTQ.ASSA | 568 | X | | | Prodomain | 76.77 | SVRR.DAPA | 639 | |
| 67.68 | TTOA.SSAF | 569 | X | X | X | Prodomain | 77.78 | VRRD.APAF | 640 | |
| 69.70 | QASS.AFYQ | 570 | | X | | Prodomain | 79.80 | RDAP.AFYE | 641 | |
| 70.71 | ASSA.FYQL | 571 | X | X | X | Prodomain | 80.81 | DAPA.FYEL | 642 | |
| 71.72 | SSAF.YQLQ | 572 | X | X | X | Prodomain | 81.82 | APAF.YELQ | 643 | |
| 72.73 | SAFY.QLQY | 573 | X | X | | Prodomain | 82.83 | PAFY.ELQY | 644 | 2 substitutions |
| 73.74 | AFYQ.LQYQ | 574 | | X | | Prodomain | 83.84 | AFYE.LQYR | 645 | 2 substitutions |
| 89.90 | TTNP.YLMA | 575 | X | X | X | Prodomain | 99.100 | TANQ.HLLA | 646 | |
| 90.91 | TNPY.LMAP | 576 | X | X | X | Prodomain | 100.101 | ANQH.LLAP | 647 | |
| 102.103 | EIRR.HSTL | 577 | | X | | Prodomain | 112.113 | ETRR.RGGL | 648 | |
| 107.108 | STLG.HAHI | 578 | | X | | Prodomain | 117.118 | GGLG.RAHI | 649 | |
| 108.109 | TLGH.AHIQ | 579 | X | | X | Prodomain | 118.119 | GLGR.AHIR | 650 | |
| 109.110 | LGHA.HIQT | 580 | X | X | X | Prodomain | 119.120 | LGRA.HIRA | 651 | |
| 110.111 | GHAH.IQTS | 581 | X | | X | Prodomain | 120.121 | GRAH.IRAH | 652 | |
| 111.112 | HAHI.QTSV | 582 | | | X | Prodomain | 121.122 | RAHI.RAHT | 653 | |
| 122.123 | HLLG.DVQD | 583 | X | | | Prodomain | 132.133 | HLLG.EVQD | 654 | 1 substitution |
| 132.133 | LEGG.FAAI | 584 | X | | X | Prodomain | 142.143 | LEGG.LAAI | 655 | 1 substitution |
| 135.136 | GFAA.ISAC | 585 | X | | | Prodomain | 145.146 | GLAA.ISAC | 656 | 1 substitution |
| 144.145 | GLRG.VFQL | 586 | | X | | Prodomain | 154.155 | GLKG.VFQL | 657 | 1 substitution |
| 145.146 | LRGV.FQLS | 587 | | X | | Prodomain | 155.156 | LKGV.FQLS | 658 | 1 substitution |
| 153.154 | NEDY.FIEP | 588 | X | | | Prodomain | 163.164 | NEDY.FIEP | 588 | IDENTICAL |
| 157.158 | FIEP.LDGV | 589 | X | | | Prodomain | 167.168 | FIEP.LDSA | 659 | 2 substitutions |
| 160.161 | PLDG.VSAQ | 590 | X | | | Prodomain | 170.171 | PLDS.APAR | 660 | |
| 162.163 | DGVS.AQPG | 591 | | X | X | Prodomain | 172.173 | DSAP.ARPG | 661 | |
| 163.164 | GVSA.QPGH | 592 | X | X | | Prodomain | 173.174 | SAPA.RPGH | 662 | |
| 165.166 | SAQP.GHAQ | 593 | | X | | Prodomain | 175.176 | PARP.GHAQ | 663 | 2 substitutions |
| 170.171 | HAOP.HVVY | 5 | X | X | X | Prodomain | 180.181 | HAQP.HVVY | 5 | IDENTICAL |
| 204.205 | DLEQ.QREH | 594 | X | | | Prodomain | 214.215 | ELES.RRER | 664 | |
| 442.443 | KDVI.ALPS | 595 | | | X | Metallo- | 457.458 | KDII.DFPS | 665 | |
| | | | | | | proteinase | | | | |
| 445.446 | IALP.SVLP | 596 | | X | X | Metallo- | 460.461 | IDFP.SVPP | 666 | |
| | | | | | | proteinase | | | | |
| 600.601 | YKGK.LHKW | 597 | X | | | Cysteine- | 615.616 | YKGQ.LHTW | 667 | 2 substitutions |
| | | | | | | rich | | | | |
| 632.633 | LRDA.VVDG | 598 | X | | | Cysteine- | 647.648 | LRDA.VVDG | 598 | IDENTICAL |
| | | | | | | rich | | | | |
| 688.689 | VSRT.FKET | 599 | | X | X | Spacer | 703.704 | VSGT.FEEA | 668 | |
| 690.691 | RTFK.ETEG | 600 | X | | | Spacer | 705.706 | GTFE.EAEG | 669 | |
| 694.695 | ETEG.QGYV | 601 | X | | | Spacer | 709.710 | EAEG.LGYV | 670 | 2 substitutions |
| 697.698 | GQGY.VDIG | 602 | X | | | Spacer | 712.713 | GLGY.VDVG | 671 | 2 substitutions |
| 698.699 | QGYV.DIGL | 603 | X | | | Spacer | 713.714 | LGYV.DVGL | 672 | 2 substitutions |
| 714.715 | LIEE.VAEA | 604 | X | X | X | Spacer | 729.730 | RIQE.VAEA | 673 | 2 substitutions |
| 716.717 | EEVA.EAAN | 605 | X | | | Spacer | 731.732 | QEVA.EAAN | 674 | 1 substitution |
| 717.718 | EVAE.AANF | 606 | | X | X | Spacer | 732.733 | EVAE.AANF | 606 | IDENTICAL |
| 718.719 | VAEA.ANFL | 607 | X | X | | Spacer | 733.734 | VAEA.ANFL | 607 | IDENTICAL |
| 732.733 | PDKY.FLNG | 608 | | | X | Spacer | 747.748 | PEKY.FLNG | 675 | 1 substitution |
| 733.734 | DKYF.LNGG | 609 | X | X | X | Spacer | 748.749 | EKYF.LNGG | 676 | 1 substitution |
| 737.738 | LNGG.WTIQ | 610 | | X | X | Spacer | 752.753 | LNGG.WTIQ | 610 | IDENTICAL |
| 763.764 | NWEN.LTSP | 611 | | X | X | Spacer | 778.779 | NWEN.LTSP | 611 | IDENTICAL |
| 780.781 | QLLF.QEKN | 612 | X | X | X | Spacer | 795.796 | QLLF.QESN | 677 | 1 substitution |
| 787.788 | NPGV.HYQY | 613 | | X | X | Spacer | 802.803 | NPGV.HYEY | 678 | 1 substitution |
| 789.790 | GVHY.QYTI | 614 | | X | X | Spacer | 804.805 | GVHY.EYTI | 679 | 1 substitution |
| 808.809 | EFSW.HYGP | 615 | X | X | | TSR2 | 825.826 | VFSW.HYGP | 680 | 1 substitution |
| 812.813 | HYGP.WSKC | 616 | | X | | TSR2 | 829.830 | HYGP.WTKC | 681 | 1 substitution |
| 841.842 | VVAE.EYCN | 617 | X | X | | TSR2 | 858.859 | PVDE.EHCD | 682 | |
| 842.843 | VAEE.YCNT | 618 | X | X | | TSR2 | 859.860 | VDEE.HCDP | 683 | |
| 846.847 | YCNT.LNRP | 95 | X | | | TSR2 | 863.864 | HCDP.LGRP | 684 | |
| 901.902 | EQRA.LELS | 619 | X | | | TSR3 | 918.919 | EQSA.LEPP | 685 | |
| 903.904 | RALE.LSAC | 620 | X | | | TSR3 | 920.921 | SALE.PPAC | 686 | |
| 913.914 | LPRP.LAET | 621 | | | X | TSR3 | 930.931 | LPRP.PTET | 687 | 2 substitutions |
| 998.999 | SPEL.FNEV | 622 | X | | | Mucin | 1015.1016 | SHEL.FNEA | 688 | 2 substitutions |
| 1007.1008 | FIPN.QLAP | 623 | | X | | Mucin | 1024.1025 | FIPH.HLAP | 689 | 2 substitutions |
| 1015.1016 | RPSP.ASSP | 624 | | X | X | Mucin | 1032.1033 | RPSP.ASSP | 624 | IDENTICAL |
| 1021.1022 | SPKP.VSIS | 625 | | X | | Mucin | 1038.1039 | SPKP.GTMG | 690 | |
| 1061.1062 | SYGS.FEEP | 4 | (N/A) | X | X | Mucin | 1080.1081 | SYGP.SEEP | 3 | 2 substitutions |
| 1113.1114 | SPSP.LLSE | 626 | | X | | Mucin | 1132.1133 | SPSP.WPSQ | 691 | |
| 1118.1119 | LSEA.SYSP | 627 | | X | | Mucin | 1137.1138 | PSQA.GRSP | 692 | |
| 1132.1133 | SINP.LANF | 628 | | X | | Mucin | 1152.1153 | PGNP.LINF | 693 | |
| 1147.1148 | MGAP.ELGF | 629 | | X | X | Mucin | 1167.1168 | IGAP.DLGL | 694 | |
| 1175.1176 | NPDE.LLVK | 630 | X | | | Mucin | 1195.1196 | SQND.FPVG | 695 | |
| 1307.1308 | HLKT.LTMP | 631 | X | | X | Mucin | 1329.1330 | DLQT.VAVW | 696 | |
| 1311.1312 | LTMP.GTLL | 632 | | X | | Mucin | 1333.1334 | VAVW.GTFL | 697 | |
| 1315.1316 | GTLL.LTVP | 633 | X | | | Mucin | 1337.1338 | GTFL.PTTL | 698 | |
| 1364.1365 | EVQP.LQPS | 634 | X | | X | Mucin | 1393.1394 | ETQP.LAPS | 699 | |
| 1548.1549 | VVGP.WGQC | 635 | | | X | TSR8 | 1577.1578 | VVGP.WGQC | 635 | IDENTICAL |
| 1583.1584 | DLCS.HEAW | 636 | | X | X | TSR8 | 1612.1613 | DQCG.HEAW | 700 | 2 substitutions |
| 1600.1601 | EDCE.LVEP | 7 | X | X | X | PLAC | 1629.1630 | EDCE.PVEP | 701 | 1 substitution |

### DISCUSSION

The study performed TMT-TAILS to identify substrates for ADAMTS7 from the secretomes of vascular smooth muscle and endothelial cells. Each of the three independent TAILS experiments identified previously unknown candidate substrate cleavage sites associated with ADAMTS7 activity. To confirm the study findings, the study presented the validation of three cleavage sites identified in multiple TAILS datasets: an auto-cleavage site 1061.1062 (SYGS|FEEP) (SEQ ID NO: 4) within the mucin domain of mouse ADAMTS7 and the adjacent sites 123.124 (ASAA|AVAG) (SEQ ID NO: 1) and 124.125 (SAAA|VAGP) (SEQ ID NO: 2) within the atypical first EGF repeat of EFEMP1.

EFEMP1, commonly known as Fibulin-3, is a secreted extracellular matrix protein highly expressed in the vasculature in a pattern overlapping with ADAMTS7 (GTEx Portal V8). Targeted mutation of mouse Efemp/resulted in a viable knockout mouse with abnormal connective tissue due to impaired elastogenesis, including a propensity for hernias and early aging phenotypes. Similar connective tissue disorders were found in a human patient with EFEMP/truncating mutations. In contrast, a recurrent R345W gain-of-function mutation in the central region of EFEMP1 results autosomal dominant Doyne honeycomb retinal dystrophy. Thus, there are no readily apparent correlations between the known EFEMP1 gain- or loss-of-function phenotypes and the atheroprotection conferred from ADAMTS7 loss-of-function. While Adamts7 knockout mice do not have a reported connective tissue disorder, a recent report describes abnormal collagen fibrillogenesis in Adamts7 Adamts12 double knockout mice resulting in tendon heterotopic ossification. Compensation and substrate redundancies between the paralogs ADAMTS7 and ADAMTS12 may explain the lack of overlap from the individual enzyme loss-of-function phenotypes and the diseases associated with mutations in the candidate substrates.

Although it is presently unclear if ADAMTS7 regulated EFEMP1 cleavage will impact vascular phenotypes, experimental evidence shows that EFEMP1 124.125 cleavage by MMP may alter interacting binding partners. Within the family of fibulin proteins, EFEMP1/Fibulin-3 is similar in structure to EFEMP2/Fibulin-4 and FBLN5/Fibulin-5, however neither Fibulin-4 or Fibulin-5 were identified as ADAMTS7 candidate substrates from the study experiments. In the related family member FBLN2/Fibulin-2, adjacent sites 258.259 (TAAA|ALGP) (SEQ ID NO: 702) and 259.260 (AAAA|LGPP) (SEQ ID NO: 703) in the N-terminal cysteine-free region were identified as candidate sites in both of the SMC TAILS experiments. Cleavage by ADAMTS7 at this location would release the FBLN2 N-terminal cysteine-rich domain with reported roles in FBLN2 oligomerization.

In the active form of full-length mouse ADAMTS7, the study consistently observed a lower band at 150 kDa in the media from SMC, HUVEC and HEK293 fibroblasts. Amino terminal sequencing identified the WT 150 kDa band beginning at phenylalanine 1062 (FEEPHPDL) (SEQ ID NO: 704). In this study, TAILS experiments digested with AspN identified significantly regulated peptides in the WT/EQ comparison to support a predominant ADAMTS7 auto-cleavage event at 1061.1062 (SYGS|FEEP) (SEQ ID NO: 4) nearby the CS-GAG attachment site in the mucin domain. Removal of the amino acids 1062-1657 would preserve a CS-GAG tethered enzyme that lacks a carboxyl terminus normally thought to be required for substrate recognition, potentially changing the exosite substrate specificity for ADAMTS7.

The mouse ADAMTS7 auto-cleavage site is adjacent to one of the few highly conserved regions within the mucin domain and is partially conserved in human ADAMTS7 1080.1081 (SYGP|SEEP) (SEQ ID NO: 3), although the study were unable to confirm auto-cleavage for WT human ADAMTS7. Cleavage at this position was not identified in a TAILS experiment using a human ADAMTS7 truncated after the mucin domain, lacking the carboxyl terminal TSR repeats 5-8 and PLAC domains. From this study, they identified auto-cleavage events in the spacer domain for human ADAMTS7 (729.730 RIQE|VAEA (SEQ ID NO: 673) and 732.733 EVAE|AANF) (SEQ ID NO: 606) with confirmed amino terminal sequencing of the latter site. Within the study TAILS datasets using mouse ADAMTS7, the study identified analogous auto-cleavage sites in the spacer domain at 714.715 (LIEE|VAEA) (SEQ ID NO: 604) for all TAILS experiments and 717.718 (EVAE|AANF) (SEQ ID NO: 606) in the SMC2 and HUVEC TAILS experiments (Table 5). Analysis of the WT/EQ significantly enriched peptides revealed multiple sites in the prodomain consistent with auto-cleavage events, with the most abundant site at 170.171 (HAQP|HVVY) (SEQ ID NO: 5). Although this site is entirely conserved in human ADAMTS7, it was not identified in the previous TAILS study. The ADAMTS7 prodomain contains a cysteine switch motif which acts to maintain enzyme latency through interactions with the Zinc metal in the active site. The mouse ADAMTS7 prodomain is processed by Furin protease at 60.61 and 220.221, with only the second Furin processing site removing the inhibitory cysteine switch at Cys194. Although mouse ADAMTS7 cleavage at 170.171 would retain the cysteine switch to the catalytic domain, it is possible this may affect the progressive zymogen processing by Furin.

Analysis of P4 through P4′ positions from ADAMTS7 auto-cleavage and candidate substrate cleavage sites from the TAILS experiments suggests that ADAMTS7 is able to process substrates in a variety of contexts (FIG. 6). Visually from the iceLogo plots, a preference for Alanine in the P1 position and Leucine in the P1′ was the most common, however many candidate cleavage sites identified in multiple TAILS experiments did not conform to the consensus logo plots. ADAMTS7 TAILS substrate specificity was notably similar to those reported for MMP2 TAILS experiments showing a preference for PAA|L (SEQ ID NO: 705) in the P3 through P1′ positions without an absolute requirement for any of these residues. Compared to the preferences from other ADAMTS family members, there appear to be more similarities with the procollagen N-proteinases ADAMTS2/3/14 preference for A/P|Q than the aggrecanases ADAMTS4/5 preference for E|A/L. In peptide based enzymatic assays, cleavage of aggrecan (TEGE|ARGS (SEQ ID NO: 706) or TAQE|AGEG) (SEQ ID NO: 707) or versican (EAAE|ARRG) (SEQ ID NO: 708) has been reported for ADAMTS1, ADAMTS4, ADAMTS5, ADAMTS8, ADAMTS9 and ADAMTS20. Although ADAMTS7 activity generated many candidate E|A cleavage sites within the study TAILS datasets, the study did not detect significantly regulated peptides for aggrecan or versican at these sites to suggest similar cleavage events. This is consistent with the initial characterization of ADAMTS7 reporting no activity for aggrecan or versican using neo-epitope antibodies for the common aggrecanase cleavage sites. In the previous ADAMTS7 TAILS study, a P1 glutamate preference was observed in the latent-transforming growth factor beta-binding proteins 3 and 4, specifically at LTBP3 220.221 (ISAE|VQAP) (SEQ ID NO: 709) and LTPBP4 229.230 (HPQE|ASVV) (SEQ ID NO: 710). In the study, the LTPBP4 229.230 site was not significantly regulated in the TAILS datasets, but nearby LTBP4 233.234 (ASVV|VHQV) (SEQ ID NO: 711) was significantly regulated in the SMC1 dataset. The previously reported LTBP3 220.221 site was significant in the HUVEC TAILS experiment but not in the SMC TAILS datasets. Within this region, LTPBP3 238.239 (PPEA|SVQV) (SEQ ID NO: 23) was identified in all three TAILS experiments and alignment of LTBP3 and LTBP4 brings this cleavage site very close to the reported LTBP4 229.230 (HPQE|ASVV) (SEQ ID NO: 710) site. Collectively, these examples from the study TAILS experiments demonstrate that ADAMTS7 exhibits broad specificity for the amino acids at the site of cleavage. Nonetheless, the repeated discovery of the same diverse cleavage sites suggests that substrate specificity remains a feature of ADAMTS7 and likely relies on other non-enzymatic domains for substrate recognition.

Within the study list of candidate substrate cleavage sites identified in more than one TAILS experiment, the study observed that while some candidate substrates displayed cleavage within a specific region of the protein, others fell into a different category where multiple identified cleavage sites were present throughout the protein in multiple domains. This was the case for Fibronectin and HSPG2/Perlecan for all the TAILS experiments and for COL1A2 from the SMC TAILS experiments. One possibility is that ADAMTS7 normally associates with these proteins in the extracellular matrix and under the study over-expression conditions opportunistically cleaves these substrates in a less regulated manner. In contrast, other identified substrates displayed a more restricted pattern of cleavage confined to a particular region which may suggest a more regulated interaction and cleavage process. This appears to be the case with EFEMP1 and may hold true for other candidate substrates such as LOX which displayed a string of candidate sites in the prodomain at positions 122.123, 123.124, 124.125 and 125.126 from multiple TAILS experiments. The candidate site that was present in all three TAILS experiments was at LOX 123.124 (TARH|WFQA) (SEQ ID NO: 20) upstream from the zymogen processing site at 162.163 by the procollagen C-proteinase BMP1. The ADAMTS7 mediated prodomain LOX cleavage sites are distinct in location from those identified from the ADAMTS2/3/14 TAILS experiments or from the reported LOX catalytic domain cleavage by ADAMTS2/14. The LOX prodomain is essential for secretion and assists with substrate interaction. Following BMP1 cleavage, the LOX prodomain also has the ability to act as a bioactive product with tumor suppressor function independent from LOX enzymatic domain. Therefore, ADAMTS7 cleavage of the LOX prodomain may impact multiple functions of the pro-LOX zymogen association with substrates, pro-LOX zymogen processing or the modification/inactivation of the bioactive free LOX propeptide. In the cases of EFEMP1 and LOX, the ADAMTS7 cleavage events are at adjacent amino acid positions which may produce similar biological effects, however these phenomena may complicate the development of neo-epitope specific antibodies to a single defined cleavage site similar to the reagents developed for the aggrecanases ADAMTS4 and ADAMTS5.

In addition to inactivating structural and bioactive ECM factors, ADAMTS7 TAILS candidate cleavage sites have the potential to produce known bioactive products from unique cleavage sites in the hinge regions of COL18A1 (Collagen type XVIII alpha-1) and CTGF (Connective Tissue Growth Factor). Endostatin and endostatin-like fragments with anti-angiogenic properties originate from the carboxyl terminal region of COL18A1 following MMP/elastase/cathepsin cleavage within the hinge region (amino acids 1502-1571). The ADAMTS7 candidate cleavage site COL18A1 1504.1505 (EVAA|LQPP) (SEQ ID NO: 9) found in all three TAILS experiments is located near the beginning of the hinge region, upstream from the first known MMP cleavage site at 1511.1512 by MMP7. The study findings suggest that ADAMTS7 is capable of producing an endostatin-like fragment with similar anti-angiogenic activities. Additional significantly upregulated peptides corresponding to nearby cleavage at 1501.1502 (TDNE|VAAL) (SEQ ID NO: 712) and 1503.1504 (NEVA|ALQP) (SEQ ID NO: 713) were present in the HUVEC dataset and may correlate with the increase in COL18A1 protein levels detected in the HUVEC secretome. In fact, COL18A1 was one of the few examples a protein significantly upregulated in the WT secretome, but not in the Luc or EQ secretomes, consistent with COL18A1 upregulation in response to ADAMTS7 catalytic activity. This may represent a feed forward circuit with ADAMTS7 activity stimulating both the upregulation and cleavage of COL18A1 resulting in an endostatin-like matrikine.

CTGF, also known as CCN2, is a secreted multidomain matricellular protein with a central proteolytically sensitive hinge region (amino acids 168-197). It has been previously shown that cleavage in the hinge domain at 180.181 (PALA|AYRL) (SEQ ID NO: 714) by MMPs generates a bioactive carboxyl terminal fragment containing the TSR and cysteine rich CT domains. CTGF is highly expressed in the HUVEC cell line and specifically in the HUVEC TAILS experiment the study identified significantly regulated peptides representing CTGF hinge region cleavage sites at 172.173 (PKDQ|TVVG) (SEQ ID NO: 715), 173.174 (KDQT|VVGP) (SEQ ID NO: 716), 177.178 (VVGP|ALAA) (SEQ ID NO: 717) and 186.187 (RLED|TFGP) (SEQ ID NO: 718). Although multiple protease cleavage sites have been reported for the CTGF hinge region, very few known sites overlap with the ADAMTS7 TAILS candidates, with the exception of the 186.187 site identified in an ADAMTS3 TAILS study. CTGF was previously identified as a potential substrate for ADAMTS7 through a yeast two hybrid screen demonstrating a requirement for the ADAMTS7 mucin, TSR5-8 and PLAC domains for interaction with the CTGF amino terminal region. Furthermore, it was shown in an in vitro cleavage assay that the ADAMTS7 catalytic domain processed CTGF, producing bands compatible with cleavage in the hinge region. A similar binding interaction between CTGF and the paralog ADAMTS12 was mapped to the mucin and TSR5-8 regions of ADAMTS12, along with evidence of CTGF processing from co-transfected cells. The study ADAMTS7 TAILS study provides further evidence for a connection between ADAMTS7 and CTGF from an unbiased proteomic method. Based on the data from the study HUVEC TAILS experiment, a cleavage site preference of 172.173 or 186.187 in the CTGF hinge region would be predicted based on log FC values and total spectra intensities.

Although ADAMTS7 is characterized as a COMP protease, the study were unable to identify significantly regulated peptides consistent an ADAMTS7 candidate cleavage site. COMP protein and peptides were identified in each of the TAILS and secretome experiments, however the total peptide coverage ranged from 18-23% indicating that a significant portion of COMP was not captured and quantitated in the study TAILS experiments. In the case of COMP, this may be due to a limitation in the expression level of the substrate of interest in the study cell lines and the depth of amino acid coverage in the study experiments. Another reported ADAMTS7 substrate is thrombospondin 1 and both the SMC and HUVEC cell lines expressed much higher levels of this protein resulting in 71-78% total peptide coverage. Within THBS1, two regulated peptides were identified in separate experiments (629.630 in HUVEC and 971.972 in SMC1), however these were low abundance peptides and were not consistently identified in the other TAILS datasets.

Achieving appropriate coverage and depth for a given substrate is a challenge for any unbiased degradomics technique and the study attempted improve these traits in the study successive TAILS experiments. For instance, in the first TAILS experiment, the study digested the TMT labeled peptides with only trypsin, limiting the identification of candidate sites to peptides greater than five residues proceeding a tryptic R. or K. site that could be identified through LC-MS/MS. To improve the peptide coverage for ADAMTS7 substrates in the following TAILS experiments, the study analyzed both trypsin and AspN digested products. Additionally the study analyzed the peptides with a relaxed AspN condition to capture both .D or .E cleavage events to increase the number of identifiable spectra. Compared to an analysis of the study datasets with a strict AspN (.D only) consensus, application of relaxed AspN condition further increased the number of quantified TMT labeled peptides by 23% for the SMC2 and 17% for the HUVEC TAILS experiments. Including additional enzymes such as chymotrypsin with cleavage sites distinct from trypsin and AspN would likely increase depth and coverage even further to capture additional ADAMTS7 candidate cleavage sites.

Based on the diverse cleavage site specificity from ADAMTS7 in the study TAILS experiments, the study experimental design likely benefited from using the full-length protein with the carboxyl-terminal substrate interaction domains. Additional ADAMTS7 site specificity could be investigated using a PICS (Proteomic Identification of protease Cleavage Sites) based strategy utilizing a library of short peptides predigested with a specific enzyme like trypsin or LysC, however this may not reliably identify endogenous substrate cleavage sites driven by exosite specificity. Consistent with the study observations of ADAMTS7 cleavage site specificity, a broad specificity for the ADAMTS7 enzyme was observed in a library of internally quenched fluorogenic peptides where nearly half were appreciably cleaved.

### INCORPORATION BY REFERENCE

All publications, patents, patent applications and sequence accession numbers mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize or be able to ascertain using no more than routine experimentation, many equivalents to the specific embodiments of the invention described herein. Such equivalents are intended to be encompassed by the following claims.

1-14. (canceled)

15. A method of treating or preventing vascular disease and/or heart disease in a subject, comprising:
(a) determining whether serum of the subject comprises a level of a cleaved protein above a threshold level; and
(b) if the serum is characterized by a level above the threshold level, administering an antagonist of ADAMTS7 (A disintegrin and metalloproteinase with thrombospondin motifs 7) to the subject.

16. The method of claim 15, wherein pre-cleaved protein is expressed in the vasculature of the subject.

17. The method of claim 16, wherein determining whether the level of the cleaved protein is above a threshold level comprises measuring the level of the cleaved protein in the serum.

18. The method of claim 16, wherein the cleaved protein is encoded by a gene listed in Table 3.

19. The method of claim 18, wherein the cleaved protein is cleaved at a cleavage site listed in Table 3.

20. The method of claim 19, wherein the cleaved protein is cleaved fibulin protein.

21. The method of claim 20, wherein the cleaved fibulin protein is cleaved EGF-containing fibulin-like extracellular matrix protein 1 (EFEMP1).

22. The method of claim 21, wherein the cleaved EFEMP1 protein is cleaved at cleavage site 123.124 (ASAA|AVAG) (SEQ ID NO: 1).

23. The method of claim 21, wherein the cleaved EFEMP1 protein is cleaved at cleavage site 124.125 (SAAA|VAGP) (SEQ ID NO: 2).

24. A method of treating or preventing vascular disease and/or heart disease in a subject, comprising:
(a) determining whether serum of the subject comprises a level of auto-cleaved ADAMTS7 (A disintegrin and metalloproteinase with thrombospondin motifs 7) above a threshold level; and
(b) if the serum is characterized by a level above the threshold level, administering an antagonist of ADAMTS7 to the subject.

25. The method of claim 24, wherein pre-cleaved ADAMTS7 is expressed in the vasculature of the subject.

26. The method of claim 25, wherein determining whether the level of the auto-cleaved ADAMTS7 is above a threshold level comprises measuring the level of the auto-cleaved ADAMTS7 in the serum.

27. The method of claim 26, wherein the auto-cleaved ADAMTS7 is cleaved at a cleavage site that is at least 75% identical to a cleavage site listed in Table 6.

28. The method of claim 27, wherein the auto-cleaved ADAMTS7 is cleaved at a cleavage site that is at least 87% identical to a cleavage site listed in Table 6.

29. The method of claim 28, wherein auto-cleaved ADAMTS7 is human auto-cleaved ADAMTS7 [0.31].

30. The method of claim 29, wherein the human auto-cleaved ADAMTS7 is cleaved at cleavage site 1080.1081 (SYGP|SEEP) (SEQ ID NO: 3).

31. The method of claim 24, further comprising conjointly administering an additional cardiovascular therapeutic agent to the subject.

32. The method of claim 31, whereby the antagonist of ADAMTS7 enhances the effects of the additional cardiovascular therapeutic agent relative to the additional cardiovascular therapeutic agent alone.

33. The method of claim 15, wherein the heart disease is coronary artery disease.

34-49. (canceled)

50. The method of claim 24, wherein the heart disease is coronary artery disease.


SEQUENCE LISTING (724 sequences)

SEQ ID NO: 1 - protein, 8 aa, Homo sapiens
ASAAAVAG

SEQ ID NO: 2 - protein, 8 aa, Homo sapiens
SAAAVAGP

SEQ ID NO: 3 - protein, 8 aa, Homo sapiens
SYGPSEEP

SEQ ID NO: 4 - protein, 8 aa, Mus musculus
SYGSFEEP

SEQ ID NO: 5 - protein, 8 aa, Homo sapiens
  source 1..8: note = also present in Mus musculus
HAQPHVVY

SEQ ID NO: 6 - protein, 8 aa, Homo sapiens
SYSGFEEP

SEQ ID NO: 7 - protein, 8 aa, Homo sapiens
EDCELVEP

SEQ ID NO: 8 - protein, 8 aa, Homo sapiens
EIEEFLEG

SEQ ID NO: 9 - protein, 8 aa, Homo sapiens
EVAALQPP

SEQ ID NO: 10 - protein, 8 aa, Homo sapiens
ADANLEAG

SEQ ID NO: 11 - protein, 8 aa, Homo sapiens
DARALTQT

SEQ ID NO: 12 - protein, 8 aa, Homo sapiens
QAQQMVQP

SEQ ID NO: 13 - protein, 8 aa, Homo sapiens
MVQPQSPV

SEQ ID NO: 14 - protein, 8 aa, Homo sapiens
VVSGLTPG

SEQ ID NO: 15 - protein, 8 aa, Homo sapiens
KWLPSSSP

SEQ ID NO: 16 - protein, 8 aa, Homo sapiens
APQPLLPG

SEQ ID NO: 17 - protein, 8 aa, Homo sapiens
ASGTLSAP

SEQ ID NO: 18 - protein, 8 aa, Homo sapiens
GTQGANLA

SEQ ID NO: 19 - protein, 8 aa, Homo sapiens
KAGAAAGG

SEQ ID NO: 20 - protein, 8 aa, Homo sapiens
TARHWFQA

SEQ ID NO: 21 - protein, 8 aa, Homo sapiens
YPEYFQQP

SEQ ID NO: 22 - protein, 8 aa, Homo sapiens
AADTLTAT

SEQ ID NO: 23 - protein, 8 aa, Homo sapiens
PPEASVQV

SEQ ID NO: 24 - protein, 8 aa, Homo sapiens
TTAAAVAS

SEQ ID NO: 25 - protein, 8 aa, Homo sapiens
TAAAVAST

SEQ ID NO: 26 - protein, 8 aa, Homo sapiens
LSAAHSSV

SEQ ID NO: 27 - protein, 8 aa, Homo sapiens
QAAAQTNS

SEQ ID NO: 28 - protein, 8 aa, Homo sapiens
RPDQQLQG

SEQ ID NO: 29 - protein, 8 aa, Homo sapiens
PMFELLPG

SEQ ID NO: 30 - protein, 15 aa, Homo sapiens
TQASSAFYQLQYQGR

SEQ ID NO: 31 - protein, 14 aa, Homo sapiens
QASSAFYQLQYQGR

SEQ ID NO: 32 - protein, 13 aa, Homo sapiens
ASSAFYQLQYQGR

SEQ ID NO: 33 - protein, 12 aa, Homo sapiens
SSAFYQLQYQGR

SEQ ID NO: 34 - protein, 9 aa, Homo sapiens
FYQLQYQGR

SEQ ID NO: 35 - protein, 8 aa, Homo sapiens
YQLQYQGR

SEQ ID NO: 36 - protein, 7 aa, Homo sapiens
QLQYQGR

SEQ ID NO: 37 - protein, 12 aa, Homo sapiens
YLMAPGFVSEIR

SEQ ID NO: 38 - protein, 11 aa, Homo sapiens
LMAPGFVSEIR

SEQ ID NO: 39 - protein, 35 aa, Homo sapiens
AHIQTSVPTCHLLGDVQDPELEGGFAAISACDGLR

SEQ ID NO: 40 - protein, 34 aa, Homo sapiens
HIQTSVPTCHLLGDVQDPELEGGFAAISACDGLR

SEQ ID NO: 41 - protein, 33 aa, Homo sapiens
IQTSVPTCHLLGDVQDPELEGGFAAISACDGLR

SEQ ID NO: 42 - protein, 21 aa, Homo sapiens
DVQDPELEGGFAAISACDGLR

SEQ ID NO: 43 - protein, 11 aa, Homo sapiens
FAAISACDGLR

SEQ ID NO: 44 - protein, 8 aa, Homo sapiens
ISACDGLR

SEQ ID NO: 45 - protein, 27 aa, Homo sapiens
FIEPLDGVSAQPGHAQPHVVYKHQGSR

SEQ ID NO: 46 - protein, 23 aa, Homo sapiens
LDGVSAQPGHAQPHVVYKHQGSR

SEQ ID NO: 47 - protein, 20 aa, Homo sapiens
VSAQPGHAQPHVVYKHQGSR

SEQ ID NO: 48 - protein, 17 aa, Homo sapiens
QPGHAQPHVVYKHQGSR

SEQ ID NO: 49 - protein, 10 aa, Homo sapiens
HVVYKHQGSR

SEQ ID NO: 50 - protein, 12 aa, Homo sapiens
QREHWEQQQQKR

SEQ ID NO: 51 - protein, 19 aa, Homo sapiens
LHKWVPVPNDDNPCELHCR

SEQ ID NO: 52 - protein, 11 aa, Homo sapiens
VVDGTPCYQSR

SEQ ID NO: 53 - protein, 18 aa, Homo sapiens
ETEGQGYVDIGLIPAGAR

SEQ ID NO: 54 - protein, 14 aa, Homo sapiens
QGYVDIGLIPAGAR

SEQ ID NO: 55 - protein, 11 aa, Homo sapiens
VDIGLIPAGAR

SEQ ID NO: 56 - protein, 10 aa, Homo sapiens
DIGLIPAGAR

SEQ ID NO: 57 - protein, 11 aa, Homo sapiens
VAEAANFLALR

SEQ ID NO: 58 - protein, 9 aa, Homo sapiens
EAANFLALR

SEQ ID NO: 59 - protein, 7 aa, Homo sapiens
ANFLALR

SEQ ID NO: 60 - protein, 14 aa, Homo sapiens
LNGGWTIQWNGDYR

SEQ ID NO: 61 - protein, 15 aa, Homo sapiens
QEKNPGVHYQYTIQR

SEQ ID NO: 62 - protein, 18 aa, Homo sapiens
HYGPWSKCTVTCGTGVQR

SEQ ID NO: 63 - protein, 12 aa, Homo sapiens
EYCNTLNRPDER

SEQ ID NO: 64 - protein, 11 aa, Homo sapiens
YCNTLNRPDER

SEQ ID NO: 65 - protein, 7 aa, Homo sapiens
LNRPDER

SEQ ID NO: 66 - protein, 20 aa, Homo sapiens
LELSACEHLPRPLAETPCNR

SEQ ID NO: 67 - protein, 9 aa, Homo sapiens
LSACEHLPR

SEQ ID NO: 68 - protein, 14 aa, Homo sapiens
FNEVDFIPNQLAPR

SEQ ID NO: 69 - protein, 18 aa, Homo sapiens
LLVKEDEQSPPSTPWSDR

SEQ ID NO: 70 - protein, 16 aa, Homo sapiens
LTMPGTLLLTVPTDLR

SEQ ID NO: 71 - protein, 8 aa, Homo sapiens
LTVPTDLR

SEQ ID NO: 72 - protein, 18 aa, Homo sapiens
LQPSLEEDGDPADPLPAR

SEQ ID NO: 73 - protein, 6 aa, Homo sapiens
LVEPPR

SEQ ID NO: 74 - protein, 10 aa, Homo sapiens
AFYQLQYQGR

SEQ ID NO: 75 - protein, 6 aa, Homo sapiens
LQYQGR

SEQ ID NO: 76 - protein, 9 aa, Homo sapiens
YLMAPGFVS

SEQ ID NO: 77 - protein, 8 aa, Homo sapiens
LMAPGFVS

SEQ ID NO: 78 - protein, 20 aa, Homo sapiens
HSTLGHAHIQTSVPTCHLLG

SEQ ID NO: 79 - protein, 15 aa, Homo sapiens
HAHIQTSVPTCHLLG

SEQ ID NO: 80 - protein, 13 aa, Homo sapiens
HIQTSVPTCHLLG

SEQ ID NO: 81 - protein, 7 aa, Homo sapiens
VFQLSNE

SEQ ID NO: 82 - protein, 6 aa, Homo sapiens
FQLSNE

SEQ ID NO: 83 - protein, 18 aa, Homo sapiens
AQPGHAQPHVVYKHQGSR

SEQ ID NO: 84 - protein, 15 aa, Homo sapiens
GHAQPHVVYKHQGSR

SEQ ID NO: 85 - protein, 8 aa, Homo sapiens
SVLPGVLY

SEQ ID NO: 86 - protein, 10 aa, Homo sapiens
FKETEGQGYV

SEQ ID NO: 87 - protein, 8 aa, Homo sapiens
AANFLALR

SEQ ID NO: 88 - protein, 11 aa, Homo sapiens
LNGGWTIQWNG

SEQ ID NO: 89 - protein, 7 aa, Homo sapiens
WTIQWNG

SEQ ID NO: 90 - protein, 8 aa, Homo sapiens
LTSPGPTS

SEQ ID NO: 91 - protein, 8 aa, Homo sapiens
HYQYTIQR

SEQ ID NO: 92 - protein, 6 aa, Homo sapiens
QYTIQR

SEQ ID NO: 93 - protein, 14 aa, Homo sapiens
WSKCTVTCGTGVQR

SEQ ID NO: 94 - protein, 9 aa, Homo sapiens
EYCNTLNRP

SEQ ID NO: 95 - protein, 8 aa, Homo sapiens
YCNTLNRP

SEQ ID NO: 96 - protein, 5 aa, Homo sapiens
QLAPR

SEQ ID NO: 97 - protein, 13 aa, Homo sapiens
ASSPKPVSISNAI

SEQ ID NO: 98 - protein, 7 aa, Homo sapiens
VSISNAI

SEQ ID NO: 99 - protein, 6 aa, Homo sapiens
FEEPHP

SEQ ID NO: 100 - protein, 9 aa, Homo sapiens
FEEPHPDLV

SEQ ID NO: 101 - protein, 12 aa, Homo sapiens
LLSEASYSPPGL

SEQ ID NO: 102 - protein, 20 aa, Homo sapiens
SYSPPGLEQTSINPLANFLT

SEQ ID NO: 103 - protein, 22 aa, Homo sapiens
SYSPPGLEQTSINPLANFLTEE

SEQ ID NO: 104 - protein, 8 aa, Homo sapiens
LANFLTEE

SEQ ID NO: 105 - protein, 15 aa, Homo sapiens
ELGFPSLPWPPASVD

SEQ ID NO: 106 - protein, 9 aa, Homo sapiens
GTLLLTVPT

SEQ ID NO: 107 - protein, 14 aa, Homo sapiens
HEAWPESSRPCATE

SEQ ID NO: 108 - protein, 14 aa, Homo sapiens
AHIQTSVPTCHLLG

SEQ ID NO: 109 - protein, 12 aa, Homo sapiens
IQTSVPTCHLLG

SEQ ID NO: 110 - protein, 11 aa, Homo sapiens
QTSVPTCHLLG

SEQ ID NO: 111 - protein, 11 aa, Homo sapiens
ALPSVLPGVLY

SEQ ID NO: 112 - protein, 12 aa, Homo sapiens
FLNGGWTIQWNG

SEQ ID NO: 113 - protein, 8 aa, Homo sapiens
LAETPCNR

SEQ ID NO: 114 - protein, 14 aa, Homo sapiens
ELGFPSLPWPPASV

SEQ ID NO: 115 - protein, 13 aa, Homo sapiens
LTMPGTLLLTVPT

SEQ ID NO: 116 - protein, 12 aa, Homo sapiens
LQPSLEEDGDPA

SEQ ID NO: 117 - protein, 14 aa, Homo sapiens
WGQCSAPCGGGVQR

SEQ ID NO: 118 - protein, 21 aa, Homo sapiens
LVVDNGSGMCKAGFAGDDAPR

SEQ ID NO: 119 - protein, 8 aa, Homo sapiens
FAGDDAPR

SEQ ID NO: 120 - protein, 12 aa, Homo sapiens
LTEAPLNPKANR

SEQ ID NO: 121 - protein, 21 aa, Homo sapiens
LVIDNGSGMCKAGFAGDDAPR

SEQ ID NO: 122 - protein, 15 aa, Homo sapiens
VIQAEGKEHIIHLER

SEQ ID NO: 123 - protein, 14 aa, Homo sapiens
FLEGFLSELEPEPR

SEQ ID NO: 124 - protein, 9 aa, Homo sapiens
ISETPVDVR

SEQ ID NO: 125 - protein, 6 aa, Homo sapiens
QGTILR

SEQ ID NO: 126 - protein, 6 aa, Homo sapiens
QGTLLR

SEQ ID NO: 127 - protein, 11 aa, Homo sapiens
LEEPNLQPLQR

SEQ ID NO: 128 - protein, 10 aa, Homo sapiens
FQKEKEELDR

SEQ ID NO: 129 - protein, 6 aa, Homo sapiens
QTGIIR

SEQ ID NO: 130 - protein, 11 aa, Homo sapiens
HQEAKVPVAIR

SEQ ID NO: 131 - protein, 12 aa, Homo sapiens
HLQEALGLPAGR

SEQ ID NO: 132 - protein, 9 aa, Homo sapiens
QSEGKVYTR

SEQ ID NO: 133 - protein, 16 aa, Homo sapiens
LQPPVVQLHDSNPYPR

SEQ ID NO: 134 - protein, 6 aa, Homo sapiens
QEETVR

SEQ ID NO: 135 - protein, 20 aa, Homo sapiens
QYDGKGVGLGPGPMGLMGPR

SEQ ID NO: 136 - protein, 9 aa, Homo sapiens
GPMGLMGPR

SEQ ID NO: 137 - protein, 20 aa, Homo sapiens
FQGPAGEPGEPGQTGPAGAR

SEQ ID NO: 138 - protein, 13 aa, Homo sapiens
VGNAGPAGPAGPR

SEQ ID NO: 139 - protein, 23 aa, Homo sapiens
LTGAKGAAGLPGVAGAPGLPGPR

SEQ ID NO: 140 - protein, 11 aa, Homo sapiens
SAGPPGPPGLR

SEQ ID NO: 141 - protein, 8 aa, Homo sapiens
ASGPAGVR

SEQ ID NO: 142 - protein, 16 aa, Homo sapiens
VGTAGPSGPSGLPGER

SEQ ID NO: 143 - protein, 13 aa, Homo sapiens
VGAPGPAGATGDR

SEQ ID NO: 144 - protein, 11 aa, Homo sapiens
AAGQPGAKGER

SEQ ID NO: 145 - protein, 14 aa, Homo sapiens
LLGAPGILGLPGSR

SEQ ID NO: 146 - protein, 19 aa, Homo sapiens
AGAPGPHGPVGPAGKHGNR

SEQ ID NO: 147 - protein, 26 aa, Homo sapiens
LQGLPGIAGHHGDQGAPGSVGPAGPR

SEQ ID NO: 148 - protein, 17 aa, Homo sapiens
HHGDQGAPGSVGPAGPR

SEQ ID NO: 149 - protein, 30 aa, Homo sapiens
SLRPKDYEVDATLKSLNNQIETLLTPEGSR

SEQ ID NO: 150 - protein, 16 aa, Homo sapiens
YNGPPGLQGFPGLQGR

SEQ ID NO: 151 - protein, 8 aa, Homo sapiens
YALPKEER

SEQ ID NO: 152 - protein, 11 aa, Homo sapiens
SQAQAILQQAR

SEQ ID NO: 153 - protein, 9 aa, Homo sapiens
MKEIQPAPR

SEQ ID NO: 154 - protein, 6 aa, Homo sapiens
ISALTR

SEQ ID NO: 155 - protein, 11 aa, Homo sapiens
YAAPPSPPLSR

SEQ ID NO: 156 - protein, 22 aa, Homo sapiens
FGDQSHPEPESWNAAQHCQQDR

SEQ ID NO: 157 - protein, 11 aa, Homo sapiens
AVAGPEMQTGR

SEQ ID NO: 158 - protein, 10 aa, Homo sapiens
VAGPEMQTGR

SEQ ID NO: 159 - protein, 6 aa, Homo sapiens
NNFVIR

SEQ ID NO: 160 - protein, 11 aa, Homo sapiens
QGIGEPQSPSR

SEQ ID NO: 161 - protein, 8 aa, Homo sapiens
TAPTPAPR

SEQ ID NO: 162 - protein, 13 aa, Homo sapiens
ALGPPAPVQAKAR

SEQ ID NO: 163 - protein, 12 aa, Homo sapiens
LGPPAPVQAKAR

SEQ ID NO: 164 - protein, 10 aa, Homo sapiens
LEAGNVKETR

SEQ ID NO: 165 - protein, 10 aa, Homo sapiens
LKGPNVCGSR

SEQ ID NO: 166 - protein, 23 aa, Homo sapiens
SVEGPSQAKIECDDKGDGSCDVR

SEQ ID NO: 167 - protein, 14 aa, Homo sapiens
FTVDAKHGGKAPLR

SEQ ID NO: 168 - protein, 12 aa, Homo sapiens
LTQTGGPHVKAR

SEQ ID NO: 169 - protein, 32 aa, Homo sapiens
MVQPQSPVAVSQSKPGCYDNGKHYQINQQWER

SEQ ID NO: 170 - protein, 28 aa, Homo sapiens
QSPVAVSQSKPGCYDNGKHYQINQQWER

SEQ ID NO: 171 - protein, 23 aa, Homo sapiens
VSQSKPGCYDNGKHYQINQQWER

SEQ ID NO: 172 - protein, 21 aa, Homo sapiens
QSKPGCYDNGKHYQINQQWER

SEQ ID NO: 173 - protein, 12 aa, Homo sapiens
TSSGSGPFTDVR

SEQ ID NO: 174 - protein, 10 aa, Homo sapiens
SGSGPFTDVR

SEQ ID NO: 175 - protein, 9 aa, Homo sapiens
GSGPFTDVR

SEQ ID NO: 176 - protein, 18 aa, Homo sapiens
NQESTPVVIQQETTGTPR

SEQ ID NO: 177 - protein, 15 aa, Homo sapiens
LTPGVEYVYTIQVLR

SEQ ID NO: 178 - protein, 9 aa, Homo sapiens
SSSPVTGYR

SEQ ID NO: 179 - protein, 11 aa, Homo sapiens
LVQTAVTNIDR

SEQ ID NO: 180 - protein, 38 aa, Homo sapiens
LVQTAVTNIDRPKGLAFTDVDVDSIKIAWESPQGQVSR

SEQ ID NO: 181 - protein, 22 aa, Homo sapiens
FTDVDVDSIKIAWESPQGQVSR

SEQ ID NO: 182 - protein, 7 aa, Homo sapiens
FEEHGFR

SEQ ID NO: 183 - protein, 24 aa, Homo sapiens
FQDTSEYIISCHPVGTDEEPLQFR

SEQ ID NO: 184 - protein, 6 aa, Homo sapiens
LTGLTR

SEQ ID NO: 185 - protein, 11 aa, Homo sapiens
LAGGAGGGAGR

SEQ ID NO: 186 - protein, 9 aa, Homo sapiens
SQAGAPQGR

SEQ ID NO: 187 - protein, 7 aa, Homo sapiens
HIANVER

SEQ ID NO: 188 - protein, 12 aa, Homo sapiens
LAGAGLELQLER

SEQ ID NO: 189 - protein, 10 aa, Homo sapiens
GAGLELQLER

SEQ ID NO: 190 - protein, 8 aa, Homo sapiens
VDDTQFVR

SEQ ID NO: 191 - protein, 7 aa, Homo sapiens
LLPGSVR

SEQ ID NO: 192 - protein, 24 aa, Homo sapiens
LSAPVVSIHPPQLTVQPGQLAEFR

SEQ ID NO: 193 - protein, 9 aa, Homo sapiens
SSAGQQVAR

SEQ ID NO: 194 - protein, 14 aa, Homo sapiens
ANLAYPAGSTQPIR

SEQ ID NO: 195 - protein, 12 aa, Homo sapiens
LAYPAGSTQPIR

SEQ ID NO: 196 - protein, 10 aa, Homo sapiens
VADSGEYVCR

SEQ ID NO: 197 - protein, 8 aa, Homo sapiens
SPLPWQHR

SEQ ID NO: 198 - protein, 8 aa, Homo sapiens
SKVGGHLR

SEQ ID NO: 199 - protein, 19 aa, Homo sapiens
SKVGGHLRPGIVQSGGVVR

SEQ ID NO: 200 - protein, 13 aa, Homo sapiens
IAHVELADAGQYR

SEQ ID NO: 201 - protein, 15 aa, Homo sapiens
QGKVKAFAHLQVPER

SEQ ID NO: 202 - protein, 13 aa, Homo sapiens
SFLPLPTIKDAYR

SEQ ID NO: 203 - protein, 9 aa, Homo sapiens
SSGFIGCVR

SEQ ID NO: 204 - protein, 24 aa, Homo sapiens
SGGKSGPVEDFVSLAMVGGHLEFR

SEQ ID NO: 205 - protein, 9 aa, Homo sapiens
SAEPLALGR

SEQ ID NO: 206 - protein, 8 aa, Homo sapiens
LNKDGSLR

SEQ ID NO: 207 - protein, 12 aa, Homo sapiens
GCVGEVSVNGKR

SEQ ID NO: 208 - protein, 18 aa, Homo sapiens
DGFKGDLCEHEENPCQLR

SEQ ID NO: 209 - protein, 8 aa, Homo sapiens
LAAGCPDR

SEQ ID NO: 210 - protein, 9 aa, Homo sapiens
YKTGNLYLR

SEQ ID NO: 211 - protein, 9 aa, Homo sapiens
SAGLGPVVR

SEQ ID NO: 212 - protein, 8 aa, Homo sapiens
AGLGPVVR

SEQ ID NO: 213 - protein, 7 aa, Homo sapiens
FSSESKR

SEQ ID NO: 214 - protein, 16 aa, Homo sapiens
AAGGPGVSGVCVCKSR

SEQ ID NO: 215 - protein, 15 aa, Homo sapiens
GSHLVEALYLVCGER

SEQ ID NO: 216 - protein, 6 aa, Homo sapiens
LVELSR

SEQ ID NO: 217 - protein, 24 aa, Homo sapiens
QVLPAGQIVNCDCSAAGTQGNACR

SEQ ID NO: 218 - protein, 8 aa, Homo sapiens
HEEFEKSR

SEQ ID NO: 219 - protein, 22 aa, Homo sapiens
HGDANTIVCNSKDGGAWGTEQR

SEQ ID NO: 220 - protein, 8 aa, Homo sapiens
FQAPSDYR

SEQ ID NO: 221 - protein, 9 aa, Homo sapiens
QTEVKPSVR

SEQ ID NO: 222 - protein, 12 aa, Homo sapiens
QTQGGGSVTKKR

SEQ ID NO: 223 - protein, 11 aa, Homo sapiens
LSLGSQYQPQR

SEQ ID NO: 224 - protein, 9 aa, Homo sapiens
ANASAQQPR

SEQ ID NO: 225 - protein, 8 aa, Homo sapiens
NASAQQPR

SEQ ID NO: 226 - protein, 11 aa, Homo sapiens
HWFQAGYSTSR

SEQ ID NO: 227 - protein, 10 aa, Homo sapiens
WFQAGYSTSR

SEQ ID NO: 228 - protein, 8 aa, Homo sapiens
QAGYSTSR

SEQ ID NO: 229 - protein, 13 aa, Homo sapiens
VASAGVIYPYQPR

SEQ ID NO: 230 - protein, 23 aa, Homo sapiens
FQQPAPEYHQPQAPANVAKIQLR

SEQ ID NO: 231 - protein, 22 aa, Homo sapiens
QQPAPEYHQPQAPANVAKIQLR

SEQ ID NO: 232 - protein, 10 aa, Homo sapiens
AYKPEQPLVR

SEQ ID NO: 233 - protein, 9 aa, Homo sapiens
QSLGTTKLR

SEQ ID NO: 234 - protein, 7 aa, Homo sapiens
LTATNFR

SEQ ID NO: 235 - protein, 9 aa, Homo sapiens
SVQIHQVSR

SEQ ID NO: 236 - protein, 19 aa, Homo sapiens
HQQVIPHVYPVAAKTQLGR

SEQ ID NO: 237 - protein, 13 aa, Homo sapiens
HVYPVAAKTQLGR

SEQ ID NO: 238 - protein, 16 aa, Homo sapiens
SEQYTPEADPYFIQDR

SEQ ID NO: 239 - protein, 11 aa, Homo sapiens
SVQIHQVAQVR

SEQ ID NO: 240 - protein, 14 aa, Homo sapiens
SGEPPRPLPPAAPR

SEQ ID NO: 241 - protein, 14 aa, Homo sapiens
SLGPGTCTLPLAQR

SEQ ID NO: 242 - protein, 11 aa, Homo sapiens
SSGALPGPAER

SEQ ID NO: 243 - protein, 11 aa, Homo sapiens
GKCTNLEGSFR

SEQ ID NO: 244 - protein, 15 aa, Homo sapiens
SGQKGHAPCSSVLGR

SEQ ID NO: 245 - protein, 24 aa, Homo sapiens
TTYTECCCQDGEAWSQQCALCPPR

SEQ ID NO: 246 - protein, 13 aa, Homo sapiens
SSEVYAQLCNVAR

SEQ ID NO: 247 - protein, 7 aa, Homo sapiens
EAGVHFR

SEQ ID NO: 248 - protein, 6 aa, Homo sapiens
SVQVHR

SEQ ID NO: 249 - protein, 6 aa, Homo sapiens
VHQVER

SEQ ID NO: 250 - protein, 10 aa, Homo sapiens
LQEKIDHLER

SEQ ID NO: 251 - protein, 15 aa, Homo sapiens
LTTPTKQLGAQSPGR

SEQ ID NO: 252 - protein, 10 aa, Homo sapiens
AVASTGPSSR

SEQ ID NO: 253 - protein, 9 aa, Homo sapiens
VASTGPSSR

SEQ ID NO: 254 - protein, 14 aa, Homo sapiens
LSGQKGDVVDVYQR

SEQ ID NO: 255 - protein, 26 aa, Homo sapiens
LETQEQDVDLVQKYLEKYYNLKNDGR

SEQ ID NO: 256 - protein, 27 aa, Homo sapiens
GHAMGLEHSQDPGALMAPIYTYTKNFR

SEQ ID NO: 257 - protein, 6 aa, Homo sapiens
IASLTR

SEQ ID NO: 258 - protein, 13 aa, Homo sapiens
NSSELVKCLIHPR

SEQ ID NO: 259 - protein, 8 aa, Homo sapiens
HSSVPLGR

SEQ ID NO: 260 - protein, 16 aa, Homo sapiens
VGGPDLKGQVEPWDER

SEQ ID NO: 261 - protein, 27 aa, Homo sapiens
LEDNIGSNTEVFTYNAANKETCEHNHR

SEQ ID NO: 262 - protein, 16 aa, Homo sapiens
FTYNAANKETCEHNHR

SEQ ID NO: 263 - protein, 7 aa, Homo sapiens
YAYPGAR

SEQ ID NO: 264 - protein, 15 aa, Homo sapiens
LKEVWEELDGLDPNR

SEQ ID NO: 265 - protein, 7 aa, Homo sapiens
FLASTQR

SEQ ID NO: 266 - protein, 11 aa, Homo sapiens
LQQAVLHMEQR

SEQ ID NO: 267 - protein, 17 aa, Homo sapiens
QSQSSDVVIVSSKYKQR

SEQ ID NO: 268 - protein, 16 aa, Homo sapiens
SQSSDVVIVSSKYKQR

SEQ ID NO: 269 - protein, 10 aa, Homo sapiens
AAGPATCATR

SEQ ID NO: 270 - protein, 14 aa, Homo sapiens
IDPEIQDGSPTTSR

SEQ ID NO: 271 - protein, 6 aa, Homo sapiens
LSAITR

SEQ ID NO: 272 - protein, 6 aa, Homo sapiens
HSVGDR

SEQ ID NO: 273 - protein, 13 aa, Homo sapiens
LFCSHWSHIPGFK

SEQ ID NO: 274 - protein, 11 aa, Homo sapiens
FSDDTLSMDQR

SEQ ID NO: 275 - protein, 11 aa, Homo sapiens
IITGPEIKYTR

SEQ ID NO: 276 - protein, 17 aa, Homo sapiens
FIEGGDGHLFEDEEIKR

SEQ ID NO: 277 - protein, 9 aa, Homo sapiens
LLQGDTPVR

SEQ ID NO: 278 - protein, 6 aa, Homo sapiens
IVEISR

SEQ ID NO: 279 - protein, 7 aa, Homo sapiens
LSETLTR

SEQ ID NO: 280 - protein, 6 aa, Homo sapiens
IGTLTR

SEQ ID NO: 281 - protein, 16 aa, Homo sapiens
FMANIPLLLYPQDGPR

SEQ ID NO: 282 - protein, 12 aa, Homo sapiens
SYQEDKPTKKTR

SEQ ID NO: 283 - protein, 8 aa, Homo sapiens
QKTITDLR

SEQ ID NO: 284 - protein, 17 aa, Homo sapiens
VAAAPELAMGALELESR

SEQ ID NO: 285 - protein, 6 aa, Homo sapiens
ITHSVR

SEQ ID NO: 286 - protein, 6 aa, Homo sapiens
LASITR

SEQ ID NO: 287 - protein, 10 aa, Homo sapiens
LEENEVIPKR

SEQ ID NO: 288 - protein, 16 aa, Homo sapiens
QAAAQTNSNAAGKQLR

SEQ ID NO: 289 - protein, 12 aa, Homo sapiens
QTNSNAAGKQLR

SEQ ID NO: 290 - protein, 11 aa, Homo sapiens
QLQGEGKIIDR

SEQ ID NO: 291 - protein, 20 aa, Homo sapiens
AAAPGTAEKLSPKAATLAER

SEQ ID NO: 292 - protein, 15 aa, Homo sapiens
LISDRDPQCNLHCSR

SEQ ID NO: 293 - protein, 10 aa, Homo sapiens
HWVGPSNLVK

SEQ ID NO: 294 - protein, 20 aa, Homo sapiens
LEEKGPMFELLPGESNKIPR

SEQ ID NO: 295 - protein, 19 aa, Homo sapiens
EEKGPMFELLPGESNKIPR

SEQ ID NO: 296 - protein, 11 aa, Homo sapiens
LLPGESNKIPR

SEQ ID NO: 297 - protein, 10 aa, Homo sapiens
LPGESNKIPR

SEQ ID NO: 298 - protein, 8 aa, Homo sapiens
GESNKIPR

SEQ ID NO: 299 - protein, 6 aa, Homo sapiens
FHDNLR

SEQ ID NO: 300 - protein, 8 aa, Homo sapiens
QNSAEVVR

SEQ ID NO: 301 - protein, 14 aa, Homo sapiens
HGEAGHHLPEPSSR

SEQ ID NO: 302 - protein, 9 aa, Homo sapiens
HHLPEPSSR

SEQ ID NO: 303 - protein, 6 aa, Homo sapiens
ITGLTR

SEQ ID NO: 304 - protein, 6 aa, Homo sapiens
LSALTR

SEQ ID NO: 305 - protein, 16 aa, Homo sapiens
LQEGKHVIGLQMGSNR

SEQ ID NO: 306 - protein, 6 aa, Homo sapiens
QMGSNR

SEQ ID NO: 307 - protein, 16 aa, Homo sapiens
LQEGKNVIGLQMGTNR

SEQ ID NO: 308 - protein, 7 aa, Homo sapiens
GASALLR

SEQ ID NO: 309 - protein, 15 aa, Homo sapiens
GPAKSPYQLVLQHSR

SEQ ID NO: 310 - protein, 17 aa, Homo sapiens
QHGPNVCAVQKVIGTNR

SEQ ID NO: 311 - protein, 10 aa, Homo sapiens
AVQKVIGTNR

SEQ ID NO: 312 - protein, 9 aa, Homo sapiens
GIVTVNCAR

SEQ ID NO: 313 - protein, 20 aa, Homo sapiens
TLLAPTNEAFEKIPSETLNR

SEQ ID NO: 314 - protein, 18 aa, Homo sapiens
VLTPPMGTVMDVLKGDNR

SEQ ID NO: 315 - protein, 10 aa, Homo sapiens
SAGLTETLNR

SEQ ID NO: 316 - protein, 15 aa, Homo sapiens
EGVYTVFAPTNEAFR

SEQ ID NO: 317 - protein, 9 aa, Homo sapiens
QNDPNWVVR

SEQ ID NO: 318 - protein, 7 aa, Homo sapiens
LSENLKR

SEQ ID NO: 319 - protein, 10 aa, Homo sapiens
HTSALCNSCR

SEQ ID NO: 320 - protein, 9 aa, Homo sapiens
LLDAPEHFR

SEQ ID NO: 321 - protein, 6 aa, Homo sapiens
LGTLTR

SEQ ID NO: 322 - protein, 12 aa, Homo sapiens
HIQPGAFDTLDR

SEQ ID NO: 323 - protein, 8 aa, Homo sapiens
AIAGAKLR

SEQ ID NO: 324 - protein, 7 aa, Homo sapiens
QESTEYR

SEQ ID NO: 325 - protein, 10 aa, Homo sapiens
LKGTNESLER

SEQ ID NO: 326 - protein, 8 aa, Homo sapiens
LVDTHSKR

SEQ ID NO: 327 - protein, 25 aa, Homo sapiens
HAANQSYQYGPSSAGNGAGGGGSMG

SEQ ID NO: 328 - protein, 10 aa, Homo sapiens
FKVETSNKVL

SEQ ID NO: 329 - protein, 13 aa, Homo sapiens
VIQAEGKEHIIHL

SEQ ID NO: 330 - protein, 7 aa, Homo sapiens
VIQAEGK

SEQ ID NO: 331 - protein, 10 aa, Homo sapiens
GKEHIIHLER

SEQ ID NO: 332 - protein, 7 aa, Homo sapiens
HVVGPVR

SEQ ID NO: 333 - protein, 9 aa, Homo sapiens
SQPVGPVNK

SEQ ID NO: 334 - protein, 15 aa, Homo sapiens
FTQGKVVGQTTPESF

SEQ ID NO: 335 - protein, 12 aa, Homo sapiens
LAGTTIRATACH

SEQ ID NO: 336 - protein, 8 aa, Homo sapiens
LVETHLAR

SEQ ID NO: 337 - protein, 11 aa, Homo sapiens
EQLGPVTQEFW

SEQ ID NO: 338 - protein, 41 aa, Homo sapiens
PITAKAIAKGVGIISEGNETVEDIAARLNIPMSQVNPREAK

SEQ ID NO: 339 - protein, 6 aa, Homo sapiens
DFIALR

SEQ ID NO: 340 - protein, 9 aa, Homo sapiens
LKSAPLFML

SEQ ID NO: 341 - protein, 12 aa, Homo sapiens
LTGSVAKCENEG

SEQ ID NO: 342 - protein, 10 aa, Homo sapiens
FTAFLHPEEY

SEQ ID NO: 343 - protein, 14 aa, Homo sapiens
FSAPKPQTSPSPKR

SEQ ID NO: 344 - protein, 8 aa, Homo sapiens
ASQGPVYK

SEQ ID NO: 345 - protein, 11 aa, Homo sapiens
QVGNVPLKKAK

SEQ ID NO: 346 - protein, 14 aa, Homo sapiens
INAPVTCCYNFTNR

SEQ ID NO: 347 - protein, 7 aa, Homo sapiens
DKSTAGR

SEQ ID NO: 348 - protein, 8 aa, Homo sapiens
KGGNTMTG

SEQ ID NO: 349 - protein, 9 aa, Homo sapiens
LQPPVVQLH

SEQ ID NO: 350 - protein, 20 aa, Homo sapiens
FQGPPGEPGEPGASGPMGPR

SEQ ID NO: 351 - protein, 15 aa, Homo sapiens
LTGPIGPPGPAGAPG

SEQ ID NO: 352 - protein, 9 aa, Homo sapiens
LSQQIENIR

SEQ ID NO: 353 - protein, 11 aa, Homo sapiens
LLQGSNEIEIR

SEQ ID NO: 354 - protein, 9 aa, Homo sapiens
FQGPAGEPG

SEQ ID NO: 355 - protein, 8 aa, Homo sapiens
VMGPPGSR

SEQ ID NO: 356 - protein, 10 aa, Homo sapiens
FGLPGPAGPR

SEQ ID NO: 357 - protein, 17 aa, Homo sapiens
FAGPAGAAGQPGAKGER

SEQ ID NO: 358 - protein, 13 aa, Homo sapiens
LGAPGILGLPGSR

SEQ ID NO: 359 - protein, 17 aa, Homo sapiens
AVGEPGPLGIAGPPGAR

SEQ ID NO: 360 - protein, 15 aa, Homo sapiens
VGSPGVNGAPGEAGR

SEQ ID NO: 361 - protein, 12 aa, Homo sapiens
LQGLPGIAGHHG

SEQ ID NO: 362 - protein, 24 aa, Homo sapiens
HQGPAGPPGPPGPPGPPGVSGGGY

SEQ ID NO: 363 - protein, 8 aa, Homo sapiens
LLTPEGSR

SEQ ID NO: 364 - protein, 14 aa, Homo sapiens
QQGAIGSPGPAGPR

SEQ ID NO: 365 - protein, 25 aa, Homo sapiens
HQGPIGQEGAPGRPGSPGLPGMPGR

SEQ ID NO: 366 - protein, 13 aa, Homo sapiens
QESQAQAILQQAR

SEQ ID NO: 367 - protein, 22 aa, Homo sapiens
LTGNPGVQGPEGKLGPLGAPGE

SEQ ID NO: 368 - protein, 8 aa, Homo sapiens
FQGCPGQR

SEQ ID NO: 369 - protein, 6 aa, Homo sapiens
ISSSQR

SEQ ID NO: 370 - protein, 10 aa, Homo sapiens
WAFGAVEAIS

SEQ ID NO: 371 - protein, 8 aa, Homo sapiens
APVEPATA

SEQ ID NO: 372 - protein, 6 aa, Homo sapiens
AGLAPR

SEQ ID NO: 373 - protein, 21 aa, Homo sapiens
FPELKGIIFMQTACQAVQHQR

SEQ ID NO: 374 - protein, 16 aa, Homo sapiens
FQEVGYAAPPSPPLSR

SEQ ID NO: 375 - protein, 10 aa, Homo sapiens
AAPPSPPLSR

SEQ ID NO: 376 - protein, 8 aa, Homo sapiens
QEAVPLQK

SEQ ID NO: 377 - protein, 8 aa, Homo sapiens
QYSGAQVR

SEQ ID NO: 378 - protein, 10 aa, Homo sapiens
QTGRNNFVIR

SEQ ID NO: 379 - protein, 10 aa, Homo sapiens
QTGTSTTSSR

SEQ ID NO: 380 - protein, 19 aa, Homo sapiens
QISLRPAQSFLMNKNQVPK

SEQ ID NO: 381 - protein, 5 aa, Homo sapiens
QIAPR

SEQ ID NO: 382 - protein, 8 aa, Homo sapiens
QVSSQGSR

SEQ ID NO: 383 - protein, 10 aa, Homo sapiens
QTVVQVLAVK

SEQ ID NO: 384 - protein, 7 aa, Homo sapiens
LSENKIR

SEQ ID NO: 385 - protein, 6 aa, Homo sapiens
IKLPER

SEQ ID NO: 386 - protein, 20 aa, Homo sapiens
FSQVASNTIPLPLPQPNTCK

SEQ ID NO: 387 - protein, 7 aa, Homo sapiens
LEAGNVK

SEQ ID NO: 388 - protein, 28 aa, Homo sapiens
RKNFLQNVANAFACVIMEMLQSIMSGDR

SEQ ID NO: 389 - protein, 11 aa, Homo sapiens
LQSSLRKWNPL

SEQ ID NO: 390 - protein, 9 aa, Homo sapiens
VNKPAEFTV

SEQ ID NO: 391 - protein, 10 aa, Homo sapiens
FVGQKNSFTV

SEQ ID NO: 392 - protein, 19 aa, Homo sapiens
QMVQPQSPVAVSQSKPGCY

SEQ ID NO: 393 - protein, 18 aa, Homo sapiens
MVQPQSPVAVSQSKPGCY

SEQ ID NO: 394 - protein, 14 aa, Homo sapiens
QSPVAVSQSKPGCY

SEQ ID NO: 395 - protein, 9 aa, Homo sapiens
DKYTGNTYR

SEQ ID NO: 396 - protein, 9 aa, Homo sapiens
TSSGSGPFT

SEQ ID NO: 397 - protein, 11 aa, Homo sapiens
SSSGPVEVFIT

SEQ ID NO: 398 - protein, 18 aa, Homo sapiens
LQPTVEYVVSVYAQNPSG

SEQ ID NO: 399 - protein, 9 aa, Homo sapiens
LVQTAVTNI

SEQ ID NO: 400 - protein, 18 aa, Homo sapiens
LQGLRPGSEYTVSVVALH

SEQ ID NO: 401 - protein, 13 aa, Homo sapiens
LIGTQSTAIPAPT

SEQ ID NO: 402 - protein, 7 aa, Homo sapiens
FVTHPGY

SEQ ID NO: 403 - protein, 8 aa, Homo sapiens
SVGQQMIF

SEQ ID NO: 404 - protein, 12 aa, Homo sapiens
LNKCFVKVPRHY

SEQ ID NO: 405 - protein, 8 aa, Homo sapiens
LQKHQETA

SEQ ID NO: 406 - protein, 6 aa, Homo sapiens
FKVDAC

SEQ ID NO: 407 - protein, 10 aa, Homo sapiens
LAGAGLELQL

SEQ ID NO: 408 - protein, 8 aa, Homo sapiens
GLELQLER

SEQ ID NO: 409 - protein, 9 aa, Homo sapiens
FQTKSGTAS

SEQ ID NO: 410 - protein, 14 aa, Homo sapiens
LLPGSVRPLPCGPQ

SEQ ID NO: 411 - protein, 21 aa, Homo sapiens
LSAPVVSIHPPQLTVQPGQLA

SEQ ID NO: 412 - protein, 10 aa, Homo sapiens
HSSAGQQVAR

SEQ ID NO: 413 - protein, 7 aa, Homo sapiens
GVAYPVR

SEQ ID NO: 414 - protein, 17 aa, Homo sapiens
LAQNALGTAQKQVEVIV

SEQ ID NO: 415 - protein, 9 aa, Homo sapiens
FAHLQVPER

SEQ ID NO: 416 - protein, 6 aa, Homo sapiens
EKYSFE

SEQ ID NO: 417 - protein, 6 aa, Homo sapiens
GALAPR

SEQ ID NO: 418 - protein, 21 aa, Homo sapiens
RLAKSGTTNAFLQYKQNGGLE

SEQ ID NO: 419 - protein, 9 aa, Homo sapiens
SLQFSNGSR

SEQ ID NO: 420 - protein, 11 aa, Homo sapiens
QVLPAGQIVNC

SEQ ID NO: 421 - protein, 7 aa, Homo sapiens
QSYGGYK

SEQ ID NO: 422 - protein, 16 aa, Homo sapiens
SYGGYKEPAAPVSIQR

SEQ ID NO: 423 - protein, 9 aa, Homo sapiens
LGSQYQPQR

SEQ ID NO: 424 - protein, 9 aa, Homo sapiens
FQAGYSTSR

SEQ ID NO: 425 - protein, 12 aa, Homo sapiens
SVSASAFASTYR

SEQ ID NO: 426 - protein, 14 aa, Homo sapiens
AVASAGVIYPYQPR

SEQ ID NO: 427 - protein, 15 aa, Homo sapiens
HQPQAPANVAKIQLR

SEQ ID NO: 428 - protein, 13 aa, Homo sapiens
QVSGRPPVIKPEV

SEQ ID NO: 429 - protein, 8 aa, Homo sapiens
SEQYTPEA

SEQ ID NO: 430 - protein, 9 aa, Homo sapiens
VAGLQPVER

SEQ ID NO: 431 - protein, 10 aa, Homo sapiens
SAAGEGTLAR

SEQ ID NO: 432 - protein, 7 aa, Homo sapiens
QHVGLSR

SEQ ID NO: 433 - protein, 23 aa, Homo sapiens
LVENSVETRPPPWLPASPGHSLW

SEQ ID NO: 434 - protein, 7 aa, Homo sapiens
LTGSGFR

SEQ ID NO: 435 - protein, 12 aa, Homo sapiens
FLVPLGPGQISA

SEQ ID NO: 436 - protein, 9 aa, Homo sapiens
LLTGEIPYK

SEQ ID NO: 437 - protein, 6 aa, Homo sapiens
AESGAK

SEQ ID NO: 438 - protein, 13 aa, Homo sapiens
SLEKHMLSHTEER

SEQ ID NO: 439 - protein, 6 aa, Homo sapiens
EYKSFE

SEQ ID NO: 440 - protein, 8 aa, Homo sapiens
FQYLLNHR

SEQ ID NO: 441 - protein, 14 aa, Homo sapiens
VVIMKRSLRTSAKR

SEQ ID NO: 442 - protein, 9 aa, Homo sapiens
FVLTEGNPR

SEQ ID NO: 443 - protein, 9 aa, Homo sapiens
FQPGPGIGG

SEQ ID NO: 444 - protein, 13 aa, Homo sapiens
LMAPIYTYTKNFR

SEQ ID NO: 445 - protein, 9 aa, Homo sapiens
LEKVAPLLR

SEQ ID NO: 446 - protein, 6 aa, Homo sapiens
KYESFE

SEQ ID NO: 447 - protein, 11 aa, Homo sapiens
FQAGLHNALMK

SEQ ID NO: 448 - protein, 7 aa, Homo sapiens
FNELSHK

SEQ ID NO: 449 - protein, 6 aa, Homo sapiens
LSSSQR

SEQ ID NO: 450 - protein, 9 aa, Homo sapiens
FHQQHPQVI

SEQ ID NO: 451 - protein, 8 aa, Homo sapiens
FTYNAANK

SEQ ID NO: 452 - protein, 9 aa, Homo sapiens
HSEAGGGSG

SEQ ID NO: 453 - protein, 12 aa, Homo sapiens
VPGTLIALIKIH

SEQ ID NO: 454 - protein, 12 aa, Homo sapiens
FTASPASSTTAR

SEQ ID NO: 455 - protein, 7 aa, Homo sapiens
ASSTTAR

SEQ ID NO: 456 - protein, 12 aa, Homo sapiens
LSAGKTAVNVPR

SEQ ID NO: 457 - protein, 11 aa, Homo sapiens
VSGAQPILSKL

SEQ ID NO: 458 - protein, 12 aa, Homo sapiens
LLGAARNPRRGR

SEQ ID NO: 459 - protein, 17 aa, Homo sapiens
MRYEGVVDMFQTVKTLR

SEQ ID NO: 460 - protein, 7 aa, Homo sapiens
HKGVAVR

SEQ ID NO: 461 - protein, 12 aa, Homo sapiens
QKQPTIFQNKKR

SEQ ID NO: 462 - protein, 9 aa, Homo sapiens
QLQGEGKII

SEQ ID NO: 463 - protein, 14 aa, Homo sapiens
VAKDPINQRACHIL

SEQ ID NO: 464 - protein, 6 aa, Homo sapiens
KAPSLE

SEQ ID NO: 465 - protein, 6 aa, Homo sapiens
FHGCIH

SEQ ID NO: 466 - protein, 13 aa, Homo sapiens
LAGGAGPNHGNFL

SEQ ID NO: 467 - protein, 7 aa, Homo sapiens
SILPICK

SEQ ID NO: 468 - protein, 12 aa, Homo sapiens
KNGTSSNKNKED

SEQ ID NO: 469 - protein, 14 aa, Homo sapiens
LTRIRQGWERNSKH

SEQ ID NO: 470 - protein, 6 aa, Homo sapiens
LNSVFK

SEQ ID NO: 471 - protein, 9 aa, Homo sapiens
AKASVPTIQ

SEQ ID NO: 472 - protein, 16 aa, Homo sapiens
PEHHSVPGEGMRHPWK

SEQ ID NO: 473 - protein, 23 aa, Homo sapiens
VQMVLNNLVEIINRPNTPKTLLE

SEQ ID NO: 474 - protein, 9 aa, Homo sapiens
QEGLAGYQR

SEQ ID NO: 475 - protein, 10 aa, Homo sapiens
LSTVGSAISR

SEQ ID NO: 476 - protein, 11 aa, Homo sapiens
HRCIVRHENNK

SEQ ID NO: 477 - protein, 12 aa, Homo sapiens
VKEPPVEFTKPL

SEQ ID NO: 478 - protein, 8 aa, Homo sapiens
AASPPLLR

SEQ ID NO: 479 - protein, 6 aa, Homo sapiens
YTQATH

SEQ ID NO: 480 - protein, 8 aa, Homo sapiens
SEQQVAAR

SEQ ID NO: 481 - protein, 8 aa, Homo sapiens
FKNSSSAK

SEQ ID NO: 482 - protein, 10 aa, Homo sapiens
YESVAAKNLQ

SEQ ID NO: 483 - protein, 9 aa, Homo sapiens
QVQSLTCEV

SEQ ID NO: 484 - protein, 6 aa, Homo sapiens
KDGHQR

SEQ ID NO: 485 - protein, 11 aa, Homo sapiens
FQQSQGPAVQR

SEQ ID NO: 486 - protein, 9 aa, Homo sapiens
QQAGSKTVR

SEQ ID NO: 487 - protein, 6 aa, Homo sapiens
AQGAPR

SEQ ID NO: 488 - protein, 7 aa, Homo sapiens
LSSAGFR

SEQ ID NO: 489 - protein, 22 aa, Homo sapiens
NNDDKMNGHIHGPLVKLNGDYR

SEQ ID NO: 490 - protein, 9 aa, Homo sapiens
QSSSGFLYR

SEQ ID NO: 491 - protein, 10 aa, Homo sapiens
FQHPPTEFIR

SEQ ID NO: 492 - protein, 8 aa, Homo sapiens
DKFQGVIA

SEQ ID NO: 493 - protein, 10 aa, Homo sapiens
EETGAALKPR

SEQ ID NO: 494 - protein, 8 aa, Homo sapiens
TGAALKPR

SEQ ID NO: 495 - protein, 7 aa, Homo sapiens
KVQLEAR

SEQ ID NO: 496 - protein, 12 aa, Homo sapiens
VAALQPPVVQLH

SEQ ID NO: 497 - protein, 10 aa, Homo sapiens
ALQPPVVQLH

SEQ ID NO: 498 - protein, 9 aa, Homo sapiens
AAVPIVNLK

SEQ ID NO: 499 - protein, 16 aa, Homo sapiens
EALFSGSEGPLKPGAR

SEQ ID NO: 500 - protein, 12 aa, Homo sapiens
SGSEGPLKPGAR

SEQ ID NO: 501 - protein, 17 aa, Homo sapiens
TEAPSATGQASSLLGGR

SEQ ID NO: 502 - protein, 8 aa, Homo sapiens
AGSGCGKC

SEQ ID NO: 503 - protein, 11 aa, Homo sapiens
TVVGPALAAYR

SEQ ID NO: 504 - protein, 10 aa, Homo sapiens
VVGPALAAYR

SEQ ID NO: 505 - protein, 6 aa, Homo sapiens
ALAAYR

SEQ ID NO: 506 - protein, 10 aa, Homo sapiens
TFGPDPTMIR

SEQ ID NO: 507 - protein, 25 aa, Homo sapiens
NPEHFLDANQSFKKSPAFMPFSAGR

SEQ ID NO: 508 - protein, 23 aa, Homo sapiens
AAPAPVPVPAASLRPLSPGTPSG

SEQ ID NO: 509 - protein, 6 aa, Homo sapiens
AQAGPR

SEQ ID NO: 510 - protein, 6 aa, Homo sapiens
EMQTGR

SEQ ID NO: 511 - protein, 7 aa, Homo sapiens
ISENLKR

SEQ ID NO: 512 - protein, 13 aa, Homo sapiens
YSQPVQGYGTGAY

SEQ ID NO: 513 - protein, 9 aa, Homo sapiens
EPSFPEIPL

SEQ ID NO: 514 - protein, 9 aa, Homo sapiens
STGASKSKR

SEQ ID NO: 515 - protein, 6 aa, Homo sapiens
STGASK

SEQ ID NO: 516 - protein, 20 aa, Homo sapiens
QQMVQPQSPVAVSQSKPGCY

SEQ ID NO: 517 - protein, 18 aa, Homo sapiens
VYQPQPHPQPPPYGHCVT

SEQ ID NO: 518 - protein, 14 aa, Homo sapiens
TPGVEYVYTIQVLR

SEQ ID NO: 519 - protein, 13 aa, Homo sapiens
LVQTAVTTIPAPT

SEQ ID NO: 520 - protein, 10 aa, Homo sapiens
WESPQGQVSR

SEQ ID NO: 521 - protein, 12 aa, Homo sapiens
HAHLHTPGNCSR

SEQ ID NO: 522 - protein, 7 aa, Homo sapiens
LSEPQPR

SEQ ID NO: 523 - protein, 12 aa, Homo sapiens
QGQPAAPAPQKR

SEQ ID NO: 524 - protein, 13 aa, Homo sapiens
NLAYPAGSTQPIR

SEQ ID NO: 525 - protein, 9 aa, Homo sapiens
ALGVTPTVR

SEQ ID NO: 526 - protein, 6 aa, Homo sapiens
VAYPVR

SEQ ID NO: 527 - protein, 6 aa, Homo sapiens
VAGGAR

SEQ ID NO: 528 - protein, 6 aa, Homo sapiens
NAPPIR

SEQ ID NO: 529 - protein, 8 aa, Homo sapiens
LEALPAGK

SEQ ID NO: 530 - protein, 5 aa, Homo sapiens
AQQPR

SEQ ID NO: 531 - protein, 7 aa, Homo sapiens
QQQPAPR

SEQ ID NO: 532 - protein, 7 aa, Homo sapiens
GEGTLAR

SEQ ID NO: 533 - protein, 6 aa, Homo sapiens
HVGLSR

SEQ ID NO: 534 - protein, 6 aa, Homo sapiens
LEAPLK

SEQ ID NO: 535 - protein, 13 aa, Homo sapiens
LSTGALPPLAPEG

SEQ ID NO: 536 - protein, 15 aa, Homo sapiens
HAAFLVPLGPGQISA

SEQ ID NO: 537 - protein, 11 aa, Homo sapiens
VQAPPPVVNVR

SEQ ID NO: 538 - protein, 8 aa, Homo sapiens
LEQPEVER

SEQ ID NO: 539 - protein, 11 aa, Homo sapiens
HLEGVINKPEA

SEQ ID NO: 540 - protein, 11 aa, Homo sapiens
HAFAPGTGVGG

SEQ ID NO: 541 - protein, 7 aa, Homo sapiens
LIHTNQA

SEQ ID NO: 542 - protein, 9 aa, Homo sapiens
LQKGLTEFV

SEQ ID NO: 543 - protein, 7 aa, Homo sapiens
LIQTATR

SEQ ID NO: 544 - protein, 8 aa, Homo sapiens
LQQAVLHM

SEQ ID NO: 545 - protein, 9 aa, Homo sapiens
AAAATPAVR

SEQ ID NO: 546 - protein, 9 aa, Homo sapiens
QITGTEHLK

SEQ ID NO: 547 - protein, 6 aa, Homo sapiens
AGAQPR

SEQ ID NO: 548 - protein, 8 aa, Homo sapiens
LVEPIKKH

SEQ ID NO: 549 - protein, 7 aa, Homo sapiens
AAAAAGR

SEQ ID NO: 550 - protein, 17 aa, Homo sapiens
HAHLQVAAAPQSLCALR

SEQ ID NO: 551 - protein, 7 aa, Homo sapiens
EGGTLAR

SEQ ID NO: 552 - protein, 6 aa, Homo sapiens
NAPLPR

SEQ ID NO: 553 - protein, 6 aa, Homo sapiens
AAGQPR

SEQ ID NO: 554 - protein, 7 aa, Homo sapiens
SFQVWNK

SEQ ID NO: 555 - protein, 7 aa, Homo sapiens
MSSGLPR

SEQ ID NO: 556 - protein, 6 aa, Homo sapiens
HTAVGR

SEQ ID NO: 557 - protein, 12 aa, Homo sapiens
FGQGVEHATANK

SEQ ID NO: 558 - protein, 11 aa, Homo sapiens
STAISCIGAYR

SEQ ID NO: 559 - protein, 6 aa, Homo sapiens
LKNSAC

SEQ ID NO: 560 - protein, 6 aa, Homo sapiens
ASASCH

SEQ ID NO: 561 - protein, 25 aa, Homo sapiens
HGSPNNSERAMRAVGEMCKGPGAAR

SEQ ID NO: 562 - protein, 11 aa, Homo sapiens
QYESVAAKNLQ

SEQ ID NO: 563 - protein, 13 aa, Homo sapiens
LQPLPPRYLFQMH

SEQ ID NO: 564 - protein, 12 aa, Homo sapiens
HLQTHSGEKPFK

SEQ ID NO: 565 - protein, 10 aa, Homo sapiens
PVSLANTQPR

SEQ ID NO: 566 - protein, 8 aa, Mus musculus
DVSTTQAS

SEQ ID NO: 567 - protein, 8 aa, Mus musculus
VSTTQASS

SEQ ID NO: 568 - protein, 8 aa, Mus musculus
STTQASSA

SEQ ID NO: 569 - protein, 8 aa, Mus musculus
TTQASSAF

SEQ ID NO: 570 - protein, 8 aa, Mus musculus
QASSAFYQ

SEQ ID NO: 571 - protein, 8 aa, Mus musculus
ASSAFYQL

SEQ ID NO: 572 - protein, 8 aa, Mus musculus
SSAFYQLQ

SEQ ID NO: 573 - protein, 8 aa, Mus musculus
SAFYQLQY

SEQ ID NO: 574 - protein, 8 aa, Mus musculus
AFYQLQYQ

SEQ ID NO: 575 - protein, 8 aa, Mus musculus
TTNPYLMA

SEQ ID NO: 576 - protein, 8 aa, Mus musculus
TNPYLMAP

SEQ ID NO: 577 - protein, 8 aa, Mus musculus
EIRRHSTL

SEQ ID NO: 578 - protein, 8 aa, Mus musculus
STLGHAHI

SEQ ID NO: 579 - protein, 8 aa, Mus musculus
TLGHAHIQ

SEQ ID NO: 580 - protein, 8 aa, Mus musculus
LGHAHIQT

SEQ ID NO: 581 - protein, 8 aa, Mus musculus
GHAHIQTS

SEQ ID NO: 582 - protein, 8 aa, Mus musculus
HAHIQTSV

SEQ ID NO: 583 - protein, 8 aa, Mus musculus
HLLGDVQD

SEQ ID NO: 584 - protein, 8 aa, Mus musculus
LEGGFAAI

SEQ ID NO: 585 - protein, 8 aa, Mus musculus
GFAAISAC

SEQ ID NO: 586 - protein, 8 aa, Mus musculus
GLRGVFQL

SEQ ID NO: 587 - protein, 8 aa, Mus musculus
LRGVFQLS

SEQ ID NO: 588 - protein, 8 aa, Mus musculus
  source 1..8: note = also present in Homo sapiens
NEDYFIEP

SEQ ID NO: 589 - protein, 8 aa, Mus musculus
FIEPLDGV

SEQ ID NO: 590 - protein, 8 aa, Mus musculus
PLDGVSAQ

SEQ ID NO: 591 - protein, 8 aa, Mus musculus
DGVSAQPG

SEQ ID NO: 592 - protein, 8 aa, Mus musculus
GVSAQPGH

SEQ ID NO: 593 - protein, 8 aa, Mus musculus
SAQPGHAQ

SEQ ID NO: 594 - protein, 8 aa, Mus musculus
DLEQQREH

SEQ ID NO: 595 - protein, 8 aa, Mus musculus
KDVIALPS

SEQ ID NO: 596 - protein, 8 aa, Mus musculus
IALPSVLP

SEQ ID NO: 597 - protein, 8 aa, Mus musculus
YKGKLHKW

SEQ ID NO: 598 - protein, 8 aa, Mus musculus
  source 1..8: note = also present in Homo sapiens
LRDAVVDG

SEQ ID NO: 599 - protein, 8 aa, Mus musculus
VSRTFKET

SEQ ID NO: 600 - protein, 8 aa, Mus musculus
RTFKETEG

SEQ ID NO: 601 - protein, 8 aa, Mus musculus
ETEGQGYV

SEQ ID NO: 602 - protein, 8 aa, Mus musculus
GQGYVDIG

SEQ ID NO: 603 - protein, 8 aa, Mus musculus
QGYVDIGL

SEQ ID NO: 604 - protein, 8 aa, Mus musculus
LIEEVAEA

SEQ ID NO: 605 - protein, 8 aa, Mus musculus
EEVAEAAN

SEQ ID NO: 606 - protein, 8 aa, Mus musculus
EVAEAANF

SEQ ID NO: 607 - protein, 8 aa, Mus musculus
VAEAANFL

SEQ ID NO: 608 - protein, 8 aa, Mus musculus
PDKYFLNG

SEQ ID NO: 609 - protein, 8 aa, Mus musculus
DKYFLNGG

SEQ ID NO: 610 - protein, 8 aa, Mus musculus
  source 1..8: note = also present in Homo sapiens
LNGGWTIQ

SEQ ID NO: 611 - protein, 8 aa, Mus musculus
  source 1..8: note = also present in Homo sapiens
NWENLTSP

SEQ ID NO: 612 - protein, 8 aa, Mus musculus
QLLFQEKN

SEQ ID NO: 613 - protein, 8 aa, Mus musculus
NPGVHYQY

SEQ ID NO: 614 - protein, 8 aa, Mus musculus
GVHYQYTI

SEQ ID NO: 615 - protein, 8 aa, Mus musculus
EFSWHYGP

SEQ ID NO: 616 - protein, 8 aa, Mus musculus
HYGPWSKC

SEQ ID NO: 617 - protein, 8 aa, Mus musculus
VVAEEYCN

SEQ ID NO: 618 - protein, 8 aa, Mus musculus
VAEEYCNT

SEQ ID NO: 619 - protein, 8 aa, Mus musculus
EQRALELS

SEQ ID NO: 620 - protein, 8 aa, Mus musculus
RALELSAC

SEQ ID NO: 621 - protein, 8 aa, Mus musculus
LPRPLAET

SEQ ID NO: 622 - protein, 8 aa, Mus musculus
SPELFNEV

SEQ ID NO: 623 - protein, 8 aa, Mus musculus
FIPNQLAP

SEQ ID NO: 624 - protein, 8 aa, Mus musculus
  source 1..8: note = also present in Homo sapiens
RPSPASSP

SEQ ID NO: 625 - protein, 8 aa, Mus musculus
SPKPVSIS

SEQ ID NO: 626 - protein, 8 aa, Mus musculus
SPSPLLSE

SEQ ID NO: 627 - protein, 8 aa, Mus musculus
LSEASYSP

SEQ ID NO: 628 - protein, 8 aa, Mus musculus
SINPLANF

SEQ ID NO: 629 - protein, 8 aa, Mus musculus
MGAPELGF

SEQ ID NO: 630 - protein, 8 aa, Mus musculus
NPDELLVK

SEQ ID NO: 631 - protein, 8 aa, Mus musculus
HLKTLTMP

SEQ ID NO: 632 - protein, 8 aa, Mus musculus
LTMPGTLL

SEQ ID NO: 633 - protein, 8 aa, Mus musculus
GTLLLTVP

SEQ ID NO: 634 - protein, 8 aa, Mus musculus
EVQPLQPS

SEQ ID NO: 635 - protein, 8 aa, Mus musculus
  source 1..8: note = also present in Homo sapiens
VVGPWGQC

SEQ ID NO: 636 - protein, 8 aa, Mus musculus
DLCSHEAW

SEQ ID NO: 637 - protein, 8 aa, Homo sapiens
DVSVRRDA

SEQ ID NO: 638 - protein, 8 aa, Homo sapiens
VSVRRDAP

SEQ ID NO: 639 - protein, 8 aa, Homo sapiens
SVRRDAPA

SEQ ID NO: 640 - protein, 8 aa, Homo sapiens
VRRDAPAF

SEQ ID NO: 641 - protein, 8 aa, Homo sapiens
RDAPAFYE

SEQ ID NO: 642 - protein, 8 aa, Homo sapiens
DAPAFYEL

SEQ ID NO: 643 - protein, 8 aa, Homo sapiens
APAFYELQ

SEQ ID NO: 644 - protein, 8 aa, Homo sapiens
PAFYELQY

SEQ ID NO: 645 - protein, 8 aa, Homo sapiens
AFYELQYR

SEQ ID NO: 646 - protein, 8 aa, Homo sapiens
TANQHLLA

SEQ ID NO: 647 - protein, 8 aa, Homo sapiens
ANQHLLAP

SEQ ID NO: 648 - protein, 8 aa, Homo sapiens
ETRRRGGL

SEQ ID NO: 649 - protein, 8 aa, Homo sapiens
GGLGRAHI

SEQ ID NO: 650 - protein, 8 aa, Homo sapiens
GLGRAHIR

SEQ ID NO: 651 - protein, 8 aa, Homo sapiens
LGRAHIRA

SEQ ID NO: 652 - protein, 8 aa, Homo sapiens
GRAHIRAH

SEQ ID NO: 653 - protein, 8 aa, Homo sapiens
RAHIRAHT

SEQ ID NO: 654 - protein, 8 aa, Homo sapiens
HLLGEVQD

SEQ ID NO: 655 - protein, 8 aa, Homo sapiens
LEGGLAAI

SEQ ID NO: 656 - protein, 8 aa, Homo sapiens
GLAAISAC

SEQ ID NO: 657 - protein, 8 aa, Homo sapiens
GLKGVFQL

SEQ ID NO: 658 - protein, 8 aa, Homo sapiens
LKGVFQLS

SEQ ID NO: 659 - protein, 8 aa, Homo sapiens
FIEPLDSA

SEQ ID NO: 660 - protein, 8 aa, Homo sapiens
PLDSAPAR

SEQ ID NO: 661 - protein, 8 aa, Homo sapiens
DSAPARPG

SEQ ID NO: 662 - protein, 8 aa, Homo sapiens
SAPARPGH

SEQ ID NO: 663 - protein, 8 aa, Homo sapiens
PARPGHAQ

SEQ ID NO: 664 - protein, 8 aa, Homo sapiens
ELESRRER

SEQ ID NO: 665 - protein, 8 aa, Homo sapiens
KDIIDFPS

SEQ ID NO: 666 - protein, 8 aa, Homo sapiens
IDFPSVPP

SEQ ID NO: 667 - protein, 8 aa, Homo sapiens
YKGQLHTW

SEQ ID NO: 668 - protein, 8 aa, Homo sapiens
VSGTFEEA

SEQ ID NO: 669 - protein, 8 aa, Homo sapiens
GTFEEAEG

SEQ ID NO: 670 - protein, 8 aa, Homo sapiens
EAEGLGYV

SEQ ID NO: 671 - protein, 8 aa, Homo sapiens
GLGYVDVG

SEQ ID NO: 672 - protein, 8 aa, Homo sapiens
LGYVDVGL

SEQ ID NO: 673 - protein, 8 aa, Homo sapiens
RIQEVAEA

SEQ ID NO: 674 - protein, 8 aa, Homo sapiens
QEVAEAAN

SEQ ID NO: 675 - protein, 8 aa, Homo sapiens
PEKYFLNG

SEQ ID NO: 676 - protein, 8 aa, Homo sapiens
EKYFLNGG

SEQ ID NO: 677 - protein, 8 aa, Homo sapiens
QLLFQESN

SEQ ID NO: 678 - protein, 8 aa, Homo sapiens
NPGVHYEY

SEQ ID NO: 679 - protein, 8 aa, Homo sapiens
GVHYEYTI

SEQ ID NO: 680 - protein, 8 aa, Homo sapiens
VFSWHYGP

SEQ ID NO: 681 - protein, 8 aa, Homo sapiens
HYGPWTKC

SEQ ID NO: 682 - protein, 8 aa, Homo sapiens
PVDEEHCD

SEQ ID NO: 683 - protein, 8 aa, Homo sapiens
VDEEHCDP

SEQ ID NO: 684 - protein, 8 aa, Homo sapiens
HCDPLGRP

SEQ ID NO: 685 - protein, 8 aa, Homo sapiens
EQSALEPP

SEQ ID NO: 686 - protein, 8 aa, Homo sapiens
SALEPPAC

SEQ ID NO: 687 - protein, 8 aa, Homo sapiens
LPRPPTET

SEQ ID NO: 688 - protein, 8 aa, Homo sapiens
SHELFNEA

SEQ ID NO: 689 - protein, 8 aa, Homo sapiens
FIPHHLAP

SEQ ID NO: 690 - protein, 8 aa, Homo sapiens
SPKPGTMG

SEQ ID NO: 691 - protein, 8 aa, Homo sapiens
SPSPWPSQ

SEQ ID NO: 692 - protein, 8 aa, Homo sapiens
PSQAGRSP

SEQ ID NO: 693 - protein, 8 aa, Homo sapiens
PGNPLINF

SEQ ID NO: 694 - protein, 8 aa, Homo sapiens
IGAPDLGL

SEQ ID NO: 695 - protein, 8 aa, Homo sapiens
SQNDFPVG

SEQ ID NO: 696 - protein, 8 aa, Homo sapiens
DLQTVAVW

SEQ ID NO: 697 - protein, 8 aa, Homo sapiens
VAVWGTFL

SEQ ID NO: 698 - protein, 8 aa, Homo sapiens
GTFLPTTL

SEQ ID NO: 699 - protein, 8 aa, Homo sapiens
ETQPLAPS

SEQ ID NO: 700 - protein, 8 aa, Homo sapiens
DQCGHEAW

SEQ ID NO: 701 - protein, 8 aa, Homo sapiens
EDCEPVEP

SEQ ID NO: 702 - protein, 8 aa, Homo sapiens
TAAAALGP

SEQ ID NO: 703 - protein, 8 aa, Homo sapiens
AAAALGPP

SEQ ID NO: 704 - protein, 8 aa, Mus musculus
FEEPHPDL

SEQ ID NO: 705 - protein, 4 aa, Homo sapiens
PAAL

SEQ ID NO: 706 - protein, 8 aa, Homo sapiens
TEGEARGS

SEQ ID NO: 707 - protein, 8 aa, Homo sapiens
TAQEAGEG

SEQ ID NO: 708 - protein, 8 aa, Homo sapiens
EAAEARRG

SEQ ID NO: 709 - protein, 8 aa, Homo sapiens
ISAEVQAP

SEQ ID NO: 710 - protein, 8 aa, Homo sapiens
HPQEASVV

SEQ ID NO: 711 - protein, 8 aa, Homo sapiens
ASVVVHQV

SEQ ID NO: 712 - protein, 8 aa, Homo sapiens
TDNEVAAL

SEQ ID NO: 713 - protein, 8 aa, Homo sapiens
NEVAALQP

SEQ ID NO: 714 - protein, 8 aa, Homo sapiens
PALAAYRL

SEQ ID NO: 715 - protein, 8 aa, Homo sapiens
PKDQTVVG

SEQ ID NO: 716 - protein, 8 aa, Homo sapiens
KDQTVVGP

SEQ ID NO: 717 - protein, 8 aa, Homo sapiens
VVGPALAA

SEQ ID NO: 718 - protein, 8 aa, Homo sapiens
RLEDTFGP

SEQ ID NO: 719 - protein, 27 aa, Homo sapiens
GVLPGGGFVASAAAVAGPEMQTGRNNF

SEQ ID NO: 720 - protein, 5 aa, Rattus norvegicus
LRKRD

SEQ ID NO: 721 - protein, 5 aa, Rattus norvegicus
LRKAD

SEQ ID NO: 722 - protein, 5 aa, Rattus norvegicus
RQQRS

SEQ ID NO: 723 - protein, 5 aa, Rattus norvegicus
RQQAS

SEQ ID NO: 724 - protein, 15 aa, Homo sapiens
DELSSMVLELRGLRT